SEQUENCE LISTING (25 sequences)

SEQ ID NO: 1 - protein, 5 aa, synthetic construct
SFGMH

SEQ ID NO: 2 - protein, 17 aa, synthetic construct
YISSGSSTIYYGDTVKG

SEQ ID NO: 3 - protein, 15 aa, synthetic construct
EGGYYYGRSYYTMDY

SEQ ID NO: 4 - protein, 16 aa, synthetic construct
RSSQSIVHSNGNTYLE

SEQ ID NO: 5 - protein, 7 aa, synthetic construct
KVSNRFS

SEQ ID NO: 6 - protein, 9 aa, synthetic construct
FQGSHVPPT

SEQ ID NO: 7 - protein, 124 aa, synthetic construct
EVQLVESGGGLVQPGGSLRLSCSASGFTFSSFGMHWVRQAPGKGLEWVAYISSGSSTIYYGDTVKGRFTISRDNAKNSLFLQMSSLRAEDTAVYYCAREGGYYYGRSYYTMDYWGQGTTVTVSS

SEQ ID NO: 8 - protein, 112 aa, synthetic construct
DVVMTQSPLSLPVTPGAPASISCRSSQSIVHSNGNTYLEWYLQKPGQSPKLLIYKVSNRFSGVPDRFSGSGSGTDFTLRISRVEAEDVGIYYCFQGSHVPPTFGPGTKLEIK

SEQ ID NO: 9 - protein, 454 aa, synthetic construct
EVQLVESGGGLVQPGGSLRLSCSASGFTFSSFGMHWVRQAPGKGLEWVAYISSGSSTIYYGDTVKGRFTISRDNAKNSLFLQMSSLRAEDTAVYYCAREGGYYYGRSYYTMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 219 aa, synthetic construct
DVVMTQSPLSLPVTPGAPASISCRSSQSIVHSNGNTYLEWYLQKPGQSPKLLIYKVSNRFSGVPDRFSGSGSGTDFTLRISRVEAEDVGIYYCFQGSHVPPTFGPGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 107 aa, synthetic construct [sequence omitted]

SEQ ID NO: 13 - protein, 42 aa, unidentified [sequence omitted]

SEQ ID NO: 14 - protein, 40 aa, unidentified
DAEFRHDSGYEVHHQKLVFFAEDVGSNKGAIIGLMVGGVV

SEQ ID NO: 15 - protein, 5 aa, synthetic construct
TYWIT

SEQ ID NO: 16 - protein, 17 aa, synthetic construct
DIYPGSSISNYNEKFKS

SEQ ID NO: 17 - protein, 10 aa, synthetic construct
EDGYDAWFAY

SEQ ID NO: 18 - protein, 16 aa, synthetic construct
RSSQSILHSNGNTYLE

SEQ ID NO: 19 - protein, 7 aa, synthetic construct
KVSNRFS

SEQ ID NO: 20 - protein, 9 aa, synthetic construct
FQGSHVPFT

SEQ ID NO: 21 - protein, 119 aa, synthetic construct
EVQLLESGGGLVQPGGSLRLSCAASGYTFTTYWITWVRQAPGKGLEWVSDIYPGSSISNYNEKFKSRFTISVDNSKNTLYLQMNSLRAEDTAVYYCAREDGYDAWFAYWGQGTLVTVSS

SEQ ID NO: 22 - protein, 112 aa, synthetic construct
DIQMTQSPSSLSASVGDRVTITCRSSQSILHSNGNTYLEWYQQKPGKAPKLLISKVSNRFSGVPSRFSGSGSGTDFTLTISSLQPEDFATYYCFQGSHVPFTFGQGTKLEIK

SEQ ID NO: 23 - protein, 330 aa, synthetic construct [sequence omitted]

SEQ ID NO: 24 - protein, 107 aa, synthetic construct [sequence omitted]

SEQ ID NO: 25 - protein, 441 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

(19) United States
(12) Patent Application Publication
(10) Pub. No.: US 2026/0184770 A1
(43) Pub. Date: Jul. 2, 2026

(54) METHODS OF TREATMENT USING A TAU PET LEVEL

(71) Applicant: EISAI R&D MANAGEMENT CO., LTD., Tokyo (JP)

(72) Inventors: Chad SWANSON, Nutley, NJ (US); Michael IRIZARRY, Nutley, NJ (US); Lynn KRAMER, Nutley, NJ (US); Shobha DHADDA, Nutley, NJ (US); Arnaud CHARIL, Nutley, NJ (US)

(21) Appl. No.: 19/133,053

(22) PCT Filed: Nov. 28, 2023
(86) PCT No.: PCT/US2023/081441

§ 371 Date: May 27, 2025

Related U.S. Application Data
(60) Provisional application No. 63/385,188, filed on Nov. 28, 2022.
(60) Provisional application No. 63/506,576, filed on Jun. 6, 2023.
(60) Provisional application No. 63/513,797, filed on Jul. 14, 2023.
(60) Provisional application No. 63/592,515, filed on Oct. 23, 2023.

(51) Int. Cl. C07K 16/18; A61K 39/00; A61P 25/28; G01N 33/68

(52) U.S. Cl. CPC C07K 16/18; A61P 25/28; G01N 33/6896; A61K 2039/505; A61K 2039/545; C07K 2317/565; G01N 2333/4709; G01N 2800/2821; G01N 2800/52

(57) ABSTRACT

Disclosed herein are methods of diagnosing, selecting, monitoring, and treating subjects with Alzheimer's disease (AD) or suspected of having AD or another disorder associated with amyloid accumulation in the brain using a tau PET level.

This application claims the benefit of and priority to US Provisional Patent Application Nos. 63/385,188, filed Nov. 28, 2022; 63/506,576, filed Jun. 6, 2023; 63/513,797, filed Jul. 14, 2023; and 63/592,515, filed Oct. 23, 2023.

### FIELD

Described herein are methods for treating Alzheimer's disease (AD) in a subject, relating to use of measurements of tau PET levels in a region of the subject's brain. Tau PET levels may be used to determine a stage of AD in a subject, identify a subject who will receive treatment for AD, select a treatment and regimen, monitor treatment efficacy, and/or predict a clinical outcome of treatment.

### BACKGROUND

Alzheimer's disease (AD) is a progressive, neurodegenerative disorder of unknown etiology and the most common form of dementia among older people. In 2006, there were 26.6 million cases of AD in the world (range: 11.4-59.4 million) (Brookmeyer, R., et al., Forecasting the global burden of Alzheimer's Disease. Alzheimer Dement. 2007; 3:186-91), while there were more than 5 million people in the United States reportedly living with AD (Alzheimer's Association, Alzheimer's Association report, 2010 Alzheimer's disease facts and figures. Alzheimer Dement. 2010; 6:158-94). By the year 2050, the worldwide prevalence of AD is predicted to grow to 106.8 million (range: 47.2-221.2 million), while in the United States alone the prevalence is estimated to be 11 to 16 million. (Brookmeyer, supra, and 2010 Alzheimer's disease facts and figures, supra).

The disease generally involves a global decline of cognitive function that progresses slowly and leaves end-stage subjects bedridden. AD subjects typically survive for only 3 to 10 years after symptom onset, although extremes of 2 and 20 years are known. (Hebert, L. E., et al., Alzheimer disease in the U.S. population: prevalence estimates using the 2000 census. Arch Neurol. 2003; 60:1119-1122.) AD is the seventh leading cause of all deaths in the United States and the fifth leading cause of death in Americans older than the age of 65 years, despite the fact that mortality due to AD is greatly underestimated because death certificates rarely attribute the cause of death to AD. (Alzheimer's Association. Alzheimer's Association report. 2010 Alzheimer's disease facts and figures. Alzheimer Dement. 2010; 6:158-94.)

AD represents a significant economic burden across industrialized countries with a substantial impact on healthcare systems and the public purse as well as on subjects and their families. In the United States alone, total payments for 2010 were estimated at $172 billion, including $123 billion for Medicare and Medicaid.

Histologically, the disease is characterized by neuritic plaques, found primarily in the association cortex, limbic system and basal ganglia. The major constituent of these plaques is amyloid beta peptide (Aβ). Aβ exists in various conformational states—monomers, oligomers, protofibrils, and insoluble fibrils. Details of the mechanistic relationship between onset of Alzheimer's disease and Aβ production is unknown. However, some anti-amyloid β antibodies (also called “anti-Aβ antibodies”) are undergoing clinical study now as potential therapeutic agents for Alzheimer's disease.

Despite the recent development of treatments for AD, including those targeting Aβ, there remains a need for better monitoring of treatments, including more refined measurements of markers predictive of treatment responsiveness or measuring response to treatment. Such markers may include combinations of different measurements.

Accordingly, disclosed herein are improved methods of selecting, monitoring, and treating patients (also referred to as “subjects”) with AD. In some embodiments, a treatment comprising an anti-Aβ protofibril antibody such as BAN2401 can lead to a reduced rate of tau accumulation, as measured by a tau PET level, also referred to as a “tau-PET” level, (e.g., as measured by tau PET imaging), e.g., in a brain region such as a temporal region, as compared to a control patient. In some embodiments, this correlates with reduced brain amyloid load and improved cognitive outcomes in subjects. Without being bound by theory, a tau PET level (e.g., a tau PET standard uptake value ratio (SUVr, also “SUVR”)) may be used, in various embodiments, as a less invasive and/or additional biomarker for refining the measurement of treatment efficacy and/or to allow for monitoring and treatment decisions. Such decision may include whether to increase or decrease the amount of an anti-Aβ protofibril antibody being administered, whether to increase or decrease the frequency of administration, whether to introduce a further therapeutic agent, and/or whether to discontinue treatment with the anti-Aβ protofibril antibody.

### SUMMARY

One aspect of the present disclosure relates to a method of treating Alzheimer's disease (AD) in a subject having or suspected of having AD, comprising (a) selecting a subject having a low level of tau in a global brain measurement, preferably as measured by tau PET (a low tau PET level), and (b) administering to the subject a therapeutically effective dose of an anti-amyloid β (Aβ) protofibril antibody.

In some embodiments, treating AD comprises reducing, slowing, and/or reversing decline in a measure of cognitive function, e.g., in the subject receiving a therapeutically effective dose of an anti-Aβ protofibril antibody, as compared to a control. In some embodiments, a measure of cognitive function in a subject who has been treated is compared to a baseline measure obtained from the same subject before treatment, or to a reference control. In some embodiments, the decline occurs between the time point when cognitive function is first measured (e.g., a baseline measure) and one or more later time points when cognitive function is measured again. In some embodiments, the later time point is at least 6 months, 12 months, 18 months, 21 months, or 24 months after the baseline measure. In some embodiments, the control is a subject who does not receive a therapeutically effective dose of an Aβ protofibril antibody, e.g., a subject who receives no treatment, or a subject who receives a placebo. In some embodiments, the control is a subject who does not receive lecanemab. In some embodiments, a control is a reference measurement, such as an averaged measurement that combines population data from more than one subject and is representative of a subject who does not receive treatment. In some embodiments, the baseline measurement is a measurement made prior to starting a treatment, e.g., a treatment with an anti-Aβ protofibril antibody, in a subject. In some embodiments, the measure of cognitive function is CDR-SB, ADAS-Cog14, and/or ADCS MCI-ADL.

In some embodiments, treating AD comprises effecting a change (e.g., slowing, delaying, or reducing) in at least one marker of AD pathology, e.g., in the subject receiving the therapeutically effective dose of the anti-Aβ protofibril antibody, as compared to a control. In some embodiments, the change in the marker occurs between the time point when the marker is first measured (e.g., a baseline measure) and one or more later time points when the marker is measured again. In some embodiments, the later time point is at least 6 months, 12 months, 18 months, 21 months, or 24 months after the baseline measure. In some embodiments, the control is a subject who does not receive a therapeutically effective dose of an Aβ protofibril antibody, e.g., a subject who receives no treatment, or a subject who receives a placebo. In some embodiments, the control is a subject who does not receive lecanemab. In some embodiments, a control is a reference measurement, such as an averaged measurement that combines population data from more than one subject and is representative of a subject who does not receive treatment. In some embodiments, the marker is a plasma Aβ42/40 ratio, a plasma p-tau 181 level, a plasma GFAP level, and/or a plasma NfL level.

In some embodiments, the Aβ protofibril antibody increases the plasma Aβ42/40 ratio, as measured by an adjusted mean change from a baseline plasma Aβ42/40 ratio of at least about 0.003, 0.006, 0.007, 0.008, or 0.009. In some embodiments, the change is observed after the Aβ protofibril antibody has been administered over a period of time. For example, the plasma Aβ42/40 ratio may increase by about 0.003 at or after about 6 months, about 0.006 at or after about 12 months, about 0.007 at or after about 18 months, about 0.008 at or after about 21 months, or about 0.009 at or after about 24 months of treatment with the Aβ protofibril antibody. In some embodiments, the increase in the plasma Aβ42/40 ratio from baseline is greater in a subject who receives lecanemab than in a control. In some embodiments, the Aβ protofibril antibody increases the Aβ42/40 ratio to about 0.092 or above.

In some embodiments, the Aβ protofibril antibody lowers plasma p-tau 181, as measured by an adjusted mean change from a baseline p-tau 181 level by at least about 0.2 pg/ml, 0.5 pg/ml, 0.6 pg/ml, 0.7 pg/ml, or 0.8 pg/ml. In some embodiments, the change is observed after the Aβ protofibril antibody has been administered over a period of time. For example, p-tau 181 may be lowered by about 0.2 pg/ml at or after about 6 months, 0.5 pg/ml at or after about 12 months, 0.6 pg/ml at or after about 18 months, 0.8 pg/ml at or after about 21 months, or 0.8 pg/ml at or after about 21 months of treatment with the Aβ protofibril antibody. In some embodiments, the decrease in plasma p-tau 181 from baseline is greater in a subject who receives lecanemab than in a control. In some embodiments, the Aβ protofibril antibody decreases the p-tau 181 level to about 2.3 pg/mL or below, or to about 2.2 pg/mL or below (e.g., as measured using a Quanterix Simoa p-tau assay).

In some embodiments, the Aβ protofibril antibody lowers plasma GFAP, as measured by an adjusted mean change from a baseline GFAP level by at least about 20 pg/ml, 30 pg/ml, 50 pg/ml, 60 pg/ml, or 80 pg/ml. In some embodiments, the change is observed after the Aβ protofibril antibody has been administered over a period of time. For example, plasma GFAP may be lowered from baseline by about 20 pg/ml at or after about 6 months, about 30 pg/ml at or after about 12 months, about 50 pg/ml at or after about 18 months, about 80 pg/ml at or after about 21 months, and about 60 mg/ml at or after about 24 months of treatment with the Aβ protofibril antibody. In some embodiments, the decrease in plasma GFAP from baseline is greater in a subject who receives lecanemab than in a control.

In some embodiments, the Aβ protofibril antibody increases plasma NfL by less than about 2 pg/ml or less than about 3 pg/ml, as measured by an adjusted mean change from a baseline NfL level. In some embodiments, the change is observed after the Aβ protofibril antibody has been administered over a period of time. In some embodiments, the increase in plasma NfL from baseline is lower in a subject who receives lecanemab than in a control. In some embodiments, plasma NfL in a subject who receives lecanemab does not change from baseline, or decreases over time.

In some embodiments, the marker is a tau PET level or an amyloid PET level.

In some embodiments, the Aβ protofibril antibody reduces the tau PET level, as measured by an adjusted mean change from baseline tau PET SUVr level of less than about 0.1, e.g., 0.05. In some embodiments, the reduction in tau PET from baseline is greater in a subject who receives lecanemab than in a control.

In some embodiments, the Aβ protofibril antibody reduces the amyloid PET level to about 55, 40, 25, or 20 centiloids (CL). In some embodiments, the reduction in amyloid PET from baseline is greater in a subject who receives lecanemab than in a control.

In some embodiments, the Aβ protofibril antibody reduces the tau PET and/or the amyloid PET level in a local brain region. In some embodiments, the reduction in plasma p-tau 181 from baseline is greater in a subject who receives lecanemab than in a control.

In some embodiments, the local brain region is an early Braak region (e.g., Braak regions I, II, or III).

In some embodiments, the early Braak region comprises transentorhinal cortex, entorhinal cortex, hippocampus, amygdala, parahippocampal gyrus, fusiform gyrus, and/or lingual gyrus.

In some embodiments, the local brain region is a composite of regions that accumulate tau in early AD.

In some embodiments, the composite region comprises a temporal region, a medial temporal region, and/or a meta-temporal region.

In some embodiments, the local brain region is the medial temporal region (e.g., the entorhinal cortex).

In some embodiments, the subject has mild cognitive impairment or mild dementia.

In some embodiments, the subject is at risk for developing AD.

In some embodiments, the subject has or is suspected of having, pre-AD.

In some embodiments, the subject has or is suspected of having, early AD.

In some embodiments, the subject has an amyloid PET level <20 CL, <40 CL, or <60 CL.

In some embodiments, the subject has an amyloid PET level >20 CL, >40 CL, or >60 CL).

In some embodiments, the subject has elevated amyloid measured by amyloid PET (e.g., is amyloid positive).

In some embodiments, the subject has a p-tau 181 level at or above around 2.2 to 2.3 pg/mL (e.g., as measured using a Quanterix Simoa p-tau assay).

In some embodiments, the subject is an ApoE4 carrier.

In some embodiments, the low level of tau is a tau PET level (e.g., a tau-PET standardized uptake value ratio (SUVR)) below a threshold in the global brain measurement.

In some embodiments, the global brain measurement is a measurement of tau PET in the whole cortical gray matter.

In some embodiments, the tau PET level is measured using an MK6240 radiotracer.

In some embodiments, the threshold tau PET level is a tau PET SUVR of about 1.1, preferably about 1.06, as measured with a MK6240 PET scan, e.g., of whole cortical gray matter.

In some embodiments, the subject has a low level of tau in the global brain measurement (e.g., whole cortical gray matter) and a higher level of tau in a local brain region (e.g., medial temporal region, meta-temporal region, and/or temporal region).

In some embodiments, the subject further exhibits tau in a local brain region, as measured by tau PET, e.g., an early Braak region (e.g., Braak regions I, II, or III).

Another aspect of the present disclosure relates to a method of treating Alzheimer's disease (AD) in a subject having or suspected of having AD, comprising (a) selecting a subject having tau in a local brain region, preferably as measured by PET, and (b) administering to the subject a therapeutically effective dose of an anti-amyloid β (Aβ) protofibril antibody.

In some embodiments, the local brain region is an early Braak region (e.g., Braak regions I, II, or III).

In some embodiments, the early Braak region comprises transentorhinal cortex, entorhinal cortex, hippocampus, amygdala, parahippocampal gyrus, fusiform gyrus, and/or lingual gyrus.

In some embodiments, the local brain region is a composite of regions that accumulate tau in early AD.

In some embodiments, the composite region comprises a temporal region, a medial temporal region, and/or a meta-temporal region.

In some embodiments, the local brain region is the medial temporal region (e.g., the entorhinal cortex).

In some embodiments, the anti-Aβ protofibril antibody comprises three heavy chain complementarity determining regions (HCDR1, HCDR2, and HCDR3) comprising amino acid sequences of SEQ ID NO: 1 (HCDR1), SEQ ID NO: 2 (HCDR2), and SEQ ID NO: 3 (HCDR3); and three light chain complementarity determining regions (LCDR1, LCDR2, and LCDR3) comprising amino acid sequences of SEQ ID NO: 4 (LCDR1), SEQ ID NO: 5 (LCDR2), and SEQ ID NO: 6 (LCDR3).

In some embodiments, the anti-Aβ protofibril antibody comprises a heavy chain variable region comprising an amino acid sequence of SEQ ID NO: 7 and a light chain variable region comprising an amino acid sequence of SEQ ID NO: 8.

In some embodiments, the anti-Aβ protofibril antibody comprises lecanemab.

In some embodiments, the therapeutically effective dose of the anti-Aβ protofibril antibody comprises an intravenous infusion of 10 mg/kg relative to the weight of the subject.

In some embodiments, the therapeutically effective dose of the anti-Aβ protofibril antibody comprises a subcutaneous administration of about 250 to 720 mg.

In some embodiments, the therapeutically effective dose is administered weekly.

In some embodiments, the therapeutically effective dose is administered every 2 weeks.

In some embodiments, the therapeutically effective dose is administered for at least 13 months, at least 18 months, or at least 24 months.

In some embodiments, the frequency of administration is reduced after 13 months of treatment, e.g., to a frequency of every 4, 6, 8, 10, or 12 weeks.

In some embodiments, the frequency of administration is reduced after 18 months of treatment, e.g., to a frequency of every 4, 6, 8, 10, or 12 weeks.

In some embodiments, frequency of administration is reduced after 24 months of treatment, e.g., to a frequency of every 4, 6, 8, 10, or 12 weeks.

In some embodiments, the treatment further comprises administering at least one additional therapy for AD (e.g., an anti-tau antibody such as E2814).

In some embodiments, the treatment further comprises administering an anti-tau antibody, preferably E2814.

An aspect of the present disclosure relates to a method of selecting a subject for treatment with an anti-amyloid β (Aβ) protofibril antibody, comprising (a) obtaining a tau PET level from a global brain measurement of the subject and (b) selecting the subject for treatment if the tau PET level is less than a threshold level.

In some embodiments, the global brain measurement is a tau PET level in whole cortical gray matter.

In some embodiments, the tau PET level is measured using an MK6240 radiotracer.

In some embodiments, the threshold tau PET level is a tau PET SUVR of about 1.1, preferably about 1.06, as measured in a MK6240 PET scan, e.g., of whole cortical gray matter.

### ENUMERATED EMBODIMENTS

1. A method of treating Alzheimer's disease (AD) in a subject having or suspected of having AD, comprising
   a. measuring a tau PET level in a region of a brain of the subject;
   b. administering to the subject a first therapeutically effective dose of an anti-amyloid β (Aβ) protofibril antibody;
   c. measuring or having measured a second tau PET level in the subject; and
   d. administering a second therapeutically effective dose comprising the same or a lower amount of the anti-Aβ protofibril antibody than in the first dose to the subject having a lower rate of tau PET increase relative to a control subject.
2. A method of treating Alzheimer's disease (AD) in a subject having or suspected of having AD, comprising
   a. measuring a tau PET level in a region of a brain of the subject;
   b. administering to the subject a first therapeutically effective dose of an anti-amyloid β (Aβ) protofibril antibody;
   c. measuring or having measured a second tau PET level in the subject; and
   d. administering a second therapeutically effective dose comprising the same or a higher amount of the anti-Aβ protofibril antibody than in the first dose and/or administering a second therapeutic agent to the subject having an equivalent or higher rate of tau PET increase relative to a control subject.
3. The method of embodiment 2, wherein the second therapeutic agent comprises an anti-tau antibody.
4. The method of any one of embodiments 1 to 3, wherein the second tau PET level is measured at 13 months.
5. The method of any one of embodiments 1 to 3, wherein the second tau PET level is measured at 18 months.
6. A method of treating Alzheimer's disease (AD) in a subject having or suspected of having AD, comprising
   a. measuring a tau PET level in a temporal region of a brain of the subject;
   b. administering to the subject a therapeutically effective dose of an anti-amyloid β (Aβ) protofibril antibody;
   c. measuring or having measured a second tau PET level in the subject in the temporal region of the brain at 13 or 18 months after the first administration; and
   d. continuing to administer the anti-Aβ protofibril antibody to the subject having a tau PET level that increased by no more than 0.05-0.1 relative to the measurement before administration of the Aβ protofibril antibody, as evaluated by tau PET SUVR in the temporal region.
7. The method of embodiment 6, wherein the temporal region is a medial temporal, meta-temporal, or temporal region (e.g., a lateral temporal region).
8. A method of treating Alzheimer's disease (AD) in a subject having or suspected of having AD, comprising:
   a. measuring a tau PET level in a temporal region of a brain of the subject; and
   b. administering a treatment comprising a therapeutically effective dose of an anti-amyloid β (Aβ) protofibril antibody to the subject having a tau PET level greater than in a subject who does not have AD.
9. The method of embodiment 8, further comprising:
   a. measuring a second tau PET level from the subject after the first sampling to determine a second tau PET level; and
   b. administering a second therapeutically effective dose comprising the same amount or lower of the anti-Aβ protofibril antibody as in the first dose to the subject having a lower rate of tau PET increase relative to a control subject who has AD but is not treated with the Aβ protofibril antibody.
10. The method of embodiment 8, further comprising:
   a. measuring a second tau PET level from the subject after the first sampling to determine a second tau PET level; and
   b. administering a second therapeutically effective dose comprising the same amount or higher of the anti-Aβ protofibril antibody as in the first dose to the subject having a higher rate of tau PET increase relative to a control subject and/or a tau PET level that increased by no more than 0.05-0.1 as evaluated by tau PET SUVR in the temporal region.
11. The method of embodiment 8, further comprising:
   a. measuring a second tau PET level from the subject after the first sampling to determine a second tau PET level; and
   b. administering a second therapeutic agent to the subject having a higher rate of tau PET increase relative to a control subject.
12. The method of embodiment 1, 2, 6, or 8 to 11, wherein the second tau PET level is measured at least 13 months after the first tau PET level measurement.
13. The method of embodiment 1, 2, 6, or 8 to 11, wherein the second tau PET level is measured at least 18 months after the first tau PET level measurement.
14. A method of treating Alzheimer's disease in a subject, comprising
   a. measuring a tau PET level in a region of a brain of the subject suspected of having pre-AD; and
   b. administering a treatment comprising a therapeutically effective dose of an anti-amyloid β (Aβ) protofibril antibody to the subject having a tau PET level greater than a subject who does not have AD,
      wherein the subject exhibits at least one biomarker of AD (e.g., a reduced Aβ42/40 ratio relative to a subject who does not have AD, e.g., a ratio below a threshold of about 0.092, and/or an elevated p-tau217 level relative to the level in a subject who does not have AD), and optionally wherein the subject is otherwise cognitively normal.
15. A method of reducing brain amyloid beta in a subject having or suspected of having AD, comprising
   a. measuring a tau PET level in a region of a brain of the subject;
   b. administering to the subject a first therapeutically effective dose of an anti-amyloid β (Aβ) protofibril antibody;
   c. measuring or having measured a second tau PET level in the subject; and
   d. administering a second therapeutically effective dose comprising the same or a lower amount of the anti-Aβ protofibril antibody than in the first dose to the subject having a lower rate of tau PET increase relative to a control subject.
16. A method of reducing brain amyloid beta in a subject having or suspected of having AD, comprising
   a. measuring a tau PET level in a region of a brain of the subject;
   b. administering to the subject a first therapeutically effective dose of an anti-amyloid β (Aβ) protofibril antibody;
   c. measuring or having measured a second tau PET level in the subject; and
   d. administering a second therapeutically effective dose comprising the same or a higher amount of the anti-Aβ protofibril antibody than in the first dose and/or administering a second therapeutic agent to the subject having an equivalent or higher rate of tau PET increase relative to a control subject.
17. The method of any one of embodiments 10, 11, or 16, wherein the second therapeutic agent comprises an anti-tau antibody.
18. The method of any one of embodiments 15 to 17, wherein the second tau PET level is measured at 13 or 18 months.
19. A method of reducing brain amyloid beta in a subject having or suspected of having AD, comprising
   a. measuring a tau PET level in a temporal region of a brain of the subject;
   b. administering to the subject a therapeutically effective dose of an anti-amyloid β (Aβ) protofibril antibody;
   c. measuring or having measured a second tau PET level in the subject in the temporal region of the brain at 13 or 18 months after the first administration; and
   d. continuing to administer the anti-Aβ protofibril antibody to the subject having a tau PET level that increased by no more than 0.05-0.1 as evaluated by tau PET SUVR in the temporal region.
20. The method of embodiment 19, wherein the temporal region is a medial temporal, meta-temporal, or temporal lobe.
21. A method of reducing brain amyloid beta in a subject having or suspected of having AD, comprising:
   a. measuring a tau PET level in a temporal region of a brain of the subject; and
   b. administering a treatment comprising a therapeutically effective dose of an anti-amyloid β (Aβ) protofibril antibody to the subject having a tau PET level greater than in a subject who does not have AD.
22. The method of embodiment 21, further comprising:
   a. measuring a second tau PET level from the subject after the first sampling to determine a second tau PET level; and
   b. administering a second therapeutically effective dose comprising the same amount or lower of the anti-Aβ protofibril antibody as in the first dose to the subject having a lower rate of tau PET increase relative to a control subject.
23. The method of embodiment 21, further comprising:
   a. measuring a second tau PET level from the subject after the first sampling to determine a second tau PET level; and
   b. administering a second therapeutically effective dose comprising the same amount or higher of the anti-Aβ protofibril antibody as in the first dose to the subject having a lower rate of tau PET increase relative to a control subject.
24. The method of embodiment 21, further comprising:
   a. measuring a second tau PET level from the subject after the first sampling to determine a second tau PET level; and
   b. administering a second therapeutic agent to the subject having a lower rate of tau PET increase relative to a control subject.
25. The method of embodiment 21, further comprising:
   a. measuring a second tau PET level from the subject after the first sampling to determine a second tau PET level; and
   b. administering a second therapeutic agent to the subject having a higher rate of tau PET increase relative to a control subject.
26. A method of reducing brain amyloid beta in a subject having or suspected of having AD, comprising
   a. measuring a tau PET level in a region of a brain of a subject suspected of having pre-AD; and
   b. administering a treatment comprising a therapeutically effective dose of an anti-amyloid 3 (Aβ) protofibril antibody to the subject having a tau PET level greater than a subject who does not have AD,
      wherein the subject exhibits at least one biomarker of AD (e.g., a reduced Aβ42/40 ratio relative to a subject who does not have AD, e.g., a ratio below a threshold of about 0.092, and/or an elevated p-tau217 level relative to the level in a subject who does not have AD) but is cognitively normal.
27. The method of any one of embodiments 1-26, further comprising administering a second therapeutic agent in conjunction with the anti-AP protofibril antibody.
28. The method of embodiment 24, 25, or 27, wherein the second therapeutic agent is an anti-tau antibody.
29. The method of embodiment 28, wherein the anti-tau antibody comprises three heavy chain complementarity determining regions (HCDR1, HCDR2, and HCDR3) comprising amino acid sequences of SEQ ID NO:15 (HCDR1), SEQ ID NO:16 (HCDR2), SEQ ID NO:17 (HCDR3); and three light chain complementarity determining regions (LCDR1, LCDR2, and LCDR3) comprising amino acid sequences of SEQ ID NO:18 (LCDR1), SEQ ID NO:19 (LCDR2), and SEQ ID NO:20 (LCDR3).
30. The method of embodiment 28 or 29, wherein the anti-tau antibody comprises a heavy chain variable region of SEQ ID NO: 21 and a light chain variable region of SEQ ID NO: 22.
31. The method of any one of embodiments 1-30, wherein the anti-AP protofibril antibody comprises three heavy chain complementarity determining regions (HCDR1, HCDR2, and HCDR3) comprising amino acid sequences of SEQ ID NO: 1 (HCDR1), SEQ ID NO: 2 (HCDR2), and SEQ ID NO: 3 (HCDR3); and three light chain complementarity determining regions (LCDR1, LCDR2, and LCDR3) comprising amino acid sequences of SEQ ID NO: 4 (LCDR1), SEQ ID NO: 5 (LCDR2), and SEQ ID NO: 6 (LCDR3).
32. The method of any one of embodiments 1-31, wherein the anti-AP protofibril antibody comprises a heavy chain variable region comprising an amino acid sequence of SEQ ID NO: 7 and a light chain variable region comprising an amino acid sequence of SEQ ID NO: 8.
33. The method of any one of embodiments 1-32, wherein the subject has or is suspected of having, pre-AD.
34. The method of embodiment 33, wherein the subject is administered an anti-tau antibody before the anti-Aβ protofibril.
35. The method of any one of embodiments 1-32, wherein the subject has or is suspected of having, early AD.
36. The method of embodiment 35, wherein the subject is administered the anti-Aβ protofibril before an anti-tau antibody.
37. The method of any one of embodiments 1-36, wherein the therapeutically effective dose of the anti-Aβ protofibril antibody comprises an intravenous infusion of 10 mg/kg relative to the weight of the subject.
38. The method of any one of embodiments 1-36, wherein the therapeutically effective dose of the anti-Aβ protofibril antibody comprises a subcutaneous administration of about 250 to 720 mg.
39. The method of any one of embodiments 1-36, wherein the therapeutically effective dose of the anti-Aβ protofibril antibody comprises a subcutaneous administration of 360 mg.
40. The method of any one of embodiments 1-36, wherein the therapeutically effective dose of the anti-Aβ protofibril antibody comprises a subcutaneous administration of 720 mg.
41. A method of selecting a subject for treatment with an anti-amyloid β (Aβ) protofibril antibody, comprising:
   a. measuring a tau PET level from a region of a brain of the subject; and
   b. selecting the subject for treatment if the tau PET level is greater than a subject who does not have AD.
42. The method of embodiment 41, wherein the anti-Aβ protofibril antibody comprises three heavy chain complementarity determining regions (HCDR1, HCDR2, and HCDR3) comprising amino acid sequences of SEQ ID NO: 1 (HCDR1), SEQ ID NO: 2 (HCDR2), and SEQ ID NO: 3 (HCDR3); and three light chain complementarity determining regions (LCDR1, LCDR2, and LCDR3) comprising amino acid sequences of SEQ ID NO: 4 (LCDR1), SEQ ID NO: 5 (LCDR2), and SEQ ID NO: 6 (LCDR3).
43. The method of embodiment 41 or 42, wherein the anti-Aβ protofibril antibody comprises a heavy chain variable region comprising an amino acid sequence of SEQ ID NO: 7 and a light chain variable region comprising an amino acid sequence of SEQ ID NO: 8
44. The method of any one of embodiments 41-43, wherein the tau PET level is about 1.4.
45. The method of any one of embodiments 41-43, wherein the tau PET level is about 1.5.
46. A method of monitoring treatment efficacy in a subject having or suspected of having AD, comprising:
   a. administering to the subject a therapeutically effective dose of an anti-Aβ protofibril antibody;
   b. measuring a tau PET level from a region of a brain of the subject; and
   c. comparing the tau PET level to a tau PET level from the subject prior to treatment, wherein a lower tau PET level relative to a control subject indicates effective treatment.
47. A method of monitoring treatment efficacy in a subject having or suspected of having AD, comprising
   a. measuring a tau PET level in a temporal region of a brain of the subject;
   b. administering to the subject a therapeutically effective dose of an anti-amyloid β (Aβ) protofibril antibody;
   c. measuring or having measured a second tau PET level in the subject in the temporal region of the brain at 13 or 18 months after the first administration; and
   d. comparing the tau PET level to a tau PET level from the subject prior to treatment, wherein a tau PET level that increased by no more than 0.05-0.1 as evaluated by tau PET SUVR in the temporal region indicates effective treatment.
48. The method of embodiment 46 or 47, wherein the anti-Aβ protofibril antibody comprises three heavy chain complementarity determining regions (HCDR1, HCDR2, and HCDR3) comprising amino acid sequences of SEQ ID NO: 1 (HCDR1), SEQ ID NO: 2 (HCDR2), and SEQ ID NO: 3 (HCDR3); and three light chain complementarity determining regions (LCDR1, LCDR2, and LCDR3) comprising amino acid sequences of SEQ ID NO: 4 (LCDR1), SEQ ID NO: 5 (LCDR2), and SEQ ID NO: 6 (LCDR3).
49. The method of any one of embodiments 46 to 48, wherein the anti-Aβ protofibril antibody comprises a heavy chain variable region comprising an amino acid sequence of SEQ ID NO: 7 and a light chain variable region comprising an amino acid sequence of SEQ ID NO: 8
50. A method of treating Alzheimer's disease (AD) in a subject having or suspected of having AD, comprising:
   a. measuring a tau PET level in a region of a brain of the subject;
   b. administering a treatment dosing regimen comprising a therapeutically effective dose of an anti-amyloid β (Aβ) protofibril antibody to the subject;
   c. measuring a subsequent tau PET level; and
   d. determining that a subsequent tau PET level is lower than a control level and/or a tau PET level that increased by no more than 0.05-0.1 and switching to a maintenance dosing regimen.
51. The method of embodiment 50, wherein the subject has a tau PET level greater 1.4 as evaluated by tau PET SUVR.
52. The method of embodiment 50, wherein the subject has a tau PET level greater 1.5 as evaluated by tau PET SUVR.
53. The method of any one of embodiments 50-52, wherein the region of the brain is the temporal region.
54. The method of any one of embodiments 50-53, wherein the anti-Aβ protofibril antibody comprises three heavy chain complementarity determining regions (HCDR1, HCDR2, and HCDR3) comprising amino acid sequences of SEQ ID NO: 1 (HCDR1), SEQ ID NO: 2 (HCDR2), and SEQ ID NO: 3 (HCDR3); and three light chain complementarity determining regions (LCDR1, LCDR2, and LCDR3) comprising amino acid sequences of SEQ ID NO: 4 (LCDR1), SEQ ID NO: 5 (LCDR2), and SEQ ID NO: 6 (LCDR3).
55. The method of any one of embodiments 50-54, wherein the anti-Aβ protofibril antibody comprises a heavy chain variable region comprising an amino acid sequence of SEQ ID NO: 7 and a light chain variable region comprising an amino acid sequence of SEQ ID NO: 8
56. The method of any one of embodiments 50-55, wherein the switch to a maintenance dose occurs after at least 6 months (e.g., 6 months, or 13 months, or 18 months) following the start of the treatment dosing regimen or after the subject has converted to amyloid negative status, e.g., as determined by amyloid PET.
57. The method of any one of embodiments 50-55, wherein the treatment dosing regimen comprises administering the anti-Aβ protofibril antibody as an intravenous infusion at a therapeutically effective dose of 10 mg/kg relative to the weight of the subject every week.
58. The method of any one of embodiments 50-55, wherein the treatment dosing regimen comprises administering the anti-Aβ protofibril antibody as an intravenous infusion at a therapeutically effective dose of 10 mg/kg relative to the weight of the subject every two weeks.
59. The method of any one of embodiments 50-55, wherein the treatment dosing regimen comprises administering the anti-Aβ protofibril antibody subcutaneously at a therapeutically effective dose of 720 mg weekly.
60. The method of any one of embodiments 50-55, wherein the treatment dosing regimen comprises administering the anti-Aβ protofibril antibody subcutaneously at a therapeutically effective dose of 360 mg weekly.
61. The method of any one of embodiments 50-55, wherein the treatment dosing regimen comprises administering the anti-Aβ protofibril antibody subcutaneously at a therapeutically effective dose of 720 mg biweekly.
62. The method of any one of embodiments 50-61, wherein the maintenance dosing regimen comprises an intravenous infusion at a therapeutically effective dose of 10 mg/kg relative to the weight of the subject every two weeks.
63. The method of any one of embodiments 50-61, wherein the maintenance dosing regimen comprises an intravenous infusion at a therapeutically effective dose of 10 mg/kg relative to the weight of the subject every month.
64. The method of any one of embodiments 50-61, wherein the maintenance dosing regimen comprises an intravenous infusion at a therapeutically effective dose of 10 mg/kg relative to the weight of the subject every three months.
65. The method of any one of embodiments 50-61, wherein the maintenance dosing regimen comprises administering the anti-Aβ protofibril antibody subcutaneously at a therapeutically effective dose of 720 mg biweekly.
66. The method of any one of embodiments 50-61, wherein the maintenance dosing regimen comprises administering the anti-Aβ protofibril antibody subcutaneously at a therapeutically effective dose of 720 mg monthly.
67. The method of any one of embodiments 50-61, wherein the maintenance dosing regimen comprises administering the anti-Aβ protofibril antibody subcutaneously at a therapeutically effective dose of 360 mg weekly.
68. The method of any one of embodiments 50-67, wherein the maintenance dosing regimen comprises the same dosing regimen as the treatment regimen.
69. A method of monitoring treatment efficacy in a subject having or suspected of having AD, comprising:
   a. measuring a tau PET level from a region of a brain of the subject;
   b. measuring a level of second biomarker from the subject;
   c. administering to the subject a therapeutically effective dose of an anti-Aβ protofibril antibody;
   d. measuring a second tau PET level from the region of the brain of the subject after the first sampling;
   e. measuring a second level of the second biomarker from the subject after the first sampling;
   f. comparing the tau PET level and the level of a second biomarker from a control subject, wherein a lower tau PET level and/or an improved level in a second biomarker relative to a control patient indicates effective treatment.
70. A method of monitoring treatment efficacy in a subject having or suspected of having AD, comprising:
   a. measuring a tau PET level from a region of a brain of the subject;
   b. measuring a level of second biomarker from the subject;
   c. administering to the subject a therapeutically effective dose of an anti-Aβ protofibril antibody;
   d. measuring a second tau PET level from the region of the brain of the subject after the first sampling;
   e. measuring a second level of the second biomarker from the subject after the first sampling;
   f. comparing the tau PET level to a tau PET level from the subject prior to treatment;
   g. comparing the second biomarker level of the subject to a biomarker level from the subject prior to treatment, wherein a reduced rate of tau PET increase relative to a control subject and/or an improvement in a second biomarker level indicates effective treatment.
71. A method of treating Alzheimer's disease (AD) in a subject having or suspected of having AD, comprising
   a. measuring a tau PET level from a region of a brain of the subject;
   b. measuring a level of a second biomarker from the subject;
   c. administering to the subject a first therapeutically effective dose of an anti-amyloid β (Aβ) protofibril antibody;
   d. measuring a second tau PET level in the subject after the first sampling after the first sampling;
   e. measuring a level of the second biomarker from the subject after the first sampling to determine a second level; and
   f. administering a second therapeutically effective dose comprising the same or a lower amount of the anti-Aβ protofibril antibody than in the first dose to the subject having a lower rate of tau PET increase relative to a control subject and/or an improvement in a second biomarker level.
72. A method of treating Alzheimer's disease (AD) in a subject having or suspected of having AD, comprising
   a. measuring a tau PET level from the region of a brain of the subject;
   b. measuring a level of a second biomarker from the subject;
   c. administering to the subject a first therapeutically effective dose of an anti-amyloid β (Aβ) protofibril antibody;
   d. measuring a second tau PET level in the subject after the first sampling;
   e. measuring a level of the second biomarker from the subject after the first sampling to determine a second level; and
   f. administering a second therapeutically effective dose comprising the same or a higher amount of the anti-Aβ protofibril antibody than in the first dose to the subject having a higher rate of tau PET increase relative to a control subject and/or no improvement or worsening in a second biomarker level.
73. The method of any one of embodiments 69-72, wherein the second biomarker comprises one or more of:
volumetric MRI (vMRI) including whole brain volume, cortical thickness, total hippocampal volume, lateral ventricle volume, an amyloid PET level, a fluorodeoxyglucose (FDG) PET level, a cerebrospinal fluid level of Aβ1-42, Aβ1-40 (including a ratio of Aβ1-42 to Aβ1-40), total tau, neurogranin, neurofilament light chain (NfL), microtubule binding region (MTBR)-tau, or a serum or plasma level of Aβ1-42, Aβ1-40 (including a ratio of Aβ1-42 to Aβ1-40), total tau, phosphorylated tau (P-tau) (including tau phosphorylated at 181 (P-tau181), 217 (P-tau217), and/or 231 (P-tau231)), glial fibrillary acidic protein (GFAP), and/or neurofilament light chain (NfL).
74. The method of any one of embodiments 69-73, wherein the second biomarker is GFAP and/or a combination of p-Tau217 and the ratio of Aβ1-42 to Aβ1-40.
75. The method of any one of embodiments 69-73, wherein a lower level of tau PET and a lower level of amyloid PET indicates effective treatment.
76. The method of any one of embodiments 69-73, wherein the anti-Aβ protofibril antibody comprises three heavy chain complementarity determining regions (HCDR1, HCDR2, and HCDR3) comprising amino acid sequences of SEQ ID NO: 1 (HCDR1), SEQ ID NO: 2 (HCDR2), and SEQ ID NO: 3 (HCDR3); and three light chain complementarity determining regions (LCDR1, LCDR2, and LCDR3) comprising amino acid sequences of SEQ ID NO: 4 (LCDR1), SEQ ID NO: 5 (LCDR2), and SEQ ID NO: 6 (LCDR3).
77. The method of any one of embodiments 69-73, wherein the anti-Aβ protofibril antibody comprises a heavy chain variable region comprising an amino acid sequence of SEQ ID NO: 7 and a light chain variable region comprising an amino acid sequence of SEQ ID NO: 8.
78. The method of any one of embodiments 1-77, wherein the control subject has Alzheimer's disease, early Alzheimer's disease, or pre-Alzheimer's disease and wherein the control subject is not treated with an anti-Aβ protofibril antibody.
79. The method of any one of embodiments 1-78, wherein the region of the brain is the temporal lobe.
80. The method of any one of embodiments 1-78, wherein the region of the brain is the meta-temporal lobe.
81. The method of any one of embodiments 1-78, wherein the region of the brain is the medial-temporal lobe.
82. The method of any one of embodiments 1-13, 15-25, or 27-88, wherein the subject has Alzheimer's disease.
83. The method of any one of embodiments 1-13, 15-25, or 27-88, wherein the subject has early Alzheimer's disease.
84. The method of any one of embodiments 1-83, wherein the subject has pre-Alzheimer's disease (pre-AD).
85. The method of any one of embodiments 1-84, wherein the subject has Alzheimer's disease, Down's Syndrome, chronic traumatic encephalopathy, cerebral amyloid angiopathy, Lewy Body Dementia, or another brain disease or conditions with Aβ peptide-containing soluble and/or insoluble AP aggregates.
86. The method of any one of embodiments 1-85, wherein the subject has been diagnosed with
   a. mild cognitive impairment due to Alzheimer's disease—intermediate likelihood and/or has been diagnosed as having mild Alzheimer's disease dementia;
   b. mild cognitive impairment due to Alzheimer's disease—intermediate likelihood by National Institute of Aging—Alzheimer's Association (NIA-AA) core clinical criteria;
   c. mild cognitive impairment due to Alzheimer's disease—intermediate likelihood by a CDR global score of 0.5 and a Memory Box score of 0.5 or greater before treatment;
   d. mild cognitive impairment due to Alzheimer's disease—intermediate likelihood by a history of subjective memory decline with gradual onset and slow progression over the last 1 year before treatment, e.g., as corroborated by an informant;
   e. mild Alzheimer's disease dementia by the NIA-AA core clinical criteria for probable Alzheimer's disease dementia; or
   f. mild Alzheimer's disease dementia by a CDR score of 0.5 to 1.0 and a Memory Box score of 0.5 or greater before treatment.
87. The method of any one of embodiments 1-86, wherein the subject is amyloid-positive prior to administration, e.g., as indicated by a PET assessment, a CSF assessment of Aβ(1-42), MRI, retinal amyloid accumulation, and/or specific behavioral/cognitive phenotypes.
88. The method any one of embodiments 1-87, wherein treatment is discontinued if a tau PET level has increased by more than 0.05-0.1 relative to a control subject.
89. The method any one of embodiments 1-88, wherein treatment is discontinued if it does not result in a lower rate of tau PET level increase relative to a control subject.
90. The method of any one of embodiments 1-37, 41-58, or 62-89, wherein the anti-Aβ protofibril antibody is administered as an intravenous infusion at a therapeutically effective dose of 10 mg/kg relative to the weight of the subject.
91. The method of any one of embodiments 1-36, 40-56, 59, or 61-89, wherein the anti-Aβ protofibril antibody is subcutaneously administered at a therapeutically effective dose of 720 mg.
92. The method of any one of embodiments 1-36, 39, 41-56, 60, or 62-89, wherein the anti-Aβ protofibril antibody is subcutaneously administered at a therapeutically effective dose of 360 mg.
93. The method of embodiment 1-57, 59, 60, 62-92, wherein the therapeutically effective dose is administered weekly.
94. The method of embodiment 1-56, 58, 61-92, wherein the therapeutically effective dose is administered every 2 weeks.
95. The method of embodiment 1-94, wherein the frequency of administration is reduced after 13 months of treatment, e.g., to a frequency of every 4, 6, 8, 10, or 12 weeks.
96. The method of any one of embodiments 90-92, wherein the therapeutically effective dose is reduced after 13 months of treatment.
97. The method of embodiment 1-96, wherein the frequency of administration is reduced after 18 months of treatment, e.g., to a frequency of every 4, 6, 8, 10, or 12 weeks.
98. The method of any one of embodiments 90-92, wherein the therapeutically effective dose is reduced after 18 months of treatment.
99. The method of any one of embodiments 1-98, wherein the anti-Aβ protofibril antibody comprises three heavy chain complementarity determining regions (HCDR1, HCDR2, and HCDR3) comprising amino acid sequences of SEQ ID NO: 1 (HCDR1), SEQ ID NO: 2 (HCDR2), and SEQ ID NO: 3 (HCDR3); and three light chain complementarity determining regions (LCDR1, LCDR2, and LCDR3) comprising amino acid sequences of SEQ ID NO: 4 (LCDR1), SEQ ID NO: 5 (LCDR2), and SEQ ID NO: 6 (LCDR3).
100. The method of any one of embodiments 1-99, wherein the anti-Aβ protofibril antibody comprises a heavy chain variable region comprising an amino acid sequence of SEQ ID NO: 7 and a light chain variable region comprising an amino acid sequence of SEQ ID NO: 8
101. The method of any one of embodiments 1-100, wherein the subject is sequentially or simultaneously administered a second therapeutic agent.
102. The method of embodiment 101, wherein the second therapeutic agent is an anti-tau antibody.
103. The method of embodiment 102, wherein the anti-tau antibody comprises three heavy chain complementarity determining regions (HCDR1, HCDR2, and HCDR3) comprising amino acid sequences of SEQ ID NO:15 (HCDR1), SEQ ID NO:16 (HCDR2), SEQ ID NO:17 (HCDR3); and three light chain complementarity determining regions (LCDR1, LCDR2, and LCDR3) comprising amino acid sequences of SEQ ID NO:18 (LCDR1), SEQ ID NO:19 (LCDR2), and SEQ ID NO:20 (LCDR3).
104. The method of embodiment 102 or 103, wherein the anti-tau antibody or antigen binding fragment thereof comprises a heavy chain variable region of SEQ ID NO: 21 and a light chain variable region of SEQ ID NO: 22.
105. The method of any one of embodiments 1-104, wherein the method results in:
   a. an improvement or slowing of worsening of one or more cerebrospinal fluid biomarkers, e.g., Aβ1-42, Aβ1-40 (including a ratio of Aβ1-42 to Aβ1-40), total tau, neurogranin, neurofilament light chain (NfL), phosphorylated tau; and/or
   b. a reduction or a slowing of increase of plasma or serum biomarkers, e.g., Aβ1-42, Aβ1-40 (including a ratio of Aβ1-42 to Aβ1-40), total tau, phosphorylated tau (P-tau) (including tau phosphorylated at 181 (P-tau181), 217 (P-tau217), and/or 231 (P-tau231)), glial fibrillary acidic protein (GFAP), and/or neurofilament light chain (NfL); as compared to before treatment and/or as compared to an untreated control subject.
106. The method of any one of embodiments 1-105, wherein the treatment
   a. delays clinical decline as determined by ADCOMS;
   b. delays clinical decline as determined by ADAS MCI-ADL;
   c. delays clinical decline as determined by modified iADRS;
   d. delays clinical decline as measured by a CDR-SB; or
   e. delays clinical decline as measured by an ADAS-Cog.
107. The method of any one of embodiments 1-106, wherein the method further comprises monitoring for ARIA, e.g., ARIA-E and/or ARIA-H, e.g., as observed by MRI.
108. The method of any one of the embodiments 1-107, wherein the method does not require a titration step prior to administering to the subject a first therapeutically effective dose of the anti-Aβ protofibril antibody.
109. The method of any one of embodiments 1-108, wherein the method results in at least 24% less (e.g., at least 29% less) cognitive decline as measured by ADCOMS compared to an untreated subject.
110. The method of any one of embodiments 1-109, wherein the method results in at least 26% less (e.g. at least 27%) cognitive decline as measured by CDR-SB compared to an untreated subject.
111. The method of any one of embodiments 1-110, wherein the method results in at least 26% less (e.g., at least 47% less) cognitive decline as measured by ADAS-Cog14 compared to an untreated subject.
112. The method of any one of embodiments 1-111, wherein the method results in at least 37% less cognitive decline as measured by ADCS MCI-ADL compared to an untreated subject.
113. The method of any one of embodiments 1-112, wherein the method results in at least a 0.2 PET SUVr reduction from baseline as measured by amyloid beta PET compared to an untreated subject.
114. The method of any one of embodiments 1-113, wherein the method results in at least a 50 centiloid reduction from baseline as measured by amyloid beta PET compared to an untreated subject.
115. The method of any one of embodiments 1-114, wherein the method results in at least a 0.005 increase from baseline of plasma Aβ42/40 compared to an untreated subject.
116. The method of any one of embodiments 1-115, wherein the method results in at least a 1 pg/ml decrease from baseline of plasma p-tau181 compared to an untreated subject.
117. The method of any one of embodiments 1-116, wherein the method results in a subject converted from amyloid positive to amyloid negative.
118. The method of any one of embodiments 1-117, wherein the method results in a reduced risk of progression to the next stage of AD as measured by Clinical Dementia Rating (CDR) global score, e.g. by at least 10%, by at least 20%, by at least 30%, by at least 31%.
119. The method of any one of embodiments 109-118, wherein the result is measured at least 6 months after the first therapeutically effective dose.
120. The method of any one of embodiments 109-118, wherein the result is measured at least 12 months after the first therapeutically effective dose.
121. The method of any one of embodiments 109-118, wherein the result is measured at least 13 months after the first therapeutically effective dose.
122. The method of any one of embodiments 109-118, wherein the result is measured at least 18 months after the first therapeutically effective dose.
123. The method of any one of embodiments 109-122, wherein the therapeutically effective dose of the anti-Aβ protofibril antibody comprises an intravenous infusion of 10 mg/kg relative to the weight of the subject.
124. The method of any one of embodiments 109-123, wherein the therapeutically effective dose is administered biweekly.
125. The method of any one of embodiments 109-122, wherein the therapeutically effective dose of the anti-Aβ protofibril antibody comprises administering subcutaneously 720 mg weekly.
126. A method of treating Alzheimer's disease (AD) in a subject having or suspected of having AD, comprising
   a. selecting a subject having a low level of tau in a global brain measurement, preferably as measured by tau PET (a low tau PET level), and
   b. administering to the subject a therapeutically effective dose of an anti-amyloid β (Aβ) protofibril antibody.
127. The method of embodiment 126, wherein treating AD comprises reducing, slowing, and/or reversing decline in a measure of cognitive function.
128. The method of embodiment 127, wherein the measure of cognitive function is CDR-SB, ADAS-Cog14, and/or ADCS MCI-ADL.
129. The method of embodiment 126, wherein treating AD comprises effecting a change (e.g., slowing, delaying, or reducing) in at least one marker of AD pathology.
130. The method of embodiment 129, wherein the marker is a plasma Aβ42/40 ratio, a plasma p-tau 181 level, a plasma GFAP level, and/or a plasma NfL level.
131. The method of embodiment 130, wherein the Aβ protofibril antibody increases the plasma Aβ42/40 ratio, as measured by an adjusted mean change from a baseline plasma Aβ42/40 ratio of at least about 0.003, 0.006, 0.007, 0.008, or 0.009.
132. The method of embodiment 130 or embodiment 131, wherein the Aβ protofibril antibody increases the Aβ42/40 ratio to about 0.092 or above.
133. The method of embodiment 130, wherein the Aβ protofibril antibody lowers plasma p-tau 181, as measured by an adjusted mean change from a baseline p-tau 181 level by at least about 0.2 pg/ml, 0.5 pg/ml, 0.6 pg/ml, 0.7 pg/ml, or 0.8 pg/ml.
134. The method of embodiment 130, wherein the Aβ protofibril antibody lowers plasma GFAP, as measured by an adjusted mean change from a baseline GFAP level by at least about 20 pg/ml, 30 pg/ml, 50 pg/ml, 60 pg/ml, or 80 pg/ml.
135. The method of embodiment 130, wherein the Aβ protofibril antibody increases plasma NfL by less than about 2 pg/mL or less than about 3 pg/ml, as measured by an adjusted mean change from a baseline NfLlevel.
136. The method of embodiment 129, wherein the marker is a tau PET level or an amyloid PET level.
137. The method of embodiment 136, wherein the Aβ protofibril antibody reduces the tau PET level, as measured by an adjusted mean change from baseline tau PET SUVr level of less than about 0.1, e.g., 0.05.
138. The method of embodiment 136, wherein the Aβ protofibril antibody reduces the amyloid PET level to about 55, 40, 25, or 20 centiloids.
139. The method of embodiment 136, wherein the Aβ protofibril antibody reduces the tau PET and/or the amyloid PET level in a local brain region.
140. The method of embodiment 139, wherein the local brain region is an early Braak region (e.g., Braak regions I, II, or III).
141. The method of embodiment 140, wherein the early Braak region comprises entorhinal cortex, hippocampus, amygdala, parahippocampal gyrus, fusiform gyrus, and/or lingual gyrus.
142. The method of embodiment 139, wherein the local brain region is a composite of regions that accumulate tau in early AD.
143. The method of embodiment 142, wherein the composite region comprises a temporal region, a medial temporal region, and/or a meta-temporal region.
144. The method of embodiment 139, wherein the local brain region is the medial temporal region (e.g., the entorhinal cortex, hippocampus, parahippocampal gyrus and/or temporal pole (medial and inferio-lateral tip of the temporal lobe).
145. The method of embodiment 126, wherein the subject has mild cognitive impairment or mild dementia.
146. The method of embodiment 126, wherein the subject is at risk for developing AD.
147. The method of embodiment 126, wherein the subject has or is suspected of having, pre-AD.
148. The method of embodiment 126, wherein the subject has or is suspected of having, early AD.
149. The method of embodiment 126, wherein the subject has an amyloid PET level <20 CL, <40 CL, or <60 CL.
150. The method of embodiment 126, wherein the subject has an amyloid PET level >20 CL, >40 CL, or >60 CL).
151. The method of embodiment 126, wherein the subject has elevated amyloid measured by amyloid PET (e.g., is amyloid positive).
152. The method of embodiment 126, wherein the subject is an ApoE4 carrier.
153. The method of embodiment 126, wherein the low level of tau is a tau PET level (e.g., a tau-PET standardized uptake value ratio (SUVR)) below a threshold in the global brain measurement.
154. The method of embodiment 153, wherein the global brain measurement is a measurement of tau PET in the whole cortical gray matter.
155. The method of embodiment 153 or 154, wherein the tau PET level is measured using an MK6240 radiotracer.
156. The method of any one of embodiments 153-155, wherein the threshold tau PET level is a tau PET SUVR of about 1.1, preferably about 1.06, as measured with a MK6240 PET scan, e.g., of whole cortical gray matter.
157. The method of any one of embodiments 1-156, wherein the subject has a low level of tau in the global brain measurement (e.g., whole cortical gray matter) and a higher level of tau in a local brain region (e.g., medial temporal region, meta-temporal region, and/or temporal region), as measured by tau PET.
158. The method of any one of embodiments 1-157, wherein the subject further exhibits tau in a local brain region, as measured by tau PET, e.g., an early Braak region (e.g., Braak regions I, II, or III).
159. A method of treating Alzheimer's disease (AD) in a subject having or suspected of having AD, comprising
   a. selecting a subject having tau in a local brain region, preferably as measured by PET, and
   b. administering to the subject a therapeutically effective dose of an anti-amyloid β (Aβ) protofibril antibody.
160. The method of embodiment 159, wherein the local brain region is an early Braak region (e.g., Braak regions I, II, or III).
161. The method of embodiment 160, wherein the early Braak region comprises transentorhinal cortex, entorhinal cortex, hippocampus, amygdala, parahippocampal gyrus, fusiform gyrus, and/or lingual gyrus.
162. The method of embodiment 159, wherein the local brain region is a composite of regions that accumulate tau in early AD.
163. The method of embodiment 162, wherein the composite region comprises a temporal region, a medial temporal region, and/or a meta-temporal region.
164. The method of embodiment 159, wherein the local brain region is the medial temporal region (e.g., the entorhinal cortex).
165. The method of any one of embodiments 1-164, wherein the anti-Aβ protofibril antibody comprises three heavy chain complementarity determining regions (HCDR1, HCDR2, and HCDR3) comprising amino acid sequences of SEQ ID NO: 1 (HCDR1), SEQ ID NO: 2 (HCDR2), and SEQ ID NO: 3 (HCDR3); and three light chain complementarity determining regions (LCDR1, LCDR2, and LCDR3) comprising amino acid sequences of SEQ ID NO: 4 (LCDR1), SEQ ID NO: 5 (LCDR2), and SEQ ID NO: 6 (LCDR3).
166. The method of any one of embodiments 1-165, wherein the anti-Aβ protofibril antibody comprises a heavy chain variable region comprising an amino acid sequence of SEQ ID NO: 7 and a light chain variable region comprising an amino acid sequence of SEQ ID NO: 8.
167. The method of any one of embodiments 1-166, wherein the anti-Aβ protofibril antibody comprises lecanemab.
168. The method of any one of embodiments 1-167, wherein the therapeutically effective dose of the anti-Aβ protofibril antibody comprises an intravenous infusion of 10 mg/kg relative to the weight of the subject.
169. The method of any one of embodiments 1-167, wherein the therapeutically effective dose of the anti-Aβ protofibril antibody comprises a subcutaneous administration of about 250 to 720 mg.
170. The method of any one of embodiments 1-168, wherein the therapeutically effective dose is administered weekly.
171. The method of any one of embodiments 1-168, wherein the therapeutically effective dose is administered every 2 weeks.
172. The method of any one of embodiments 1-171, wherein the therapeutically effective dose is administered for at least 13 months, at least 18 months, or at least 24 months.
173. The method of embodiment 172, wherein the frequency of administration is reduced after 13 months of treatment, e.g., to a frequency of every 4, 6, 8, 10, or 12 weeks.
174. The method of embodiment 172, wherein the frequency of administration is reduced after 18 months of treatment, e.g., to a frequency of every 4, 6, 8, 10, or 12 weeks.
175. The method of embodiment 172, wherein the frequency of administration is reduced after 24 months of treatment, e.g., to a frequency of every 4, 6, 8, 10, or 12 weeks.
176. The method of any one of embodiments 1-175, wherein the treatment further comprises administering at least one additional therapy for AD (e.g., an anti-tau antibody such as E2814).
177. The method of any one of embodiments 1-176, wherein the treatment further comprises administering an anti-tau antibody, preferably E2814.
178. A method of selecting a subject for treatment with an anti-amyloid β (Aβ) protofibril antibody, comprising:
   a. obtaining a tau PET level from a global brain measurement of the subject;
   b. selecting the subject for treatment if the tau PET level is less than a threshold level.
179. The method of embodiment 178, wherein the global brain measurement is a tau PET level in whole cortical gray matter.
180. The method of embodiment 178 or 179, wherein the tau PET level is measured using an MK6240 radiotracer.
181. The method of embodiment 179, wherein the threshold tau PET level is a tau PET SUVR of about 1.1, preferably about 1.06, as measured in a MK6240 PET scan, e.g., of whole cortical gray matter.
182. An anti-amyloid β (Aβ) protofibril antibody for use in a method of treatment of Alzheimer's disease (AD) in a subject having or suspected of having AD, wherein the method is the method of any one of embodiments 126-181.
183. The use of an anti-amyloid β (Aβ) protofibril antibody in the manufacture of a medicament for treating Alzheimer's disease (AD) in a subject having or suspected of having AD, wherein the medicament is administered by the method of any one of embodiments 126-181.
184. A kit for performing a method of treating Alzheimer's disease (AD) in a subject having or suspected of having AD according to any one of embodiments 126-181.
185. A therapeutically effective dose of an anti-amyloid β (Aβ) protofibril antibody for use in a method of treatment of Alzheimer's disease (AD) in a subject having or suspected of having AD, wherein the subject has a low level of tau in a global brain measurement, preferably as measured by tau PET (a low tau PET level).
186. A therapeutically effective dose of an anti-amyloid β (Aβ) protofibril antibody for use in a method of treatment of Alzheimer's disease (AD) in a subject having or suspected of having AD, wherein the subject has tau in a local brain region, preferably as measured by PET.
187. A therapeutically effective dose of an anti-amyloid β (Aβ) protofibril antibody for use in accordance with any of embodiments 185-186, wherein treatment of AD comprises reducing, slowing, and/or reversing decline in a measure of cognitive function.
188. A therapeutically effective dose of an anti-amyloid β (Aβ) protofibril antibody for use in accordance with embodiment 187, wherein the measure of cognitive function is CDR-SB, ADAS-Cog14, and/or ADCS MCI-ADL.
189. A therapeutically effective dose of an anti-amyloid β (Aβ) protofibril antibody for use in accordance with any of embodiments 185-188, wherein treating AD comprises effecting a change (e.g., slowing, delaying, or reducing) in at least one marker of AD pathology.
190. A therapeutically effective dose of an anti-amyloid β (Aβ) protofibril antibody for use in accordance with embodiment 189, wherein the marker is a plasma Aβ42/40 ratio, a plasma p-tau 181 level, a plasma GFAP level, and/or a plasma NfL level.
191. A therapeutically effective dose of an anti-amyloid β (Aβ) protofibril antibody for use in accordance with embodiment 190, wherein the Aβ protofibril antibody increases the plasma Aβ42/40 ratio, as measured by an adjusted mean change from a baseline plasma Aβ42/40 ratio of at least about 0.003, 0.006, 0.007, 0.008, or 0.009.
192. A therapeutically effective dose of an anti-amyloid β (Aβ) protofibril antibody for use in accordance with any of embodiments 190-191, wherein the Aβ protofibril antibody increases the Aβ42/40 ratio to about 0.092 or above.
193. A therapeutically effective dose of an anti-amyloid β (Aβ) protofibril antibody for use in accordance with any of embodiments 190-192, wherein the Aβ protofibril antibody lowers plasma p-tau 181, as measured by an adjusted mean change from a baseline p-tau 181 level by at least about 0.2 pg/ml, 0.5 pg/ml, 0.6 pg/ml, 0.7 pg/ml, or 0.8 pg/ml.
194. A therapeutically effective dose of an anti-amyloid β (Aβ) protofibril antibody for use in accordance with any of embodiments 190-193, wherein the Aβ protofibril antibody lowers plasma GFAP, as measured by an adjusted mean change from a baseline GFAP level by at least about 20 pg/ml, 30 pg/ml, 50 pg/ml, 60 pg/ml, or 80 pg/ml.
195. A therapeutically effective dose of an anti-amyloid β (Aβ) protofibril antibody for use in accordance with any of embodiments 190-194, wherein the Aβ protofibril antibody increases plasma NfL by less than about 2 pg/ml or less than about 3 pg/ml, as measured by an adjusted mean change from a baseline NfL level.
196. A therapeutically effective dose of an anti-amyloid β (Aβ) protofibril antibody for use in accordance with any of embodiments 189-195, wherein the marker is a tau PET level or an amyloid PET level.
197. A therapeutically effective dose of an anti-amyloid β (Aβ) protofibril antibody for use in accordance with any of embodiments 185-196, wherein the Aβ protofibril antibody reduces the tau PET level, as measured by an adjusted mean change from baseline tau PET SUVr level of less than about 0.1, e.g., 0.05.
198. A therapeutically effective dose of an anti-amyloid β (Aβ) protofibril antibody for use in accordance with any of embodiments 185-197, wherein the Aβ protofibril antibody reduces the amyloid PET level to about 55, 40, 25, or 20 centiloids.
199. A therapeutically effective dose of an anti-amyloid β (Aβ) protofibril antibody for use in accordance with any of embodiments 197-198, wherein the Aβ protofibril antibody reduces the tau PET and/or the amyloid PET level in a local brain region.
200. A therapeutically effective dose of an anti-amyloid β (Aβ) protofibril antibody for use in accordance with embodiment 199, wherein the local brain region is an early Braak region (e.g., Braak regions I, II, or III).
201. A therapeutically effective dose of an anti-amyloid β (Aβ) protofibril antibody for use in accordance with embodiment 200, wherein the early Braak region comprises entorhinal cortex, hippocampus, amygdala, parahippocampal gyrus, fusiform gyrus, and lingual gyrus.
202. A therapeutically effective dose of an anti-amyloid β (Aβ) protofibril antibody for use in accordance with embodiment 199, wherein the local brain region is a composite of regions that accumulate tau in early AD.
203. A therapeutically effective dose of an anti-amyloid β (Aβ) protofibril antibody for use in accordance with embodiment 202, wherein the composite region comprises a temporal region, a medial temporal region, and/or a meta-temporal region.
204. A therapeutically effective dose of an anti-amyloid β (Aβ) protofibril antibody for use in accordance with embodiment 199, wherein the local brain region is the medial temporal region (e.g., the entorhinal cortex, hippocampus, parahippocampal gyrus and temporal pole (medial and inferio-lateral tip of the temporal lobe)).
205. A therapeutically effective dose of an anti-amyloid β (Aβ) protofibril antibody for use in accordance with any of embodiments 185-204, wherein the subject has mild cognitive impairment or mild dementia.
206. A therapeutically effective dose of an anti-amyloid β (Aβ) protofibril antibody for use in accordance with any of embodiments 185-205, wherein the subject is at risk for developing AD.
207. A therapeutically effective dose of an anti-amyloid β (Aβ) protofibril antibody for use in accordance with any of embodiments 185-206, wherein the subject has or is suspected of having, pre-AD.
208. A therapeutically effective dose of an anti-amyloid β (Aβ) protofibril antibody for use in accordance with any of embodiments 185-205, wherein the subject has or is suspected of having, early AD.
209. A therapeutically effective dose of an anti-amyloid β (Aβ) protofibril antibody for use in accordance with any of embodiments 185-208, wherein the subject has an amyloid PET level <20 CL, <40 CL, or <60 CL.
210. A therapeutically effective dose of an anti-amyloid β (Aβ) protofibril antibody for use in accordance with any of embodiments 185-208, wherein the subject has an amyloid PET level >20 CL, >40 CL, or >60 CL).
211. A therapeutically effective dose of an anti-amyloid β (Aβ) protofibril antibody for use in accordance with any of embodiments 185-208, wherein the subject has elevated amyloid measured by amyloid PET (e.g., is amyloid positive).
212. A therapeutically effective dose of an anti-amyloid β (Aβ) protofibril antibody for use in accordance with any of embodiments 185-211, wherein the subject is an ApoE4 carrier.
213. A therapeutically effective dose of an anti-amyloid β (Aβ) protofibril antibody for use in accordance with any of embodiments 185 or 187-212, wherein the low level of tau is a tau PET level (e.g., a tau-PET standardized uptake value ratio (SUVR)) below a threshold in the global brain measurement.
214. A therapeutically effective dose of an anti-amyloid β (Aβ) protofibril antibody for use in accordance with embodiment 213, wherein the global brain measurement is a measurement of tau PET in the whole cortical gray matter.
215. A therapeutically effective dose of an anti-amyloid β (Aβ) protofibril antibody for use in accordance with any of embodiments 185-214, wherein the tau PET level is measured using an MK6240 radiotracer.
216. A therapeutically effective dose of an anti-amyloid β (Aβ) protofibril antibody for use in accordance with any of embodiments 185 or 187-215, wherein the threshold tau PET level is a tau PET SUVR of about 1.1, preferably about 1.06, as measured with a MK6240 PET scan, e.g., of whole cortical gray matter.
217. A therapeutically effective dose of an anti-amyloid β (Aβ) protofibril antibody for use in accordance with any of embodiments 185 or 187-216, wherein the subject has a low level of tau in the global brain measurement (e.g., whole cortical gray matter) and a higher level of tau in a local brain region (e.g., medial temporal region, meta-temporal region, and/or temporal region), as measured by tau PET.
218. A therapeutically effective dose of an anti-amyloid β (Aβ) protofibril antibody for use in accordance with any of embodiments 185 or 187-217, wherein the subject further exhibits tau in a local brain region, as measured by tau PET, e.g., an early Braak region (e.g., Braak regions I, II, or III).
219. A therapeutically effective dose of an anti-amyloid β (Aβ) protofibril antibody for use in accordance with any of embodiments 186 or 218, wherein the local brain region is an early Braak region (e.g., Braak regions I, II, or III).
220. A therapeutically effective dose of an anti-amyloid β (Aβ) protofibril antibody for use in accordance with embodiment 219, wherein the early Braak region comprises entorhinal cortex, hippocampus, amygdala, parahippocampal gyrus, fusiform gyrus, and lingual gyrus.
221. A therapeutically effective dose of an anti-amyloid β (Aβ) protofibril antibody for use in accordance with any of embodiments 186 or 218, wherein the local brain region is a composite of regions that accumulate tau in early AD.
222. A therapeutically effective dose of an anti-amyloid β (Aβ) protofibril antibody for use in accordance with embodiment 221, wherein the composite region comprises a temporal region, a medial temporal region, and/or a meta-temporal region.
223. A therapeutically effective dose of an anti-amyloid β (Aβ) protofibril antibody for use in accordance with any of embodiments 186 or 218, wherein the local brain region is the medial temporal region (e.g., the entorhinal cortex).
224. A therapeutically effective dose of an anti-amyloid β (Aβ) protofibril antibody for use in accordance with any of embodiments 185-223, wherein the anti-Aβ protofibril antibody comprises three heavy chain complementarity determining regions (HCDR1, HCDR2, and HCDR3) comprising amino acid sequences of SEQ ID NO: 1 (HCDR1), SEQ ID NO: 2 (HCDR2), and SEQ ID NO: 3 (HCDR3); and three light chain complementarity determining regions (LCDR1, LCDR2, and LCDR3) comprising amino acid sequences of SEQ ID NO: 4 (LCDR1), SEQ ID NO: 5 (LCDR2), and SEQ ID NO: 6 (LCDR3).
225. A therapeutically effective dose of an anti-amyloid β (Aβ) protofibril antibody for use in accordance with any of embodiments 185-224, wherein the anti-Aβ protofibril antibody comprises a heavy chain variable region comprising an amino acid sequence of SEQ ID NO: 7 and a light chain variable region comprising an amino acid sequence of SEQ ID NO: 8.
226. A therapeutically effective dose of an anti-amyloid β (Aβ) protofibril antibody for use in accordance with any of embodiments 185-225, wherein the anti-Aβ protofibril antibody comprises lecanemab.
227. A therapeutically effective dose of an anti-amyloid β (Aβ) protofibril antibody for use in accordance with any of embodiments 185-226, wherein the therapeutically effective dose of the anti-Aβ protofibril antibody comprises an intravenous infusion of 10 mg/kg relative to the weight of the subject.
228. A therapeutically effective dose of an anti-amyloid β (Aβ) protofibril antibody for use in accordance with any of embodiments 185-227, wherein the therapeutically effective dose of the anti-Aβ protofibril antibody comprises a subcutaneous administration of about 250 to 720 mg.
229. A therapeutically effective dose of an anti-amyloid β (Aβ) protofibril antibody for use in accordance with any of embodiments 185-228, wherein the therapeutically effective dose is administered weekly.
230. A therapeutically effective dose of an anti-amyloid β (Aβ) protofibril antibody for use in accordance with any of embodiments 185-229, wherein the therapeutically effective dose is administered every 2 weeks.
231. A therapeutically effective dose of an anti-amyloid β (Aβ) protofibril antibody for use in accordance with any of embodiments 185-230, wherein the therapeutically effective dose is administered for at least 13 months, at least 18 months, or at least 24 months.
232. A therapeutically effective dose of an anti-amyloid β (Aβ) protofibril antibody for use in accordance with embodiment 231, wherein the frequency of administration is reduced after 13 months of treatment, e.g., to a frequency of every 4, 6, 8, 10, or 12 weeks.
232. A therapeutically effective dose of an anti-amyloid β (Aβ) protofibril antibody for use in accordance with embodiment 231, wherein the frequency of administration is reduced after 18 months of treatment, e.g., to a frequency of every 4, 6, 8, 10, or 12 weeks.
234. A therapeutically effective dose of an anti-amyloid β (Aβ) protofibril antibody for use in accordance with embodiment 231, wherein the frequency of administration is reduced after 24 months of treatment, e.g., to a frequency of every 4, 6, 8, 10, or 12 weeks.
235. A therapeutically effective dose of an anti-amyloid β (Aβ) protofibril antibody for use in accordance with any of embodiments 185-234, wherein the treatment further comprises administering at least one additional therapy for AD (e.g., an anti-tau antibody such as E2814).
236. A therapeutically effective dose of an anti-amyloid β (Aβ) protofibril antibody for use in accordance with any of embodiments 185-235, wherein the treatment further comprises administering an anti-tau antibody, preferably E2814.
237. Use of a therapeutically effective dose of an anti-amyloid β (Aβ) protofibril antibody for the manufacture of a medicament for treating Alzheimer's disease (AD) in a subject having or suspected of having AD, wherein the subject has a low level of tau in a global brain measurement, preferably as measured by tau PET (a low tau PET level).
238. Use of a therapeutically effective dose of an anti-amyloid β (Aβ) protofibril antibody for the manufacture of a medicament for treating Alzheimer's disease (AD) in a subject having or suspected of having AD, wherein the subject has tau in a local brain region, preferably as measured by PET.
237. A method for diagnosing early AD in a subject suspected of having Alzheimer's disease (AD) and/or identifying a subject suspected of having Alzheimer's disease who is suitable for treatment with an anti-amyloid protofibril antibody such as lecanemab, comprising:
   a. obtaining a first tau PET measurement from a global brain region and optionally a second tau PET measurement from a local brain region in the subject; and
   b. diagnosing AD in the subject and/or identifying a subject who is suitable for treatment with an anti-amyloid protofibril antibody such as lecanemab based on a first tau PET level in the global brain measurement that is less than a threshold level and optionally by the presence of tau PET in the local brain region measurement, preferably wherein the local brain region comprises one or more of an early Braak region (e.g., Braak regions I, II, or III), a composite of regions that accumulate tau in early AD (e.g., a temporal region, a medial temporal region, and/or a meta-temporal region), or a medial temporal region (e.g., the entorhinal cortex, hippocampus, parahippocampal gyrus and/or temporal pole (medial and inferio-lateral tip of the temporal lobe.
238. A method of predicting prognosis of a subject having or suspected of having Alzheimer's disease, comprising:
   a. obtaining a tau PET level from a global brain measurement in the subject; and
   b. comparing the tau PET level to a threshold tau PET level, wherein a subject having a tau PET level that is less than a threshold level is expected to have an improved prognosis for treatment of AD as compared to a subject having a tau PET level that is at or above the threshold level.
239. The method of any of embodiments 237-238, wherein the global brain measurement is a tau PET level in whole cortical gray matter.
240. The method of any of embodiments 237-239, wherein the tau PET level is measured using an MK6240 radiotracer.
241. The method of embodiment 240, wherein the threshold tau PET level is a tau PET SUVR of about 1.1, preferably about 1.06, as measured in a MK6240 PET scan, e.g., of whole cortical gray matter.

### BRIEF DESCRIPTION OF DRAWINGS

FIG. 1 shows a plot of the adjusted mean change from baseline tau PET SUVR (standard error, SE) over time in subjects administered lecanemab or placebo.

FIG. 2 shows the effect of lecanemab administration in the entorhinal cortex of subjects.

FIG. 3 shows plots of the adjusted change from baseline tau PET SUVR at 18 months as a function of the baseline tau PET SUVR

FIG. 4 shows effects of lecanemab on tau PET SUVR in select brain regions.

FIG. 5 shows baseline tau PET SURV for composite regions in subjects who received lecanemab and subjects who received placebo.

FIG. 6 shows that baseline tau PET SUVR increases in subjects in clusters 1 to 4.

FIG. 7 shows the effects of lecanemab on adjusted mean change from baseline in Tau PET SUVR in subjects with different APOE4 status.

FIG. 8 shows a comparison of the effects of lecanemab on adjusted mean change from baseline in Tau PET SUVR in subjects who are homozygous for the APOE4 allele and heterozygous for APOE4 allele.

FIG. 9 compares the magnitude of the effect of lecanemab on adjusted mean change from baseline in Tau PET SUVR in subjects homozygous for the APOE4 allele, as compared to subjects who are noncarriers and subjects who are heterozygous for the APOE4 allele.

FIG. 10 shows the effects of lecanemab at 18 months across all brain regions and Global tau load (TauIQ) in subjects homozygous for the APOE4 allele carrier.

FIG. 11 shows the effects of lecanemab at 18 months in subjects who are noncarriers for APOE4.

FIG. 12 shows the effects of lecanemab at 18 months in subjects who are carriers for APOE4.

FIG. 13 shows the effects of lecanemab at 18 months in subjects who are heterozygous for APOE4.

FIG. 14 shows the effects of lecanemab in subjects from below Quartile 1 (<Q1), at 13 months and 18 months in brain regions and Global tau load (TauIQ).

FIG. 15 shows the effects of lecanemab in subjects in Quartile 1 (Q1) up to Quartile 3 (<Q3) over the same time points and regions as FIG. 14.

FIG. 16 shows the effects of lecanemab in subjects in above Quartile 3 (>Q3) over the same time points and regions as FIGS. 14 and 15.

FIG. 17 shows changes from baseline in Amyloid PET levels.

FIG. 18 shows the results obtained from an Open Label Extension of Study 201.

FIG. 19 shows the results of clinical endpoints (measures of ADCOMS, CDR-SB, ADAS-Cog14) in Study 201.

FIG. 20 shows the adjusted mean change from baseline CDR-SB in Study 301.

FIG. 21 shows the adjusted mean change from baseline ADAS-Cog14 in Study 301.

FIG. 22 shows the adjusted mean change from baseline ADCS MCI-ADL in Study 301.

FIG. 23 shows that lecanemab administration resulted in a reduction in brain amyloid beta plaque (adjusted mean change from baseline in amyloid beta PET centiloids) in Study 301.

FIG. 24 shows health-related Quality of Life measures—EQ-5D-5L (Health Today Subject)

FIG. 25 shows health-related Quality of Life measures—QOL-AD (Total Score Subject)

FIG. 26 shows health-related Quality of Life measures—QOL-AD (Subject by Proxy)

FIG. 27 shows health-related Quality of Life measures—Zarit Burden Interview—Study Partner Burden (Total Score)

FIG. 28 shows time to worsening of global CDR scores.

FIG. 29 shows a slope analysis using CDR-SB: observed data and extrapolation to 2 years.

FIG. 30 shows the change in plasma GFAP level with treatment by lecanemab.

FIG. 31 shows averaged scans from patients in the Tau PET substudy.

FIG. 32 shows the tau PET SUVr in Braak stage regions in subjects with low whole cortical tau aggregation.

FIG. 33 shows an ordering by decreasing median baseline tau PET SUVr.

FIG. 34 shows regional tau PET SUVr from subjects in the low tau PET subgroup who are amyloid positive or negative based on a 30 CL cut-off.

FIG. 35 shows regional tau PET SUVr from subjects in the low tau PET subgroup who are ApoE4 carriers or noncarriers.

FIG. 36 shows that lecanemab slowed tau pathology in the medial temporal lobe, meta temporal lobe, and temporal lobe, as compared to placebo.

FIG. 37 summarizes the adjusted mean difference in tau pathology, as measured by tau-PET SUVr across brain regions for patients who received lecanemab as compared to patients who received a placebo.

FIG. 38 shows the efficacy of lecanemab on cognitive and functional outcomes in subjects from the overall early AD patients studied in CLARITY, the representative tau PET substudy, and in subjects in low tau and intermediate-high tau subgroups.

FIG. 39 shows that lecanemab impacts different brain regions in the low tau PET group, as compared to the intermediate+high tau PET group.

FIG. 40 shows effects of lecanemab on CDR-SB, ADAS-Cog14, and ADCS MCI-ADL in subjects in the Clarity AD study.

FIG. 41 shows effects of lecanemab on CDR-SB, ADAS-Cog14, and ADCS MCI-ADL in subjects from the tau PET substudy.

FIG. 42 shows effects of lecanemab on CDR-SB, ADAS-Cog14, and ADCS MCI-ADL in subjects with low tau PET levels.

FIG. 43 shows effects of lecanemab on CDR-SB in subjects with low tau PET levels at 18 months, showing the percentage of subjects who show no CDR-SB decline and the percentage of subjects who show CDR-SB improvement.

FIG. 44 shows amyloid PET levels and amyloid PET clearance rates in subjects from the Tau PET substudy.

FIG. 45 shows fluid biomarkers in patients from the tau PET substudy.

FIG. 46 shows the results of CDR-SB measurements in subjects in the early start and late start groups.

FIG. 47 shows the test results from the OLE in the context of an observational cohort.

FIG. 48 shows the results of ADAS-Cog14 measurements in subjects in the early start and late start groups.

FIG. 49 shows the results of ADCS MCI-ADL measurements in subjects in the early start and late start groups.

FIG. 50 shows the results of CDR-SB measurements in subjects from the low tau-PET subgroup through 24 months.

FIG. 51 shows the results of ADAS-Cog14 measurements in subjects from the low tau-PET subgroup through 24 months.

FIG. 52 shows the results of ADCS MCI-ADL measurements in subjects from the low tau-PET subgroup through 24 months.

FIG. 53 shows effects of lecanemab on CDR-SB in subjects with low tau PET levels at 18-24 months, showing the percentage of subjects who show no CDR-SB decline and the percentage of subjects who show CDR-SB improvement.

FIG. 54 shows the percentages of patients showing either “No Decline” or “Improvement” in CDR-SB, ADAS-Cog 14 and ADCS MCI-ADL analyses at up to 24 months on lecanemab.

FIG. 55 shows that low tau PET levels are associated with lower levels of amyloid PET.

FIG. 56 shows clinical outcomes in subjects with a baseline amyloid PET of <60 CL.

FIG. 57 shows results of biomarker analyses in patients receiving lecanemab for 24 months.

### DETAILED DESCRIPTION

The “amyloid hypothesis” proposes that amyloid β (Aβ) peptides play a central role in the pathogenesis of AD. Specifically, it is hypothesized that neurodegeneration in AD may be caused by deposition of Aβ plaques in brain tissue due to an imbalance between Aβ production and Aβ clearance, leading to formation of neurofibrillary tangles containing tau protein. Aβ peptides generally exist in a dynamic continuum of conformational states such that species tend to progress from monomeric Aβ, to soluble Aβ assemblies that include a range of low molecular weight oligomers to higher molecular weight protofibrils, and finally to insoluble fibrils (plaques). Targeting these soluble and insoluble Aβ tangles and plaques may provide therapeutic benefit.

A number of immunotherapies have been developed with the intent to reduce the amount of insoluble Aβ fibrils deposited in the brain. However, a simple correlation between the quantity and progressive accumulation of insoluble amyloid plaques and the clinical course of AD has not been determined. While therapeutic strategies continue to focus on removal of insoluble amyloid plaques, an additional approach to therapy may include reducing the toxic Aβ aggregates, such as protofibrils, that may contribute to the neuronal degeneration characteristic of AD. (See, e.g., Dodort, J.-C. and May, P., “Overview on rodent models of Alzheimer's disease.” Curr. Protocols Neurosci. 2005; 9.22-1-9.22-6; Englund, H. et al., “Sensitive ELISA detection of amyloid-β protofibrils in biological samples.” J. Neurochem. 2007; 103:334-45; and Gotz, J. et al., “Transgenic animal models of Alzheimer's disease and related disorders: histopathology, behavior and therapy.” Mol. Psychiat. 2004; 9:664-83.)

In various embodiments, anti-Aβ protofibril antibodies, such as BAN2401 and other anti-Aβ protofibril antibodies, may be used to treat AD, e.g., by slowing AD progression in subjects, e.g., those at early stages of the disease when amyloid has been deposited in the brain but where the downstream neurodegenerative cascade thought to be triggered by the amyloid deposition is still relatively early in its course (i.e., limited brain tissue loss has been produced and associated clinical deficits are at a minimum).

### A. Definitions

The following are definitions of terms used in the present application.

As used herein, the singular terms “a,” “an,” and “the” include the plural reference unless the context clearly indicates otherwise.

The phrase “and/or,” as used herein, means “either or both” of the elements so conjoined, i.e., elements that are conjunctively present in some cases and disjunctively present in other cases. Thus, as a non-limiting example, “A and/or B”, when used in conjunction with open-ended language such as “comprising” can refer, in some embodiments, to A only (optionally including elements other than B); in other embodiments, to B only (optionally including elements other than A); in yet other embodiments, to both A and B (optionally including other elements); etc.

As used herein, “at least one” means one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, “about” when used in connection with doses, amounts, or ratios, include the value of a specified dose, amount, or ratio or a range of the dose, amount, or ratio that is recognized by one of ordinary skill in the art to provide a therapeutic effect equivalent to that obtained from the specified dose, amount, or ratio. The term “about” may refer to an acceptable error for a particular value as determined by one of skill in the art, which depends in part on how the values is measured or determined. In some embodiments, the term “about” means within 5% of a given value or range.

When a number is recited, either alone or as part of a numerical range, it should be understood that the numerical value can vary above and below the stated value by up to a variance of +/−10% of the stated value.

When a range of values is listed herein, it is intended to encompass each value and sub-range within that range. For example, “2.5 mg/kg to 10 mg/kg” is intended to encompass, for example, 2.5 mg/kg, 3 mg/kg, 3.5 mg/kg, 4 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg, 2.5 mg/kg to 3 mg/kg, 2.5 mg/kg to 4.5 mg/kg, 3 mg/kg to 4.5 mg/kg, 4.5 mg/kg to 8 mg/kg, 2.5 mg/kg to 9 mg/kg, and so forth.

As used herein, “adjusted mean change from baseline” refers to the use of a statistical analysis to calculate the change in a biomarker value over time. In some embodiments, a linear mixed-effects model (MMRM) is used to account for at least one additional covariate to determine the adjusted mean change from baseline.

Amyloid β 1-42 (Aβ42) refers to an amyloid beta monomer from amino acid 1 to 42 of the full-length protein (Table 5, SEQ ID NO:13). Amyloid β 1-40 (Aβ1-40) refers to an amyloid beta monomer from amino acid 1 to 42 of the full-length protein (Table 5, SEQ ID NO:14).

P-tau181 is human tau protein phosphorylated at threonine in position 181. P-tau217 is human tau protein phosphorylated at threonine in position 217. P-tau231 is human tau protein phosphorylated at threonine in position 231.

Total tau or t-tau as used herein is a measure of total tau in a sample, e.g. a CSF sample, a plasma sample, a serum sample.

Patients with “preclinical AD” or “pre-AD” as described herein, are cognitively normal individuals with intermediate or elevated levels of amyloid in the brain and can be identified by asymptomatic stages with or without memory complaints and emerging episodic memory and executive function deficits. Cognitively normal can include individuals who are CDR 0, or individuals within the normal ranges of cognitive test scores (MMSE, International Shopping List Task, Logical Memory, etc.). Preclinical AD occurs prior to significant irreversible neurodegeneration and cognitive impairment and is typically characterized by the appearance of in vivo molecular biomarkers of AD and the absence clinical symptoms. Preclinical AD biomarkers that may suggest the future development of Alzheimer's disease include, but are not limited to, one or more of intermediate or elevated levels of amyloid in the brain by amyloid PET (e.g., a centiloid measure of about 20-40, e.g., a centiloid measure of about 20-32), fluorodeoxyglucose (FDG) PET, or tau positron emission tomography (PET), cerebrospinal fluid level of Aβ1-42 and/or Aβ1-42/1-40 ratio, cerebrospinal fluid level of total tau, cerebrospinal fluid level of microtubule binding region (MTBR)-tau, cerebrospinal fluid level of neurogranin, cerebrospinal fluid level of neurofilament light chain (NfL), and blood biomarkers as measured in the serum or plasma (e.g. levels of A31-42, the ratio of two forms of amyloid-β peptide (Aβ1-42/1-40 ratio, e.g., a ratio of between about 0.092-0.094 or below about 0.092), plasma levels of plasma total tau (T-tau), levels of phosphorylated tau (P-tau) isoforms (including tau phosphorylated at 181 (P-tau181), 217 (P-tau217), and 231 (P-tau231)), glial fibrillary acidic protein (GFAP), and neurofilament light chain (NfL)). For example, it has been found that subjects treated with elenbecestat (E2609), a β-site amyloid precursor protein cleaving enzyme (BACE) inhibitor, who had amyloid baseline positron emission tomography (PET) standard uptake value ratios (SUVr values) of 1.4 to 1.9, exhibited the greatest slowing of cognitive decline while on treatment. See Lynch, S. Y. et al. “Elenbecestat, a BACE inhibitor: results from a Phase 2 study in subjects with mild cognitive impairment and mild-to-moderate dementia due to Alzheimer's disease.” Poster P4-389, Alzheimer's Association International Conference, Jul. 22-26, 2018, Chicago, IL, USA. Similarly, it has been found that subjects having a baseline florbetapir amyloid PET SUVr levels below 1.2 do not exhibit enough cognitive decline to be detectable, whereas subjects having SUVr levels above 1.6 appear to correlate with a plateau effect in which amyloid level has reached a saturation level and treatment does not result in a change of cognitive measures. See Dhadda, S. et al., “Baseline florbetapir amyloid PET standard update value ratio (SUVr) can predict clinical progression in prodromal Alzheimer's disease (pAD).” Poster P4-291, Alzheimer's Association International Conference, Jul. 22-26, 2018, Chicago, IL, USA.

“Early AD” or “early Alzheimer's disease,” as used herein, is a continuum of AD severity from mild cognitive impairment due to AD—intermediate likelihood to mild Alzheimer's disease dementia. Subjects with early AD include subjects with mild Alzheimer's disease dementia as defined herein and subjects with mild cognitive impairment (MCI) due to AD—intermediate likelihood as defined herein. In some embodiments, subjects with early AD have MMSE scores of 22 to 30 and Clinical Dementia Rating (CDR) global range 0.5 to 1.0. Other methods for detecting early AD disease may employ the tests and assays specified below, including the National Institute of Aging-Alzheimer's Association (NIA-AA) core clinical criteria for probable Alzheimer's disease dementia in McKhann, G. M. et al., “The diagnosis of dementia due to Alzheimer's disease: Recommendations from the National Institute on Aging—Alzheimer's Association workgroups on diagnostic guidelines for Alzheimer's disease.” Alzheimer Dement. 2011; 7:263-9. Other methods include CDR-SB, ADCOMS Composite Clinical Score, the Mini-Mental State Examination, ADAS-Cog, ADAS MCI-ADL, modified iADRS, Wechsler Memory Scale-IV Logical Memory (subscale) I (WMS-IV LMI), and Wechsler Memory Scale-IV Logical Memory (subscale) II (WMS-IV LMII). In some embodiments, a subject with early AD has evidence of elevated amyloid in the brain or a positive amyloid load. In some embodiments, elevated amyloid in the brain or a positive amyloid load is indicated and/or confirmed by PET assessment. In some embodiments, elevated amyloid in the brain or a positive amyloid load is indicated and/or confirmed by a CSF assessment of markers such as Aβ1-42 (e.g., a soluble CSF biomarker analysis). In some embodiments, elevated amyloid in the brain or a positive amyloid load is indicated and/or confirmed by measuring the level of p-tau181. In some embodiments, elevated amyloid in the brain or a positive amyloid load is indicated and/or confirmed by an MRI. In some embodiments, elevated amyloid in the brain or a positive amyloid load is indicated by retinal amyloid accumulation. In some embodiments, more than one assessment method is used.

“Amyloid” refers to fibers that are unbranched, usually extracellular, and found in vivo; in addition, the fibers bind the dye Congo Red and then show green birefringence when viewed between crossed polarizers. Amyloid-forming proteins have been identified and associated with serious diseases, including amyloid-β peptide (Aβ) with Alzheimer's disease (AD), islet amyloid polypeptide (IAPP) with diabetes type 2, and prion protein (PrP) with the spongiform encephalopathies. As used herein, “amyloid,” “brain amyloid,” and “amyloid-β peptide (Aβ)” are used interchangeably.

In some embodiments, the subject has “elevated amyloid” or “intermediate amyloid.” As one of ordinary skill in the art will recognize, amyloid levels from amyloid PET can be reported using the Centiloid method in “centiloid” units (CL). (Klunk W E et al. The Centiloid Project: standardizing quantitative amyloid plaque estimation by PET. Alzheimer's Dement. 2015; 11:1-15 e1-4). The Centiloid method measures a tracer on a scale of 0 CL to 100 CL, where 0 is deemed the anchor-point and represents the mean in young healthy controls and 100 CL represents the mean amyloid burden present in subjects with mild to moderate severity dementia due to AD. (Id.) As is known to one of ordinary skill in the art, centiloid thresholds may vary, for example may be refined, based on new or additional scientific information. (See, e.g., http://www.gaain.org/centiloid-project.) An elevated level of amyloid can be set relative to a baseline threshold in a healthy control determined according to methods known to a person of ordinary skill in the art (POSA). For example, a centiloid value of 32.5 can be used as a threshold value for “elevated amyloid,” and an “intermediate amyloid” level refers to an Aβ amyloid PET in the range of 20-32.5 CL (e.g., 30 CL). In another example, a centiloid value of 40 can be used as a threshold value for “elevated amyloid,” and an “intermediate amyloid” level refers to an Aβ amyloid PET in the range of 20-40 CL.

Subjects with “mild Alzheimer's disease dementia,” or “mild AD dementia” as used herein, are subjects meeting the National Institute of Aging-Alzheimer's Association (NIA-AA) core clinical criteria for probable Alzheimer's disease dementia in McKhann, G. M. et al., “The diagnosis of dementia due to Alzheimer's disease: Recommendations from the National Institute on Aging—Alzheimer's Association workgroups on diagnostic guidelines for Alzheimer's disease.” Alzheimer Dement. 2011; 7:263-9. Also included herein are subjects who have a CDR score of 0.5 to 1.0 and a Memory Box score of 0.5 or greater at screening and baseline and subjects that exhibit change in the score on the Wechsler Memory Scale-Revised Logical Memory subscale II (WMS-R LM II).

Subjects with “MCI due to AD—intermediate likelihood,” as used herein are those identified as such in accordance with the NIA-AA core clinical criteria for mild cognitive impairment due to Alzheimer's disease—intermediate likelihood (see McKhann supra). For example, a subject may be symptomatic but not demented, with evidence of brain amyloid pathology making them less heterogeneous and more similar to mild Alzheimer's disease dementia subjects in cognitive and functional decline as measured by the ADCOMS Composite Clinical Score defined herein. Also included are subjects who have a CDR score of 0.5 and a Memory Box score of 0.5 or greater at screening and baseline. Furthermore, subjects who report a history of subjective memory decline with gradual onset and slow progression over the last 1 year before screening, which is corroborated by an informant, are also included herein. Memory decline and/or episodic memory impairment can be assessed in a subject by change in the score on the Wechsler Memory Scale-Revised Logical Memory subscale II (WMS-R LM II).

As used herein, a “control subject”, “untreated AD subject”, or an “untreated control subject” is a subject that is not being treated or has been treated for Alzheimer's disease. In some embodiments, a control subject has Alzheimer's disease. In some embodiments, the control subject has early Alzheimer's disease, or pre-Alzheimer's disease. In some embodiments, the control subject has Alzheimer's disease and is not treated with an anti-Aβ protofibril antibody.

The terms “patient” and “subject” are used interchangeably.

As used herein, “MMSE” refers to the Mini-Mental State Examination, a cognitive instrument commonly used for screening purposes, but also often measured longitudinally in AD clinical trials having a 30 point scale with higher scores indicating less impairment and lower scores indicating more impairment, ranging from 0 (most impaired) to 30 (no impairment). In some embodiments, seven items measuring orientation to time and place, registration, recall, attention, language, and drawing may be assessed as part of the MMSE score. (Folstein, M. F. et al., “Mini-mental state. A practical method for grading the cognitive state of patients for the clinician.” J. Psychiatr. Res. 1975; 12:189-98.)

As used herein, “ADAS-Cog” refers to Alzheimer's Disease Assessment Scale-Cognitive. The ADAS-Cog is a widely used cognitive scale in Alzheimer's disease trials having a structured scale that evaluates memory (word recall, delayed word recall, and word recognition), reasoning (following commands), language (naming, comprehension), orientation, ideational praxis (placing letter in envelope) and constructional praxis (copying geometric designs). (Rosen, W. G. et al., “A new rating scale for Alzheimer's disease.” Am. J. Psychiatry 1984; 141:1356-64.) Ratings of spoken language, language comprehension, word finding difficulty, ability to remember test instructions, maze, and number cancellation may also be obtained. In some embodiments, ADAS-Cog refers to the use of the Alzheimer Disease Assessment Scale-Cognitive Subscale₁₄ (ADAS-Cog14). In some embodiments, a modified version may be used herein and is scored from 0 to 90 points with a score of 0 indicating no impairment, and a score of 90 indicating maximum impairment. In some embodiments, the ADAS-Cog14 tasks include memory (word recall, delayed word recall, and word recognition), reasoning (following commands), language (naming, comprehension), orientation, ideational praxis (placing letter in envelope), constructional praxis (copying geometric designs), spoken language, language comprehension, word finding difficulty, ability to remember test instructions, maze, and number cancellation (Rosen et al, 1984).

As used herein, “CDR-SB” refers to clinical dementia rating—sum of boxes. The CDR is a clinical scale that describes 5 degrees of impairment in performance on each of 6 categories of function including memory, orientation, judgment and problem solving, community affairs, home and hobbies, and personal care. (Berg, L. et al., “Mild senile dementia of the Alzheimer type: 2. Longitudinal assessment.” Ann. Neurol. 1988; 23:477-84.) A sum of boxes score provides a measure of change where each category has a maximum possible score of 3 points and the total score is a sum of the category scores giving a total possible score of 0 to 18 with higher scores indicating more impairment.

As used herein, “CDR global”, “global CDR” score and “global rating of dementia CDR” score is used interchangeably. As used herein, CDR global score is a rating of the degree of impairment obtained on each of the 6 categories of function from the 6 categories of the CDR scale and is synthesized into 1 global rating of dementia CDR score, (ranging from 0 to 3) where 0 indicates no cognitive impairment, 0.5 indicates mild cognitive impairment, and 1-3 indicates mild, moderate, severe dementia respectively. The global CDR score may be used as a clinical measure of severity of dementia. In some embodiments, a global CDR score may be used to determine if a patient has progressed or maintained a stage of AD, e.g., a higher score on a subsequent evaluation indicating progression of AD, e.g., an unchanged score indicating no progression of AD.

As used herein, “ADCOMS” refers to Alzheimer's Disease Composite Score, a composite clinical score based on an analysis of four ADAS-Cog items (delayed word recall, orientation, word recognition, and word finding difficulty), two Mini Mental State Examination (MMSE) items (orientation to time, and drawing), and all six CDR-SB items (personal care, community affairs, home and hobbies, memory, orientation, and judgment and problem solving), as discussed in the Examples and in Wang, J. et al., “ADCOMS: a composite clinical outcome for prodromal Alzheimer's disease trials.” J. Neurol. Neurosurg. Psychiatry. 2016; 87:993-999. ADCOMS was developed to be particularly sensitive to disease progression during early stages of AD (i.e., preclinical AD or early AD).

In some embodiments, ADCOMS can be calculated using the following formula:

$$S ( t ) = {\sum }_{i = 1}^{12} {a}_{i} {A}_{i} ( t ) + {\sum }_{i = 1}^{7} {b}_{i} {B}_{i} ( t ) + {\sum }_{i = 1}^{6} {c}_{i} {C}_{i} ( t )$$

where Aᵢ(t), Bᵢ(t) and Cᵢ(t) are item scores at time t corresponding to items from ADAS-cog, reversed MMSE scores, and CDR-SB, respectively (Wang, J. et al., “ADCOMS: a composite clinical outcome for prodromal Alzheimer's disease trials). ADCOMS is particularly sensitive to disease progression during early stages of AD, i.e., prodromal and mild AD.

As used herein, “ADCS MCI-ADL” refers to the Alzheimer's Disease Cooperative Study-Activities of Daily Living Scale for Mild Cognitive Impairment (ADCS MCI-ADL). The ADCS MCI-ADL is a clinical scale that assesses the competence level of a patient at six basic activities of daily living. Additional examples are discussed in Kreutzer J. S., DeLuca J., Caplan B. (eds) Encyclopedia of Clinical Neuropsychology. Springer, New York, NY.

As used herein, “modified iADRS” or “iADRS” refers to a composite tool that combines scores from the ADAS Cog14 (all items) and the ADCS MCI-ADL (all items). The modified iADRS score can be used to evaluate disease progression:

$$Modified iADRS score = [ - 1 ( A DAS - cog 14 ) + 90 ] + ADCS MCI - ADL .$$

As used herein, “ApoE4-positive” subjects and “ApoE4 carriers” refer to subjects who harbor the ε4 variant of the apolipoprotein (APOE) gene. The ε4 variant is one of several major alleles of the apolipoprotein gene. The gene is generally responsible for metabolism of fats. It has been found that carriers of the apolipoprotein ε4 show significantly greater rates of amyloid retention when compared to non-carriers. (Drzezga, A. et al, “Effect of APOE genotype on amyloid plaque load and gray matter volume in Alzheimer disease.” Neurology. 2009; 72:1487-94.) In some embodiments, a subject treated herein is a heterozygous carrier of the apolipoprotein E ε4 gene allele. In some embodiments, the subject is a homozygous carrier of the apolipoprotein E ε4 gene allele. The terms “ApoE4-negative” and “ApoE4 non-carriers” are used interchangeably.

As used herein, whether an early AD subject is “amyloid positive” or “amyloid negative” may be determined based on whether the subject has a positive amyloid load. In some embodiments, a subject is determined to be amyloid-positive or amyloid-negative as indicated by longitudinal positron emission tomography (PET) assessment of an imaging agent uptake into the brain, e.g., an amyloid imaging agent or a tau imaging agent. In some embodiments, a subject is determined to be amyloid-positive or amyloid-negative by evaluation of a tau PET imaging assessment. In some embodiments, the subject is “amyloid negative” if PET SUVr negativity is below a threshold determined for an amyloid PET tracer. In some embodiments, the amyloid PET tracer may be florbetaben (e.g., 18F-Florbetaben (Neuraceq®)), florbetapir (e.g., 18F-Florbetapir (Amyvid®)), and/or flutametamol (e.g., 18F-Flutemetamol (Vizamyl®)). In some embodiments, the threshold for PET SUVr for an amyloid PET tracer is about 1.17, and a measurement below this threshold may indicate that the subject is “amyloid negative.” In some embodiments, the florbetapir amyloid PET SUVr threshold is about 1.17. In some embodiments, the florbetaben amyloid PET SUVr threshold is about 1.17. In some embodiments, the flutemetamol amyloid PET SUVr threshold is about 1.17. In some embodiments, a subject is determined to be amyloid-positive or amyloid-negative by evaluation of the level of a biomarker in a sample (e.g., a Aβ42/40 ratio) from a subject, alone or in combination with another method such as PET measurement of brain amyloid. In some embodiments, a subject is “amyloid negative” if the Aβ42/40 ratio in a sample is at or about above 0.092-0.094 e.g., at about 0.092. In some embodiments, a subject is “amyloid negative” if the Aβ42/40 ratio in a sample is above 0.092. In some embodiments, a subject is determined to be amyloid-positive or amyloid-negative by a CSF assessment of the presence of amyloid pathology using assessments of markers such as p-tau181, alone or in combination with another method such as PET measurement of brain amyloid. In some embodiments, a qualitative visual read of PET scans may be used to determine amyloid positive and amyloid negative by categorizing subjects as having either “normal” or “abnormal” uptake on the basis of the PET image pattern. Readers will have been trained and certified to recognize brain PET images with abnormal or normal patterns of uptake, or the detection of amyloid is done through a semi-quantitative or quantitative approach. In some embodiments, a threshold will be set for quantitatively determining from a biomarker (e.g., serum or CSF) and/or PET scan whether an Aβ brain load indicates a subject is amyloid-positive or negative. In some embodiments, a subject is determined to be amyloid-positive or amyloid-negative by an imaging method. An imaging method may be used to determine, calculate, or predict whether a subject is amyloid-positive or negative, even when the imaging method is not used to visualize amyloid directly. In some embodiments, the method uses MRI), and/or combines MRI and other imaging modalities such as PET. In some embodiments, a subject is determined to be amyloid-positive or amyloid-negative by retinal amyloid accumulation. In some embodiments, a subject is determined to be amyloid-positive or amyloid-negative by behavioral/cognitive phenotypes.

As would be understood by one of ordinary skill in the art, digital, computerized, and/or conventional (e.g., pen and paper) cognitive tests may be used to detect early cognitive changes that may signal mild cognitive impairment and/or a risk for developing dementia, and thus may be used to identify subject in need of treatment as disclosed herein. Such tests, for example, may screen for cognitive impairment, and potentially identify individuals with MCI. Tests may use artificial intelligence to analyze cognitive test results to determine whether a case of mild cognitive impairment will escalate into Alzheimer's within a year. Diagnosing the condition early, before symptoms have begun to appear, may be used to assist physicians identify subjects in need of treatment as disclosed herein sooner, potentially delaying onset or lessening the severity of the neurodegenerative disease.

As used herein, the term “treat” refers to any administration or application of a therapeutic agent for a disease or disorder in a subject, and includes inhibiting the disease, slowing progression of the disease, delaying progression, arresting its development, reversing progression of disease (e.g., reversing build up of Aβ fibrils), preventing the onset or development of the disease, relieving or ameliorating one or more symptoms or underlying condition(s) of the disease, curing the disease, improving one or more clinical metrics, or preventing reoccurrence of one or more symptoms of the disease. In some embodiments, treatment of AD in a subject comprises an administration, e.g., an intravenous infusion, of an anti-amyloid β (Aβ) protofibril antibody. In some embodiments, treatment of AD in a subject comprises a therapeutically effective dose by administration, e.g., an intravenous infusion, of an anti-amyloid β (Aβ) protofibril antibody.

As used herein, the term “infusion” refers to an active administration of one or more agents with an infusion time of, for example, approximately 60 minutes. In some embodiments, an anti-amyloid β (Aβ) protofibril antibody, described herein is systemically administered to a human subject via infusion. In some embodiments, an anti-amyloid β (Aβ) protofibril antibody is alternatively administered to the human subject, e.g., by subcutaneous injection. In some embodiments, the subcutaneous injection is a weekly injection. In some embodiments, the subcutaneous injection is a biweekly injection. In some embodiments, an anti-amyloid β (Aβ) protofibril antibody is administered to the human subject by intravenous infusion.

In some embodiments, the subject is administered a maintenance dose of a treatment. As used herein, the term “maintenance dose” refers to a dosage administered to a subject to maintain the desired therapeutic effect. In some embodiments, the maintenance dose is administered weekly, every two weeks, monthly, every two months, or every three months (quarterly) or every 24 weeks (every six months or semi-annually). In some embodiments, the maintenance dose comprises an anti-Aβ protofibril antibody. In some embodiments, the maintenance dose is administered as an intravenous infusion. In some embodiments, the intravenous infusion is a 10 mg/kg dose of BAN2401 administered biweekly. In some embodiments, the maintenance dose is administered subcutaneously, orally, or nasally. In some embodiments, the maintenance dose is administered subcutaneously.

In some embodiments, the maintenance dose is administered as a subcutaneous injection. In some embodiments, the maintenance dose is administered as a weekly, subcutaneous injection. In some embodiments, the maintenance dose is administered as a biweekly, subcutaneous injection. In some embodiments, the maintenance dose is administered as a monthly, subcutaneous injection. In some embodiments, the maintenance dose is administered as a quarterly, subcutaneous injection. In some embodiments, the maintenance dose is administered weekly or less frequently, e.g., every two weeks (biweekly), every four weeks, monthly, every six weeks, every eight weeks (2 months), every three months (quarterly) or every six monthly (semi-annually). In some embodiments, the maintenance dose is administered as a biweekly, subcutaneous injection of 720 mg. In some embodiments, the maintenance dose is administered as a biweekly, subcutaneous injection of 720 mg comprising two concurrent, e.g., sequential, injections of 360 mg (2×1.8 mL of 400 mg/2 mL) of the subcutaneous formulation.

In some embodiments, the maintenance dose is administered once or multiple times. In some embodiments, the maintenance dose is administered at a lower dose than during an earlier course of treatment and/or is administered less frequently than during the earlier course of treatment.

In some embodiments, after switching to a maintenance dose, a subject's biomarker levels may indicate increasing levels of amyloid in the brain. In some embodiments, after switching to a maintenance dose, a subject's biomarker levels may begin to worsen, e.g. an increasing plasma Aβ42/40 ratio, indicating increasing levels of amyloid in the brain. In some embodiments, a subject on a maintenance dose may have a decrease in the Aβ42/40 ratio. In some embodiments, a subject is put on a maintenance dose chosen such that the subject may have a decrease in the Aβ42/40 ratio but the Aβ42/40 ratio may remain above the threshold for amyloid positivity, e.g. for at least one year (e.g., at least 1, 2, 3, 4, 5, 6, 7, 8, 9, or 10 years).

In some embodiments, after switching to a maintenance dose, a subject's biomarker levels, e.g. a tau PET level, may begin to increase or a rate of increase may increase. In some embodiments, such a subject may be moved back to a treatment regimen. In some embodiments, a subject may remain on a maintenance dose, e.g., if the increase remains below a tau PET level or rate of increase seen in a control subject who has AD but does not receive an anti-Aβ protofibril antibody.

As used herein, the term “prevent” refers to obtaining beneficial or desired results including, but not limited to, prophylactic benefit. For prophylactic benefit, the composition may be administered to a subject at risk of developing Alzheimer's disease, to a subject having one or more preclinical symptoms but not clinical symptoms of Alzheimer's disease, or to a subject reporting one or more of the physiological symptoms of Alzheimer's disease, even though a clinical diagnosis of having Alzheimer's has not been made. As used herein “prevention” may further include therapeutic benefit, by which is meant eradication or amelioration of the underlying condition being treated or of one or more of the physiological symptoms associated therewith.

As used herein, the term “ARIA” refers to amyloid-related imaging abnormality as evaluated using MRI. In some embodiments, ARIA includes amyloid related imaging abnormality edema/effusion (ARIA-E). In some embodiments, ARIA includes amyloid related imaging abnormality hemorrhage (ARIA-H). In some embodiments, subjects with ARIA experience headache, confusion, and/or seizure and these may be used to identify a subject with ARIA or to indicate further evaluation for ARIA. In some embodiments, ARIA is evaluated at specified intervals during treatment. In some embodiments, ARIA is evaluated when the subject experiences symptoms of ARIA. In some embodiments, maximum serum concentration (Cmax) of anti-Aβ protofibril antibody can be used as a predictor of the risk of ARIA-E. In some embodiments, the use of a subcutaneous formulation may provide a reduced risk of ARIA-E (e.g., due to a lower Cmax) compared to an IV administration.

As used herein, the term “clinical decline” refers to a worsening of one or more clinical symptoms of AD. Methods for measuring clinical decline may employ the tests and assays specified herein. In some embodiments, clinical decline is determined by a worsening of ADCOMS. In some embodiments, clinical decline is determined by a worsening of MMSE. In some embodiments, clinical decline is determined by a worsening of ADAS-Cog. In some embodiments, clinical decline is determined by a worsening of FAQ. In some embodiments, clinical decline is determined by a worsening of CDR-SB. In some embodiments, clinical decline is determined by a worsening of Wechsler Memory Scale-IV Logical Memory (subscale) I and/or (subscale) II. In some embodiments, clinical decline is determined by a worsening of CDR score. In some embodiments, clinical decline refers to a worsening in one or more biomarkers of AD or brain measurement (e.g., by PET or MRI), e.g., of brain atrophy and/or amyloid accumulation.

As used herein, the term “blood sample” or “blood” refers to a sample of blood, including serum and/or blood plasma from a human subject. In some embodiments, blood will be collected from subjects to evaluate potential biomarkers of AD that may include amyloid fragments and isoforms, tau, and other protein biomarkers (e.g., neurofilament light chain or NfL) for association with AD diagnosis, amyloid or tau load, or disease modification. In some embodiments, subjects are required to fast if possible before collection at Week 96 and Week 216. In other embodiments and/or at other time points, subjects do not require fasting. Pre-AD biomarker levels that may suggest the development of Alzheimer's disease include, but are not limited to, brain amyloid level, cerebrospinal fluid level of Aβ1-42, cerebrospinal fluid level of total tau, cerebrospinal fluid level of neurogranin, and cerebrospinal fluid level of neurofilament light chain (NfL).

### B. Anti-Aβ Protofibril Antibodies and Tau PET Levels

In various embodiments, methods are disclosed herein for selecting a patient, treating, monitoring treatment, and making decisions regarding maintenance dosing, in patients receiving an anti-amyloid β protofibril antibody (also called an “anti-amyloid β (Aβ) protofibril antibody” or an “anti-Aβ protofibril antibody”), such as BAN2401. Without being bound by theory, it has been surprisingly found that effective treatment with an anti-amyloid protofibril antibody, such as BAN2401 (i.e., lecanemab) may result in a reduced rate of increase or an overall reduction in tau levels as measured by PET, e.g., in the temporal regions of the brain. In some embodiments, the methods comprise measuring a tau PET level from a subject. In some embodiments, the methods comprise measuring the tau PET level from a subject having or suspected of having AD before treatment and/or again in another sample during treatment (although it is to be understood that additional doses may be administered in between the sampling time points). In some embodiments, the measurement comprises measuring a temporal region of the brain. In some embodiments, an increased tau PET level (e.g., in a temporal region) as compared to a control subject (e.g., one who does not have AD), indicates a subject suitable for treatment with an anti-Aβ protofibril antibody such as BAN2401. In some embodiments, a decrease in the tau PET level and/or in the rate of increase after receiving one or more doses of an anti-Aβ protofibril antibody, such as BAN2401, indicates treatment efficacy. In some embodiments, a subsequent dose of treatment is given after the second sampling if a decrease in the tau PET level or a reduction in the rate of increase in tau PET is detected. In some embodiments, treatment may be titrated on the basis of a change in a tau PET level.

In some embodiments, additional patient demographics, such as age and if the subject is a carrier of the apolipoprotein E ε4 gene allele, may be used to select a patient for treatment in combination with measuring tau PET, e.g., they may be used to predict amyloid positivity (e.g. West et al, Mol Neurodegen (2021) 16-30, Jansen et al, JAMA (2015) 1924-1938, Ossenkoppele et al, JAMA (2015) 1939-1950). In some embodiments, one or more additional biomarker, e.g., one or more blood biomarkers (such as the ratio of Aβ 42 to Aβ 40 and/or a p-tau181) may be used in conjunction with a tau PET measurement. In some embodiments, an age and/or apolipoprotein E ε4 gene allele normalized measurement of tau PET and/or the at least one additional biomarker from a subject is used to evaluate whether a sample (e.g., a plasma sample) from a subject indicates that the subject is suitable for treatment with a protofibril antibody such as BAN2401 (e.g., if the subject is amyloid positive) and/or to monitor treatment. For example, in some embodiments, a patient who is a carrier of an apolipoprotein E ε4 gene allele may be considered amyloid positive at a lower level of a biomarker than needed to indicate amyloid positivity in a subject who is not a carrier. Likewise, in another example, an older subject may be considered amyloid positive at a lower biomarker level than the level required to indicate positivity in a younger subject. In some embodiments, biomarker level is used in a Receiver Operating Characteristic (ROC) analysis to predict amyloid positivity. In some embodiments, additional patient demographics, such as age and if the subject is a carrier of an apolipoprotein E ε4 gene allele, may be used with a biomarker level in an ROC analysis to predict amyloid positivity. In some embodiments, the prediction of amyloid positivity in a patient is used to determine the dosage or frequency of treatment.

In some embodiments, the methods disclosed herein comprise measuring a tau PET level from a subject having or suspected of having AD before treatment with an anti-Aβ protofibril antibody to identify a patient suitable for treatment and/or again in another sample during treatment to monitor treatment efficacy (although it is to be understood that additional doses may be administered in between the sampling time points). In some embodiments, treatment may be stopped and/or reduced (e.g., reduced frequency and/or dosage) if the tau PET level or rate of increase has decreased relative to a control subject, e.g., an AD subject not receiving an anti-Aβ protofibril antibody. In some embodiments, after treatment has been stopped or reduced, a further measurement of the tau PET level may be made from the subject. In some embodiments, treatment is restarted, dosage is increased, and/or the frequency of administration is increased if the tau PET level has increased relative to a control subject. In some embodiments, the dosage or frequency of treatment is increased to return to the dosage and/or frequency used in a prior treatment, e.g., before a dose reduction and/or lengthening of the dose frequency had commenced. In some embodiments, the methods comprise measuring a tau PET level from a subject during treatment and again after stopping treatment or after the dosage or frequency of treatment has been reduced (it is to be understood that additional doses may be administered in between the sampling time points). In some embodiments, if an increase in the tau PET level or rate of increase is detected, treatment is resumed, or the dosage or frequency of treatment is increased, in comparison to the dose or frequency during the period in which the level increased. In some embodiments, multiple measurements may be made during a treatment prior to a decision to stop treatment and/or reduce treatment based on a reduced rate of increase in a tau PET level relative to a control (e.g., based on a trend showing a decreased rate of tau accumulation at each subsequent measurement). In some embodiments, multiple measurements may be taken after treatment has stopped or been reduced, and a decision to resume treatment and/or increase treatment may be taken based on a tau PET level (e.g., based on a trend showing an increased rate of tau accumulation relative to a control). In some embodiments, following the resumption of treatment or the increased treatment regimen, one or more additional measurements may be made of the tau PET level from a subject. In some embodiments, the tau PET level measurement is done in conjunction with measuring one or more additional biomarkers (e.g., using a reduction in amyloid PET SUVr as an indicator of amyloid plaque reduction during and/or after treatment). In some embodiments, treatment may be stopped if the tau PET level does not change relative to a control, e.g., relative to an untreated AD subject. In some embodiments, treatment may be stopped due to a low therapeutic effect.

In some embodiments, any of the methods that comprise measuring a tau PET level may further comprise, or may comprise in lieu of measuring a tau PET level, measuring one or more additional biomarkers. In some embodiments, any of the methods that comprise measuring a tau PET level may further comprise measuring one or more additional biomarkers. In some embodiments, the additional biomarker comprises volumetric MRI (vMRI), e.g., measuring whole brain volume, cortical thickness, total hippocampal volume, and/or lateral ventricle volume. In some embodiments, the one or more additional biomarkers comprises a PET level, such as an amyloid PET level and/or a fluorodeoxyglucose (FDG) PET level. In some embodiments, the one or more additional biomarkers comprises a cerebrospinal fluid and or blood level of one or more biomarker including Aβ1-42, Aβ1-40 (including a ratio of Aβ1-42 to Aβ1-40), total tau, phosphorylated tau (P-tau) (including tau phosphorylated at 181 (P-tau181), 205 (P-tau205), 217 (P-tau217), and 231 (P-tau231)), neurogranin, neurofilament light chain (NfL), and/or microtubule binding region (MTBR)-tau containing the residue 243 (MTBR-tau243). In some embodiments, the one or more additional biomarkers comprises a serum or plasma level of a biomarker, including Aβ1-42, Aβ1-40 (including a ratio of Aβ1-42 to Aβ1-40), total tau, phosphorylated tau (P-tau) (including tau phosphorylated at 181 (P-tau181), 205 (P-tau205), 217 (P-tau217), and 231 (P-tau231)), glial fibrillary acidic protein (GFAP), and/or neurofilament light chain (NfL). In some embodiments, the one or more additional biomarkers comprise the microtubule-binding region of tau containing the residue 243 (MTBR-tau243). Recent studies indicate that expression of MTBR-tau243 in both CSF and plasma may correlate with tau PET levels, cognitive measures, and AD disease progression (Horie et al., Brain, 2021, 144(2): 515-527) (Horie et al., Nature Medicine, 2023, 29: 1954-1963) (Horie et al., The Journal of Prevention of Alzheimer's Disease 2023,10: S36).

In some embodiments, provided herein is a method of reducing and/or slowing clinical decline in a subject, e.g., one having Alzheimer's disease, Pre-AD, or early Alzheimer's disease, comprising administering a therapeutically effective amount of at least one anti-Aβ protofibril antibody (e.g., BAN2401) to a patient with a tau PET level greater than a subject who does not have AD. In some embodiments, the anti-Aβ protofibril antibody (e.g., BAN2401) is administered in a therapeutically effective amount to a patient with a tau PET level greater than a subject who does not have AD. In some embodiments, a reduced rate of tau PET level increase indicates a slowing of the cognitive decline of a patient (e.g., one having pre-AD or early AD) relative to the decline in the absence of treatment. In some embodiments, a reduced rate of tau accumulation indicates a slowing of the cognitive decline of a patient (e.g., one having pre-AD or early AD) relative to the decline in the absence of treatment. In some embodiments, after a reduced rate of tau PET increase is detected, e.g., when measured at 18 months after the start of treatment, a subject may be moved to a maintenance dosing regimen. In some embodiments, after a reduced rate of tau PET increase is detected in combination with a reduction in one or more additional biomarkers of AD progression, e.g., when measured at 18 months after the start of treatment, a subject may be moved to a maintenance dosing regimen.

In some embodiments, provided herein is a method of improving cognitive outcomes in a subject, e.g., one having Alzheimer's disease, Pre-AD, or early Alzheimer's disease, comprising administering a therapeutically effective amount of at least one anti-Aβ protofibril antibody (e.g., BAN2401) to a patient with a tau PET level greater than a subject who does not have AD. Cognitive and functional decline can be measured by techniques known in the art, including scoring methods such as CDR-SB, ADCOMS Composite Clinical Score, the Mini-Mental State Examination, ADAS-Cog, ADAS MCI-ADL, modified iADRS, Wechsler Memory Scale-IV Logical Memory (subscale) I (WMS-IV LMI), and Wechsler Memory Scale-IV Logical Memory (subscale) II (WMS-IV LMII). In some embodiments, the anti-Aβ protofibril antibody (e.g., BAN2401) is administered in a therapeutically effective amount to a patient with a tau PET level greater than a subject who does not have AD. In some embodiments, the anti-Aβ protofibril antibody (e.g., BAN2401) is administered as a therapeutically effective dose comprising an intravenous infusion of 10 mg/kg relative to the weight of the subject. In some embodiments, the anti-Aβ protofibril antibody (e.g., BAN2401) is administered as a therapeutically effective dose comprising a subcutaneous administration of 720 mg. In some embodiments, the method results in less cognitive decline as measured by ADCOMS compared to an untreated subject. In some embodiments, the method results in at least 24% less (e.g., at least 29% less) cognitive decline as measured by ADCOMS compared to an untreated subject. In some embodiments, the method results in less cognitive decline as measured by CDR-SB compared to an untreated subject. In some embodiments, the method results in at least 26% less (e.g., at least 27% less) cognitive decline as measured by CDR-SB compared to an untreated subject. In some embodiments, the method results in less cognitive decline as measured by ADAS-Cog14 compared to an untreated subject. In some embodiments, the method results in at least 26% less (e.g., at least 47% less) cognitive decline as measured by ADAS-Cog14 compared to an untreated subject. In some embodiments, the method results in less cognitive decline as measured by ADCS MCI-ADL compared to an untreated subject. In some embodiments, the method results in at least 37% less cognitive decline as measured by ADCS MCI-ADL compared to an untreated subject. In some embodiments, the method results in a reduced risk of progression to a subsequent stage of AD as measured by a CDR global score, e.g., a higher score on a subsequent evaluation indicating progression to the next stage of AD, e.g., an unchanged score indicating remaining in the same stage of AD. In some embodiments, the result is measured at least 6 months after administering a therapeutically effective amount of at least one anti-Aβ protofibril antibody. In some embodiments, the result is measured at least 12 months after administering a therapeutically effective amount of at least one anti-Aβ protofibril antibody. In some embodiments, the result is measured at least 13 months after administering a therapeutically effective amount of at least one anti-Aβ protofibril antibody. In some embodiments, the result is measured at least 18 months after administering a therapeutically effective amount of at least one anti-Aβ protofibril antibody.

In some embodiments, provided herein, is a method of treatment using a tau PET level or a rate of change in a tau PET level from a region of the brain (e.g., the temporal region). For example, in some embodiments, a treatment comprises administering an anti-Aβ protofibril antibody, e.g., BAN2401, intravenously or subcutaneously. In some embodiments, the method of treatment comprises using a biomarker level, e.g., a tau PET level or a rate of change in a tau PET level, to determine switching to a maintenance intravenous or subcutaneous dose at a set time point (e.g., after 18 months). In some embodiments, the method of treatment comprises using a biomarker level, e.g., a tau PET level or a rate of change in a tau PET level, in combination with one or more biomarker criteria (e.g., a ratio of Aβ1-42 to Aβ1-40 and/or a p-tau181 measurements in a fluid sample, e.g., a blood sample). In some embodiments, the combination comprises an amyloid PET measurement. In some embodiments, the combination comprises a serum or plasma GFAP measurement. In some embodiments, the same marker or combination of markers may be used to select a patient for treatment with the protofibril antibody, e.g., BAN2401, by comparison to the level in a control subject, e.g., one who does not have AD.

An anti-Aβ protofibril antibody, such as BAN2401, may be formulated in a pharmaceutical composition as disclosed in PCT/IB2021/000155 (WO2021/186245), which is incorporated herein by reference. In some embodiments, the composition comprises 80 mg/mL to 120 mg/mL BAN2401, 240 mM to 360 mM arginine, 0.03% w/v to 0.08% w/v polysorbate 80, and 30 mM to 70 mM citrate buffer. In some embodiments, the arginine is arginine, arginine hydrochloride, or a combination thereof. In some embodiments, the composition comprises a liquid dosage form comprising 100 mg/mL BAN2401, 50 mmol/L citrate, 350 mmol/L arginine, and 0.05% polysorbate 80. In some embodiments, the composition comprises 80 mg/mL to 240 mg/mL BAN2401, 140 mM to 260 mM arginine hydrochloride, 0.01% w/v to 0.1% w/v polysorbate 80, and 15 mM to 35 mM histidine buffer. In some embodiments, the composition comprises a liquid dosage form comprising 100 mg/mL BAN2401, 25 mmol/L histidine, 200 mmol/L arginine, and 0.05% polysorbate 80.

In some embodiments, BAN2401, is formulated as disclosed in PCT/IB2021/000155 (WO2021/186245), which is incorporated herein by reference. In some embodiments, the composition comprises 80 mg/mL to 240 mg/mL BAN2401, 140 mM to 260 mM arginine hydrochloride, 0.01% w/v to 0.1% w/v polysorbate 80, and 15 mM to 35 mM histidine buffer. In some embodiments, the composition comprises a liquid dosage form comprising 200 mg/mL BAN2401, 25 mmol/L histidine, 200 mmol/L arginine, and 0.05% polysorbate 80.

In some embodiments, provided herein is a method of treatment comprising administering a therapeutically effective dose of the anti-Aβ protofibril antibody until a desired improvement in one or more biomarkers, e.g. a tau PET level or a rate of change in a tau PET level, or another treatment outcome measures is achieved. In some embodiments, treatment is continued until a desired improvement in one or more biomarkers or other treatment outcome measures is achieved, e.g., when a tau PET level that increased by no more than 0.05-0.1 after 13 or 18 months of treatment as evaluated by tau PET SUVR in the temporal region. In some embodiments, treatment is continued until a tau PET level has improved relative to an untreated control subject and/or treatment is continued until an increase in the ratio of Aβ1-42 to Aβ1-40 in a fluid sample, e.g., a blood sample. In some embodiments, treatment is continued until a tau PET level has improved relative to an untreated control subject and/or treatment is continued until the ratio of Aβ1-42 to Aβ1-40 in a fluid sample, e.g., a blood sample is at or above 0.092. In some embodiments, treatment is continued until a tau PET level has improved relative to an untreated control subject and/or a decrease in the level of p-tau181 in a fluid sample, e.g., a blood sample. In some embodiments, treatment is continued until a tau PET level has improved relative to an untreated control subject and/or the amyloid PET SUVr negativity is below a threshold of about 1.17. In some embodiments, the amyloid PET tracer may be florbetaben (e.g., 18F-Florbetaben (Neuraceq®)), florbetapir (e.g., 18F-Florbetapir (Amyvid®)), and/or flutametamol (e.g., 18F-Flutemetamol (Vizamyl®)). In some embodiments, the threshold for PET SUVr for an amyloid PET tracer is about 1.17, and measurement below this threshold may indicate that the subject is “amyloid negative.” In some embodiments, the florbetapir amyloid PET SUVr threshold is about 1.17. In some embodiments, the florbetaben amyloid PET SUVr threshold is about 1.17. In some embodiments, the flutemetamol amyloid PET SUVr threshold is about 1.17.

In some embodiments, the one or more biomarkers comprises a serum or plasma GFAP measurement. In some embodiments, treatment is continued until a subject is amyloid negative. In some embodiments, a subject will switch to a maintenance dose after a desired improvement in one or more biomarkers, e.g., a tau PET level or other treatment outcome measures, is achieved. In some embodiments, a maintenance dosing regimen may further comprise one or more additional treatments in addition to an anti-Aβ protofibril antibody, e.g., it may comprise administering E2814.

In some embodiments, provided herein is a method of treatment comprising administering a therapeutically effective dose of the anti-Aβ protofibril antibody until a desired improvement in the cognitive outcomes in a subject, e.g., one having Alzheimer's disease, Pre-AD, or early Alzheimer's disease, or other treatment outcome measures is achieved in combination with a tau PET level or rate of change in tau PET. In some embodiments, the method of treatment comprising administering a therapeutically effective dose of the anti-Aβ protofibril antibody until a tau PET level has improved relative to an untreated control subject and a desired improvement in the cognitive outcomes in a subject or other treatment outcome measures is achieved. In some embodiments, the method of treatment comprising administering a therapeutically effective dose of the anti-Aβ protofibril antibody for at least 18 months or e.g., until a patient has a tau PET level that has increased by no more than 0.05-0.1 as evaluated by tau PET SUVR in the temporal region, and a desired improvement in the cognitive outcomes in a subject is achieved. Cognitive and functional decline can be measured by techniques known in the art, including scoring methods such as CDR-SB, ADCOMS Composite Clinical Score, the Mini-Mental State Examination, ADAS-Cog, ADAS MCI-ADL, modified iADRS, Wechsler Memory Scale-IV Logical Memory (subscale) I (WMS-IV LMI), and Wechsler Memory Scale-IV Logical Memory (subscale) II (WMS-IV LMII). In some embodiments, a method of treatment comprising administering a therapeutically effective dose of the anti-Aβ protofibril antibody continues until the method results in less cognitive decline as measured by ADCOMS compared to an untreated subject. In some embodiments, the efficacy of a method of treatment may be evaluated using a CDR global score, wherein a CDR global score may be used to determine if a patient has progressed or maintained a stage of AD during treatment, e.g., a higher score on a subsequent evaluation indicating progression of AD, e.g., an unchanged score indicating no progression of AD. In some embodiments, the method of treatment continues until the method results in at least 24% less (e.g., at least 29% less) cognitive decline as measured by ADCOMS compared to an untreated subject. In some embodiments, the method of treatment continues until the method results in less cognitive decline as measured by CDR-SB compared to an untreated subject. In some embodiments, the method of treatment continues until the method results in at least 26% less (e.g., at least 27% less) cognitive decline as measured by CDR-SB compared to an untreated subject. In some embodiments, the method of treatment continues until the method results in less cognitive decline as measured by ADAS-Cog14 compared to an untreated subject. In some embodiments, the method of treatment continues until the method results in at least 26% less (e.g., at least 47% less) cognitive decline as measured by ADAS-Cog14 compared to an untreated subject. In some embodiments, the method results in less cognitive decline as measured by ADCS MCI-ADL compared to an untreated subject. In some embodiments, the method results in at least 37% less cognitive decline as measured by ADCS MCI-ADL compared to an untreated subject. In some embodiments, the result is measured at least 6 months after administering a therapeutically effective amount of at least one anti-Aβ protofibril antibody. In some embodiments, the result is measured at least 12 months after administering a therapeutically effective amount of at least one anti-Aβ protofibril antibody. In some embodiments, the result is measured at least 13 months after administering a therapeutically effective amount of at least one anti-Aβ protofibril antibody. In some embodiments, the result is measured at least 18 months after administering a therapeutically effective amount of at least one anti-Aβ protofibril antibody.

Methods of treatment, including dosing, by an intravenous administration of an anti-Aβ protofibril antibody are disclosed in PCT/US2022/073576 and PCT/US2022/079571 and are incorporated herein by reference. In some embodiments, a treatment comprises administering intravenously an anti-Aβ protofibril antibody at 10 mg/kg (e.g., administering BAN2401 at 10 mg/kg), biweekly, e.g., for at least 18 months or e.g., until a patient is amyloid-negative. In some embodiments, a treatment comprises administering intravenously an anti-Aβ protofibril antibody at 10 mg/kg (e.g., administering BAN2401 at 10 mg/kg), biweekly, e.g., for at least 18 months or e.g., until a patient is amyloid-negative, before switching to a maintenance dose. In some embodiments, the method of treatment comprises administering intravenously an anti-Aβ protofibril antibody at 10 mg/kg (e.g., administering BAN2401 at 10 mg/kg), biweekly, e.g., for at least 18 months or e.g., until a patient has a tau PET level that has increased by no more than 0.05-0.1 after 13 or 18 months of treatment as evaluated by tau PET SUVR in the temporal region, before switching to a maintenance dose. In some embodiments, the method of treatment comprises administering intravenously an anti-Aβ protofibril antibody at 10 mg/kg (e.g., administering BAN2401 at 10 mg/kg), biweekly, e.g., for at least 18 months or e.g., until a tau PET level has improved relative to an untreated control subject, before switching to a maintenance dose. In some embodiments, the maintenance dose may be the same as the treatment dose, or it may involve a reduced dosage and/or frequency of administration.

Methods of treatment, including dosing, by a subcutaneously administered anti-Aβ protofibril antibody are disclosed in PCT/US2022/073576; PCT/US2022/079571; and PCT/US2022/041926 and are incorporated herein by reference. In some embodiments, a treatment comprises subcutaneously administering an anti-Aβ protofibril antibody (e.g., administering BAN2401 at 720 mg), weekly, e.g., for at least 18 months or e.g., until a patient is amyloid negative. In some embodiments, a treatment comprises subcutaneously administering BAN2401 weekly, e.g., weekly subcutaneous injection of 720 mg in two concurrent, e.g., sequential, injections of 360 mg (2×1.8 mL of 400 mg/2 mL) of the subcutaneous formulation, e.g., until a patient is amyloid-negative or e.g., for at least 18 months or e.g., until a patient has a tau PET level that has increased by no more than 0.05-0.1 after 13 or 18 months of treatment as evaluated by tau PET SUVR in the temporal region. In some embodiments, a treatment comprises subcutaneously administering BAN2401 weekly, e.g., weekly subcutaneous injection of 720 mg in two concurrent, e.g., sequential, injections of 360 mg (2×1.8 mL of 400 mg/2 mL) of the subcutaneous formulation, e.g., until a patient is amyloid-negative or e.g., for at least 18 months or e.g., until a tau PET level has improved relative to an untreated control subject. In some embodiments, a treatment comprises subcutaneously administering BAN2401 weekly, e.g., at a dose of 720 mg, e.g., for at least 18 months or e.g., until a patient is amyloid-negative, before switching to a weekly, subcutaneous maintenance dose, e.g., a dose of 360 mg. In some embodiments, a treatment comprises subcutaneously administering BAN2401 weekly, e.g., at a dose of 720 mg, e.g., for at least 18 months or e.g., until a patient is amyloid-negative, before switching to a biweekly subcutaneous maintenance dose, e.g., a dose of 720 mg. In some embodiments, the method of treatment comprises subcutaneously administering BAN2401 weekly, e.g., weekly subcutaneous injection of 720 mg in two concurrent, e.g., sequential, injections of 360 mg (2×1.8 mL of 400 mg/2 mL) of the subcutaneous formulation, e.g., for at least 18 months or e.g., until a patient has a tau PET level that has increased by no more than 0.05-0.1 after 13 or 18 months of treatment as evaluated by tau PET SUVR in the temporal region, before switching to a maintenance dose. In some embodiments, the method of treatment comprises subcutaneously administering BAN2401 weekly, e.g., weekly subcutaneous injection of 720 mg in two concurrent, e.g., sequential, injections of 360 mg (2×1.8 mL of 400 mg/2 mL) of the subcutaneous formulation, e.g., for at least 18 months or e.g., until a tau PET level has improved relative to an untreated control subject, before switching to a maintenance dose. In some embodiments, the maintenance dose may be the same as the treatment dose, or it may involve a reduced dosage and/or frequency of administration.

In some embodiments, the method of treatment comprises using a biomarker level, e.g., a tau PET level or a rate of change in a tau PET level, to determine switching to a maintenance intravenous or subcutaneous dose at a set time point (e.g., after 18 months). In some embodiments, following a treatment period, a maintenance dose is administered. In some embodiments, a treatment comprises administering intravenously an anti-Aβ protofibril antibody before switching to an intravenous maintenance dose. In some embodiments, a treatment comprises administering intravenously an anti-Aβ protofibril antibody at 10 mg/kg (e.g., administering BAN2401 at 10 mg/kg), biweekly, e.g., until a patient has a tau PET level that has increased by no more than 0.05-0.1 after 13 or 18 months of treatment as evaluated by tau PET SUVR in the temporal region, before switching to an intravenous maintenance dose. In some embodiments, a treatment comprises administering intravenously an anti-Aβ protofibril antibody at 10 mg/kg (e.g., administering BAN2401 at 10 mg/kg), biweekly, e.g., for at least 18 months or until a tau PET level has improved relative to an untreated control subject, before switching to an intravenous maintenance dose. In some embodiments, a treatment comprises administering intravenously an anti-Aβ protofibril antibody at 10 mg/kg (e.g., administering BAN2401 at 10 mg/kg), biweekly, e.g., for at least 18 months or e.g., until a patient is amyloid-negative, before switching to a biweekly intravenous maintenance dose. In some embodiments, a treatment comprises administering intravenously an anti-Aβ protofibril antibody at 10 mg/kg (e.g., administering BAN2401 at 10 mg/kg), biweekly, e.g., until a patient has a tau PET level that has increased by no more than 0.05-0.1 after 13 or 18 months of treatment as evaluated by tau PET SUVR in the temporal region or e.g., until a tau PET level has improved relative to an untreated control subject, before switching to an monthly intravenous maintenance dose. In some embodiments, a treatment comprises administering intravenously an anti-Aβ protofibril antibody at 10 mg/kg (e.g., administering BAN2401 at 10 mg/kg), biweekly, e.g., until a patient has a tau PET level that has increased by no more than 0.05-0.1 after 13 or 18 months of treatment as evaluated by tau PET SUVR in the temporal region or e.g., until a tau PET level has improved relative to an untreated control subject, before switching to a quarterly intravenous maintenance dose. In some embodiments, a treatment comprises administering intravenously an anti-Aβ protofibril antibody before switching to a subcutaneous maintenance dose. In some embodiments, a treatment comprises administering intravenously an anti-Aβ protofibril antibody at 10 mg/kg (e.g., administering BAN2401 at 10 mg/kg), biweekly, e.g., until a patient has a tau PET level that has increased by no more than 0.05-0.1 after 13 or 18 months of treatment as evaluated by tau PET SUVR in the temporal region or e.g., until a tau PET level has improved relative to an untreated control subject, before switching to a subcutaneous maintenance dose, e.g., 720 mg administered weekly or biweekly, 360 mg administered weekly, or 250 mg administered weekly. In some embodiments, the intravenous maintenance dose is administered every two weeks. In some embodiments, the intravenous maintenance dose is administered every four weeks. In some embodiments, the intravenous maintenance dose is administered every six weeks. In some embodiments, the intravenous maintenance dose is administered every eight weeks (2 months). In some embodiments, the intravenous maintenance dose is administered every three months (quarterly). In some embodiments, the intravenous maintenance dose is administered every 24 weeks (every six months or semi-annually). In some embodiments, a treatment comprises administering intravenously an anti-Aβ protofibril antibody at 10 mg/kg, biweekly, until a patient has a tau PET level that has increased by no more than 0.05-0.1 after 13 or 18 months of treatment as evaluated by tau PET SUVR in the temporal region or e.g., until a tau PET level has improved relative to an untreated control subject, before switching to a biweekly intravenous maintenance dose.

In some embodiments, the method of treatment comprises using a biomarker level, e.g., a tau PET level or a rate of change in a tau PET level, to determine switching from a subcutaneous treatment to a subcutaneous maintenance dose. In some embodiments, the maintenance dose is administered as a subcutaneous injection of the anti-Aβ protofibril antibody (e.g., BAN2401). In some embodiments, a treatment comprises subcutaneously administering an anti-Aβ protofibril antibody before switching to a subcutaneous maintenance dose. In some embodiments, a treatment comprises subcutaneously administering an anti-Aβ protofibril antibody at 720 mg biweekly (e.g., administering sequential, injections of 360 mg (2×1.8 mL of 400 mg/2 mL) of the subcutaneous formulation), e.g., until a patient has a tau PET level that has increased by no more than 0.05-0.1 after 13 or 18 months of treatment as evaluated by tau PET SUVR in the temporal region, before switching to a weekly or biweekly subcutaneous injection of 720 mg. In some embodiments, a treatment comprises subcutaneously administering an anti-Aβ protofibril antibody at 720 mg biweekly (e.g., administering sequential, injections of 360 mg (2×1.8 mL of 400 mg/2 mL) of the subcutaneous formulation), e.g., for at least 18 months or until a tau PET level has improved relative to an untreated control subject, before switching to a weekly or biweekly subcutaneous injection of 720 mg. In some embodiments, the maintenance dose is administered as a weekly subcutaneous injection of the subcutaneous formulation of the anti-Aβ protofibril antibody. In some embodiments, the maintenance dose is administered as a weekly, subcutaneous injection of 720 mg comprising two concurrent, e.g., sequential, injections of 360 mg (2×1.8 mL of 400 mg/2 mL) of the subcutaneous formulation. In some embodiments, the maintenance dose is administered as a monthly, subcutaneous injection of 720 mg comprising two concurrent, e.g., sequential, injections of 360 mg (2×1.8 mL of 400 mg/2 mL) of the subcutaneous formulation. In some embodiments, a treatment comprises subcutaneously administering an anti-Aβ protofibril antibody at 720 mg weekly (e.g., administering sequential, injections of 360 mg (2×1.8 mL of 400 mg/2 mL) of the subcutaneous formulation), e.g., for at least 18 months or e.g., until a patient is amyloid-negative, before switching to a biweekly subcutaneous injection of 720 mg.

In some embodiments, the method of treatment comprises using a biomarker level, e.g., a tau PET level or a rate of change in a tau PET level, to determine switching from an intravenous treatment to a maintenance dose. In some embodiments, a maintenance dose is administered subcutaneously (e.g., as one or more subcutaneous injections). In some embodiments, a treatment comprises administering intravenously an anti-Aβ protofibril antibody at 10 mg/kg (e.g., administering BAN2401 at 10 mg/kg), biweekly, e.g., until a patient has a tau PET level that has increased by no more than 0.05-0.1 after 13 or 18 months of treatment as evaluated by tau PET SUVR in the temporal region or e.g., until a tau PET level has improved relative to an untreated control subject before switching to a subcutaneous maintenance dose. In some embodiments, a treatment comprises administering intravenously an anti-Aβ protofibril antibody at 10 mg/kg, biweekly, e.g., until a patient has a tau PET level that has increased by no more than 0.05-0.1 after 13 or 18 months of treatment as evaluated by tau PET SUVR in the temporal region or e.g., until a tau PET level has improved relative to an untreated control subject, before switching to a weekly subcutaneous maintenance dose. In some embodiments, a treatment comprises administering intravenously an anti-Aβ protofibril antibody at 10 mg/kg, biweekly, e.g., until a patient has a tau PET level that has increased by no more than 0.05-0.1 after 13 or 18 months of treatment as evaluated by tau PET SUVR in the temporal region or e.g., until a tau PET level has improved relative to an untreated control subject, before switching to a weekly, 720 mg, subcutaneous maintenance dose. In some embodiments, a treatment comprises administering intravenously an anti-Aβ protofibril antibody at 10 mg/kg, biweekly, e.g., until a patient has a tau PET level that has increased by no more than 0.05-0.1 after 13 or 18 months of treatment as evaluated by tau PET SUVR in the temporal region or e.g., until a tau PET level has improved relative to an untreated control subject, before switching to a biweekly, 720 mg, subcutaneous maintenance dose.

In some embodiments, the method of treatment comprises using a biomarker level, e.g., a tau PET level or a rate of change in a tau PET level, to determine switching from a subcutaneous treatment to a maintenance dose. In some embodiments, a treatment comprises subcutaneously administering an anti-Aβ protofibril antibody, e.g., BAN2401, before switching to an intravenous maintenance dose. In some embodiments, a treatment comprises subcutaneously administering BAN2401 weekly, e.g., a subcutaneous injection of 720 mg comprising two concurrent, e.g., sequential, injections of 360 mg (2×1.8 mL of 400 mg/2 mL), e.g., until a patient has a tau PET level that has increased by no more than 0.05-0.1 after 13 or 18 months of treatment as evaluated by tau PET SUVR in the temporal region or e.g., until a tau PET level has improved relative to an untreated control subject, before switching to a maintenance dose. In some embodiments, a treatment comprises subcutaneously administering BAN2401 weekly, e.g., at a dose of 720 mg, e.g., until a patient has a tau PET level that has increased by no more than 0.05-0.1 after 13 or 18 months of treatment as evaluated by tau PET SUVR in the temporal region or e.g., until a tau PET level has improved relative to an untreated control subject, before switching to a maintenance dose. In some embodiments, a treatment comprises subcutaneously administering BAN2401 weekly, e.g., until a patient has a tau PET level that has increased by no more than 0.05-0.1 after 13 or 18 months of treatment as evaluated by tau PET SUVR in the temporal region or e.g., until a tau PET level has improved relative to an untreated control subject, before switching to an intravenous maintenance dose of 10 mg/kg biweekly. In some embodiments, a subject's maintenance dose is administered at the same amount and/or frequency as the dose during the treatment period. In some embodiments, a subject's maintenance dose is 50% of the dose during the treatment period.

In some embodiments, a patient starts on an intravenous maintenance dose, e.g., a dosing of 10 mg/kg BAN2401 as disclosed above before switching to a subcutaneous maintenance dose, e.g., a subcutaneous injection of 720 mg comprising two concurrent, e.g., sequential, injections of 360 mg (2×1.8 mL of 400 mg/2 mL) of the subcutaneous formulation. In some embodiments, a patient starts on a subcutaneous maintenance dose, e.g., a subcutaneous injection of 720 mg comprising two concurrent, e.g., sequential, injections of 360 mg (2×1.8 mL of 400 mg/2 mL) of the subcutaneous formulation before switching to an intravenous maintenance dose, e.g., a dosing of 10 mg/kg BAN2401 as disclosed above.

In some embodiments, a patient is moved back from a maintenance dose to the initial treatment dose if the patient is determined to no longer be amyloid negative, e.g., as assessed by blood, serum, or CSF biomarker and/or as determined by amyloid PET SUVr.

In some embodiments, a subject's maintenance dose is administered at the same amount and/or frequency as the dose during the treatment period. In some embodiments, a subject's maintenance dose is 50% of the dose during the treatment period. In some embodiments, the maintenance dose comprises two or more dosings, in which a first dosing is selected from the maintenance dose as exemplified above and a second and/or subsequent dosing comprising a lower amount and/or frequency of dosing than the first or previous dosing, respectively. In some embodiments, the switching to the second or subsequent dosing is determined based on one or more biomarkers as exemplified above, where the levels of the biomarkers are different from (e.g., improved over) the levels used in switching from initial dose to the first dosing in the maintenance dose.

In some embodiments, a patient's treatment is discontinued if a patient no longer has early AD, e.g., as assessed by cognitive evaluation, PET SUVr, and/or blood, CSF, or plasma biomarkers.

In some embodiments, a patient's amyloid level may be monitored for regression after treatment discontinuation by measuring a tau PET level and one or more biomarkers such as volumetric MRI (vMRI), comprising whole brain volume, cortical thickness, total hippocampal volume, and/or lateral ventricle volume. In some embodiments, a patient's amyloid level may be monitored for regression after treatment discontinuation by measuring a tau PET level and one or more biomarkers such as a PET level, comprising an amyloid PET level and/or a fluorodeoxyglucose (FDG) PET level. In some embodiments, a patient's amyloid level may be monitored for regression after treatment discontinuation by measuring a tau PET level and one or more biomarkers such as a cerebrospinal fluid level of a biomarker comprising the CSF level of Aβ1-42, Aβ1-40 (including a ratio of Aβ1-42 to Aβ1-40), total tau, neurogranin, neurofilament light chain (NfL), and/or microtubule binding region (MTBR)-tau. In some embodiments, a patient's amyloid level may be monitored for regression after treatment discontinuation by measuring a tau PET level and one or more biomarkers such as a serum or plasma level of a biomarker comprising Aβ1-42, Aβ1-40 (including a ratio of Aβ1-42 to Aβ1-40), total tau, phosphorylated tau (P-tau) (including tau phosphorylated at 181 (P-tau181), 217 (P-tau217), and 231 (P-tau231)), glial fibrillary acidic protein (GFAP), and/or neurofilament light chain (NfL).

In some embodiments, a patient's biomarkers may be monitored at least once after the discontinuation of treatment. In some embodiments, a patient's biomarkers are monitored at least 1 week, 2 weeks, 3 weeks, 1 month, 2 months, 3 months, 6 months, 12 month, 18 months, or 24 months after treatment discontinuation. In some embodiments, treatment is reinitiated if a patient's biomarker level becomes less favorable, e.g., a tau PET level increases at the same rate as an untreated control.

In some embodiments, the subject has been diagnosed with early AD. In some embodiments, the subject has been diagnosed as having mild cognitive impairment due to Alzheimer's disease—intermediate likelihood and/or has been diagnosed as having mild Alzheimer's disease dementia.

Methods of treatment using an anti-Aβ protofibril antibody, including a therapeutically effective dosage, are disclosed in PCT/US2022/073576; PCT/US2022/079571; and PCT/US2022/041926, which is herein incorporated by reference. In some embodiments, the method of treatment comprises using a biomarker level, e.g., a tau PET level or a rate of change in a tau PET level, to allow for monitoring and treatment decisions. In some embodiments, the method of treatment comprises using a biomarker level, e.g., a tau PET level or a rate of change in a tau PET level, to determine switching from a treatment to a maintenance dose. In some embodiments, a first therapeutically effective dose comprises administering intravenously an anti-Aβ protofibril antibody at 10 mg/kg (e.g., administering BAN2401 at 10 mg/kg), biweekly, e.g., until a patient has a tau PET level that has increased by no more than 0.05-0.1 after 13 or 18 months of treatment as evaluated by tau PET SUVR in the temporal region or e.g., until a tau PET level has improved relative to an untreated control subject, before switching to an intravenous maintenance dose (e.g., at 10 mg/kg, e.g., biweekly, or every 4, 6, 8, 10, or 12 weeks). In some embodiments, a first therapeutically effective dose comprises administering intravenously an anti-Aβ protofibril antibody at 10 mg/kg (e.g., administering BAN2401 at 10 mg/kg), biweekly, e.g., until a patient has a tau PET level that has increased by no more than 0.05-0.1 after 13 or 18 months of treatment as evaluated by tau PET SUVR in the temporal region or e.g., until a tau PET level has improved relative to an untreated control subject, before switching to a biweekly intravenous maintenance dose. In some embodiments, a first therapeutically effective dose comprises administering intravenously an anti-Aβ protofibril antibody at 10 mg/kg (e.g., administering BAN2401 at 10 mg/kg), biweekly, e.g., until a patient has a tau PET level that has increased by no more than 0.05-0.1 after 13 or 18 months of treatment as evaluated by tau PET SUVR in the temporal region or e.g., until a tau PET level has improved relative to an untreated control subject, before switching to a monthly intravenous maintenance dose. In some embodiments, a first therapeutically effective dose comprises administering intravenously an anti-Aβ protofibril antibody at 10 mg/kg (e.g., administering BAN2401 at 10 mg/kg), biweekly, e.g., until a patient has a tau PET level that has increased by no more than 0.05-0.1 after 13 or 18 months of treatment as evaluated by tau PET SUVR in the temporal region or e.g., until a tau PET level has improved relative to an untreated control subject, before switching to a quarterly intravenous maintenance dose.

In some embodiments, a first therapeutically effective dose comprises subcutaneously administering an anti-Aβ protofibril antibody at 720 mg (e.g., administering BAN2401 at 720 mg) weekly, e.g., until a patient has a tau PET level that has increased by no more than 0.05-0.1 after 13 or 18 months of treatment as evaluated by tau PET SUVR in the temporal region or e.g., until a tau PET level has improved relative to an untreated control subject, before switching to a subcutaneous maintenance dose (e.g., at 720 mg, e.g., weekly, biweekly, or every 4, 6, 8, 10, or 12 weeks). In some embodiments, a first therapeutically effective dose comprises subcutaneously administering an anti-Aβ protofibril antibody at 720 mg (e.g., administering BAN2401 at 720 mg) weekly, e.g., until a patient has a tau PET level that has increased by no more than 0.05-0.1 after 13 or 18 months of treatment as evaluated by tau PET SUVR in the temporal region or e.g., until a tau PET level has improved relative to an untreated control subject, before switching to a biweekly subcutaneous maintenance dose (e.g., at 720 mg).

In some embodiments, a first therapeutically effective dose comprises administering intravenously an anti-Aβ protofibril antibody at 10 mg/kg (e.g., administering BAN2401 at 10 mg/kg), biweekly, e.g., until a patient has a tau PET level that has increased by no more than 0.05-0.1 after 13 or 18 months of treatment as evaluated by tau PET SUVR in the temporal region or e.g., until a tau PET level has improved relative to an untreated control subject, before switching to a weekly subcutaneous maintenance dose (e.g., at a dose of 720 mg). In some embodiments, a first therapeutically effective dose comprises administering intravenously an anti-Aβ protofibril antibody at 10 mg/kg (e.g., administering BAN2401 at 10 mg/kg), biweekly, e.g., until a patient has a tau PET level that has increased by no more than 0.05-0.1 after 13 or 18 months of treatment as evaluated by tau PET SUVR in the temporal region or e.g., until a tau PET level has improved relative to an untreated control subject, before switching to a biweekly subcutaneous maintenance dose (e.g., at a dose of 720 mg or at a dose of 360 mg).

In some embodiments, a first therapeutically effective dose comprises subcutaneously administering an anti-Aβ protofibril antibody weekly, e.g., subcutaneous injection of 720 mg comprising two concurrent, e.g., sequential, injections in a given week of 360 mg (2×1.8 mL of 400 mg/2 mL) of the subcutaneous formulation, e.g., until a patient has a tau PET level that has increased by no more than 0.05-0.1 after 13 or 18 months of treatment as evaluated by tau PET SUVR in the temporal region or e.g., until a tau PET level has improved relative to an untreated control subject, before switching to a weekly subcutaneous maintenance dose of 720 mg. In some embodiments, a first therapeutically effective dose comprises subcutaneously administering an anti-Aβ protofibril antibody weekly, e.g., subcutaneous injection of 720 mg of the subcutaneous formulation, e.g., until a patient has a tau PET level that has increased by no more than 0.05-0.1 after 13 or 18 months of treatment as evaluated by tau PET SUVR in the temporal region or e.g., until a tau PET level has improved relative to an untreated control subject, before switching to a weekly subcutaneous maintenance dose of 360 mg. In some embodiments, a first therapeutically effective dose comprises subcutaneously administering an anti-Aβ protofibril antibody weekly, e.g., subcutaneous injection of 720 mg of the subcutaneous formulation, e.g., until a patient has a tau PET level that has increased by no more than 0.05-0.1 after 13 or 18 months of treatment as evaluated by tau PET SUVR in the temporal region or e.g., until a tau PET level has improved relative to an untreated control subject, before switching to a biweekly subcutaneous maintenance dose (e.g., at a dose of 720 mg). In some embodiments, a first therapeutically effective dose comprises subcutaneously administering an anti-Aβ protofibril antibody weekly, e.g., subcutaneous injection of 720 mg of the subcutaneous formulation, e.g., until a patient has a tau PET level that has increased by no more than 0.05-0.1 after 13 or 18 months of treatment as evaluated by tau PET SUVR in the temporal region or e.g., until a tau PET level has improved relative to an untreated control subject, before switching to a monthly subcutaneous maintenance dose of 720 mg.

### C. Subjects with Low Tau PET

In various embodiments, methods are disclosed herein for selecting a subject, treating, monitoring treatment, and making decisions regarding further treatment, e.g., maintenance dosing, in patients receiving an anti-amyloid β protofibril antibody (also called an “anti-amyloid R (Aβ) protofibril antibody” or an “anti-Aβ protofibril antibody”), such as BAN2401. In some embodiments, the subject has a low tau level in a global brain measurement, for example, as measured by tau PET. A low level of tau PET may refer to a low level of tau aggregation as imaged by PET scan imaging, e.g., a low level of cortical tau aggregation. In some embodiments, a subject with low tau PET also has accumulation of tau in certain brain regions, e.g., one or more early Braak regions or a composite of regions where tau accumulates in early AD. In some embodiments, the presence of tau in these regions (e.g., as measured by PET) may be measured in conjunction with, or in lieu of, measuring low tau in a global brain measurement (e.g., as measured by PET) to identify a subject suitable for treatment as disclosed herein. In some embodiments, one or more additional biomarker (e.g., a plasma and/or CSF marker such as an amyloid beta 42:40 ratio, a phospho-tau, and/or MTBR-tau243), may be measured in conjunction with, or in lieu of, measuring low tau in a global brain measurement (e.g., as measured by PET).

Without being bound by theory, a surprising finding is that an anti-amyloid protofibril antibody, such as BAN2401 (i.e., lecanemab) may be especially efficacious in subjects having a low level of tau, e.g., a low tau PET level as measured in a global brain measurement and/or as measured by any of the proxy measurements for low global tau discussed herein (e.g., a serum measure such as an amyloid beta 42:40 ratio or a phospho-tau, or a CSF measure such as MTBR-tau243, or a plasma marker such as MTBR-tau243). In previous studies, the effects of anti-amyloid antibodies were hypothesized to be greatest in subjects having at least an intermediate level of tau, corresponding to early symptomatic AD and tau pathology. For example, the Trailblazer clinical study enrolled subjects with an intermediate level of tau as defined by threshold tau PET levels defined in the study, as well as a smaller number of subjects with high levels of tau, representing a later stage of disease progression. However, subjects with tau PET levels below a threshold were not included in the Trailblazer study.

In contrast, results from the present studies indicate the surprising effects of anti-A protofibril antibodies such as lecanemab, in treating subjects with low tau PET. In some embodiments, treatment with anti-amyloid antibodies results in improved outcomes, as determined by measurements of clinical function and/or biomarkers associated with AD, in subjects with low tau PET levels when compared to subjects with intermediate and/or high tau PET levels.

In some embodiments, the thresholds for determining low, intermediate, and high tau PET levels are determined for a given PET scanning protocol and tau PET tracer. In some embodiments, the threshold value may be determined using an MK6240 radiotracer. Comparable thresholds may be readily identified using other tracers. In some embodiments, the threshold value may be determined using a common scale for tau PET which reflects analysis methods and/or measurements obtained with different tau PET tracers.

In some embodiments, the low level of tau is a level tau as measured by PET, such as a tau-PET standardized uptake value ratio (SUVR), that is below a threshold, e.g., a threshold set in a global brain measurement.

In some embodiments, the global brain measurement is a measurement of tau PET in the whole cortical gray matter (e.g., a tau PET measurement of cortical tau aggregation). In some embodiments, the tau PET level is measured using an MK6240 radiotracer. In some embodiments, the threshold tau PET level is a tau PET SUVR of about 1.1, preferably about 1.06, as measured with a MK6240 PET scan, e.g., of whole cortical gray matter.

A subject having a low level of tau in the global brain measurement (e.g., whole cortical gray matter) may have a higher level of tau in a local brain region (e.g., medial temporal region, meta-temporal region, and/or temporal region).

In some embodiments, the local brain region is an early Braak region (e.g., Braak regions I, II, or III). In some embodiments, the early Braak region comprises transentorhinal cortex, entorhinal cortex, hippocampus, amygdala, parahippocampal gyrus, fusiform gyrus, and/or lingual gyrus. In some embodiments, the local brain region is a composite of regions that accumulate tau in early AD. In some embodiments, the composite region comprises a temporal region, a medial temporal region, and/or a meta-temporal region. In some embodiments, the local brain region is the medial temporal region (e.g., the entorhinal cortex).

In some embodiments, treating AD in a subject having low tau PET levels comprises reducing, slowing, and/or reversing decline in a measure of cognitive function, e.g., in the subject receiving the therapeutically effective dose of the anti-Aβ protofibril antibody, as compared to a control. In some embodiments, a measure of cognitive function in a subject who has been treated is compared to a baseline measure obtained from the same subject before treatment, or to a reference control. In some embodiments, the decline occurs between the time point when cognitive function is first measured (e.g., a baseline measure) and one or more later time points when cognitive function is measured again. In some embodiments, the later time point is at least 6 months, 12 months, 18 months, 21 months, or 24 months after the baseline measure. In some embodiments, the control is a subject who does not receive a therapeutically effective dose of an Aβ protofibril antibody, e.g., a subject who receives no treatment, or a subject who receives a placebo. In some embodiments, the control is a subject who does not receive lecanemab. In some embodiments, a control is a reference measurement, such as an averaged measurement that combines population data from more than one subject and is representative of a subject who does not receive treatment. In some embodiments, the measure of cognitive function is CDR-SB, ADAS-Cog14, and/or ADCS MCI-ADL.

In some embodiments, patients with low tau PET levels may have less tau accumulation than patients with higher tau PET levels. The disclosure herein shows that, surprisingly, these patients may respond better to treatment, e.g., treatment with an anti-Aβ protofibril antibody, e.g., BAN2401, as compared to patients with more tau PET as studied in clinical trials by others (e.g., TRAILBLAZER—ALZ3). In some embodiments, a patient may be classified as having a low level of tau if the tau PET level is below a threshold value. In some embodiments, a level of tau is calculated from a global tau load from a whole brain signal, e.g., using the Tau^{IQ} (also called “TauIQ”) algorithm. In some embodiments, a patient may be classified as having a low level of tau if a level of tau as measured by PET using an MK tracer is below about 1.1, e.g., below about 1.0. In some embodiments, a patient may be classified as having a low level of tau if the tau PET level as measured using an MK tracer is below 1.06. In some embodiments, a patient may be classified as having an intermediate level of tau if the tau PET level as measured by MK tracer is between about 1.1 and 3.0, e.g., between 1.06 and 2.91. In some embodiments, a patient may be classified as having a high level of tau if the tau PET level as measured by MK tracer is above about 3.0, e.g., above 2.91. In some embodiments, a patient may be classified as having a low level of tau based on a percentile ranking relative to other patients selected for treatment with an anti-Aβ protofibril antibody, e.g., BAN2401. In some embodiments, a cut-off value may be used to remove outliers before determining a percentile ranking. In some embodiments, a patient with a low level of tau, e.g., a low tau PET level, may be identified as having a level of tau at least one standard deviation below the mean level of tau for a patient population. As would be understood by one of ordinary skill in the art, the threshold values for classifying patients as having low, intermediate, or high tau PET levels may vary based on the methodology used to determine a tau level in the brain, e.g., depending on the tau PET methodology and tracer used in the tau PET imaging. In some embodiments, a patient may be classified as having a low level of tau if the tau PET level, as measured by the tracer MK6240 in the whole cortical gray matter (e.g., a measure of cortical tau aggregation) is below a threshold value. In some embodiments, a level of tau is calculated from a global tau load from the whole cortical gray matter, e.g., using the Tau^{IQ} algorithm. In some embodiments, a patient may be classified as having a low level of tau if a level of tau as measured by PET as measured by MK6240 tracer in the whole cortical gray matter is below about 1.1, e.g., below about 1.0. In some embodiments, a patient may be classified as having a low level of tau if the tau PET level as measured by PET as measured by MK6240 tracer in the whole cortical gray matter is below 1.06. In some embodiments, a patient may be classified as having an intermediate level of tau if the tau PET level as measured by MK6240 tracer in the whole cortical gray matter is between about 1.1 and 3.0, e.g., between 1.06 and 2.91. In some embodiments, a patient may be classified as having a high level of tau if the tau PET level as measured by MK6240 tracer in the whole cortical gray matter is above about 3.0, e.g., above 2.91. In some embodiments, tau PET levels as measured by MK6240 in the whole cortical gray matter may be referred to as “total tau” or “total tau aggregation.”

### D. Measurement of Tau PET Level

The disclosure and methods discussed herein turn in part on the discovery that treatment comprising an anti-Aβ protofibril antibody such as BAN2401 can lead to a reduced rate of tau accumulation, as measured by a tau PET level (e.g., as measured by tau PET imaging), e.g., in a brain region such as a temporal region, as compared to a control patient.

In some embodiments, the treatment is administered to a subject having a low tau PET level in a global brain measurement (e.g., whole cortical gray matter) and/or who has been identified by any of the proxy measures for low global tau PET discussed herein. A subject having a low tau PET in the global brain measurement may have a higher level of tau PET in a local brain region. In some embodiments, the local brain region is an early Braak region (e.g., Braak regions I, II, or III). For example, the early Braak region may comprise entorhinal cortex, hippocampus, amygdala, parahippocampal gyrus, fusiform gyrus, and/or lingual gyrus. In some embodiments, the local brain region is a composite of regions that accumulate tau in early AD. In some embodiments, the composite region comprises a temporal region, a medial temporal region, and/or a meta-temporal region. In some embodiments, the local brain region is the medial temporal region (e.g., the entorhinal cortex, hippocampus, parahippocampal gyrus and temporal pole (medial and/or inferio-lateral tip of the temporal lobe). Accordingly, reducing tau accumulation may refer to reducing, slowing, and/or reversing tau accumulation in a local brain region.

In some embodiments, treatment with an Aβ protofibril antibody reduces the tau PET level (e.g., tau aggregation or accumulation), as measured by an adjusted mean change from baseline tau PET SUVr level of less than about 0.1, e.g., 0.05. In some embodiments, the reduction in tau PET from baseline is greater in a subject who receives lecanemab than in a control.

In some embodiments, a measure of tau aggregation in a subject who has been treated is compared to a baseline measure obtained from the same subject before treatment, or to a reference control. In some embodiments, the reference control is a control subject who has not been treated (e.g., a subject who received a placebo). In some embodiments, the reference control is a measurement obtained from a population of control subjects.

In some embodiments, this correlates with reduced brain amyloid load and improved cognitive outcomes in subjects. Without being bound by theory, a tau PET level (e.g., a tau PET standard uptake value ratio (SUVr)) may be used, in various embodiments, as a less invasive and/or additional biomarker for refining the measurement of treatment efficacy and/or to allow for monitoring and treatment decisions. Such decision may include whether to increase or decrease the amount of an anti-Aβ protofibril antibody being administered, whether to increase or decrease the frequency of administration, whether to introduce a further therapeutic agent, and/or whether to discontinue treatment with the anti-Aβ protofibril antibody.

As used herein, the term “tau PET” refers to tau positron emission tomography imaging. In some embodiments, tau PET imaging (also referred to as a tau PET scan) is performed to assess for tau pathology. In some embodiments, tau PET is assessed with a PET tracer and uses the same tracer in follow-up assessments. In preferred embodiments, the PET imaging uses a [18F]MK-6240 (also called “florquinitau”) tracer and referred to herein as the “MK Tracer”). In some embodiments, the PET tracer is an arylquinoline derivatives (e.g., [18F]THK5317 and [18F]THK5351), a pyridoindole derivative (e.g., [18F]AV-1451 also known as [18F]-flortaucipir), or a phenyl/pyridinyl-butadienyl-benzothiazone/benzothiazolium (PBB) derivative such as [11C]PBB3. In some embodiments, the PET tracer is [18F]-RO-948, [18F]-PI-2620, [18F]-JNJ-311, and [18F]-GTP1.

Tau positron emission tomography (PET) imaging can be used to confirm the presence of tau pathology in the brain of early AD subjects in the screening phase of the study and/or to evaluate the effects of the at least one anti-Aβ protofibril antibody on tau levels in the brain, by whole brain analysis (e.g., whole cortical gray matter, or the average of 5-6 cortical regions) and/or brain region analysis (e.g., in a temporal brain region).

In some embodiments, tau PET imaging across different regions of the brain may be used to determine tau PET levels at a global or local level. A global brain measurement may comprise a tau PET imaging analysis of a whole brain, most of the whole brain, gray matter of the brain, or a large section of the brain which may be considered representative for a global brain measurement (e.g., whole cortical gray matter). In some embodiments, the global brain measurement is a tau PET measurement of 5-6 cortical regions. In some embodiments, the global brain measurement is a tau PET measurement of whole cortical gray matter. In some embodiments, a global brain measurement is a tau PET measurement of cortical tau aggregation. A local brain measurement may comprise tau PET imaging analysis of a local brain region. In some embodiments, the local brain region comprises one or more brain structures, e.g., brain structures defined by structures or morphology. In some embodiments, a subject may have a first tau PET level in a global brain measurement, and a second tau PET level in a local brain region. In some embodiments, the first tau PET level is lower than the second tau PET level. In some embodiments, the first tau PET level is higher than the second PET level. In some embodiments, the first tau PET level is equal to the second tau PET level.

In some embodiments, the PET scan uses a [18F]MK-6240 (florquinitau) tracer. In some embodiments, the PET scan uses a tau PET tracer such as an arylquinoline derivatives (e.g., [18F]THK5317 and [18F]THK5351), a pyridoindole derivative (e.g., [18F]AV-1451 also known as [18F]-flortaucipir), or a phenyl/pyridinyl-butadienyl-benzothiazone/benzothiazolium (PBB) derivative such as [11C]PBB3. In some embodiments, the PET tracer is [18F]-RO-948, [18F]-PI-2620, [18F]-JNJ-311, and [18F]-GTP1. In some embodiments, tau load can be identified by a PET imaging uptake visual read, e.g., by a trained radiologist. In embodiments, regions of the brain (e.g., lobes of the brain) are assessed for uptake of the imaging agent (e.g., the tau PET tracer).

A region of the brain is an area, part, portion, or division of the brain. A region of the brain may be a region defined in a standard neuroanatomy atlas. A region may correspond to a brain lobe, or it may constitute a larger or smaller portion of the brain. A region may require or be defined by specific anatomical landmarks. A region of the brain may be defined according to characteristics that are specific to a particular experiment or study. A region of the brain may be defined according to its location, function, anatomy, connections to other brain regions, and/or properties of PET tracer uptake. In some embodiments, the region of the brain is the whole cortex.

In some embodiments, the region of the brain is a composite of more than one brain region, e.g., regions that accumulate tau in early AD. In some embodiments, the region of the brain is a composite of more than one lobe, subregion, and/or structure in the brain.

For example, a cortical composite may comprise more than one brain structure from the cortex. In some embodiments, a composite region in a PET scan is region of interest (ROI) comprising more than one brain region, e.g., a volume-weighted average of more than one brain region.

In some embodiments, the region of the brain is a temporal region. In some embodiments, the region of the brain is a medial temporal region. In some embodiments, the region of the brain is primarily a lateral temporal region. In some embodiments, a region of the brain may be called a meta-temporal region.

The temporal region as used herein comprises at least one part of the temporal lobe. For example, the temporal region may comprise the superior posterior part of the temporal lobe, the superior anterior part of the temporal lobe, the posterior part of the temporal lobe, the middle inferior part of the temporal lobe, and the fusiform gyrus. The temporal region may comprise these structures from both the left and right hemispheres of the brain. In some embodiments, the temporal region comprises a lateral temporal region.

In some embodiments, the medial temporal region comprises the hippocampus, the anterior medial part of the temporal lobe, the anterior inferior lateral part of the temporal lobe, the parahippocampal cortex, and the entorhinal cortex. The medial temporal region may comprise these structures from both the left and right hemispheres of the brain.

In some embodiments, the medial temporal region comprises the entorhinal cortex, hippocampus, parahippocampal gyrus, and/or temporal pole (medial and inferio-lateral tip of the temporal lobe). The medial temporal region may comprise these structures from both the left and right hemispheres of the brain.

In some embodiments, meta-regions of interest (ROIs) are defined as those regions of the brain that have certain properties, such as the regions having most tau deposition in AD patients or the regions where tau PET imaging differs between groups of patients (e.g., cognitively unimpaired individuals, e.g., those with normal amyloid PET as compared with cognitively unimpaired individuals with abnormal amyloid PET). Examples of a meta-ROI in a temporal region may be found in Jack et al., Alzheimer's Dementia 13, 205-216 (2017). In some embodiments a meta-ROI may comprise a meta-temporal region (also called a temporal meta region). In some embodiments, the meta-temporal region comprises the amygdala, the parahippocampal cortex, the middle inferior part of the temporal lobe, the fusiform gyrus, the posterior part of the temporal lobe, and the entorhinal cortex. The meta-temporal region may comprise these structures from both the left and right hemispheres of the brain.

Accordingly, PET levels may be assessed in any of the regions of the brain described herein and/or other brain regions. in some embodiments, a temporal region is assessed for a tau PET level. In some embodiments, a frontal region is assessed for a tau PET level. In some embodiments, a parietal region is assessed for a tau PET level. In some embodiments, an occipital region is assessed for a tau PET level. In some embodiments, a cingulate region is assessed for a tau PET level.

In some embodiments, the whole cortical gray matter is assessed for a tau PET level. In some embodiments, a region, e.g., a reference region, used for assessing a tau PET level is the ventral cerebellum (Cb).

As used herein, a “tau level in a brain”, “tau level”, and “tau load” are used interchangeably. A tau level may be assessed by PET imaging (“tau PET level”). As used herein, a tau PET level refers to a measurement of a level of tau in a brain region, e.g., a temporal region, by PET. In some embodiments, a tau PET level is calculated from a global tau load from a whole brain signal, e.g., using the Tau^{IQ} algorithm (also called “TauIQ”). In some embodiments, a “tau PET level” can be identified in tau PET imaging by a standard uptake value ratio (SUVr or SUVR). The SUVr may be a measurement of tau PET tracer uptake, e.g., in a region of a patient's brain, as compared (e.g, normalized) to a reference region in the same patient. Methods for calculating tau PET SUVr are known in the art and may include those described herein. One exemplary method for quantitative analysis (e.g, computing) of SUVr is the PMOD PNEURO Biomedical Image Quantification Software (PMOD Technologies, Zurich, Switzerland). In some embodiments, PET images are first assessed for subject movement in the X, Y, and Z planes and corrected for motion, if needed, before individual images (e.g., 5-minute emission frames) are averaged, e.g., using a PMOD Averaging Function (PET frames averaged to increase the signal to noise ratio). In some embodiments, corresponding MRIs from subjects are prepared (e.g., using matrix size reduction processing, cropping of the MRI to include only the brain, segmentation to separate images into binary maps of gray matter, white matter, and CSF, and stripping the image of skull leaving only brain mask). In some embodiments, the averaged PET images and prepared MRIs are matched using the PMOD Matching Function, placing the images in the same orientation. In some embodiments, a Brain Normalization function, e.g., as provided by PMOD software, is used along with Brain Norm and Rigid Matching transformation matrices, to produce an averaged PET. In some embodiments, this averaged PET which is normalized to the MNInst space (Senjem et al, 2005) that is in the same orientation as the subject's segmented MRI for quantitative analysis. In some embodiments, the PMOD Mask Function is used to mask the brain and zero the image outside of the mask to create a Normalized Gray Matter PET and a Normalized White Matter PET. Standard uptake values (SUVs) may be calculated for all gray matter mapped regions and the 3 white matter regions (pons, cerebellar white, and subcortical white) using PMOD software calculated using the normalized PET, subject weight, and injected dose of tracer to arrive at the units of SUVs. In some embodiments, the SUVr is the ratio of the global cortical average as compared to a reference region of choice. In some embodiments, a whole cerebellum mask is used as the reference region. In some embodiments, the reference region is subcortical white matter, ventral cerebellum, derived whole cerebellum, whole cerebellum adjusted by subcortical white matter, cerebellar gray matter, and composite reference regions consisting of cerebellar cortex, pons subcortical white matter, and cerebella white matter.

In some embodiments, a tau PET level is assessed with a PET tracer. In some embodiments, the PET tracer is [18F]MK-6240. In some embodiments, a tau PET level can be used to classify patients as having different levels of tau. For example, in some embodiments, a patient may be classified as having a low level of tau if the tau PET level is below a threshold value. The threshold value may be identified as tau levels in the whole cortex, or, alternatively, in a particular region of the interest. In some embodiments, the threshold value may be a tau PET level measured in the whole cortex (e.g., whole cortical gray matter). In some embodiments, the threshold values are cut-offs for classifying a patient as having a low tau PET level, an intermediate tau PET level, or a high tau PET level. In some embodiments, a patient may be classified as having a low level of tau if a level of tau as measured in a particular region of the interest, e.g., whole cortical gray matter, by PET using, e.g., an MK tracer, is below about 1.1, e.g., below about 1.0. In some embodiments, a patient may be classified as having a low level of tau if the tau PET level as measured using an MK tracer is below 1.06. In some embodiments, a patient may be classified as having an intermediate level of tau if the tau PET level as measured by MK tracer is between about 1.1 and 3.0, or between 1.06 and 2.91. In some embodiments, a patient may be classified as having a high level of tau if the tau PET level as measured by MK tracer is above about 3.0, e.g., above 2.91. In some embodiments, the thresholds for tau PET levels are <1.06 (low levels), 1.06 to 2.91 (intermediate levels), and >2.91 (high levels), when the tau PET levels are determined in the whole cortex, e.g., using the PET tracer is [18F]MK-6240. In some embodiments, cut-offs may be determined according to tau PET levels in a particular brain region. As would be understood by one of ordinary skill in the art, the threshold values for classifying patients as having low, intermediate, or high tau PET levels may vary based on the methodology used to determine a tau level in the brain, e.g., depending on the tau PET methodology and tracer used in the tau PET imaging. In some embodiments, the threshold value may be determined using a common scale for tau PET which reflects analysis methods and/or measurements obtained with different tau PET tracers.

In some embodiments, the adjusted mean change from baseline is measured prior to treatment and at least once after the start of treatment, e.g., over a period of at least 6 months after an initial dose of a treatment. In some embodiments, the adjusted mean change from baseline is measured over a period of at least 12 months after an initial dose of a treatment. In some embodiments, the adjusted mean change from baseline is measured over a period of at least 13 months after an initial dose of a treatment. In some embodiments, the adjusted mean change from baseline is measured over a period of at least 18 months after an initial dose of a treatment. In some embodiments, the adjusted mean change from baseline is measured over a period of at least 24 months after an initial dose of a treatment.

In some embodiments, after administration of the first dose of the composition the adjusted mean change from baseline in a subject's tau PET SUVr value is less than 0.15. In some embodiments, after administration of the first dose of the composition the adjusted mean change from baseline in a subject's tau PET SUVr value is less than 0.10. In some embodiments, after administration of the first dose of the composition the adjusted mean change from baseline in a subject's tau PET SUVr value is less than 0.05. In some embodiments, 13 months after an initial dose of a treatment, the adjusted mean change from baseline in a subject's tau PET SUVr value is less than 0.15. In some embodiments, after administration of the first dose of the composition the adjusted mean change from baseline in a subject's tau PET SUVr value is less than 0.05. In some embodiments, 18 months after an initial dose of a treatment, the adjusted mean change from baseline in a subject's tau PET SUVr value is less than 0.15.

In some embodiments, the adjusted mean change from baseline in a subject's tau PET SUVr is measured in a local brain region. For example, the tau PET SUVr may be measured in an early Braak region (e.g., Braak regions I, II, or III). The early Braak region may comprise transentorhinal cortex, entorhinal cortex, hippocampus, amygdala, parahippocampal gyrus, fusiform gyrus, and/or lingual gyrus. In some embodiments, the local brain region is a composite of regions that accumulate tau in early AD. In some embodiments, the composite region comprises a temporal region, a medial temporal region, and/or a meta-temporal region. In some embodiments, the local brain region is the medial temporal region (e.g., the entorhinal cortex). In some embodiments, the subject may have a low tau PET level in a global brain measurement but may have a higher tau PET level in a local brain region. In some embodiments, the subject may have a higher tau PET level in a first local brain region but may not have the higher tau PET level in a second local brain region. For example, the first local brain region having the higher tau PET level may be an early Braak region (e.g., Braak region I, II, or III), while the second local brain region not having the higher PET level may be a later Braak region (e.g., Braak region IV, V, or VI), reflecting progression of AD.

Additional methods for measuring tau by PET are known in the art. Methods may include the Tau^{IQ} algorithm (see, e.g., Whittington et al., J. Nucl Med. 2021 Sep. 1; 62(9):1292-1300 for quantitatively measuring tau PET radiotracers).

The measurement of a tau PET level may be used alone to evaluate treatment efficacy, or in conjunction with one or more additional criteria, such as one or more measurement in a biofluid (e.g., p-tau181 and/or a ratio of Aβ1-42 to Aβ1-40), PET measurement of Aβ (e.g., via radiotracer uptake), indication of AD based on MRI evaluation of brain anatomy (e.g., MRI-based predictions of Aβ plaque formation), and/or behavioral measures, as discussed herein. Such assays may also be used to diagnose patients eligible for treatment (e.g. by measuring a tau PET level and determining a subject is suitable for treatment because of a higher tau PET level than observed in a healthy control subject, alone or in conjunction with measuring one or more additional marker of AD pathology in the subject). In some embodiments, a subject is selected for treatment because of a high tau PET level in a region of the brain of the subject, wherein the tau PET level is greater than a subject who does not have AD. In some embodiments, a subject is selected for treatment because of a low tau PET level in the temporal region of the brain of the subject. For example, the, wherein the tau PET level may be less than about 1.1, e.g., less than 1.06, e.g., as measured using the PET tracer [18F]MK-6240. In some embodiments, a subject is selected for treatment because of a high tau PET level in the temporal region of the brain of the subject. For example, wherein the tau PET level may be greater than about 1.1. In some embodiments, a subject is selected for treatment because of a low tau PET level in the temporal region of the brain of the subject. For example, the tau PET level may be less than about 1.1. In some embodiments, a subject is selected for treatment because of a high tau PET level in the temporal region of the brain of the subject. For example, the tau PET level may be greater than about 1.1. In some embodiments, the measurement of a tau PET level may be used in place of another method of measuring brain tau levels and/or in place of another marker of Aβ. In some embodiments, the measurement of a tau PET level may be used in conjunction with measuring one or more additional markers. In some embodiments, a patient may be monitored by one or more additional biomarkers such as, but not limited to: (a) tau detected by PET scan from either a visual read or semiquantitative thresholds (SUVr); (b) cerebrospinal fluid (CSF) total tau (t-tau); and/or (c) blood biomarkers (such as plasma total tau (T-tau), and/or phosphorylated tau (P-tau)(e.g., p-tau181)). In some embodiments, a subject's tau PET level may be monitored by measuring a CSF MTBR-tau243 or a plasma MTBR-tau243 species alone or in conjunction with one or more additional biomarker. In some embodiments, a patient's tau PET level may be monitored in conjunction with one or more of a ratio of Aβ1-42 to Aβ1-40 and/or a p-tau181 measurements in a fluid sample, e.g., a blood sample. In some embodiments, the combination comprises a serum or plasma GFAP measurement. In some embodiments, the measurement of a tau PET level may be used in place of another method of measuring brain tau levels for determining treatment efficacy and/or making treatment decisions such as whether to continue treatment, switch to a maintenance dose, etc.

In some embodiments, a tau PET level changes during the course of disease progression or treatment in manner that correlates with other biomarkers. Accordingly, a tau PET level (e.g., a low tau PET level as measured in a global brain measurement) may be used to select subjects for treatment and/or a quantity (e.g., level) of a biomarker may be used to select a subject. For example, a biomarker such as an amyloid PET level of <60 CL may be used to select a subject. One or more of CSF t-tau; CSF p-tau; CSF MTBR-tau243; CSF NfL; or a ratio of Aβ1-42 to Aβ1-40, NfL, p-tau181, MTBR-tau243 or GFAP in a fluid sample, e.g., a blood sample such as blood serum or blood plasma may be used to select a subject. In some embodiments, one or more of these biomarkers may serve as a proxy for a tau PET measurement, e.g., a low tau PET, to select a subject for treatment.

In some embodiments, measurements of tau PET may be at one or more time points in subjects receiving treatment for AD and compared to a baseline tau PET measurement (e.g., a measurement from the subject before treatment) in order to monitor treatment efficacy and/or determine whether changes, if any, should be made to the treatment regimen. In some embodiments, a change in tau PET level may indicate that the treatment is efficacious. For example, a decrease in the tau PET level as compared to a baseline tau PET level may indicate whether the treatment is efficacious. In some embodiments, a treatment regimen may be changed so that treatment is administered at a maintenance dose. In some embodiments, the treatment regimen may be changed to increase a dose or a frequency of administration. In some embodiments, measures of one or more biomarkers may be used to monitor treatment efficacy and/or determine changes, if any, to the treatment regimen. In some embodiments, the one or more biomarkers are amyloid PET, CSF t-tau; CSF p-tau; CSF MTBR-tau243; CSF NfL; or a ratio of Aβ1-42 to Aβ1-40, NfL, p-tau181, MTBR-tau243 or GFAP in a fluid sample, e.g., a blood sample such as blood serum or blood plasma. For example, a change in one or more of the biomarkers as compared to a baseline level may indicate whether the treatment is efficacious. In some embodiments, a treatment regimen may be changed so that treatment is administered at a maintenance dose. In some embodiments, the treatment regimen may be changed to increase a dose or a frequency of administration. In some embodiments, a tau PET level may be used to calculate a relative change from a baseline measurement (e.g., a measurement of a tau PET level before beginning a treatment). In some embodiments, a tau PET level measurement may be repeated after the start of a treatment regimen to monitor treatment efficacy. In some embodiments, a tau PET level is measured prior to treatment and at least once after the start of treatment, e.g., over a period of at least 6 months after an initial dose of a treatment. In some embodiments, a tau PET level is measured over a period of at least 12 months after an initial dose of a treatment. In some embodiments, a tau PET level is measured over a period of at least 13 months after an initial dose of a treatment. In some embodiments, a tau PET level is measured over a period of at least 18 months after an initial dose of a treatment. In some embodiments, a rate of change of a tau PET level is calculated based on the measurements from a subject. In some embodiments, a rate of change of a tau PET level is calculated based on the measurements from a subject, wherein one measurement is taken from a subject before a treatment, and at least a second measurement is taken after a treatment. In some embodiments, a rate of change of a tau PET level is calculated based on the measurements from a subject, wherein one measurement is taken from a subject before a treatment, and at least a second measurement is taken after a treatment, and wherein the tau PET level is measured at least 6 months after an initial dose of a treatment. In some embodiments, a rate of change of a tau PET level is calculated based on the measurements from a subject, wherein one measurement is taken from a subject before a treatment, and at least a second measurement is taken after a treatment, and wherein the tau PET level is measured at least 12 months after an initial dose of a treatment. In some embodiments, a rate of change of a tau PET level is calculated based on the measurements from a subject, wherein one measurement is taken from a subject before a treatment, and at least a second measurement is taken after a treatment, and wherein the tau PET level is measured at least 13 months after an initial dose of a treatment. In some embodiments, a rate of change of a tau PET level is calculated based on the measurements from a subject, wherein one measurement is taken from a subject before a treatment, and at least a second measurement is taken after a treatment, and wherein the tau PET level is measured at least 18 months after an initial dose of a treatment. In some embodiments, a tau PET level that increased by no more than 0.05-0.1 over a 13-month period in a region of the brain indicates treatment efficacy. In some embodiments, a tau PET level that increased by no more than 0.05-0.1 over an 18-month period in a region of the brain indicates treatment efficacy. In some embodiments, a tau PET level that increased by no more than 0.05-0.1 over a 13-month period in the temporal region of the brain indicates treatment efficacy. In some embodiments, a tau PET level that increased by no more than 0.05-0.1 over an 18-month period in the temporal region of the brain indicates treatment efficacy.

In some embodiments, a rate of change of a tau PET level is calculated based on two measurements from a subject. In some embodiments, a rate of change of a tau PET level is calculated based on more than two measurements from a subject. In some embodiments, a rate of change of a tau PET level indicates the rate of tau accumulation in the brain of a subject.

In some embodiments, a rate of change of a tau PET level is calculated based on at least two measurements from a subject, wherein one measurement is taken from a subject before a treatment, and a second measurement is taken after the start of treatment, wherein treatment continues for at least 13 or 18 months after an initial dose of a treatment. In some embodiments, the rate of change of the tau PET level is compared to the rate of change of the tau PET level in an untreated control subject who has AD and has not received treatment. In some embodiments, a lower rate of tau PET increase relative to an untreated control subject indicates treatment efficacy. In some embodiments, a lower rate of tau PET increase over a 6-month period relative to an untreated control subject indicates treatment efficacy. In some embodiments, a lower rate of tau PET increase over a 12-month period relative to an untreated control subject indicates treatment efficacy. In some embodiments, a lower rate of tau PET increase over a 13-month period relative to an untreated control subject indicates treatment efficacy. In some embodiments, a lower rate of tau PET increase over an 18-month period relative to an untreated control subject indicates treatment efficacy.

In some embodiments, an increase in a tau PET level and/or rate of change relative to an untreated control subject who has AD but does not receive treatment may indicate a need to continue treatment, e.g., beyond a 13 month or 18 month duration of treatment, or to select an increase in a dosing regimen. In some embodiments, a lower rate of change of a tau PET level relative to an untreated control subject (e.g., as measured at 13 or 18 months) may be used to indicate a treatment may be terminated (e.g., terminated in favor of a maintenance regimen) and/or to otherwise to determine a decrease in a dosing regimen or discontinuation in treatment. In some embodiments, an increased rate of change of a tau PET value relative to an untreated control subject may be used to determine whether to discontinue a maintenance dosing regimen, e.g., and return to the prior treatment regimen.

In some embodiments, a decreased rate of change in tau PET relative to an untreated control subject may indicate an effective treatment. In some embodiments, a decreased rate of change in tau PET relative to an untreated control subject may be used to determine whether to switch to a maintenance dosing regimen. In some embodiments, a tau PET level that increased by less than 0.05-0.1 over a 13-month period in a region of the brain (e.g., a temporal region) may indicate an effective treatment. In some embodiments, a tau PET level that increased by less than 0.05-0.1 over an 18-month period in a region of the brain may indicate an effective treatment. In some embodiments, a tau PET from a region of the brain (e.g., a temporal region) lower than an untreated control subject may indicate an effective treatment.

In some embodiments, an increase in a tau PET level and/or an increased rate of change in tau PET relative to an untreated control subject may be used to determine whether to discontinue a maintenance dosing regimen, e.g., and return to the prior treatment regimen. In some embodiments, a tau PET level that increased by more than 0.05-0.1 over a 13-month period in a region of the brain (e.g., a temporal region) may indicate an ineffective treatment. In some embodiments, a tau PET level that increased by more than 0.05-0.1 over an 18-month period in a region of the brain may indicate an ineffective treatment.

Methods of measuring clinical efficacy or monitoring a treatment may employ a set threshold to determine a change in brain tau levels, e.g., to identify a patient suitable for treatment, e.g., with an anti-Aβ protofibril antibody, or to determine whether to continue treatment, or to determine whether to switch to a maintenance dose, or to conclude a patient is amyloid negative. In some embodiments, the tau PET level threshold may be evaluated in conjunction with another measurement of brain amyloid load, such as an amyloid PET scan, a CSF or serum or plasma biomarker, to assist in determining whether a subject is suitable for treatment or continued treatment. In some embodiments, a tau PET level may be used in place of another method of measuring brain tau levels. In some embodiments, a tau PET level greater than a threshold level is used to determine if a patient should be treated. In some embodiments, a tau PET level of about 1 (e.g., 1.06) measured in the whole cortex indicates a patient with a low tau PET level who should be treated. In some embodiments, a tau PET level of about 1.06-2.91 measured in the whole cortex indicates a patient with an intermediate tau PET level who should be treated. In some embodiments, a tau PET level of about 2.91 or greater measured in the whole cortex indicates a patient with a high tau PET level who should be treated. In some embodiments, a subject is selected for treatment with an anti-amyloid β (Aβ) protofibril antibody, wherein the subject has a tau PET level in a region of the brain greater than about 1. In some embodiments, a subject is selected for treatment with an anti-amyloid 3 (Aβ) protofibril antibody, wherein the subject has a tau PET level in a region of the brain between about 1.06 and 3. In some embodiments, a subject is selected for treatment with an anti-amyloid 3 (Aβ) protofibril antibody, wherein the subject has a tau PET level in a region of the brain greater than about 3. In some embodiments, the region of the brain is the whole cortex. In some embodiments, the region of the brain is the temporal lobe, or a part (e.g, subdivision) of the temporal lobe. In some embodiments, the region of the brain comprises the temporal lobe and additional brain sections outside the temporal lobe. In some embodiments, the region of the brain is a temporal region. In some embodiments, the region of the brain is the meta-temporal region. In some embodiments, the region of the brain is the medial-temporal region.

In some embodiments, methods of selecting a patient for treatment and methods of treating may comprise selecting a patient and/or administering treatment to a patient having a tau brain level below a threshold, e.g., below a tau PET threshold, wherein the treatment may comprise any treatment disclosed herein, e.g., comprising an anti-Aβ protofibril antibody, e.g., BAN-2401. In some embodiments, a method to determine whether to continue treatment, or to determine whether to switch to a maintenance dose, comprises identifying a tau level, e.g., a tau PET level, below a threshold. In some embodiments, a patient with a low tau PET level may be selected for treatment. In some embodiments, a patient with a low tau PET level is provided with a treatment discussed herein, e.g., an anti-Aβ protofibril antibody, e.g., BAN2401.

### E. Measurement of Aβ42/40 Ratio

The disclosure and methods discussed herein depend in part on the discovery that not only can a tau PET level or a rate of change in a tau PET level be used to select a patient for treatment, to allow for monitoring and treatment decisions such as whether to increase or decrease the amount of antibody being administered, determine whether to increase or decrease the frequency of administration, determine whether to introduce a further therapeutic agent, determine whether to switch to a maintenance dose, and/or whether to discontinue treatment with the anti-Aβ protofibril antibody, but also that a tau PET level can be used in combination of other biomarkers, such as an Aβ42/40 ratio. Aβ42 and 40 be measured to calculate a ratio in blood samples, as disclosed in PCT/US2022/073576, which herein is incorporated by reference. A treatment comprising an anti-Aβ protofibril antibody such as BAN2401 can lead to an increase in the Aβ42/40 ratio that correlates with reduced brain amyloid load and improved cognitive outcomes in subjects.

In some embodiments, a treatment comprising an anti-Aβ protofibril antibody such as BAN2401 can lead to an increase in the Aβ42/40 ratio that correlates with reduced brain amyloid load and improved cognitive outcomes in subjects who have low tau PET levels in a global brain measurement. In some embodiments, the Aβ protofibril antibody increases the plasma Aβ42/40 ratio, as measured by an adjusted mean change from a baseline plasma Aβ42/40 ratio of at least about 0.003, 0.006, 0.007, 0.008, or 0.009. In some embodiments, the Aβ protofibril antibody increases the Aβ42/40 ratio to about 0.092 or above. In some embodiments, a measure of the Aβ42/40 ratio in a subject who has been treated is compared to a baseline measure obtained from the same subject before treatment, or to a reference control. In some embodiments, the change in the Aβ42/40 ratio occurs between the time point when the Aβ42/40 ratio is first measured (e.g., a baseline measure) and one or more later time points when the Aβ42/40 ratio is measured again. In some embodiments, the later time point is at least 6 months, 12 months, 18 months, 21 months, or 24 months after the baseline measure. In some embodiments, the control is a subject who does not receive a therapeutically effective dose of an Aβ protofibril antibody, e.g., a subject who receives no treatment, or a subject who receives a placebo. In some embodiments, the control is a subject who does not receive lecanemab. In some embodiments, a control is a reference measurement, such as an averaged measurement that combines population data from more than one subject and is representative of a subject who does not receive treatment.

Methods for measuring the Aβ42/40 ratio are known in the art, such as assays using LC MS/MS. Methods may include the PrecivityAD™ assay (see, e.g., Kirmess et al., J. Clinica Chimica Acta 519: 267-275 (2021)) and the Sysmex assay (https://www.eisai.com/news/2019/news201990.html) for measuring Aβ42 and Aβ40 in a sample to use in calculating a ratio.

The measurement of the Aβ42/40 ratio may be used alone to evaluate treatment efficacy, or in conjunction with one or more additional criteria, such as a tau PET level, a PET measurement of Aβ radiotracer update, MRI evaluation of Aβ plaque, and/or behavioral measures, as discussed herein. Such assays may also be used to diagnose patients eligible for treatment (e.g., by measuring an Aβ42/40 ratio and determining a subject is suitable for treatment because of a lower ratio than observed in a healthy control subject, alone or in conjunction with measuring one or more additional marker of AD pathology in the subject). In some embodiments, the measurement of an Aβ42/40 ratio may be used in place of another method of measuring brain amyloid levels, such as a PET scan for determining a subject is suitable for treatment. In some embodiments, the measurement of an Aβ42/40 ratio may be used in place of another method of measuring brain amyloid levels, such as a PET scan for determining treatment efficacy and/or making treatment decisions such as whether to continue treatment, switch to a maintenance dose, etc.

In some embodiments, an Aβ42/40 ratio measurement may employ a relative change from baseline measurement. In some embodiments, an Aβ42/40 ratio measurement may employ a set threshold to determine a change in brain amyloid levels, e.g., to identify a patient suitable for treatment, e.g., with an anti-Aβ protofibril antibody, or to determine whether to continue treatment, or to determine whether to switch to a maintenance dose, or to conclude a patient is amyloid negative. In some embodiments, the threshold may be evaluated in conjunction with another measurement of brain amyloid load, such as a PET scan, to assist in determining whether a subject is suitable for treatment or continued treatment. In some embodiments, an Aβ42/40 ratio threshold may be used in place of another method of measuring brain amyloid levels, such as a PET scan, to determine amyloid positivity. In some embodiments, an Aβ42/40 ratio threshold is at or about 0.09, 0.091, 0.092, 0.093, 0.094, 0.095, 0.096, 0.097, 0.099, 0.1. In some embodiments, the threshold is about 0.092. In some embodiments, the threshold is 0.092. In some embodiments, the threshold is about 0.094. In some embodiments, a decrease in the Aβ42/40 ratio below a threshold value may indicate a need to continue treatment or to select an increase in a dosing regimen. In some embodiments, an increase in the Aβ42/40 ratio above a threshold value may be used to indicate a treatment may be terminated (e.g., terminated in favor of a maintenance regimen) and/or to otherwise to determine a decrease in a dosing regimen or discontinuation in treatment. In some embodiments, a decrease in the Aβ42/40 ratio below a threshold value may be used to determine whether to discontinue a maintenance dosing regimen, e.g., and return to the prior treatment regimen.

In some embodiments, a tau PET level or a rate of change in a tau PET level and a level of an additional biomarker, e.g., an Aβ42/40 ratio, may be used to select a patient for treatment, to allow for monitoring and treatment decisions such as whether to increase or decrease the amount of antibody being administered, determine whether to increase or decrease the frequency of administration, determine whether to introduce a further therapeutic agent, determine whether to switch to a maintenance dose, and/or whether to discontinue treatment with the anti-Aβ protofibril antibody. In some embodiments, a tau PET level may be used to select a patient for treatment, e.g., a tau PET level greater than 1.4 as measured by amyloid PET SUVr in the temporal region of the brain in combination with an Aβ42/40 ratio, e.g., an Aβ42/40 ratio greater than 0.092. In some embodiments, a tau PET level may be used to select a patient for treatment, e.g., a tau PET level greater than 1.5 as measured by amyloid PET SUVr in the temporal region of the brain in combination with an Aβ42/40 ratio, e.g., an Aβ42/40 ratio at or greater than 0.092. Thresholds for determining amyloid positivity with an Aβ42/40 ratio are disclosed in PCT/US2022/073576, which herein is incorporated by reference. In some embodiments, a tau PET level that increased by less than 0.05-0.1 over a 13-month or 18-month period in a region of the brain (e.g., a temporal region) in combination with an Aβ42/40 ratio, e.g., an Aβ42/40 ratio at or greater than 0.092, may indicate an effective treatment. In some embodiments, a tau PET level that increased at a rate lower than an untreated control subject over a 13-month or 18-month period in a region of the brain (e.g., a temporal region) in combination with an Aβ42/40 ratio, e.g., an Aβ42/40 ratio at or greater than 0.092, may indicate an effective treatment. In some embodiments, a tau PET from a region of the brain (e.g., a temporal region) lower than an untreated control subject in combination with an Aβ42/40 ratio, e.g., an Aβ42/40 ratio at or greater than 0.092, may indicate an effective treatment. In some embodiments, an indication of the efficacy of treatment may be used to decrease the amount of antibody being administered, decrease the frequency of administration, or to switch to a maintenance dose. In some embodiments, a tau PET level that increased by more than 0.05-0.1 over a 13-month or 18-month period in a region of the brain (e.g., a temporal region) in combination with an Aβ42/40 ratio, e.g., an Aβ42/40 ratio at or below 0.092, may indicate an ineffective treatment. In some embodiments, a tau PET level that increased at a rate higher than an untreated control subject over a 13-month or 18-month period in a region of the brain (e.g., a temporal region) in combination with an Aβ42/40 ratio, e.g., an Aβ42/40 ratio at or below 0.092, may indicate an ineffective treatment. In some embodiments, a tau PET from a region of the brain (e.g., a temporal region) higher than an untreated control subject in combination with an Aβ42/40 ratio, e.g., an Aβ42/40 ratio at or below 0.092, may indicate an ineffective treatment. In some embodiments, an indication of the efficacy treatment may be used to increase the amount of antibody being administered, increase the frequency of administration, or to discontinue treatment.

### F. Measurement of a p-Tau Level

The disclosure and methods discussed herein depend in part on the discovery that not only can a tau PET level or a rate of change in a tau PET level be used to select a patient for treatment, to allow for monitoring and treatment decisions such as whether to increase or decrease the amount of antibody being administered, determine whether to increase or decrease the frequency of administration, determine whether to introduce a further therapeutic agent, determine whether to switch to a maintenance dose, and/or whether to discontinue treatment with the anti-Aβ protofibril antibody, but also that a tau PET level can be used in combination of other biomarkers, such as a phosphorylated tau (p-tau) level, including tau phosphorylated at 181 (P-tau181), 217 (P-tau217), and/or 231 (P-tau231). A p-tau level can be measured in CSF, serum, or plasma, as disclosed in PCT/US2022/079571, which herein is incorporated by reference. A treatment comprising an anti-Aβ protofibril antibody such as BAN2401 can lead to a decrease in a p-tau181 level that correlates with reduced brain amyloid load and improved cognitive outcomes in subjects.

In some embodiments, a treatment comprising an anti-Aβ protofibril antibody such as BAN2401 can lead to a decrease in a p-tau181 level in subjects who have low tau PET levels in a global brain measurement, e.g., in the subject receiving a therapeutically effective dose of the anti-Aβ protofibril antibody, as compared to a control. In some embodiments, a measure of p-tau 181 in a subject who has been treated is compared to a baseline measure obtained from the same subject before treatment, or to a reference control. In some embodiments, the Aβ protofibril antibody lowers plasma p-tau 181, as measured by an adjusted mean change from a baseline p-tau 181 level by at least about 0.2 pg/ml, 0.5 pg/ml, 0.6 pg/ml, 0.7 pg/ml, or 0.8 pg/ml. In some embodiments, the decrease in p-tau 181 occurs between the time point when the marker is first measured (e.g., a baseline measure) and one or more later time points when p-tau 181 is measured again. In some embodiments, the later time point is at least 6 months, 12 months, 18 months, 21 months, or 24 months after the baseline measure. In some embodiments, the control is a subject who does not receive a therapeutically effective dose of an Aβ protofibril antibody, e.g., a subject who receives no treatment, or a subject who receives a placebo. In some embodiments, the control is a subject who does not receive lecanemab. In some embodiments, a control is a reference measurement, such as an averaged measurement that combines population data from more than one subject and is representative of a subject who does not receive treatment.

Methods for measuring p-tau ratio are known in the art, such as immunoassay (e.g., a Quanterix™ Simoa® p-tau assay) and/or mass spectrophotometry (IP/LC-MS/MS) based technology methods. Plasma p-tau181 is elevated in early stages of AD as determined by Braak staging (I-II) and continues to increase as the disease progresses into Braak stage V-VI (Janelidze et al., “Plasma P-tau181 in Alzheimer's disease: relationship to other biomarkers, differential diagnosis, neuropathology and longitudinal progression to Alzheimer's dementia,” Nat. Med., 26(3):379-386 (2020)). The biomarker highly correlates with amyloid PET and Tau PET and has demonstrated a 3.5-fold elevation in AD compared to control, with an intermediate increase in the MCI group, and appears to differentiate patients with clinically diagnosed AD from other tauopathies as well (Thijssen et al., “Diagnostic value of plasma phosphorylated tau181 in Alzheimer's disease and frontotemporal lobar degeneration,” Nat. Med., 26(3):387-397 (2020); Janelidze et al.).

The measurement of the p-tau level (e.g., a p-tau181 level, a p-tau217 level, and/or a p-tau231 level) may be used alone to evaluate treatment efficacy, or in conjunction with one or more additional criteria, such as a tau PET level, a PET measurement of Aβ radiotracer update, MRI evaluation of Aβ plaque, and/or behavioral measures, as discussed herein. Such assays may also be used to diagnose patients eligible for treatment (e.g. by measuring the level of p-tau (e.g., a p-tau181 level, a p-tau217 level, and/or a p-tau231 level) and determining a subject is suitable for treatment because of a higher level than observed in a healthy control subject, alone or in conjunction with measuring one or more additional marker of AD pathology in the subject). In some embodiments, the measurement of the level of p-tau (e.g., a p-tau181 level, a p-tau217 level, and/or a p-tau231 level) may be used in place of another method of measuring brain amyloid levels, such as a PET scan for determining a subject is suitable for treatment. In some embodiments, the measurement of the level of p-tau (e.g., a p-tau181 level, a p-tau217 level, and/or a p-tau231 level) may be used in place of another method of measuring brain amyloid levels, such as a PET scan for determining treatment efficacy and/or making treatment decisions such as whether to continue treatment, switch to a maintenance dose, etc.

In some embodiments, a plasma or serum p-tau181 level measurement may employ a relative change from baseline measurement. In some embodiments, a change in the p-tau181 level may be used to evaluate treatment efficacy. In some embodiments, a decrease in the level of p-tau181 indicates treatment efficacy, e.g., a reduction in brain amyloid levels. In some embodiments, a p-tau181 level measurement may employ a set threshold to determine a change in brain amyloid levels, e.g., to identify and/or select a patient suitable for treatment, e.g., with an anti-Aβ protofibril antibody, or to determine whether to continue treatment, or to determine whether to switch to a maintenance dose, or to conclude a patient is amyloid negative. In some embodiments, the threshold may be evaluated in conjunction with another measurement of brain amyloid load, such as a PET scan, to assist in determining whether a subject is suitable for treatment or continued treatment. In some embodiments, a p-tau181 level threshold may be used in place of another method of measuring brain amyloid levels, such as a PET scan. In some embodiments, a p-tau181 level threshold at or above about 2.2 to 2.3 pg/mL is used to identify and/or select a patient suitable for treatment, e.g., with an anti-Aβ protofibril antibody. In some embodiments, a p-tau181 level threshold at or above about 2.2 pg/mL is used to identify and/or select a patient suitable for treatment, e.g., with an anti-Aβ protofibril antibody. In some embodiments, a p-tau181 level threshold at or above about 2.3 pg/mL is used to identify and/or select a patient suitable for treatment, e.g., with an anti-Aβ protofibril antibody. In certain such embodiments, the p-tau181 level is measured using a Quanterix™ Simoa® p-tau assay. In some embodiments, the threshold is about 2.3 pg/mL. In some embodiments, the threshold is about 2.2 pg/mL. In some embodiments, an increase in the p-tau181 level above a threshold value may indicate a need to continue treatment or to select an increase in a dosing regimen. In some embodiments, a decrease in the p-tau181 level below a threshold value may be used to indicate a treatment may be terminated (e.g., terminated in favor of a maintenance regimen) and/or to otherwise to determine a decrease in a dosing regimen or discontinuation in treatment. In some embodiments, an increase in a tau PET level relative to a control subject may be used to determine whether to discontinue a maintenance dosing regimen, e.g., and return to the prior treatment regimen.

In some embodiments, a tau PET level or a rate of change in a tau PET level and a level of an additional biomarker, e.g., a plasma p-tau181 level, may be used to select a patient for treatment, to allow for monitoring and treatment decisions such as whether to increase or decrease the amount of antibody being administered, determine whether to increase or decrease the frequency of administration, determine whether to introduce a further therapeutic agent, determine whether to switch to a maintenance dose, and/or whether to discontinue treatment with the anti-Aβ protofibril antibody. In some embodiments, a tau PET level may be used to select a patient for treatment, e.g., a tau PET level greater than 1.4 as measured by amyloid PET SUVr in the temporal region of the brain in combination with a plasma p-tau181 level, e.g., a plasma p-tau181 level greater than 2.2 pg/mL. In some embodiments, a tau PET level may be used to select a patient for treatment, e.g., a tau PET level greater than 1.5 as measured by amyloid PET SUVr in the temporal region of the brain in combination with an Aβ42/40 ratio, e.g., a plasma p-tau181 level greater than 2.2 pg/mL. Thresholds for determining amyloid positivity with p-tau181 are disclosed in PCT/US2022/079571, which herein is incorporated by reference. In some embodiments, a tau PET level that increased by less than 0.05-0.1 over a 13-month or 18-month period in a region of the brain (e.g., a temporal region) in combination with a p-tau181 level, e.g., a p-tau181 level at or below a threshold, may indicate an effective treatment. In some embodiments, a tau PET level that increased at a rate lower than an untreated control subject over a 13-month or 18-month period in a region of the brain (e.g., a temporal region) in combination with a p-tau181 level at or below a threshold, may indicate an effective treatment. In some embodiments, a tau PET from a region of the brain (e.g., a temporal region) lower than an untreated control subject in combination with a p-tau181 level at or below a threshold, may indicate an effective treatment. In some embodiments, an indication of the efficacy of treatment may be used to decrease the amount of antibody being administered, decrease the frequency of administration, or to switch to a maintenance dose. In some embodiments, a tau PET level that increased by more than 0.05-0.1 over a 13-month or 18-month period in a region of the brain (e.g., a temporal region) in combination with a p-tau181 level at or above a threshold (e.g., a threshold of 2.2 pg/mL), may indicate an ineffective treatment. In some embodiments, a tau PET level that increased at a rate higher than an untreated control subject over a 13-month or 18-month period in a region of the brain (e.g., a temporal region) in combination with a p-tau181 level at or above a threshold (e.g., a threshold of 2.2 pg/mL), may indicate an ineffective treatment. In some embodiments, a tau PET from a region of the brain (e.g., a temporal region) higher than an untreated control subject in combination with a p-tau181 level at or above a threshold (e.g., a threshold of 2.2 pg/mL), may indicate an ineffective treatment. In some embodiments, an indication of the efficacy treatment may be used to increase the amount of antibody being administered, increase the frequency of administration, or to discontinue treatment.

### G. Additional Biomarker Changes

### 1. Plasma or Serum Level of GFAP

In some embodiments, administration of a composition comprising a therapeutically effective amount of at least one anti-Aβ protofibril antibody disclosed herein to a subject results in a reduction in glial fibrillary acidic protein (GFAP) in the plasma or serum of a subject. Without being bound by theory, the GFAP level may be used as a marker of astrocyte activation. The GFAP level may be measured by techniques known in the art, such as an immunoassay (e.g., a Quanterix™ Simoa® assay) and/or mass spectrophotometry (IP/LC-MS/MS) based technology methods. In some embodiments, administration of a composition comprising a therapeutically effective amount of at least one anti-Aβ protofibril antibody disclosed herein to a subject results in a reduction in plasma or serum levels of GFAP in the subject. In some embodiments, the administration to a subject of a composition comprising a therapeutically effective amount of at least one anti-Aβ protofibril antibody disclosed herein results in a reduction of at least 1%, at least 2%, at least 3%, at least 4%, at least 5%, at least 6%, at least 7%, at least 8%, at least 9%, or at least 10%, relative to baseline, in the plasma or serum level of GFAP.

In some embodiments, a treatment comprising an anti-Aβ protofibril antibody such as BAN2401 can lead to a reduction in plasma or serum levels of GFAP in subjects who have low tau PET levels in a global brain measurement e.g., in the subject receiving a therapeutically effective dose of the anti-Aβ protofibril antibody, as compared to a control. In some embodiments, the reduction in plasma or serum levels of GFAP occurs between the time point when the plasma or serum level of GFAP is first measured (e.g., a baseline measure) and one or more later time points when the plasma or serum level of GFAP is measured again. In some embodiments, the later time point is at least 6 months, 12 months, 18 months, 21 months, or 24 months after the baseline measure. In some embodiments, the control is a subject who does not receive a therapeutically effective dose of an Aβ protofibril antibody, e.g., a subject who receives no treatment, or a subject who receives a placebo. In some embodiments, the control is a subject who does not receive lecanemab. In some embodiments, a control is a reference measurement, such as an averaged measurement that combines population data from more than one subject and is representative of a subject who does not receive treatment. In some embodiments, the Aβ protofibril antibody lowers plasma GFAP, as measured by an adjusted mean change from a baseline GFAP level by at least about 20 pg/ml, 30 pg/ml, 50 pg/ml, 60 pg/ml, or 80 pg/ml.

In some embodiments, a tau PET level and a plasma or serum level of GFAP, may be used to select a patient for treatment, to allow for monitoring and treatment decisions such as whether to increase or decrease the amount of antibody being administered, determine whether to increase or decrease the frequency of administration, determine whether to introduce a further therapeutic agent, determine whether to switch to a maintenance dose, and/or whether to discontinue treatment with the anti-Aβ protofibril antibody. In some embodiments, a tau PET level that increased by less than 0.05-0.1 over a 13-month or 18-month period in a region of the brain (e.g., a temporal region) in combination with a decreasing GFAP level over the same period, may indicate an effective treatment. In some embodiments, a tau PET level that increased at a rate lower than an untreated control subject over a 13-month or 18-month period in a region of the brain (e.g., a temporal region) in combination with a decreasing GFAP level over the same period, may indicate an effective treatment. In some embodiments, a tau PET from a region of the brain (e.g., a temporal region) lower than an untreated control subject in combination with a decreased GFAP level relative to an untreated control, may indicate an effective treatment. In some embodiments, an indication of the efficacy of treatment may be used to decrease the amount of antibody being administered, decrease the frequency of administration, or to switch to a maintenance dose.

### Cerebrospinal Fluid Level of Neurogranin

In some embodiments, administration of a composition comprising a therapeutically effective amount of at least one anti-Aβ protofibril antibody disclosed herein to a subject results in a reduction in cerebrospinal fluid level of neurogranin in the subject. In some embodiments, the administration to a subject of a composition comprising a therapeutically effective amount of at least one anti-Aβ protofibril antibody disclosed herein results in a reduction of at least 1%, at least 2%, at least 3%, at least 4%, at least 5%, at least 6%, at least 7%, at least 8%, at least 9%, or at least 10%, relative to baseline, in cerebrospinal fluid level of neurogranin.

In some embodiments, administration of a composition comprising a therapeutically effective amount of at least one anti-Aβ protofibril antibody disclosed herein to a subject having a low tau PET level results in a reduction in cerebrospinal fluid level of neurogranin in the subject, e.g., in the subject receiving a therapeutically effective dose of the anti-Aβ protofibril antibody, as compared to a control. In some embodiments, a measure of neurogranin in a subject who has been treated is compared to a baseline measure obtained from the same subject before treatment, or to a reference control. In some embodiments, the decrease in p-tau 181 occurs between the time point when the marker is first measured (e.g., a baseline measure) and one or more later time points when p-tau 181 is measured again. In some embodiments, the later time point is at least 6 months, 12 months, 18 months, 21 months, or 24 months after the baseline measure. In some embodiments, the control is a subject who does not receive a therapeutically effective dose of an Aβ protofibril antibody, e.g., a subject who receives no treatment, or a subject who receives a placebo. In some embodiments, the control is a subject who does not receive lecanemab. In some embodiments, a control is a reference measurement, such as an averaged measurement that combines population data from more than one subject and is representative of a subject who does not receive treatment.

In some embodiments, administration to a subject of a composition comprising a therapeutically effective amount of at least one anti-Aβ protofibril antibody disclosed herein results in a reduction in cerebrospinal fluid level of neurogranin after 18 months of administration of the composition comprising a therapeutically effective amount of at least one anti-Aβ protofibril antibody. In some embodiments, administration to a subject of a composition comprising a therapeutically effective amount of at least one anti-Aβ protofibril antibody disclosed herein results in a reduction of at least 1%, at least 2%, at least 3%, at least 4%, at least 5%, at least 6%, at least 7%, at least 8%, at least 9%, or at least 10%, relative to baseline, cerebrospinal fluid level of neurogranin after 18 months of administration of the composition.

In some embodiments, administration to a subject of a composition comprising a therapeutically effective amount of at least one anti-Aβ protofibril antibody disclosed herein results in a reduction of at least about 25 pg/mL, at least about 30 pg/mL, at least about 35 pg/mL, at least about 40 pg/mL, at least about 45 pg/mL, at least about 50 pg/mL, at least about 55 pg/mL, at least about 60 pg/mL, or at least about 65 pg/mL, relative to baseline, cerebrospinal fluid level of neurogranin. In some embodiments, administration to a subject of a composition comprising a therapeutically effective amount of at least one anti-Aβ protofibril antibody disclosed herein results in a reduction of at least about 65 pg/mL, relative to baseline, cerebrospinal fluid level of neurogranin.

In some embodiments, administration to a subject of a composition comprising a therapeutically effective amount of at least one anti-Aβ protofibril antibody disclosed herein results in a reduction of at least about 25 pg/mL, at least about 30 pg/mL, at least about 35 pg/mL, at least about 40 pg/mL, at least about 45 pg/mL, at least about 50 pg/mL, at least about 55 pg/mL, at least about 60 pg/mL, or at least about 65 pg/mL, relative to baseline, of cerebrospinal fluid level of neurogranin after 18 months of administration of the composition comprising a therapeutically effective amount of at least one anti-Aβ protofibril antibody. In some embodiments, administering to a subject a composition comprising a therapeutically effective amount of at least one anti-Aβ protofibril antibody disclosed herein results in a reduction of at least 65 pg/mL, relative to baseline, of cerebrospinal fluid level of neurogranin after 18 months of administration of the composition.

In some embodiments, the at least one anti-Aβ protofibril antibody is BAN2401.

In some embodiments, the therapeutically effective amount of at least one anti-Aβ protofibril antibody is 10 mg/kg. In some embodiments, the composition comprising a therapeutically effective amount of at least one anti-Aβ protofibril antibody disclosed herein is administered bi-weekly or monthly. In some embodiments, a composition comprising 10 mg/kg of BAN2401 is administered bi-weekly. In some embodiments, a composition comprising 10 mg/kg of BAN2401 is administered monthly.

In some embodiments, a tau PET level and a cerebrospinal fluid level of neurogranin, may be used to select a patient for treatment, to allow for monitoring and treatment decisions such as whether to increase or decrease the amount of antibody being administered, determine whether to increase or decrease the frequency of administration, determine whether to introduce a further therapeutic agent, determine whether to switch to a maintenance dose, and/or whether to discontinue treatment with the anti-Aβ protofibril antibody. In some embodiments, a tau PET level that increased by less than 0.05-0.1 over a 13-month or 18-month period in a region of the brain (e.g., a temporal region) in combination with a decreasing cerebrospinal fluid level of neurogranin over the same period, may indicate an effective treatment. In some embodiments, a tau PET level that increased at a rate lower than an untreated control subject over a 13-month or 18-month period in a region of the brain (e.g., a temporal region) in combination with a decreasing cerebrospinal fluid level of neurogranin over the same period, may indicate an effective treatment. In some embodiments, a tau PET from a region of the brain (e.g., a temporal region) lower than an untreated control subject in combination with a decreased cerebrospinal fluid level of neurogranin relative to an untreated control, may indicate an effective treatment. In some embodiments, an indication of the efficacy of treatment may be used to decrease the amount of antibody being administered, decrease the frequency of administration, or to switch to a maintenance dose.

### 2. Level of Neurofilament Light Chain

In some embodiments, administration to a subject of a composition comprising a therapeutically effective amount of at least one anti-Aβ protofibril antibody disclosed herein results in a reduction, relative to placebo, in cerebrospinal fluid level and/or the plasma or serum level of neurofilament light chain. In some embodiments, administration to a subject of a composition comprising a therapeutically effective amount of at least one anti-Aβ protofibril antibody disclosed herein results in a reduction of at least 5%, at least 10%, at least 15%, at least 20%, at least 25%, at least 30%, at least 35%, at least 40%, at least 45%, or at least 50%, relative to placebo, in cerebrospinal fluid level and/or the plasma or serum level of neurofilament light chain.

In some embodiments, administration to a subject having a low tau PET level of a composition comprising a therapeutically effective amount of at least one anti-Aβ protofibril antibody disclosed herein results in a reduction, relative to placebo, in cerebrospinal fluid level and/or the plasma or serum level of neurofilament light chain. In some embodiments, the administration of the anti-Aβ protofibril antibody results in a smaller increase over time, relative to placebo, of neurofilament light chain in plasma. In some embodiments, a measure of neurofilament light chain in a subject who has been treated is compared to a baseline measure obtained from the same subject before treatment, or to a reference control. In some embodiments, the change in neurofilament light chain occurs between the time point when the marker is first measured (e.g., a baseline measure) and one or more later time points when the marker is measured again. In some embodiments, the later time point is at least 6 months, 12 months, 18 months, 21 months, or 24 months after the baseline measure. In some embodiments, the control is a subject who does not receive a therapeutically effective dose of an Aβ protofibril antibody, e.g., a subject who receives no treatment, or a subject who receives a placebo. In some embodiments, the control is a subject who does not receive lecanemab. In some embodiments, a control is a reference measurement, such as a measurement that combines population data from more than one subject and is representative of a subject who does not receive treatment.

In some embodiments, administration to a subject of a composition comprising a therapeutically effective amount of at least one anti-Aβ protofibril antibody disclosed herein results in a reduction, relative to placebo, in cerebrospinal fluid level and/or the plasma or serum level of neurofilament light chain after 18 months of administration of the composition. In some embodiments, administration to a subject of a composition comprising a therapeutically effective amount of at least one anti-Aβ protofibril antibody disclosed herein results in a reduction, relative to placebo, of at least 5%, at least 10%, at least 15%, at least 20%, at least 25%, at least 30%, at least 35%, at least 40%, at least 45%, or at least 50%, relative to baseline, in cerebrospinal fluid level and/or the plasma or serum level of neurofilament light chain after 18 months of administration of the composition.

In some embodiments, administration to a subject of a composition comprising a therapeutically effective amount of at least one anti-Aβ protofibril antibody disclosed herein results in production of more than about 35 pg/mL, about 40 pg/mL, about 45 pg/mL, about 50 pg/mL, about 55 pg/mL, about 60 pg/mL, about 65 pg/mL, about 70 pg/mL, about 75 pg/mL, relative to baseline, of cerebrospinal fluid level of neurofilament light chain. In some embodiments, administration to a subject of a composition comprising a therapeutically effective amount of at least one anti-Aβ protofibril antibody disclosed herein results in production of no more than about 75 pg/mL, relative to baseline, of cerebrospinal fluid level of neurofilament light chain.

In some embodiments, administration to a subject of a composition comprising a therapeutically effective amount of at least one anti-Aβ protofibril antibody disclosed herein results in production of more than about 35 pg/mL, about 40 pg/mL, about 45 pg/mL, about 50 pg/mL, about 55 pg/mL, about 60 pg/mL, about 65 pg/mL, about 70 pg/mL, about 75 pg/mL, relative to baseline, of cerebrospinal fluid level of neurofilament light chain after 18 months of administration of the composition. In some embodiments, administration to a subject of a composition comprising a therapeutically effective amount of at least one anti-Aβ protofibril antibody disclosed herein results in production of no more than about 75 pg/mL, relative to baseline, of cerebrospinal fluid level of neurofilament light chain after 18 months of administration of the composition.

In some embodiments, the at least one anti-Aβ protofibril antibody is BAN2401.

In some embodiments, the therapeutically effective amount of at least one anti-Aβ protofibril antibody disclosed herein is 10 mg/kg. In some embodiments, a composition comprising a therapeutically effective amount of at least one anti-Aβ protofibril antibody disclosed herein is administered bi-weekly or monthly. In some embodiments, a composition comprising 10 mg/kg of BAN2401 is administered bi-weekly. In some embodiments, a composition comprising 10 mg/kg of BAN2401 is administered monthly.

In some embodiments, a tau PET level and a plasma or serum level of neurofilament light, may be used to select a patient for treatment, to allow for monitoring and treatment decisions such as whether to increase or decrease the amount of antibody being administered, determine whether to increase or decrease the frequency of administration, determine whether to introduce a further therapeutic agent, determine whether to switch to a maintenance dose, and/or whether to discontinue treatment with the anti-Aβ protofibril antibody. In some embodiments, a tau PET level that increased by less than 0.05-0.1 over a 13-month or 18-month period in a region of the brain (e.g., a temporal region) in combination with a decreasing plasma or serum level of neurofilament light over the same period, may indicate an effective treatment. In some embodiments, a tau PET level that increased at a rate lower than an untreated control subject over a 13-month or 18-month period in a region of the brain (e.g., a temporal region) in combination with a decreasing plasma or serum level of neurofilament light over the same period, may indicate an effective treatment. In some embodiments, a tau PET from a region of the brain (e.g., a temporal region) lower than an untreated control subject in combination with a decreased plasma or serum level of neurofilament light relative to an untreated control, may indicate an effective treatment. In some embodiments, an indication of the efficacy of treatment may be used to decrease the amount of antibody being administered, decrease the frequency of administration, or to switch to a maintenance dose.

### 3. Cerebrospinal Fluid Level of Phospho-Tau

In some embodiments, administration to a subject of a composition comprising a therapeutically effective amount of at least one anti-Aβ protofibril antibody disclosed herein results in a reduction in cerebrospinal fluid level of phospho-Tau (CSF p-tau) in a subject, e.g., as compared to a control. In some embodiments, the reduction in CSF p-tau occurs between the time point when CSF p-tau is first measured (e.g., a baseline measure) and one or more later time points when CSF p-tau is measured again. In some embodiments, the later time point is at least 6 months, 12 months, 18 months, 21 months, or 24 months after the baseline measure. In some embodiments, the control is a subject who does not receive a therapeutically effective dose of an Aβ protofibril antibody, e.g., a subject who receives no treatment, or a subject who receives a placebo. In some embodiments, the control is a subject who does not receive lecanemab. In some embodiments, a control is a reference measurement, such as a measurement that combines population data from more than one subject and is representative of a subject who does not receive treatment.

In some embodiments, administration to a subject of a composition comprising a therapeutically effective amount of at least one anti-Aβ protofibril antibody disclosed herein results in a reduction in cerebrospinal fluid level of phospho-Tau (p-tau). In some embodiments, administration to a subject of a composition comprising a therapeutically effective amount of at least one anti-Aβ protofibril antibody disclosed herein results in a reduction of at least 1%, at least 2%, at least 3%, at least 4%, at least 5%, at least 6%, at least 7%, at least 8%, at least 9%, at least 10%, at least 11%, at least 12%, or at least 13% relative to baseline, of cerebrospinal fluid level of phospho-Tau.

In some embodiments, administration to a subject having a low tau PET level of a composition comprising a therapeutically effective amount of at least one anti-Aβ protofibril antibody disclosed herein results in a reduction in cerebrospinal fluid level of phospho-Tau (p-tau). In some embodiments, administration to a subject having a low tau PET level of a composition comprising a therapeutically effective amount of at least one anti-Aβ protofibril antibody disclosed herein results in a reduction of at least 1%, at least 2%, at least 3%, at least 4%, at least 5%, at least 6%, at least 7%, at least 8%, at least 9%, at least 10%, at least 11%, at least 12%, or at least 13% relative to baseline, of cerebrospinal fluid level of phospho-Tau. In some embodiments, a measure of CSF p-tau in a subject who has been treated is compared to a baseline measure obtained from the same subject before treatment, or to a reference control. In some embodiments, the reference control is a control subject who has not been treated (e.g., a subject who received a placebo). In some embodiments, the reference control is a measurement obtained from a population of control subjects.

In some embodiments, administration to a subject of a composition comprising a therapeutically effective amount of at least one anti-Aβ protofibril antibody disclosed herein results in a reduction in cerebrospinal fluid level of phospho-Tau after 18 months of administration of the composition. In some embodiments, administration to a subject of a composition comprising a therapeutically effective amount of at least one anti-Aβ protofibril antibody disclosed herein results in a reduction of at least 1%, at least 2%, at least 3%, at least 4%, at least 5%, at least 6%, at least 7%, at least 8%, at least 9%, at least 10%, at least 11%, at least 12%, or at least 13%, relative to baseline, of cerebrospinal fluid level of phospho-Tau after 18 months of administration of the composition comprising a therapeutically effective amount of at least one anti-Aβ protofibril antibody.

In some embodiments, administration to a subject of a composition comprising a therapeutically effective amount of at least one anti-Aβ protofibril antibody disclosed herein results in a reduction of at least about 65 pg/mL, at least about 70 pg/mL, at least about 75 pg/mL, at least about 80 pg/mL, at least about 85 pg/mL, at least about 90 pg/mL, or at least about 95 pg/mL, relative to baseline, of cerebrospinal fluid level of phospho-Tau. In some embodiments, administration to a subject of a composition comprising a therapeutically effective amount of at least one anti-Aβ protofibril antibody disclosed herein results in a reduction of at least about 95 pg/mL, relative to baseline, of cerebrospinal fluid level of phospho-Tau.

In some embodiments, administration to a subject of a composition comprising a therapeutically effective amount of at least one anti-Aβ protofibril antibody disclosed herein results in a reduction of at least about 65 pg/mL, at least about 70 pg/mL, at least about 75 pg/mL, at least about 80 pg/mL, at least about 85 pg/mL, at least about 90 pg/mL, or at least about 95 pg/mL, relative to baseline, of cerebrospinal fluid level of phospho-Tau after 18 months of administration of the composition comprising a therapeutically effective amount of at least one anti-Aβ protofibril antibody. In some embodiments, administration to a subject of a composition comprising a therapeutically effective amount of at least one anti-Aβ protofibril antibody disclosed herein results in a reduction of at least 95 pg/mL, relative to baseline, of cerebrospinal fluid level of phospho-Tau after 18 months of administration of the composition comprising a therapeutically effective amount of at least one anti-Aβ protofibril antibody.

In some embodiments, the at least one anti-Aβ protofibril antibody is BAN2401.

In some embodiments, the therapeutically effective amount of at least one anti-Aβ protofibril antibody is 10 mg/kg. In some embodiments, the composition comprising a therapeutically effective amount of at least one anti-Aβ protofibril antibody disclosed herein is administered bi-weekly or monthly. In some embodiments, a composition comprising 10 mg/kg of BAN2401 is administered bi-weekly. In some embodiments, a composition comprising 10 mg/kg of BAN2401 is administered monthly.

In some embodiments, a tau PET level and cerebrospinal fluid level of phospho-Tau, may be used to select a patient for treatment, to allow for monitoring and treatment decisions such as whether to increase or decrease the amount of antibody being administered, determine whether to increase or decrease the frequency of administration, determine whether to introduce a further therapeutic agent, determine whether to switch to a maintenance dose, and/or whether to discontinue treatment with the anti-Aβ protofibril antibody. In some embodiments, a tau PET level that increased by less than 0.05-0.1 over a 13-month or 18-month period in a region of the brain (e.g., a temporal region) in combination with a decreasing cerebrospinal fluid level of phospho-Tau over the same period, may indicate an effective treatment. In some embodiments, a tau PET level that increased at a rate lower than an untreated control subject over a 13-month or 18-month period in a region of the brain (e.g., a temporal region) in combination with a decreasing cerebrospinal fluid level of phospho-Tau over the same period, may indicate an effective treatment. In some embodiments, a tau PET from a region of the brain (e.g., a temporal region) lower than an untreated control subject in combination with a decreased cerebrospinal fluid level of phospho-Tau relative to an untreated control, may indicate an effective treatment. In some embodiments, an indication of the efficacy of treatment may be used to decrease the amount of antibody being administered, decrease the frequency of administration, or to switch to a maintenance dose.

### H. Brain Volume and Brain Regions

In some embodiments, administration to a subject of a composition comprising a therapeutically effective amount of at least one anti-Aβ protofibril antibody disclosed herein results in an improvement, relative to placebo, of total hippocampal atrophy as measured by volumetric MRI (vMRI). In some embodiments, a subject's brain volume (e.g., total ventricular volume, left and/or right ventricular volume, total brain volume, right and/or left hippocampal volumes, cortical thickness) is measured before treatment. In some embodiments, a subject's brain volume (e.g., total ventricular volume, left and/or right ventricular volume, total brain volume, right and/or left hippocampal volumes, cortical thickness) is measured at 6, 12, and/or 18 months after treatment. In some embodiments, administration to a subject of a composition comprising a therapeutically effective amount of at least one anti-Aβ protofibril antibody disclosed herein results in an improvement, relative to placebo, of brain volume atrophy as measured by vMRI.

In some embodiments, brain regions affected by Alzheimer's disease may comprise the transentorhinal region of the brain, the limbic region (e.g., hippocampus), and neocortical regions. The brain regions may correspond to the Braak staging system, a standard based on anatomical localization of tau neurofibrillary tangles or phospho-tau, or a PET-based Braak staging system, e.g., as described by Therriault et al., Nature Aging volume 2, pages 526-535 (2022). Accordingly, a Braak region may refer to the region typically affected by tau aggregation at a Braak stage. For example, an early Braak region may refer to the entorhinal cortex and/or hippocampus.

In general, early Braak stages may be characterized as follows: Braak stage I may be characterized by tau aggregation in the entorhinal cortex, Braak stage II may be characterized by tau aggregation in the hippocampus, Braak stage III may be characterized by tau aggregation in the amygdala, parahippocampal gyrus, fusiform gyrus, and lingual gyrus. Later Braak stages, e.g., Braak stages IV and V may be characterized by tau aggregation in the association cortices, and Braak stage VI may be characterized by tau aggregation in the primary sensory cortices. In some embodiments, tau PET staging uses these Braak categorizations: Braak I (entorhinal cortex); Braak II (hippocampus); Braak III (amygdala, parahippocampal gyrus, fusiform gyrus, and lingual gyrus).

In some embodiments, an alternative tau-PET staging according to Therriault et al., Nature Aging volume 2, pages 526-535 (2022) may be used to categorize stages and brains regions as follows: Braak stage I (transentorhinal), Braak stage II (entorhinal and hippocampus), Braak stage III (amygdala, parahippocampal gyrus, fusiform gyrus and lingual gyrus), Braak stage IV (insula, inferior temporal, lateral temporal, posterior cingulate and inferior parietal), Braak stage V (orbitofrontal, superior temporal, inferior frontal, cuneus, anterior cingulate, supramarginal gyrus, lateral occipital, precuneus, superior parietal, superior frontal and rostromedial frontal) and Braak stage VI (paracentral, postcentral, precentral and pericalcarine).

In Braak staging (e.g., tau PET Braak staging), the categorization of stages and structures may vary slightly among methods due to natural variation among patients and/or variation in staging methods (e.g., different tau PET tracers and analysis methods). Results of tau PET Braak staging may differ from staging determined during an autopsy.

In some embodiments, tau aggregation, as measured by tau-PET and staged according to tau-PET-based Braak staging, may be associated with other indicators of AD disease progression, for example, expression of AD biomarkers such as CSF p-tau, CSF Aβ, and/or amyloid PET, presence of neurodegeneration, and impaired cognitive function. In some embodiments, tau aggregation in early Braak regions (e.g., stage I, II, or III regions) may be detected even in the absence of other AD biomarkers or the absence of evidence of neurodegeneration, either with or without cognitive impairment. Thus, tau-PET observed only in early Braak regions, such as the medial temporal region of the brain, may provide an early indicator of tau aggregation even in subjects who have few or no other signs or symptoms of AD. In some embodiments, the progression of the disease may be monitored by measuring tau-PET levels as they emerge in Braak regions over time, starting with subjects who may show tau-PET in early Braak regions, but who have few or no other signs or symptoms of AD. In some embodiments, such a subject may be selected for AD treatment, e.g., with at least one anti-Aβ protofibril antibody disclosed herein.

In some embodiments, a subject with tau-PET in early Braak regions (e.g., stage I, II, or III regions) may be treated according to the methods disclosed herein. In some embodiments, the subject is amyloid negative and/or at risk for amyloid accumulation. In some embodiments, a subject with tau-PET in early Braak regions (e.g., stage I, II, or III regions) and detectable amyloid PET at intermediate levels (e.g., 20-40 centiloids) or elevated levels (>40 centiloids) may be selected for AD treatment.

In some embodiments, a subject may have a low level of tau in a global brain measurement, preferably as measured by tau PET. In some embodiments, the global brain measurement is a measurement of tau PET in the whole cortical gray matter (e.g., a measure of cortical tau aggregation). In some embodiments, the low level of tau is a tau PET level (e.g., a tau-PET standardized uptake value ratio (SUVR)) below a threshold in the global brain measurement. In some embodiments, the global brain measurement is a measurement of tau PET in the whole cortical gray matter (e.g., a measure of cortical tau aggregation). In some embodiments, the tau PET level is measured using an MK6240 radiotracer. In some embodiments, the threshold tau PET level is a tau PET SUVR of about 1.1, preferably about 1.06, as measured with a MK6240 PET scan, e.g., of whole cortical gray matter.

In some embodiments, the subject has a low level of tau in the global brain measurement (e.g., whole cortical gray matter) and a higher level of tau in a local brain region. In some embodiments, the local brain region is an early Braak region (e.g., Braak regions I, II, or III). In some embodiments, the early Braak region comprises transentorhinal cortex, entorhinal cortex, hippocampus, amygdala, parahippocampal gyrus, fusiform gyrus, and/or lingual gyrus. In some embodiments, the local brain region is a composite of regions that accumulate tau in early AD. In some embodiments, the composite region comprises a temporal region, a medial temporal region, and/or a meta-temporal region. In some embodiments, the local brain region is the medial temporal region (e.g., the entorhinal cortex).

In some embodiments, a global brain measurement is a measure of tau aggregation in whole cortical gray matter, e.g., “cortical tau aggregation.” In some embodiments, a global brain measurement is a measure of tau aggregation in a selection of cortical regions (e.g., about 5-6 cortical regions, or cortical and subcortical regions).

In some embodiments, a subject may have a low level of cortical tau aggregation, as measured by low tau-PET levels (e.g., a SUVr <1.06 in whole cortical gray matter in MK6240 PET scans) but may have higher levels of tau aggregation in localized regions. For example, a subject categorized as low tau-PET according to measurements in whole cortical gray matter may have tau aggregation in Braak regions I, II, and III. This subject may not have tau aggregation in Braak regions IV, V, and VI.

In some embodiments, administration to the subject having a low level of cortical tau aggregation, as defined by low tau-PET levels (e.g., a SUVr <1.06 in whole cortical gray matter in MK6240 PET scans) of a composition comprising a therapeutically effective amount of at least one anti-Aβ protofibril antibody disclosed herein results in an improvement, relative to placebo, of the progression of tau pathology in the brain. For example, the improvement may be a delayed increase, stabilized level, or a reduction in tau-PET in an early Braak region(s) (e.g., temporal lobe), such as the medial temporal region, the meta temporal region, and the temporal region. The improvement may be a delayed increase, stabilized level, or a reduction in tau-Pet in the medial temporal region, which is often the earliest region where tau aggregation may be observed. In contrast, for subjects with levels of tau defined as intermediate and/or high (e.g., an intermediate level is a SUVr between 1.06-2.91 and a high level is a SUVr above 2.91, as determined in whole cortical gray matter in MK6240 PET scans), the improvement may be a delayed increase, stabilized level, or a reduction in tau-PET across more brain regions, consistent with the greater extent of tau aggregation in these subjects.

In some embodiments, administration to a subject with a low level of cortical tau aggregation, determined by low tau-PET levels (e.g., a SUVr <1.06 in whole cortical gray matter in MK6240 PET scans) of a composition comprising a therapeutically effective amount of at least one anti-Aβ protofibril antibody disclosed herein results in an improvement, relative to placebo, of at least one measure of cognitive function, e.g., a slower decline in cognitive function, a stabilized level of cognitive function, or an improvement in cognitive function. In some embodiments, cognitive function may be measured by CDR-SB, ADAS-Cog14, or ADCS MCI-ADL. In some embodiments, a measure of “no decline” or a measure of “improvement” may be higher in subjects with low tau-PET levels than in patients with intermediate or higher tau-PET levels.

For example, a patient treated according to any of the methods disclosed herein may have low levels of cortical tau aggregation, as determined by low tau-PET levels (e.g., a SUVr <1.06 in whole cortical gray matter in MK6240 PET scans) but higher levels of tau-PET observed in early Braak regions. Such a patient may also have lower levels of CSF p-tau and CSF Aβ, less neurodegeneration, and higher cognitive function than a patient with intermediate or intermediate+high cortical tau aggregation levels, as measured by whole cortical gray matter tau PET. In some embodiments, a patient may have tau-PET indicating early tau aggregation (e.g., Braak stage I, II, or III), but may not have other AD biomarkers and/or may not have cognitive impairments.

### I. Anti-Aβ Protofibril Antibodies

In some embodiments, any anti-Aβ protofibril antibody may be used in the methods disclosed herein. In some embodiments, the antibody comprises one or more of the sequences listed in Tables 1-4, e.g., comprising a complete set of 6 complementarity determining regions (CDRs) and/or a complete set of variable regions and/or a complete set of heavy and light chain sequences from the tables. In some embodiments, the anti-Aβ protofibril antibody comprises three heavy chain complementarity determining regions (HCDR1, HCDR2, and HCDR3) comprising amino acid sequences of SEQ ID NO: 1 (HCDR1), SEQ ID NO: 2 (HCDR2), and SEQ ID NO: 3 (HCDR3); and three light chain complementarity determining regions (LCDR1, LCDR2, and LCDR3) comprising amino acid sequences of SEQ ID NO: 4 (LCDR1), SEQ ID NO: 5 (LCDR2), and SEQ ID NO: 6 (LCDR3). In some embodiments, the anti-Aβ protofibril antibody comprises a heavy chain variable region comprising an amino acid sequence of SEQ ID NO: 7 and a light chain variable region comprising an amino acid sequence of SEQ ID NO: 8. In some embodiments, the anti-Aβ protofibril antibody comprises human heavy and light chain variable region frameworks. In some embodiments, the anti-Aβ protofibril antibody comprises a human IgG1 heavy chain constant region, and a human Ig kappa light chain constant region. In some embodiments, the anti-Aβ protofibril antibody comprises a heavy chain comprising an amino acid sequence of SEQ ID NO: 9 and a light chain comprising an amino acid sequence of SEQ ID NO: 10. “CDRs” used herein in the context of an antibody sequence or structure refers to complementarity determining regions, that provide the main determinants of antigen binding. Generally, the antigen-binding site has six CDRs; three in the VH (HCDR1, HCDR2, HCDR3), and three in the VL (LCDR1, LCDR2, LCDR3). The CDRs may be determined according to the Kabat numbering scheme. which may be determined by according to the Kabat numbering scheme (Kabat et al., Sequences of Proteins of Immunological Interest, 5th Ed. Public Health Service, National Institutes of Health, Bethesda, Md., 1991, hereafter referred to as “Kabat report”).

In some embodiments, the at least one anti-Aβ protofibril antibody comprises a human constant region. In some embodiments, the human constant region of the at least one anti-Aβ protofibril antibody comprises a heavy chain constant region chosen from IgG1, IgG2, IgG3, IgG4, IgM, IgA, IgE, and any allelic variation thereof as disclosed in the Kabat report. Any one or more of such sequences may be used in the present disclosure. In some embodiments, the heavy chain constant region is chosen from IgG1 and allelic variations thereof. The amino acid sequence of human IgG1 constant region is known in the art and set out in SEQ ID NO: 11.

In some embodiments, the human constant region of the at least one anti-Aβ antibody comprises a light chain constant region chosen from κ-λ-chain constant regions and any allelic variation thereof as discussed in the Kabat report. Any one or more of such sequences may be used in the present disclosure. In some embodiments, the light chain constant region is chosen from κ and allelic variations thereof. The amino acid sequence of human κ chain constant region is known in the art and set out in SEQ ID NO: 12.

In some embodiments, the at least one anti-Aβ protofibril antibody is BAN2401, also known as lecanemab. The terms “BAN2401” and “lecanemab” are used interchangeably and refer to a humanized IgG1 monoclonal version of mAb158, which is a murine monoclonal antibody raised to target protofibrils and disclosed in WO 2007/108756 and Journal of Alzheimer's Disease 43: 575-588 (2015). BAN2401 comprises three heavy chain complementarity determining regions (HCDR1, HCDR2, and HCDR3) comprising amino acid sequences of SEQ ID NO: 1 (HCDR1), SEQ ID NO: 2 (HCDR2), and SEQ ID NO: 3 (HCDR3); and three light chain complementarity determining regions (LCDR1, LCDR2, and LCDR3) comprising amino acid sequences of SEQ ID NO: 4 (LCDR1), SEQ ID NO: 5 (LCDR2), and SEQ ID NO: 6 (LCDR3) and is described in WO 2007/108756 and in Journal of Alzheimer's Disease 43:575-588 (2015). BAN2401 comprises (i) a heavy chain variable region comprising the amino acid sequence of SEQ ID NO: 7 and (ii) a light chain variable region comprising the amino acid sequence of SEQ ID NO: 8. The full length sequences of heavy chain and light chain of BAN2401 are set forth in SEQ ID NOs: 9 and 10 and are described in WO 2007/108756 and in Journal of Alzheimer's Disease 43:575-588 (2015).

In some embodiments, the isolated anti-Aβ protofibril antibody to be used in treatment is present in a concentration of at least 80 mg/mL. In some embodiments, the isolated anti-Aβ protofibril antibody is present in a concentration of at least 100 mg/mL. In some embodiments, the isolated anti-Aβ protofibril antibody is present in a concentration of at least 200 mg/mL. In some embodiments, the isolated anti-Aβ protofibril antibody is present in a concentration of at least 250 mg/mL. In some embodiments, the isolated antibody or fragment thereof is present in a concentration ranging from 80 mg/mL to 300 mg/mL. In some embodiments, the isolated anti-Aβ protofibril antibody is present in a concentration ranging from 85 mg/mL to 275 mg/mL. In some embodiments, the isolated anti-Aβ protofibril antibody is present in a concentration ranging from 90 mg/mL to 250 mg/mL. In some embodiments, the isolated anti-Aβ protofibril antibody is present in a concentration ranging from 95 mg/mL to 225 mg/mL. In some embodiments, the isolated anti-Aβ protofibril antibody is present in a concentration ranging from 100 mg/mL to 200 mg/mL. In some embodiments, the isolated antibody or fragment thereof is present in a concentration of 80 mg/mL, 90 mg/mL, 100 mg/mL, 110 mg/mL, 120 mg/mL, 130 mg/mL, 140 mg/mL, 150 mg/mL, 160 mg/mL, 170 mg/mL, 180 mg/mL, 190 mg/mL, 200 mg/mL, 210 mg/mL, 220 mg/mL, 230 mg/mL, 240 mg/mL, 250 mg/mL, 260 mg/mL, 270 mg/mL, 280 mg/mL, 290 mg/mL, or 300 mg/mL. In some embodiments, the isolated antibody or fragment thereof is present in a concentration of 100 mg/mL. In some embodiments, the isolated antibody or fragment thereof is present in a concentration of 200 mg/mL. In some embodiments, the isolated antibody or fragment thereof is present in a concentration of 250 mg/mL. In some embodiments, the isolated antibody or fragment thereof is present in a concentration of 300 mg/mL. In some embodiments, the isolated antibody or fragment thereof is BAN2401.

As used herein, a “fragment” of an antibody comprises a portion of the antibody, for example comprising an antigen-binding or a variable region thereof. Non-limiting examples of fragments include Fab fragments, Fab′ fragments, F(ab′)2 fragments, Fv fragments, diabodies, linear antibodies, and single-chain antibody molecules.

In some embodiments, the anti-Aβ protofibril antibody (e.g., BAN2401, or a fragment thereof) reduces cerebral amyloid angiopathy (CAA), amyloid beta peptide deposits in the walls of small to medium blood vessels in the central nervous system and meninges.

### J. Therapeutically Effective Amount of at Least One Anti-Aβ Protofibril Antibody

In various embodiments, the methods of the present disclosure comprise administering to a subject a composition comprising a therapeutically effective amount of at least one anti-Aβ protofibril antibody. As used herein, the term a “therapeutically effective amount” refers to an amount of a compound or pharmaceutical composition sufficient to produce a desired therapeutic effect.

One of ordinary skill in the art will understand that the therapeutically effective amount of the at least one anti-Aβ protofibril antibody administered to a subject may depend upon a number of factors including pharmacodynamic characteristics, route of administration, frequency of treatment, and health, age, and weight of the subject to be treated and, with the information disclosed herein, will be able to determine the appropriate amount for each subject.

In some embodiments, the therapeutically effective amount is a dose chosen to improve efficacy and/or maintain efficacy and improve at least one of safety and tolerability. In some embodiments, the therapeutically effective amount is chosen to lower at least one side effect and simultaneously improve efficacy and/or maintain efficacy. Therapeutically effective doses of an anti-Aβ protofibril antibody and methods for measuring treatment efficacy are disclosed in PCT/US2022/073576; PCT/US2022/079571; and PCT/US2022/041926 and are incorporated herein by reference.

In some embodiments, 0.5 mg/kg to 45 mg/kg, 0.5 mg/kg to 40 mg/kg, 0.5 mg/kg to 35 mg/kg, 0.5 mg/kg to 30 mg/kg, 0.5 mg/kg to 25 mg/kg, 0.5 mg/kg to 20 mg/kg, 0.5 mg/kg to 15 mg/kg, 0.5 mg/kg to 10 mg/kg, 0.5 mg/kg to 5 mg/kg, or 0.5 mg/kg to 2.5 mg/kg of at least one anti-Aβ protofibril antibody is administered to the subject relative to body weight of the subject.

In some embodiments, 2.5 mg/kg to 45 mg/kg, 2.5 mg/kg to 40 mg/kg, 2.5 mg/kg to 35 mg/kg, 2.5 mg/kg to 30 mg/kg, 2.5 mg/kg to 25 mg/kg, 2.5 mg/kg to 20 mg/kg, 2.5 mg/kg to 15 mg/kg, 2.5 mg/kg to 10 mg/kg, or 2.5 mg/kg to 5 mg/kg of at least one anti-Aβ protofibril antibody is administered to the subject relative to body weight of the subject.

In some embodiments, 5 mg/kg to 45 mg/kg, 5 mg/kg to 40 mg/kg, 5 mg/kg to 35 mg/kg, 5 mg/kg to 30 mg/kg, 5 mg/kg to 25 mg/kg, 5 mg/kg to 20 mg/kg, 5 mg/kg to 15 mg/kg, or 5 mg/kg to 10 mg/kg, of at least one anti-Aβ protofibril antibody is administered to the subject relative to body weight of the subject.

In some embodiments, 7.5 mg/kg to 45 mg/kg, 7.5 mg/kg to 40 mg/kg, 7.5 mg/kg to 35 mg/kg, 7.5 mg/kg to 30 mg/kg, 7.5 mg/kg to 25 mg/kg, 7.5 mg/kg to 20 mg/kg, 7.5 mg/kg to 15 mg/kg, or 7.5 mg/kg to 10 mg/kg of at least one anti-Aβ protofibril antibody is administered to the subject relative to body weight of the subject.

In some embodiments, from 0.5 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg of at least one anti-Aβ protofibril antibody is administered to the subject relative to body weight of the subject. In some embodiments, up to 20 mg/kg, 19 mg/kg, 18 mg/kg, 17 mg/kg, 16 mg/kg, 15 mg/kg, 14 mg/kg, 13 mg/kg, 12 mg/kg, 11 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, or 0.5 mg/kg of at least one anti-Aβ protofibril antibody is administered to the subject relative to body weight of the subject.

In some embodiments, 0.5 mg/kg of at least one anti-Aβ protofibril antibody is administered to the subject relative to body weight of the subject. In some embodiments, 1 mg/kg of at least one anti-Aβ protofibril antibody is administered to the subject relative to body weight of the subject. In some embodiments, 2 mg/kg of at least one anti-Aβ protofibril antibody is administered to the subject relative to body weight of the subject. In some embodiments, 2.5 mg/kg of at least one anti-Aβ protofibril antibody is administered to the subject relative to body weight of the subject. In some embodiments, 3 mg/kg of at least one anti-Aβ protofibril antibody is administered to the subject relative to body weight of the subject. In some embodiments, 4 mg/kg of at least one anti-Aβ protofibril antibody is administered to the subject relative to body weight of the subject. In some embodiments, 5 mg/kg of at least one anti-Aβ protofibril antibody is administered to the subject relative to body weight of the subject. In some embodiments, 6 mg/kg of at least one anti-Aβ protofibril antibody is administered to the subject relative to body weight of the subject. In some embodiments, 7 mg/kg of at least one anti-Aβ protofibril antibody is administered to the subject relative to body weight of the subject. In some embodiments, 7.5 mg/kg of at least one anti-Aβ protofibril antibody is administered to the subject relative to body weight of the subject. In some embodiments, 8 mg/kg of at least one anti-Aβ protofibril antibody is administered to the subject relative to body weight of the subject. In some embodiments, 9 mg/kg of at least one anti-Aβ protofibril antibody is administered to the subject relative to body weight of the subject. In some embodiments, 10 mg/kg of at least one anti-Aβ protofibril antibody is administered to the subject relative to body weight of the subject. In some embodiments, 11 mg/kg of at least one anti-Aβ protofibril antibody is administered to the subject relative to body weight of the subject. In some embodiments, 12 mg/kg of at least one anti-Aβ protofibril antibody is administered to the subject relative to body weight of the subject. In some embodiments, 12.5 mg/kg of at least one anti-Aβ protofibril antibody is administered to the subject relative to body weight of the subject. In some embodiments, 13 mg/kg of at least one anti-Aβ protofibril antibody is administered to the subject relative to body weight of the subject. In some embodiments, 14 mg/kg of at least one anti-Aβ protofibril antibody is administered to the subject relative to body weight of the subject. In some embodiments, 15 mg/kg of at least one anti-Aβ protofibril antibody is administered to the subject relative to body weight of the subject. In some embodiments, 16, 17, 18, 19, or 20 mg/kg of at least one anti-Aβ protofibril antibody is administered to the subject relative to body weight of the subject. In some embodiments, 21, 22, 23, 24, or 25 mg/kg of at least one anti-Aβ protofibril antibody is administered to the subject relative to body weight of the subject.

In some embodiments, 27.5 mg/kg, 30 mg/kg, 32.5 mg/kg, 35 mg/kg, 37.5 mg/kg, 40 mg/kg, 42.5 mg/kg, 45 mg/kg, 47.5 mg/kg, or 50 mg/kg of at least one anti-Aβ protofibril antibody is administered to the subject relative to body weight of the subject.

As mentioned, in some embodiments, the at least one anti-Aβ protofibril antibody is BAN2401. Accordingly, in some embodiments, 0.5 mg/kg to 45 mg/kg, 0.5 mg/kg to 40 mg/kg, 0.5 mg/kg to 35 mg/kg, 0.5 mg/kg to 30 mg/kg, 0.5 mg/kg to 25 mg/kg, 0.5 mg/kg to 20 mg/kg, 0.5 mg/kg to 15 mg/kg, 0.5 mg/kg to 10 mg/kg, 0.5 mg/kg to 5 mg/kg, or 0.5 mg/kg to 2.5 mg/kg of BAN2401 is administered to the subject relative to body weight of the subject.

In some embodiments, 2.5 mg/kg to 45 mg/kg, 2.5 mg/kg to 40 mg/kg, 2.5 mg/kg to 35 mg/kg, 2.5 mg/kg to 30 mg/kg, 2.5 mg/kg to 25 mg/kg, 2.5 mg/kg to 20 mg/kg, 2.5 mg/kg to 15 mg/kg, 2.5 mg/kg to 10 mg/kg, or 2.5 mg/kg to 5 mg/kg of BAN2401 is administered to the subject relative to body weight of the subject.

In some embodiments, 5 mg/kg to 45 mg/kg, 5 mg/kg to 40 mg/kg, 5 mg/kg to 35 mg/kg, 5 mg/kg to 30 mg/kg, 5 mg/kg to 25 mg/kg, 5 mg/kg to 20 mg/kg, 5 mg/kg to 15 mg/kg, or 5 mg/kg to 10 mg/kg of BAN2401 is administered to the subject relative to body weight of the subject.

In some embodiments, 7.5 mg/kg to 45 mg/kg, 7.5 mg/kg to 40 mg/kg, 7.5 mg/kg to 35 mg/kg, 7.5 mg/kg to 30 mg/kg, 7.5 mg/kg to 25 mg/kg, 7.5 mg/kg to 20 mg/kg, 7.5 mg/kg to 15 mg/kg, or 7.5 mg/kg to 10 mg/kg of BAN2401 is administered to the subject relative to body weight of the subject.

In some embodiments, from 0.5 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg of BAN2401 is administered to the subject relative to body weight of the subject. In some embodiments, up to 20 mg/kg, 19 mg/kg, 18 mg/kg, 17 mg/kg, 16 mg/kg, 15 mg/kg, 14 mg/kg, 13 mg/kg, 12 mg/kg, 11 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, or 0.5 mg/kg of BAN2401 is administered to the subject relative to body weight of the subject.

In some embodiments, 0.5 mg/kg of BAN2401 is administered to the subject relative to body weight of the subject. In some embodiments, 1 mg/kg of BAN2401 is administered to the subject relative to body weight of the subject. In some embodiments, 2 mg/kg of BAN2401 is administered to the subject relative to body weight of the subject. In some embodiments, 2.5 mg/kg of BAN2401 is administered to the subject relative to body weight of the subject. In some embodiments, 3 mg/kg of BAN2401 is administered to the subject relative to body weight of the subject. In some embodiments, 4 mg/kg of BAN2401 is administered to the subject relative to body weight of the subject. In some embodiments, 5 mg/kg of BAN2401 is administered to the subject relative to body weight of the subject. In some embodiments, 6 mg/kg of BAN2401 is administered to the subject relative to body weight of the subject. In some embodiments, 7 mg/kg of BAN2401 is administered to the subject relative to body weight of the subject. In some embodiments, 7.5 mg/kg of BAN2401 is administered to the subject relative to body weight of the subject. In some embodiments, 8 mg/kg of BAN2401 is administered to the subject relative to body weight of the subject. In some embodiments, 9 mg/kg of BAN2401 is administered to the subject relative to body weight of the subject. In some embodiments, 10 mg/kg of BAN2401 is administered to the subject relative to body weight of the subject. In some embodiments, 11 mg/kg of BAN2401 is administered to the subject relative to body weight of the subject. In some embodiments, 12 mg/kg of BAN2401 is administered to the subject relative to body weight of the subject. In some embodiments, 12.5 mg/kg of BAN2401 is administered to the subject relative to body weight of the subject. In some embodiments, 13 mg/kg of BAN2401 is administered to the subject relative to body weight of the subject. In some embodiments, 14 mg/kg of BAN2401 is administered to the subject relative to body weight of the subject. In some embodiments, 15 mg/kg of BAN2401 is administered to the subject relative to body weight of the subject. In some embodiments, 16, 17, 18, 19, or 20 mg/kg of BAN2401 is administered to the subject relative to body weight of the subject. In some embodiments, 21, 22, 23, 24, or 25 mg/kg of BAN2401 is administered to the subject relative to body weight of the subject. In some embodiments, 27.5 mg/kg, 30 mg/kg, 32.5 mg/kg, 35 mg/kg, 37.5 mg/kg, 40 mg/kg, 42.5 mg/kg, 45 mg/kg, 47.5 mg/kg, or 50 mg/kg of BAN2401 is administered to the subject relative to body weight of the subject.

In some embodiments, 2.5 mg/kg to 10 mg/kg of BAN2401 is administered to the subject relative to body weight of the subject. In some embodiments, 5 mg/kg to 10 mg/kg of BAN2401 is administered to the subject relative to body weight of the subject. In some embodiments, 2.5 mg/kg of BAN2401 is administered to the subject relative to body weight of the subject. In some embodiments, 5 mg/kg of BAN2401 is administered to the subject relative to body weight of the subject. In some embodiments, 7.5 mg/kg of BAN2401 is administered to the subject relative to body weight of the subject. In some embodiments, 10 mg/kg of BAN2401 is administered to the subject relative to body weight of the subject.

In some embodiments, a subject is administered a first dose of the anti-Aβ protofibril antibody without an initial titrating step up to the treatment dose (e.g., a subject starts treatment at 10 mg/kg with no titration). In some embodiments, a dose of BAN2401 may be used without the need of a prior titrating step. In some embodiments, a subject is switched to a maintenance dose without an initial titrating step to the maintenance dose. In certain instances, providing a therapeutic dose without a titration step may provide additional therapeutic benefits to the patient, e.g., a faster shift in plasma biomarkers toward amyloid negativity or facilitating identification sooner of patients that do not have a therapeutic change in plasma biomarkers in response to the anti-Aβ protofibril antibody (non-responders) and who would benefit from alternative treatment.

### K. Composition Comprising at Least One Anti-Aβ Protofibril Antibody

In some embodiments, the at least one anti-Aβ protofibril antibody is comprised in a composition. In some embodiments, the composition consists of at least one anti-Aβ protofibril antibody. In some embodiments, the antibody is present at a concentration of 50-250 mg/ML, e.g., 100-200 mg/mL. In some embodiments, the composition comprises at least one anti-Aβ protofibril antibody and further comprises at least one additional active and/or inactive component. In some embodiments, the at least one additional component can comprise one or more suitable physiologically acceptable excipients for human and/or veterinary use.

The compositions of the present disclosure may be in the form of a tablet, pill, capsule, solution, and/or any other suitable form deemed appropriate by one of ordinary skill in the art. The route of administration of the compositions of the present disclosure may be any suitable route, including intravenous, subcutaneous, oral, and nasal. In some embodiments, the composition is formulated as a sterile, non-pyrogenic liquid for intravenous administration. In some embodiments, the composition is a saline solution.

In some embodiments, the at least one additional component in the composition comprises buffer(s). In some embodiments, the at least one additional component comprises emulsifier(s). In some embodiments, the at least one additional component comprises sodium citrate, sodium chloride, histidine, arginine, arginine hydrochloride, and/or polysorbate 80. In some embodiments, the sodium citrate may be present at a concentration ranging from 1 mM to 150 mM. In some embodiments, the sodium citrate may be present at a concentration of 25 mM. In some embodiments, the sodium citrate may be present at a concentration of 50 mM. In some embodiments, the sodium chloride may be present at a concentration ranging from 25 mM to 250 mM. In some embodiments, the arginine may be present at a concentration ranging from 240 mM to 360 mM. In some embodiments, the arginine hydrochloride may be present at a concentration ranging from 100 mM to 250 mM. In some embodiments, the histidine may be present at a concentration ranging from 10 mM to 50 mM. In some embodiments, the sodium citrate may be present at a concentration of 125 mM. In some embodiments, the polysorbate 80 may be present at a concentration ranging from 0.001% (w/v) to 2% (w/v). In some embodiments, the polysorbate 80 may be present at a concentration of 0.02% (w/v). In some embodiments, the polysorbate 80 may be present at a concentration of 0.05% (w/v).

In some embodiments, the composition is a liquid dosage form comprising at least one anti-Aβ protofibril antibody, such as BAN2401, and further comprising, for instance, sodium citrate, sodium chloride, and polysorbate 80. In some embodiments, the composition is a liquid dosage form comprising 50 mmol/L citrate, 350 mmol/L arginine, and 0.05% polysorbate 80.

In some embodiments, the composition is a liquid dosage form comprising at least one anti-Aβ protofibril antibody, such as BAN2401, and further comprising, for instance, arginine hydrochloride, histidine, and polysorbate 80. In some embodiments, the composition is a liquid dosage form comprising 25 mmol/L histidine, 200 mmol/L arginine, 0.05% polysorbate 80. PCT/IB2021/000155 (WO2021/186245) is incorporated herein by reference for suitable intravenous and subcutaneous formulations.

### L. Concomitant Administration of at Least One Anti-Aβ Protofibril Antibody and at Least One Alzheimer's Disease Medication Other than BAN2401

In some embodiments, provided herein is a method of treating a subject, e.g., one having Pre-AD or early Alzheimer's disease, comprising administering a therapeutically effective amount of at least one anti-Aβ protofibril antibody such as BAN2401 and a therapeutically effective amount of at least one Alzheimer's disease medication other than BAN2401. In some embodiments, provided herein is a method of reducing and/or slowing clinical decline in a subject, e.g., one having Pre-AD or early Alzheimer's disease, comprising administering a therapeutically effective amount of at least one anti-Aβ protofibril antibody such as BAN2401 and a therapeutically effective amount of at least one Alzheimer's disease medication other than BAN2401. The at least one additional therapy may comprise an additional anti-Aβ antibody such as aducanumab. In some embodiments, the at least one additional therapy may comprise a BACE inhibitor and/or an anti-tau antibody.

In some embodiments, the at least one Alzheimer's disease medication is chosen from elenbecestat, donepezil, galantamine, memantine, and rivastigmine. In some embodiments, the at least one Alzheimer's disease medication is a combination of donepezil and memantine. In some embodiments, the at least one additional therapeutic agent comprises one or more of BACE inhibitors, gamma secretase inhibitors, gamma secretase modulators, Aβ peptide generation inhibitors other than said at least one anti-Aβ protofibril antibody, agents that lower Aβ peptide levels other than said at least one anti-Aβ protofibril antibody, and a combination thereof. In some embodiments, the at least one additional therapeutic agent is a BACE inhibitor. In some embodiments, the BACE inhibitor is chosen from CNP520, BI-1181181, LY2886721, LY3202626, PF-06751979, RG7129, atabecestat, elenbecestat, lanabecestat, and verubecestat. In some embodiments, the BACE inhibitor is elenbecestat. In some embodiments, the BACE inhibitor is chosen from CNP520, BI-1181181, LY2886721, LY3202626, PF-06751979, RG7129, atabecestat, elenbecestat, lanabecestat, and verubecestat.

In some embodiments, provided herein is a method of treating a subject having pre-AD, or a patient that is symptomatic for Alzheimer's disease (e.g., early Alzheimer's disease), comprising administering a therapeutically effective amount of at least one anti-Aβ protofibril antibody such as BAN2401 and a therapeutically effective amount of an anti-tau antibody or antigen binding fragment thereof that is capable of binding to human tau, e.g., the anti-tau antibody or antigen binding fragment comprises E2814 or an antigen binding fragment thereof. In some embodiments, the anti-tau antibody is an antibody that binds to the microtubule binding region of tau (MTBR-tau). In some embodiments, the anti-tau antibody is E2814. In some embodiments, the anti-tau antibody is LY3303560, ABBV-8E12, BIIB076, PNT001, UCB0107 PRX005, Lu AF87908, BIIB092, R07105705, or JNJ-63733657. E2814 is disclosed in US 2019/0112364 A1 as clone 7G6-HCzu25/LCzul8, the sequences of which are incorporated by reference herein. In some embodiments, provided herein is a method of reducing and/or slowing clinical decline in a subject, e.g., one having pre-AD, or a patient that is symptomatic for Alzheimer's disease (e.g., early Alzheimer's disease), comprising administering a therapeutically effective amount of at least one anti-Aβ protofibril antibody such as BAN2401 and a therapeutically effective amount of an anti-tau antibody or antigen binding fragment thereof that is capable of binding to human tau, e.g., the anti-tau antibody or antigen binding fragment comprises E2814 or an antigen binding fragment thereof. E2814 is disclosed in US 2019/0112364 A1 as clone 7G6-HCzu25/LCzul8, the sequences of which are incorporated by reference herein. In some embodiments, the isolated anti-tau antibody or antigen binding fragment thereof that is capable of binding to human tau comprises six CDRs (HCDR1, HCDR2, HCDR3, LCDR1, LCDR2, and LCDR3) comprising the amino acid sequences of SEQ ID NO:15 (HCDR1), SEQ ID NO:16 (HCDR2), SEQ ID NO:17 (HCDR3), SEQ ID NO:18 (LCDR1), SEQ ID NO:19 (LCDR2), and SEQ ID NO:20 (LCDR3). See, e.g., Table 6. In some embodiments, the isolated anti-tau antibody or antigen binding fragment thereof that is capable of binding to human tau comprises six CDRs (HCDR1, HCDR2, HCDR3, LCDR1, LCDR2, and LCDR3) from a heavy chain variable region of SEQ ID NO: 21 and a light chain variable region of SEQ ID NO: 22. In some embodiments, the anti-tau antibody or antigen binding fragment thereof that is capable of binding to human tau comprises a heavy chain variable region of SEQ ID NO: 21 and a light chain variable region of SEQ ID NO: 22. See, e.g., Table 7. In some embodiments, the heavy chain constant region comprises SEQ ID NO: 23. In some embodiments, the heavy chain constant region comprises SEQ ID NO: 24. See, e.g., Table 8.

In some embodiments, a patient that is symptomatic for Alzheimer's disease is administered the anti-Aβ protofibril antibody (e.g., BAN2401) for at least 24 weeks then administered the isolated anti-tau antibody or antigen binding fragment thereof that is capable of binding to human tau (e.g., E2814) in conjunction with the isolated anti-Aβ protofibril antibody. In some embodiments, a patient that is symptomatic for Alzheimer's disease is administered the anti-Aβ protofibril antibody, e.g., for 24 weeks or until the patient's rate of increase in tau PET (e.g., in a temporal region) is reduced relative to a control subject having AD who does not receive the treatment. The patient is then administered the isolated anti-tau antibody or antigen binding fragment thereof that is capable of binding to human tau in conjunction with the isolated anti-Aβ protofibril antibody. In some embodiments, a patient that is symptomatic for Alzheimer's disease is administered the anti-Aβ protofibril antibody for 24 weeks or until the patient is amyloid negative, then administered the isolated anti-tau antibody or antigen binding fragment thereof that is capable of binding to human tau in conjunction with the isolated anti-Aβ protofibril antibody.

In some embodiments, the patient is asymptomatic for Alzheimer's disease (pre-AD) and is first administered an isolated anti-tau antibody or antigen binding fragment thereof that is capable of binding to human tau (e.g., E2814), e.g., for 52 weeks before being administered the isolated anti-tau antibody or antigen binding fragment thereof that is capable of binding to human tau in conjunction with an isolated anti-Aβ protofibril antibody (e.g., BAN2401). In some embodiments, such a subject is identified based on a tau PET level higher than in a control subject who does not have pre-AD. In some embodiments, a patient that is asymptomatic for Alzheimer's disease is administered the isolated anti-tau antibody or antigen binding fragment thereof that is capable of binding to human tau for 52 weeks or until the patient's rate of increase in tau PET is reduced relative to a control subject having AD who does not receive the treatment, then administered the isolated anti-tau antibody or antigen binding fragment thereof that is capable of binding to human tau in conjunction with the isolated anti-Aβ protofibril antibody. In some embodiments, a patient that is asymptomatic for Alzheimer's disease is administered the isolated anti-tau antibody or antigen binding fragment thereof that is capable of binding to human tau for 52 weeks or until the patient is amyloid negative, then administered the isolated anti-tau antibody or antigen binding fragment thereof that is capable of binding to human tau in conjunction with the isolated anti-Aβ protofibril antibody.

As used herein, the term “Amyloid PET” refers to Amyloid positron emission tomography imaging. In some embodiments, PET imaging (also referred to as a PET scan) is performed to assess for amyloid pathology. In some embodiments, amyloid PET is assessed with a PET tracer and uses the same tracer in follow-up assessments. In some embodiments, the amyloid PET tracer may be florbetaben (e.g., 18F-Florbetaben (Neuraceq®)), florbetapir (e.g., 18F-Florbetapir (Amyvid®)), and/or flutametamol (e.g., 18F-Flutemetamol (Vizamyl®)). In some embodiments, the PET imaging uses a florbetapir tracer. In some embodiments, the PET imaging uses a flutemetamol tracer. In some embodiments, the PET imaging uses a florbetaben tracer. Other exemplary amyloid PET tracers may be [11C]PiB or [18F]NAV4694 (flutafuranol).

Amyloid positron emission tomography (PET) imaging can be used to confirm the presence of amyloid pathology in the brain of early AD subjects in the screening phase of the study and/or to evaluate the effects of the at least one anti-Aβ antibody on amyloid levels in the brain, both by whole brain analysis (e.g., the average of 5-6 cortical regions) and brain region analysis. In some embodiments, the PET scan uses florbetaben (e.g., 18F-Florbetaben (Neuraceq®)), florbetapir (e.g., 18F-Florbetapir (Amyvid®)), and/or flutametamol (e.g., 18F-Flutemetamol (Vizamyl®)). In some embodiments, the PET scan uses florbetapir. In some embodiments, the PET scan uses a flutemetamol tracer. In some embodiments, the PET scan uses a florbetaben tracer. In some embodiments, amyloid plaque load can be identified by a PET imaging uptake visual read, e.g., by a trained radiologist. In some embodiments, 2 readers (1 designated as Primary Reader) visually assess the images to determine whether the scan is positive or negative for amyloid. In further embodiments, four regions of the brain are assessed for uptake of the imaging agent: the temporal lobes, the occipital lobes, the prefrontal cortex, and the parietal cortex and a positive amyloid scan has either 1 region with intense gray matter uptake that is greater than the white matter uptake and extends to the outer edges of the brain, or 2 regions with areas of reduced gray-white contrast. In further embodiments, if disagreement occurs between 2 readers, both meet to review the scan for a consensus read.

In some embodiments, amyloid plaque load can be identified by a standard uptake value ratio (SUVr) as compared to a reference region. Methods for calculating amyloid PET SUVr are known in the art and may include those described herein. One exemplary method for quantitative analysis (e.g, computing) of SUVr is the PMOD PNEURO Biomedical Image Quantification Software (PMOD Technologies, Zurich, Switzerland. In some embodiments, PET images are first assessed for subject movement in the X, Y, and Z planes and corrected for motion, if needed, before individual images (e.g., 5-minute emission frames) are averaged, e.g., using a PMOD Averaging Function (PET frames averaged to increase the signal to noise ratio). In some embodiments, corresponding MRIs from subjects are prepared (e.g., using matrix size reduction processing, cropping of the MRI to include only the brain, segmentation to separate images into binary maps of gray matter, white matter, and CSF, and stripping the image of skull leaving only brain mask). In some embodiments, the averaged PET images and prepared MRIs are matched using the PMOD Matching Function, placing the images in the same orientation. In some embodiments, a Brain Normalization function, e.g., as provided by PMOD software, is used along with Brain Norm and Rigid Matching transformation matrices, to produce an averaged PET. In some embodiments, this averaged PET which is normalized to the MNInst space (Senjem et al, 2005) that is in the same orientation as the subject's segmented MRI for quantitative analysis. In some embodiments, the PMOD Mask Function is used to mask the brain and zero the image outside of the mask to create a Normalized Gray Matter PET and a Normalized White Matter PET. Standard uptake values (SUVs) may be calculated for all gray matter mapped regions and the 3 white matter regions (pons, cerebellar white, and subcortical white) using PMOD software calculated using the normalized PET, subject weight, and injected dose of tracer to arrive at the units of SUVs. In some embodiments, the SUVr is the ratio of the global cortical average as compared to a reference region of choice. In some embodiments, a whole cerebellum mask is used as the reference region. In some embodiments, the reference region is subcortical white matter, derived whole cerebellum, whole cerebellum adjusted by subcortical white matter, cerebellar gray matter, and composite reference regions consisting of cerebellar cortex, pons subcortical white matter, and cerebella white matter.

In some embodiments, after administration of the first dose of the composition the adjusted mean change from baseline in a subject's amyloid PET SUVr value is reduced by at least −0.10, at least −0.15, at least −0.20, at least −0.25, at least −0.30, at least −0.35, at least −0.40, at least −0.45, at least −0.50, at least −0.55, at least −0.60, at least −0.65, at least −0.70, at least −0.75, at least −0.80, at least −0.85, at least −0.90, or at least −0.95 relative to baseline. In some embodiments, the adjusted mean change from baseline in a subject's amyloid PET SUVr value is reduced by −0.20 to −0.30.

In some embodiments, the amyloid beta plaque levels in the brain are evaluated using PET imaging. In some embodiments, the PET imaging uses an amyloid PET tracer. In some embodiments, the PET imaging uses florbetaben (e.g., 18F-Florbetaben (Neuraceq®)), florbetapir (e.g., 18F-Florbetapir (Amyvid®)), and/or flutametamol (e.g., 18F-Flutemetamol (Vizamyl®)) In some embodiments, the PET imaging uses a florbetapir tracer. In some embodiments, the PET imaging used a flutemetamol tracer. In some embodiments, the PET imaging uses a florbetaben tracer. In some further embodiments, different tracers may yield different results. In some embodiments, the adjusted mean reduction threshold is dependent upon the tracer used. In some embodiments, comparing global cortical average versus whole cerebellum reference, the adjusted mean change from baseline in a subject's amyloid PET SUVr value is reduced by at least −0.20, such as at least −0.25, after 12 months of administration of the composition comprising a therapeutically effective amount of at least one anti-Aβ protofibril antibody. In some embodiments, the adjusted mean change from baseline in a subject's amyloid PET SUVr value is reduced by at least −0.25, such as at least −0.30, after 18 months of administration of the composition comprising a therapeutically effective amount of at least one anti-Aβ protofibril antibody.

In some embodiments, the reduction of amyloid in the brain is determined by imaging using binding of radiotracers for brain Aβ amyloid and visualized with PET. In some embodiments, the reduction in the adjusted mean change from baseline is at least −50, such as at least −55 or at least −59 centiloid after 12 months of administration of the composition comprising a therapeutically effective amount of at least one anti-Aβ protofibril antibody. In some embodiments, the reduction in the adjusted mean change from baseline is at least −60, such as at least −65 or at least −70 centiloid after 18 months of administration of the composition comprising a therapeutically effective amount of at least one anti-Aβ protofibril antibody.

In some embodiments, said method results in an increased cerebrospinal fluid Aβ1-42 level relative to the cerebrospinal fluid Aβ1-42 level prior to said administration. In some embodiments, said method results in an increase of cerebrospinal fluid Aβ1-42 level of at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% relative to the cerebrospinal fluid Aβ1-42 level prior to said administration.

In some embodiments, administration of the composition results in a brain amyloid level reduction of −0.20 to −0.45, such as from −0.25 to −0.35 as determined by visual reads of amyloid PET images, wherein the subject is ApoE4-positive. In some embodiments, administration of the composition results in a brain amyloid level reduction of at least −0.25, as determined by visual reads of amyloid PET images, wherein the subject is ApoE4-positive. In some embodiments, administration of the composition results in a brain amyloid level reduction of at least 0.30, as determined by visual reads of amyloid PET images, wherein the subject is ApoE4-positive.

In some embodiments, a subject's brain amyloid level is determined by visual reads of amyloid PET images and expressed as a PET standard uptake value ratio (SUVr value). In some embodiments, administration of the composition results in a brain amyloid level reduction, as measured by an amyloid PET SUVr value, of at least −0.01, at least −0.02, at least −0.03, at least −0.04, at least −0.05, at least −0.06, at least −0.07, at least −0.08, at least −0.09, at least −0.10, at least −0.11, at least −0.12, at least −0.13, at least −0.14, at least −0.15, at least −0.16, at least −0.17, at least −0.18, at least −0.19, at least −0.20, at least −0.21, at least −0.22, at least −0.23, at least −0.24, at least −0.25, at least −0.26, at least −0.27, at least −0.28, or at least −0.29 relative to placebo, wherein the subject is ApoE4-negative.

In some embodiments, administration of the composition results in a brain amyloid level reduction of −0.10 to −0.40, as measured by an amyloid PET SUVr value, wherein the subject is ApoE4-negative. In some embodiments, administration of the composition results in a brain amyloid level reduction of at least −0.20, as measured by an amyloid PET SUVr value, wherein the subject is ApoE4-negative. In some embodiments, administration of the composition results in a brain amyloid level reduction of at least −0.25, as measured by an amyloid PET SUVr value, wherein the subject is ApoE4-negative.

In some embodiments, a subject's brain amyloid level is determined by visual reads of amyloid PET images and expressed as a PET standard uptake value ratio (SUVr value). In some embodiments, administration of the composition results in a brain amyloid level reduction of −0.10 to −0.40, as measured by an amyloid PET SUVr value, wherein the subject is ApoE4-negative. In some further embodiments, the subject has an increase in the Aβ42/40 ratio after administration of the first dose of the composition comprising the anti-Aβ protofibril antibody, e.g., an increase to a ratio of about 0.05-0.1, e.g., about 0.08-0.1, e.g., about 0.092, indicating a change from amyloid positive to amyloid negative in the brain of the subject. In some further embodiments, the subject has an increase in the Aβ42/40 ratio after administration of the first dose of the composition comprising the anti-Aβ protofibril antibody, e.g., an increase to a ratio above 0.092, indicating a change from amyloid positive to amyloid negative in the brain of the subject. In some embodiments, the subject has an increase in the Aβ42/40 ratio after 6 months or after 12 months or after 18 months or after 24 months of administration of the first dose of the composition comprising the anti-Aβ protofibril antibody, e.g., an increase to a ratio of about 0.05-0.1, e.g., about 0.08-0.1, e.g., about 0.092, indicating a change from amyloid positive to amyloid negative in the brain of the subject. In some embodiments, the subject has an increase in the Aβ42/40 ratio after 6 months or after 12 months or after 18 months or after 24 months of administration of the first dose of the composition comprising the anti-Aβ protofibril antibody, e.g., an increase to a ratio above 0.092, indicating a change from amyloid positive to amyloid negative in the brain of the subject. In some embodiments, an increase in the Aβ42/40 ratio indicates a reduction in brain amyloid level, as determined by visual reads of amyloid PET images. In some embodiments, a subject with a reduction in brain amyloid level is given a reduced dose or frequency of the anti-Aβ protofibril antibody, alone or in combination with at least one additional therapy, e.g., a BACE inhibitor and/or anti-tau antibody.

### SEQUENCE TABLES

TABLE 1
Amino acid sequences of monoclonal antibody (mAb)
CDRs

| | IgG | SEQ | |
|---|---|---|---|
| mAb | chain | ID NO | Amino acid sequence |
| BAN2401 | HCDR1 | 1 | SFGMH |
| | HCDR2 | 2 | YISSGSSTIYYGDTVKG |
| | HCDR3 | 3 | EGGYYYGRSYYTMDY |
| BAN2401 | LCDR1 | 4 | RSSQSIVHSNGNTYLE |
| | LCDR2 | 5 | KVSNRFS |
| | LCDR3 | 6 | FQGSHVPPT |

TABLE 2
Amino acid sequences of mAb variable regions

| mAb | IgG chain | SEQ ID NO | Amino acid sequence |
|---|---|---|---|
| BAN2401 | Heavy chain | 7 | EVQLVESGGGLVQPGGSLRLSCSASGF |
| | variable | | TFSSFGMHWVRQAPGKGLEWVAYISS |
| | region | | GSSTIYYGDTVKGRFTISRDNAKNSLFL |
| | | | QMSSLRAEDTAVYYCAREGGYYYGRS |
| | | | YYTMDYWGQGTTVTVSS |
| BAN2401 | Light chain | 8 | DVVMTQSPLSLPVTPGAPASISCRSSQSI |
| | variable | | VHSNGNTYLEWYLQKPGQSPKLLIYKV |
| | region | | SNRFSGVPDRFSGSGSGTDFTLRISRVE |
| | | | AEDVGIYYCFQGSHVPPTFGPGTKLEIK |

TABLE 3
Amino acid sequences of mAb heavy and light chains

| mAb | IgG chain | SEQ ID NO | Amino acid sequence |
|---|---|---|---|
| BAN2401 | Heavy chain | 9 | EVQLVESGGGLVQPGGSLRLSCSASG |
| | | | FTFSSFGMHWVRQAPGKGLEWVAYI |
| | | | SSGSSTIYYGDTVKGRFTISRDNAKNS |
| | | | LFLQMSSLRAEDTAVYYCAREGGYY |
| | | | YGRSYYTMDYWGQGTTVTVSSASTK |
| | | | GPSVFPLAPSSKSTSGGTAALGCLVK |
| | | | DYFPEPVTVSWNSGALTSGVHTFPAV |
| | | | LQSSGLYSLSSVVTVPSSSLGTQTYICN |
| | | | VNHKPSNTKVDKRVEPKSCDKTHTCPP |
| | | | CPAPELLGGPSVFLFPPKPKDTLMISRTP |
| | | | EVTCVVVDVSHEDPEVKFNWYVDGV |
| | | | EVHNAKTKPREEQYNSTYRVVSVLT |
| | | | VLHQDWLNGKEYKCKVSNKALPAPI |
| | | | EKTISKAKGQPREPQVYTLPPSREEM |
| | | | TKNQVSLTCLVKGFYPSDIAVEWESN |
| | | | GQPENNYKTTPPVLDSDGSFFLYSKL |
| | | | TVDKSRWQQGNVFSCSVMHEALHN |
| | | | HYTQKSLSLSPGK |
| BAN2401 | Light chain | 10 | light chain: |
| | | | DVVMTQSPLSLPVTPGAPASISCRSSQ |
| | | | SIVHSNGNTYLEWYLQKPGQSPKLLI |
| | | | YKVSNRFSGVPDRFSGSGSGTDFTLRI |
| | | | SRVEAEDVGIYYCFQGSHVPPTFGPGT |
| | | | KLEIKRTVAAPSVFIFPPSDEQLKSGTAS |
| | | | VVCLLNNFYPREAKVQWKVDNALQSG |
| | | | NSQESVTEQDSKDSTYSLS |
| | | | STLTLSKADYEKHKVYACEVTHQGL |
| | | | SSPVTKSFNRGEC |

TABLE 4
Amino acid sequences of mAb constant regions

| mAb | IgG chain | Class | SEQ ID NO | Amino acid sequence |
|---|---|---|---|---|
| BAN2401 | Heavy chain | IgG1 | 11 | ASTKGPSVFPLAPSSKSTSGGT |
| | | | | AALGCLVKDYFPEPVTVSWN |
| | | | | SGALTSGVHTFPAVLQSSGLY |
| | | | | SLSSVVTVPSSSLGTQTYICNV |
| | | | | NHKPSNTKVDKRVEPKSCDK |
| | | | | THTCPPCPAPELLGGPSVFLFP |
| | | | | PKPKDTLMISRTPEVTCVVVD |
| | | | | VSHEDPEVKFNWYVDGVEVH |
| | | | | NAKTKPREEQYNSTYRVVSVL |
| | | | | TVLHQDWLNGKEYKCKVSNK |
| | | | | ALPAPIEKTISKAKGQPREPQV |
| | | | | YTLPPSREEMTKNQVSLTCLV |
| | | | | KGFYPSDIAVEWESNGQPENN |
| | | | | YKTTPPVLDSDGSFFLYSKLT |
| | | | | VDKSRWQQGNVFSCSVMHEA |
| | | | | LHNHYTQKSLSLSPGK |
| BAN2401 | Light chain | kappa | 12 | RTVAAPSVFIFPPSDEQLKSGT |
| | | | | ASVVCLLNNFYPREAKVQWK |
| | | | | VDNALQSGNSQESVTEQDSK |
| | | | | DSTYSLSSTLTLSKADYEKHK |
| | | | | VYACEVTHQGLSSPVTKSFNR |
| | | | | GEC |

TABLE 5
Amino acid sequences of Amyloid β

| Amyloid β | SEQ ID NO | Amino acid sequence |
|---|---|---|
| Amyloid β | 13 | DAEFRHDSGYEVHHQKLVFFAEDVGS |
| 1-42 | | NKGAIIGLMVGGVVIA |
| Amyloid β | 14 | DAEFRHDSGYEVHHQKLVFFAEDVGS |
| 1-40 | | NKGAIIGLMVGGVV |

TABLE 6
Amino acid sequences of E2814 CDRs

| | IgG | SEQ | |
|---|---|---|---|
| mAb | chain | ID NO | Amino acid sequence |
| E2814 | HCDR1 | 15 | TYWIT |
| Heavy Chain | HCDR2 | 16 | DIYPGSSISNYNEKFKS |
| | HCDR3 | 17 | EDGYDAWFAY |
| E2814 Light | LCDR1 | 18 | RSSQSILHSNGNTYLE |
| Chain | LCDR2 | 19 | KVSNRFS |
| | LCDR3 | 20 | FQGSHVPFT |

TABLE 7
Amino acid sequences of E2814 variable regions

| mAb | IgG chain | SEQ ID NO | Amino acid sequence |
|---|---|---|---|
| E2814 | Heavy chain | 21 | EVQLLESGGGLVQPGGSLRLSCAASGY |
| | variable | | TFTTYWITWVRQAPGKGLEWVSDIYPG |
| | region | | SSISNYNEKFKSRFTISVDNSKNTLYLQ |
| | | | MNSLRAEDTAVYYCAREDGYDAWFA |
| | | | YWGQGTLVTVSS |
| E2814 | Light chain | 22 | DIQMTQSPSSLSASVGDRVTITCRSSQSI |
| | variable | | LHSNGNTYLEWYQQKPGKAPKLLISKV |
| | region | | SNRFSGVPSRFSGSGSGTDFTLTISSLQP |
| | | | EDFATYYCFQGSHVPFTFGQGTKLEIK |

TABLE 8
Amino acid sequences of E2814 constant regions

| mAb | IgG chain | Class | SEQ ID NO | Amino acid sequence |
|---|---|---|---|---|
| E2814 | Heavy chain | IgG1 | 23 | ASTKGPSVFPLAPSSKSTSGGT |
| | | | | AALGCLVKDYFPEPVTVSWN |
| | | | | SGALTSGVHTFPAVLQSSGLY |
| | | | | SLSSVVTVPSSSLGTQTYICNV |
| | | | | NHKPSNTKVDKKVEPKSCDK |
| | | | | THTCPPCPAPELLGGPSVFLFP |
| | | | | PKPKDTLMISRTPEVTCVVVD |
| | | | | VSHEDPEVKFNWYVDGVEVH |
| | | | | NAKTKPREEQYNSTYRVVSVL |
| | | | | TVLHQDWLNGKEYKCKVSNK |
| | | | | ALPAPIEKTISKAKGQPREPQV |
| | | | | YTLPPSRDELTKNQVSLTCLV |
| | | | | KGFYPSDIAVEWESNGQPENN |
| | | | | YKTTPPVLDSDGSFFLYSKLT |
| | | | | VDKSRWQQGNVFSCSVMHEA |
| | | | | LHNHYTQKSLSLSPGK |
| E2814 | Light chain | kappa | 24 | RTVAAPSVFIFPPSDEQLKSGT |
| | | | | ASVVCLLNNFYPREAKVQWK |
| | | | | VDNALQSGNSQESVTEQDSK |
| | | | | DSTYSLSSTLTLSKADYEKHK |
| | | | | VYACEVTHQGLSSPVTKSFNR |
| | | | | GEC |

TABLE 9
Amino acid sequence of Tau

| | SEQ ID NO | Amino acid sequence |
|---|---|---|
| Tau | 25 | MAEPRQEFEVMEDHAGTYGLGDRKDQGGYTMHQDQEGDT |
| | | DAGLKESPLQTPTEDGSEEPGSETSDAKSTPTAEDVTAPLVD |
| | | EGAPGKQAAAQPHTEIPEGTTAEEAGIGDTPSLEDEAAGHVT |
| | | QARMVSKSKDGTGSDDKKAKGADGKTKIATPRGAAPPGQK |
| | | GQANATRIPAKTPPAPKTPPSSGEPPKSGDRSGYSSPGSPGTP |
| | | GSRSRTPSLPTPPTREPKKVAVVRTPPKSPSSAKSRLQTAPVP |
| | | MPDLKNVKSKIGSTENLKHQPGGGKVQIINKKLDLSNVQSK |
| | | CGSKDNIKHVPGGGSVQIVYKPVDLSKVTSKCGSLGNIHHKP |
| | | GGGQVEVKSEKLDFKDRVQSKIGSLDNITHVPGGGNKKIETH |
| | | KLTFRENAKAKTDHGAEIVYKSPVVSGDTSPRHLSNVSSTGS |
| | | IDMVDSPQLATLADEVSASLAKQGL |

The present disclosure is further illustrated by the following examples that should not be construed as limiting. The contents of all references, patents, and published patent applications cited throughout this application, as well as the figures, are incorporated herein by reference in their entirety for all purposes.

### EXAMPLES

### Example 1. Lecanemab Significantly Slows Tau Accumulation in Temporal Regions at 13 and 18 Months

Lecanemab (LEC) or placebo (PBO) were administered to subjects with Alzheimer's disease (patients with confirmed presence of amyloid pathology and mild cognitive impairment due to Alzheimer's disease or mild dementia due to Alzheimer's disease, consistent with Stage 3 and Stage 4 Alzheimer's disease), and tau PET SUVR was measured, as described herein.

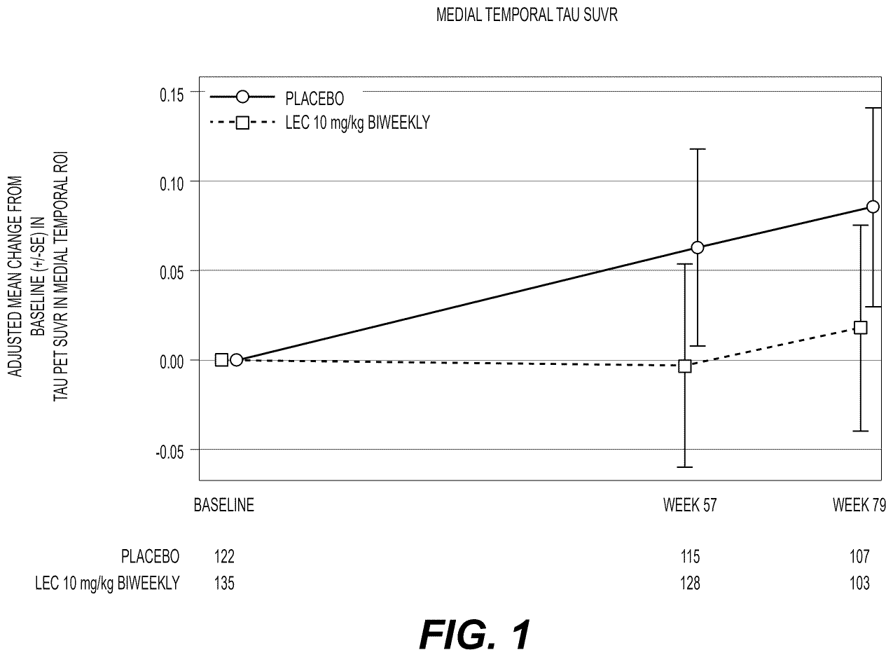

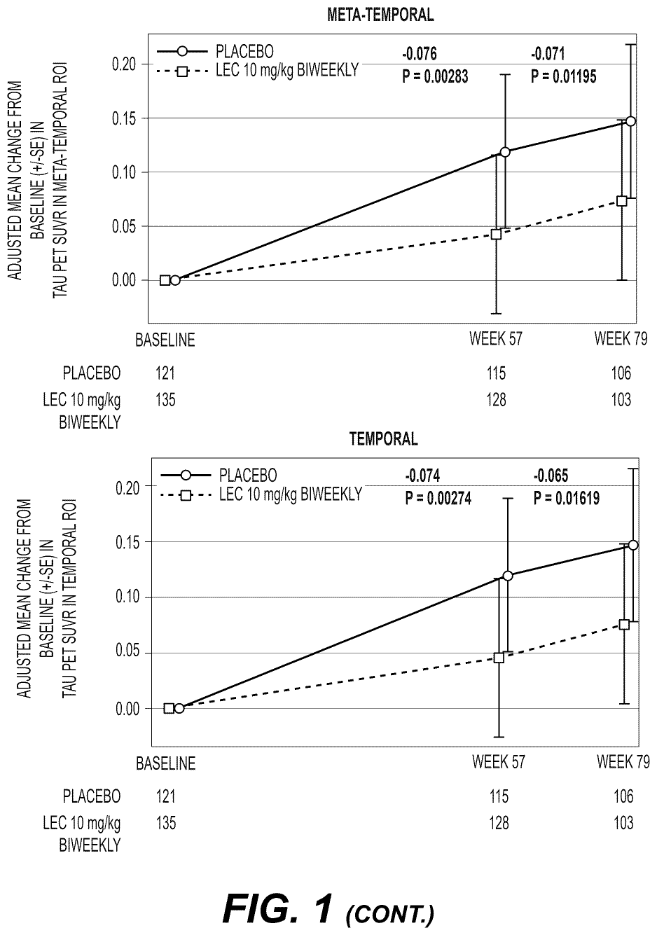

FIG. 1 shows a plot of the adjusted mean change from baseline tau PET SUVR (standard error, SE) over time in subjects administered lecanemab or placebo. Administration of lecanemab significantly slows tau accumulation in temporal regions at 13 and 18 months (indicated as Week 57 and Week 79, respectively). A similar numerical effect is observed in other brain regions but does not reach statistical significance at 18 months.

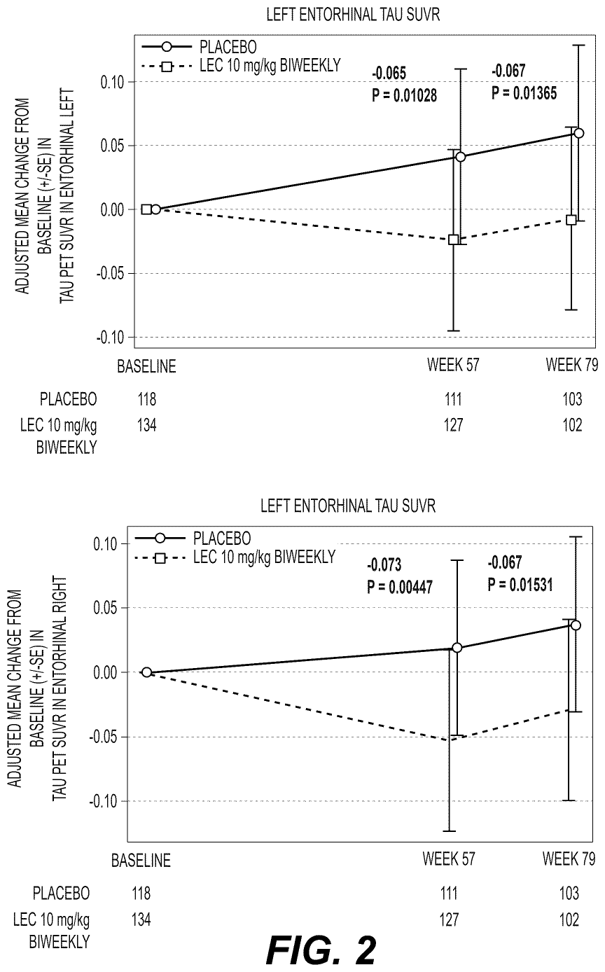

FIG. 2 shows the effect of lecanemab administration in the entorhinal cortex of subjects. Lecanemab reduces tau PET SUVR in the left and right entorhinal cortex.

### Example 2. Extent of Tau Slowing is Associated with Baseline Tau Levels (Population Level)

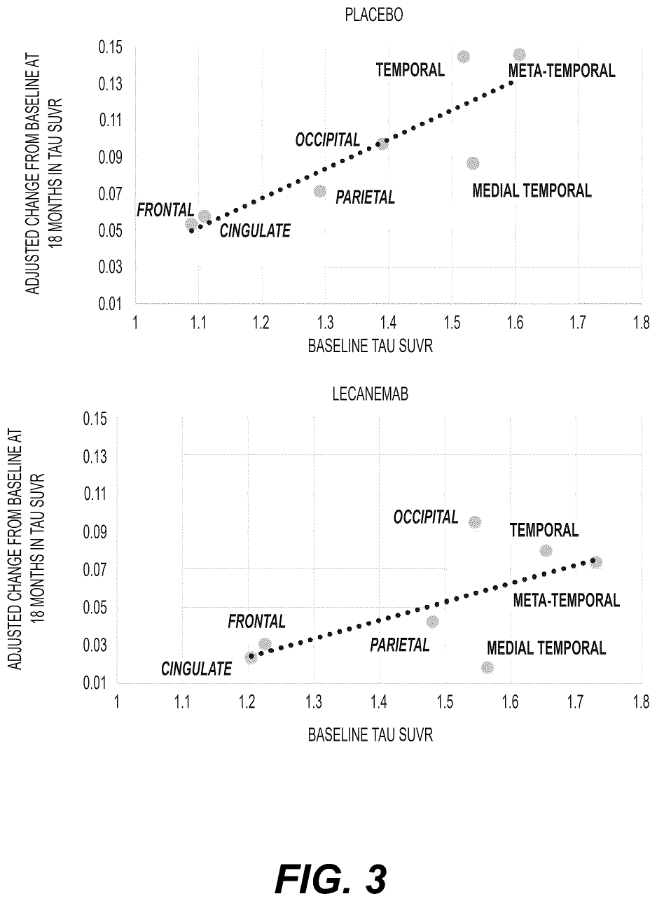

FIG. 3 shows plots of the adjusted change from baseline tau PET SUVR at 18 months as a function of the baseline tau PET SUVR (upper plot: subjects who received a placebo; lower plot: subjects who received lecanemab).

In subjects who received a placebo, regions with higher tau accumulation are those with higher baseline tau. Temporal regions have the highest baseline Tau levels and greatest Tau accumulation at 18 months. Lecanemab slows down tau accumulation at 18 months in all regions, and this is statistically significant for temporal regions (temporal, meta-temporal, and medial-temporal, indicated in bold text).

### Example 3. Effects of Lecanemab on Tau PET SUVR in Select Brain Regions

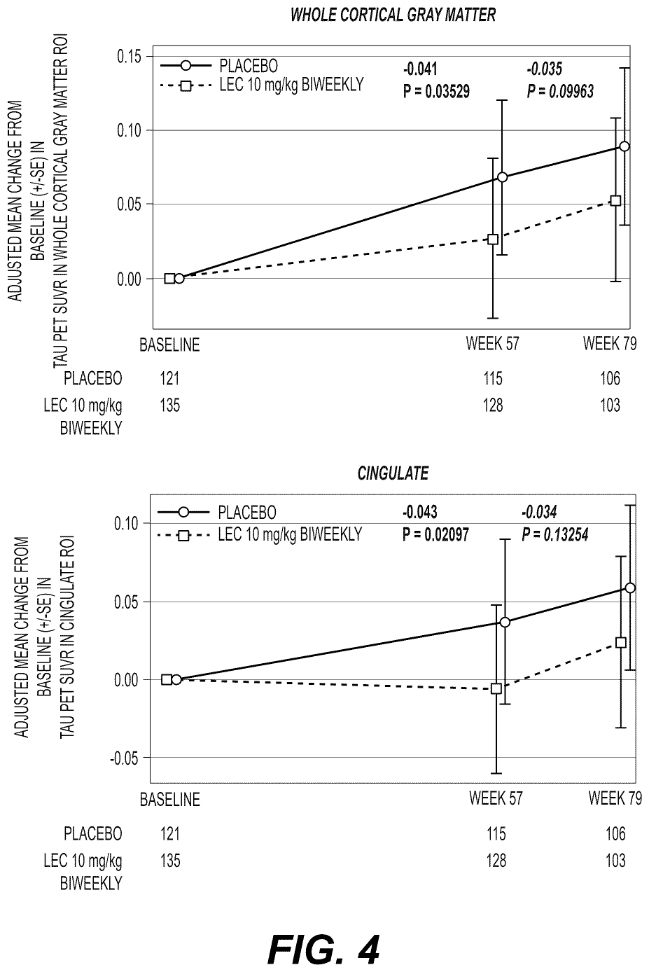

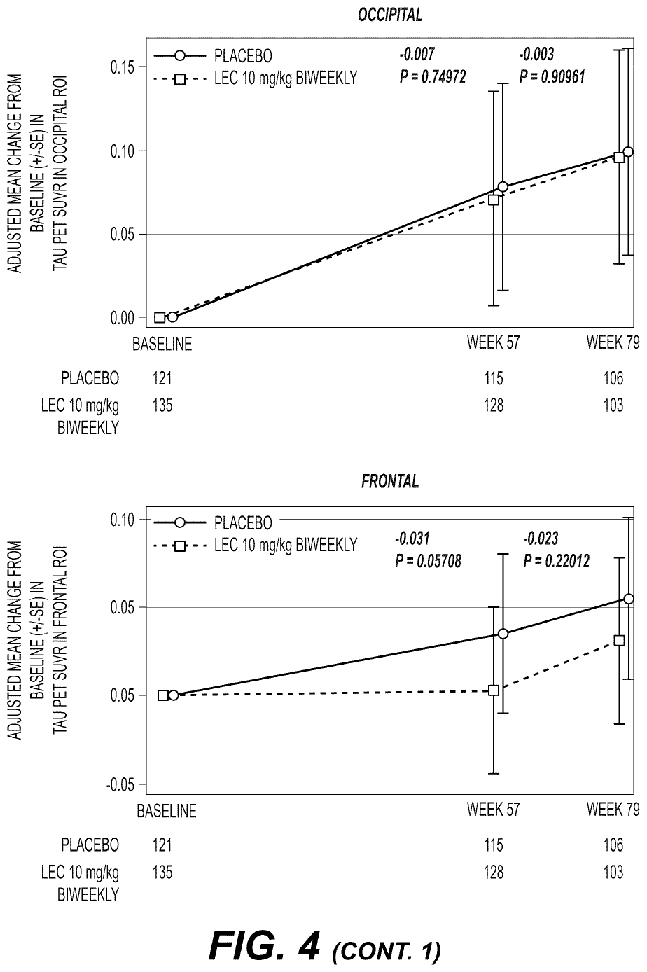

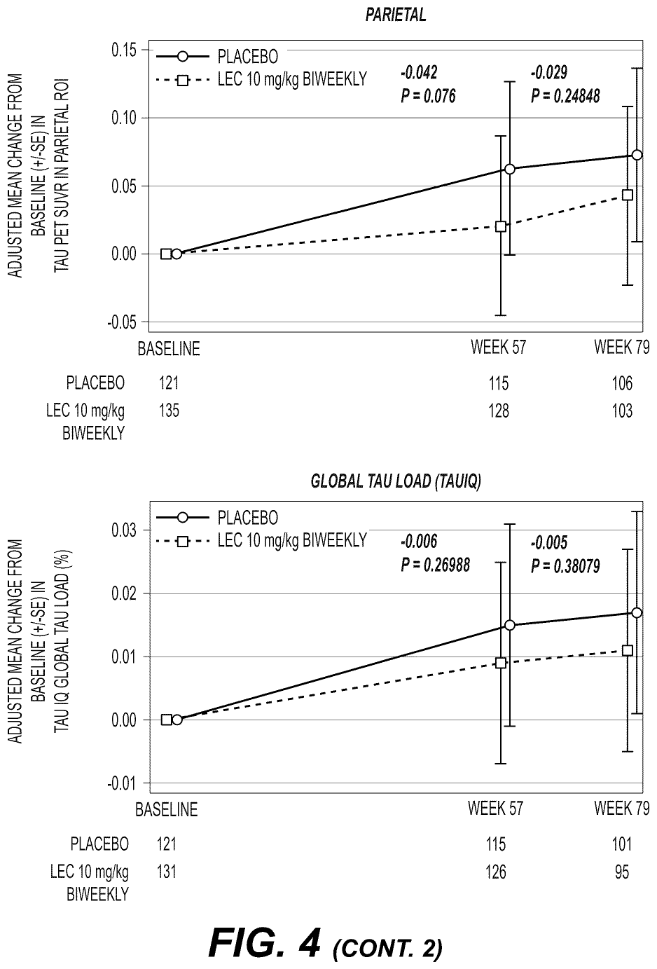

An effect of lecanemab administration on tau PET SUVR was observed at 18 months in the whole cortical gray matter, cingulate cortex, occipital cortex, frontal cortex, parietal cortex, as well as the global tau load (TauIQ). In these regions, the numerical effect of lecanemab administration did not reach statistical significance (FIG. 4).

### Example 4: Baseline Tau PET SUVR

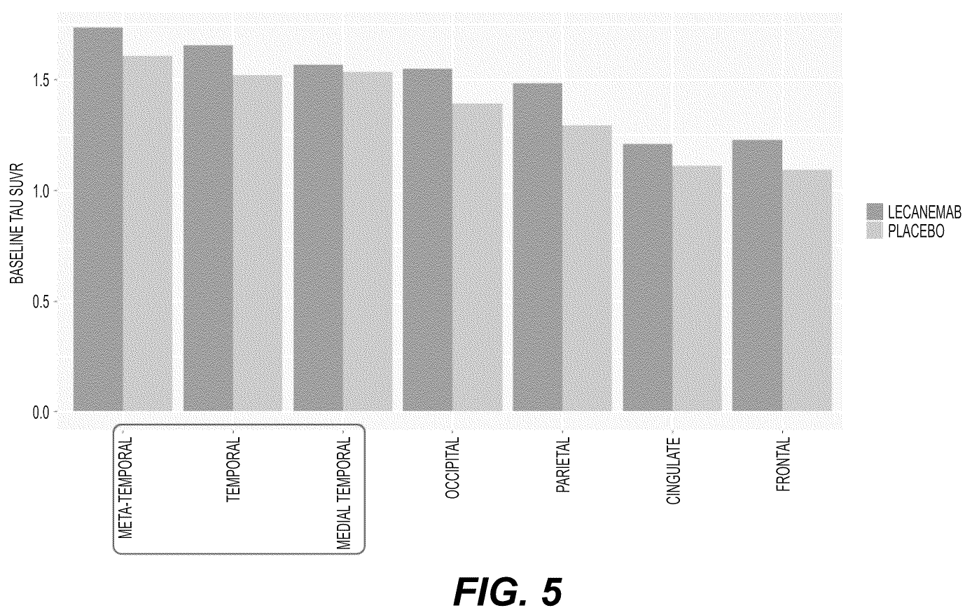

3 composite regions known to accumulate tau early in AD are the temporal region, medial temporal region, and meta-temporal region. After 18 months, there was a statistically significant reduction in change from baseline in brain tau pathology in these 3 composite regions, as measured by tau PET SUVR. FIG. 5 shows a reduction in change from baseline in the LEC10-BW subjects as compared to the PBO (medial temporal ROI: adjusted mean −0.068], P=0.00237; meta-temporal ROI: −0.071, P=0.01195; temporal ROI: adjusted mean −0.065], P=0.01619). The difference was seen as early as 13 months.

These temporal regions, known to accumulate tau early in the disease process, had the highest tau PET SUVR levels at baseline in both PBO subjects and LEC10-BW subjects. No statistically significant effects were observed at 18 months on the global tau load and on the reduction in change from baseline in occipital, parietal, cingulate, and frontal regions and in the whole cortical gray matter.

### Example 5. Baseline Tau PET SUVR Increases from Clusters 1 to 4

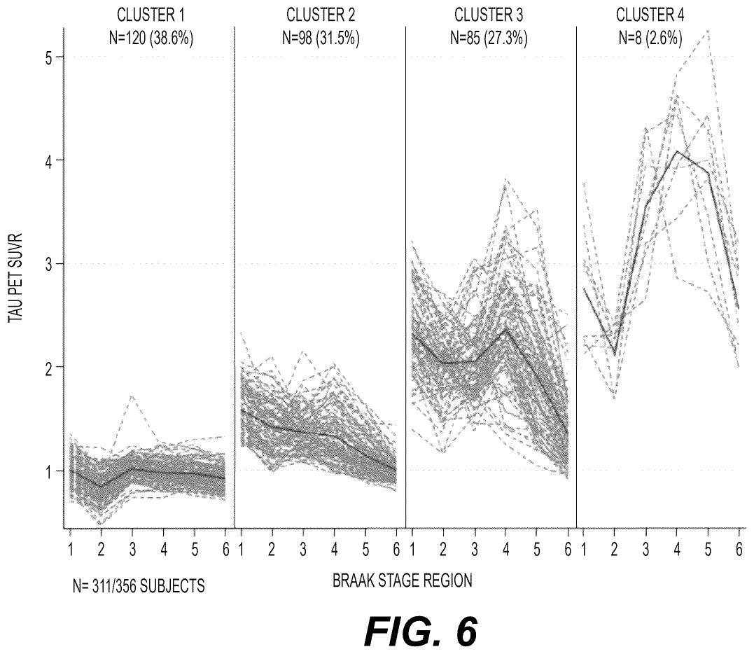

FIG. 6 shows that at baseline, about 70% of all subjects fall into clusters 1 & 2, characterized by low to early tau deposition in early Braak Stage regions. Clusters were derived according to the methods described in Charil et al., Baseline Tau in CLARITY AD: a phase 3 placebo-controlled, double-blind, parallel-group, 18-month study evaluating Lecanemab (BAN2401) in early Alzheimer's Disease (AD). International Conference on Alzheimer's and Parkinson's Diseases and related neurological disorders (ADPD). Mar. 15-20, 2022. Barcelona, Spain).

### Example 6. Tau PET SUVR Change at 13 Months and 18 Months

FIG. 3 shows the effects of lecanemab on tau PET SUVR at 13 months in the whole cortical gray matter and cingulate cortex. Subjects who received LEC showed significant group differences as compared to subjects who received PBO. Results from the whole cortical gray matter and cingulated cortex are summarized in Table 10.

The effects of lecanemab on tau PET SUVR at 13 months in other regions (occipital cortex, frontal cortex, and parietal cortex) and the global tau load (TauIQ) was measured. A numerical effect was observed in subjects who received lecanemab, but did not reach statistical significance. Results from the parietal cortex and the occipital cortex at 13 months are summarized in Table 11.

Results from the whole cortical gray matter, cingulate cortex, occipital cortex, frontal cortex, occipital cortex, parietal cortex, and global tau load (TauIQ) at 18 months are summarized in Table 12.

TABLE 10
Tau PET SUVR change in whole cortical gray matter and cingulate cortex at 13 months.

| Whole Cortical Gray Matter | | | Cingulate | | |
|---|---|---|---|---|---|
| Statistics | Placebo | Lecanemab | Statistics | Placebo | Lecanemab |
| N(OBS/MMRM/MMRM_T) | 115/115/122 | 128/128/135 | N(OBS/MMRM/MMRM_T) | 115/115/122 | 128/128/135 |
| Baseline mean(SD) | 1.284 | 1.429 | Baseline mean (SD) | 1.110 | 1.206 |
| | (0.4818) | (0.7247) | | (0.5073) | (0.6113) |
| Obs. Mean CFB (SD) | 0.069 | 0.031 | Obs. Mean CFB (SD) | 0.039 | −0.006 |
| | (0.1339) | (0.1572) | | (0.1355) | (0.1507) |
| Adj. mean (SE) | 0.067 | 0.026 | Adj. mean (SE) | 0.036 | −0.007 |
| | (0.052) | (0.054) | | (0.053) | (0.054) |
| Adj. mean diff. | — | −0.041 | Adj. mean diff. | — | −0.043 |
| 95% CI for diff. | , | −0.079, | 95% CI for diff. | , | −0.080, |
| | | −0.003 | | | −0.007 |
| P-value | — | 0.03529 | P-value | — | 0.02097 |

TABLE 11
Tau PET SUVR change in parietal cortex and occipital cortex 13 months.

| Parietal | | | Occipital | | |
|---|---|---|---|---|---|
| Statistics | Placebo | Lecanemab | Statistics | Placebo | Lecanemab |
| N(OBS/MMRM/MMRM_T) | 115/115/122 | 128/128/135 | N(OBS/MMRM/MMRM_T) | 115/115/122 | 128/128/135 |
| Baseline mean (SD) | 1.291 | 1.482 | Baseline mean (SD) | 1.390 | 1.547 |
| | (0.5981) | (0.9299) | | (0.6211) | (0.8615) |
| Obs. meanCFB (SD) | 0.070 | 0.031 | Obs. mean CFB (SD) | 0.070 | 0.070 |
| | (0.1615) | (0.1919) | | (0.1479) | (0.1831) |
| Adj. mean (SE) | 0.062 | 0.020 | Adj. mean (SE) | 0.077 | 0.069 |
| | (0.064) | (0.066) | | (0.061) | (0.064) |
| Adj. meandiff. | — | −0.042 | Adj. mean diff. | — | −0.007 |
| 95% CI for diff. | , | −0.088, | 95% CI for diff. | , | −0.053, |
| | | 0.004 | | | 0.038 |
| P-value | — | 0.07600 | P-value | — | 0.74972 |

TABLE 12
Tau PET SUVR change in whole cortical gray matter, cingulate cortex, frontal
cortex, occipital cortex, parietal cortex, and TauIQ at 18 months.

| Whole Cortical Gray Matter | | | Cingulate | | |
|---|---|---|---|---|---|
| Statistics | Placebo | Lecanemab | Statistics | Placebo | Lecanemab |
| N(OBS/MMRM/ | 107/107/ | 103/103/ | N(OBS/MMRM/ | 107/107/ | 103/103/ |
| MMRM_T) | 122 | 135 | MMRM_T) | 122 | 135 |
| Baseline mean (SD) | 1.284 | 1.429 | Baseline mean (SD) | 1.110 | 1.206 |
| | (0.4818) | (0.7247) | | (0.5073) | (0.6113) |
| Obs. mean CFB (SD) | 0.084 | 0.051 | Obs. Mean CFB (SD) | 0.056 | 0.022 |
| | (0.1497) | (0.1637) | | (0.1737) | (0.1637) |
| Adj. mean (SE) | 0.087 | 0.052 | Adj. mean (SE) | 0.057 | 0.023 |
| | (0.052) | (0.054) | | (0.053) | (0.055) |
| Adj. mean diff. | — | −0.035 | Adj. mean diff. | — | −0.034 |
| 95% CI for diff. | , | −0.076, | 95% CI for diff. | , | −0.078, |
| | | 0.007 | | | 0.010 |
| P-value | — | 0.09963 | P-value | — | 0.13254 |
| Frontal | | | Occipital | | |
| Statistics | Placebo | Lecanemab | Statistics | Placebo | Lecanemab |
| N(OBS/MMRM/ | 107/107/ | 103/103/ | N(OBS/MMRM/ | 107/107/ | 103/103/ |
| MMRM_T) | 122 | 135 | MMRM_T) | 122 | 135 |
| Baseline mean (SD) | 1.089 | 1.227 | Baseline mean (SD) | 1.390 | 1.547 |
| | (0.3951) | (0.6114) | | (0.6211) | (0.8615) |
| Obs. Mean CFB (SD) | 0.064 | 0.045 | Obs. Mean CFB (SD) | 0.085 | 0.082 |
| | (0.1396) | (0.1403) | | (0.1468) | (0.2017) |
| Adj. mean (SE) | 0.053 | 0.030 | Adj. mean (SE) | 0.097 | 0.094 |
| | (0.045) | (0.047) | | (0.061) | (0.064) |
| Adj. mean diff. | — | −0.023 | Adj. mean diff. | — | −0.003 |
| 95% CI for diff. | , | −0.060, | 95% CI for diff. | , | −0.049, |
| | | 0.014 | | | 0.044 |
| P-value | — | 0.22012 | P-value | — | 0.90961 |
| Parietal | | | Global tau load (TauIQ) | | |
| Statistics | Placebo | Lecanemab | Statistics | Placebo | Lecanemab |
| N(OBS/MMRM/ | 107/107/ | 103/103/ | N(OBS/MMRM/ | 102/102/ | 95/95/ |
| MMRM_T) | 122 | 135 | MMRM_T) | 122 | 131 |
| Baseline mean (SD) | 1.291 | 1.482 | Baseline mean (SD) | 0.197 | 0.241 |
| | (0.5981) | (0.9299) | | (0.1511) | (0.2241) |
| Obs. Mean CFB (SD) | 0.075 | 0.044 | Obs. Mean CFB (SD) | 0.020 | 0.011 |
| | (0.1628) | (0.2099) | | (0.0431) | (0.0488) |
| Adj. mean (SE) | 0.071 | 0.042 | Adj. mean (SE) | 0.016 | 0.011 |
| | (0.064) | (0.066) | | (0.016) | (0.016) |
| Adj. mean diff. | — | −0.029 | Adj. mean diff. | — | −0.005 |
| 95% CI for diff. | , | −0.078, | 95% CI for diff. | , | −0.017, |
| | | 0.020 | | | 0.007 |
| P-value | — | 0.24848 | P-value | — | 0.38079 |

### Example 7: APOE4 Status in Subjects Administered Lecanemab

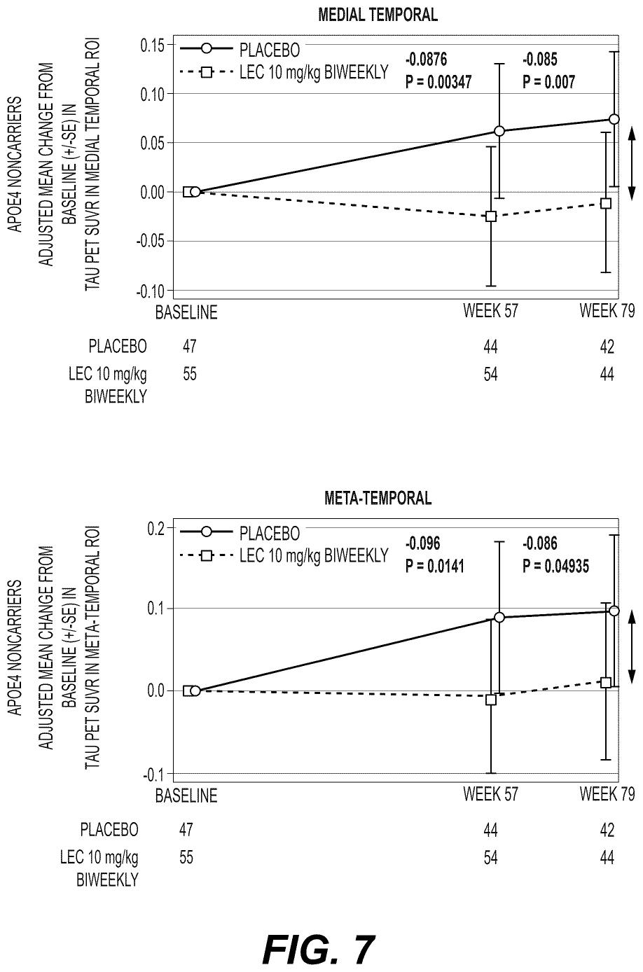

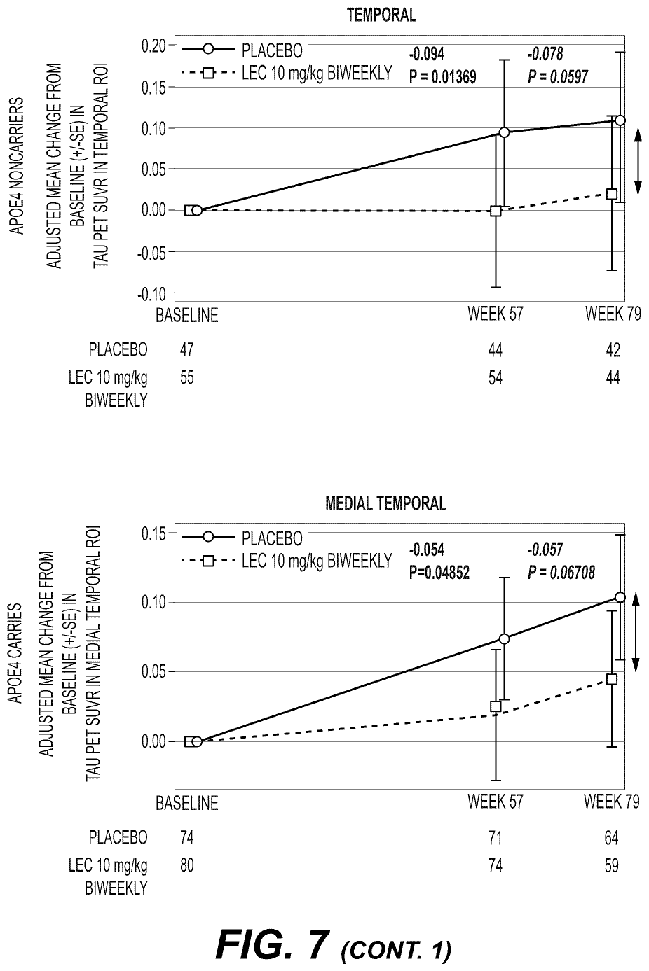

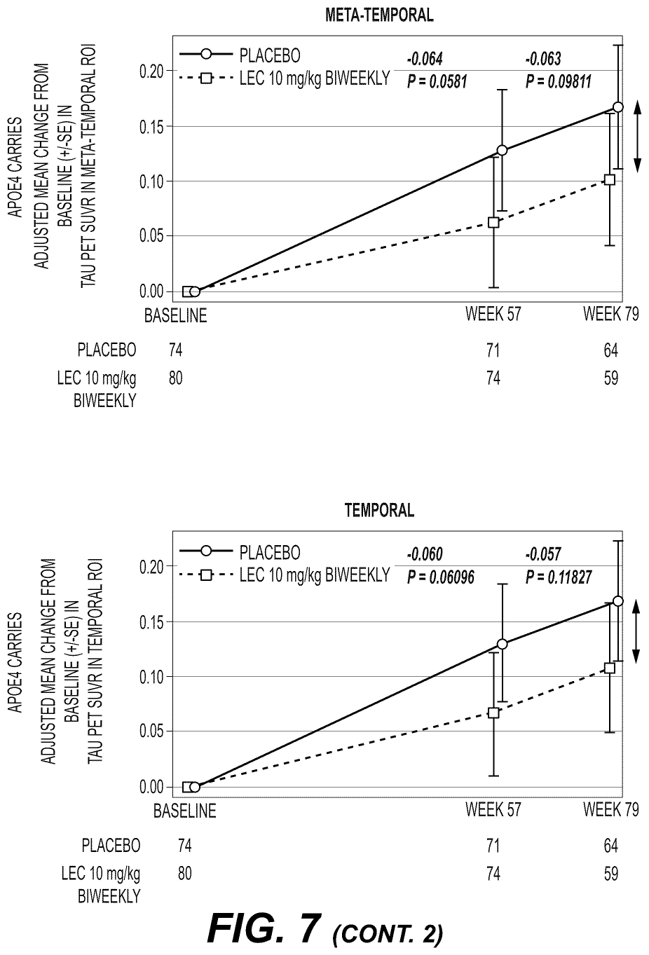

The ApoE ε4 (also called APOE4 or ApoE4) allele of the APOE gene is known to increase risk for AD and is associated with an earlier age of disease onset in some populations. Effects of lecanemab in subjects with different APOE4 status was determined at 13 and 18 months. Results are shown in FIGS. 7-13, where the adjusted mean change from baseline tau PET SUVR (±standard error, SE) in temporal brain regions of subjects administered lecanemab or placebo is plotted over time.

FIG. 7 shows that the magnitude of the effect of lecanemab was slightly higher in noncarriers compared to carriers.

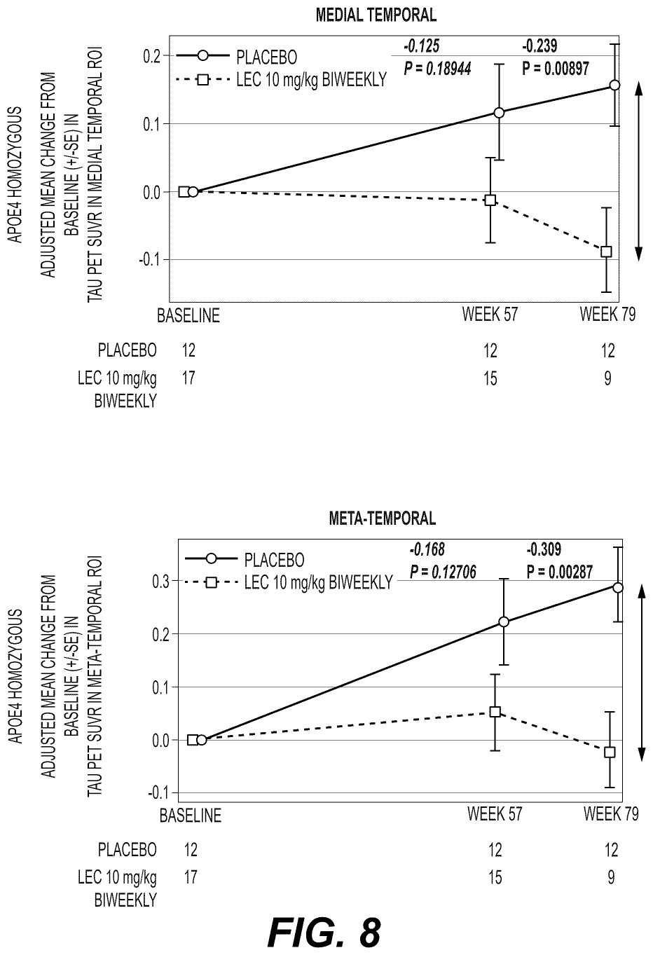

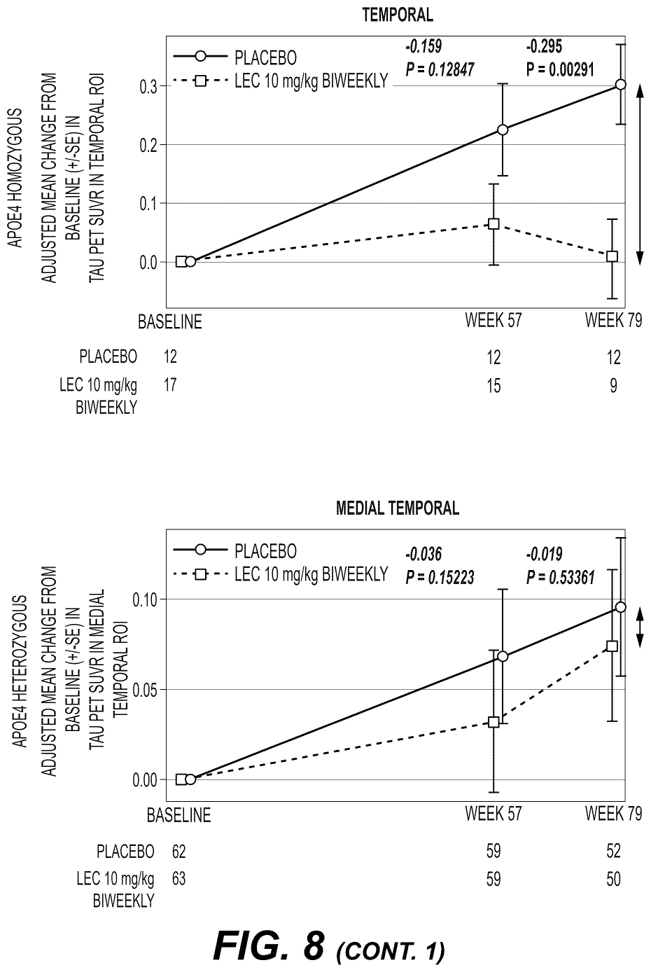

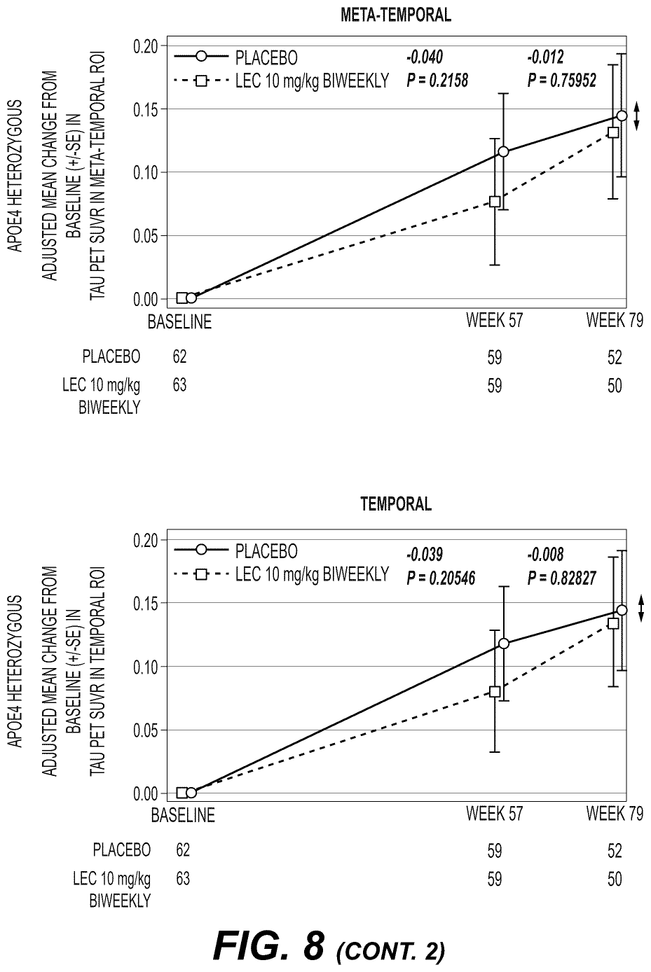

FIG. 8 shows a comparison of subjects who are homozygous for the APOE4 allele and heterozygous for APOE4 allele. In subjects homozygous for APOE4, there is a highly significant effect at 18 months.

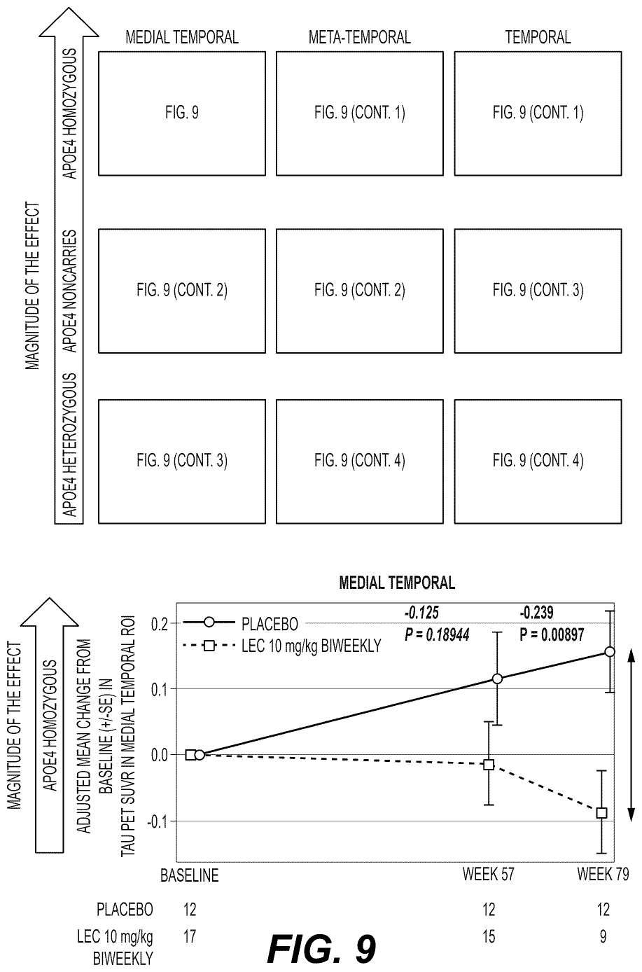

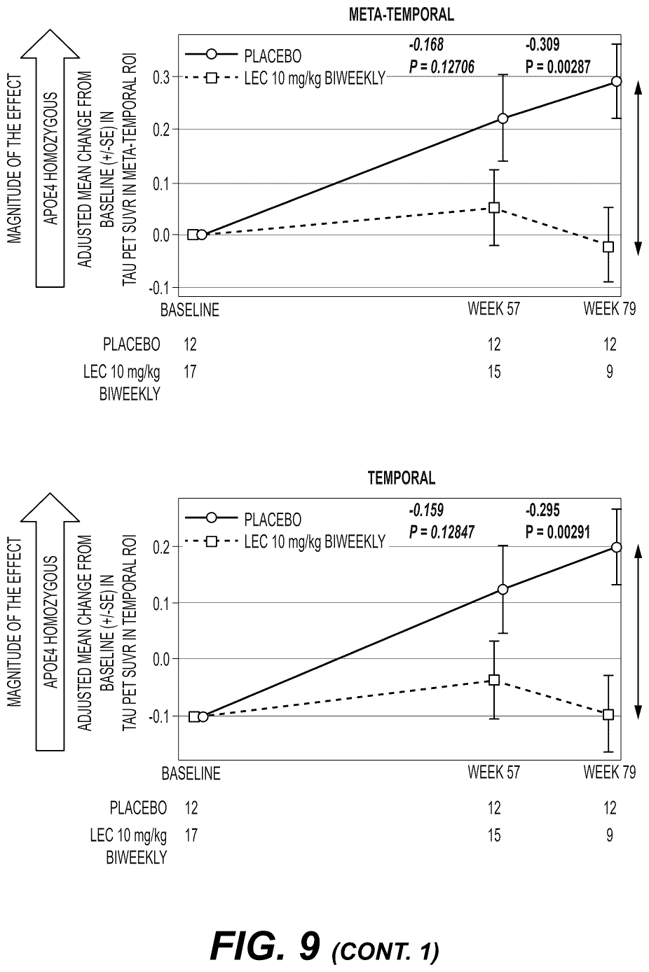

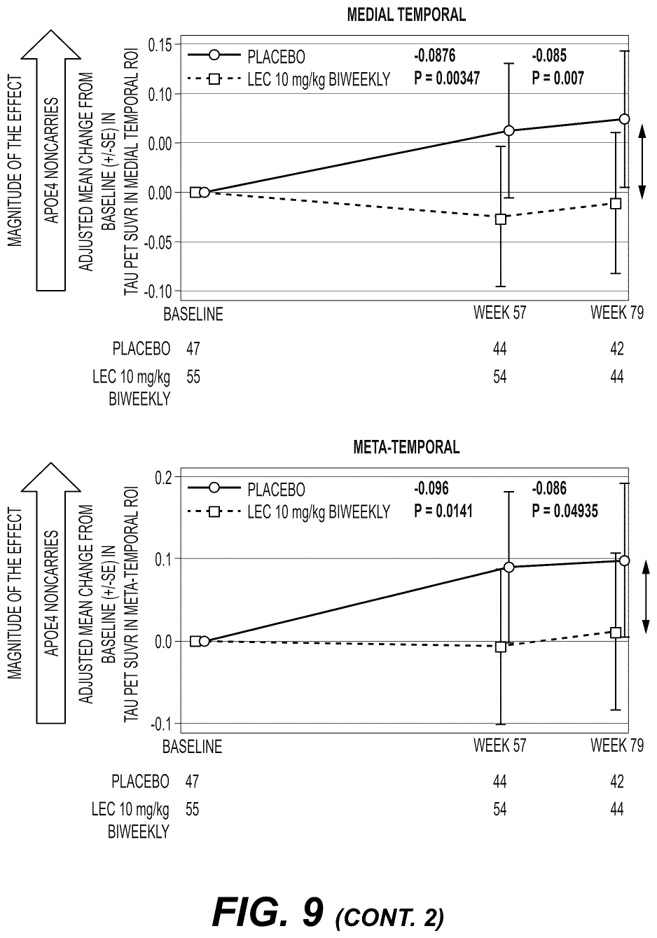

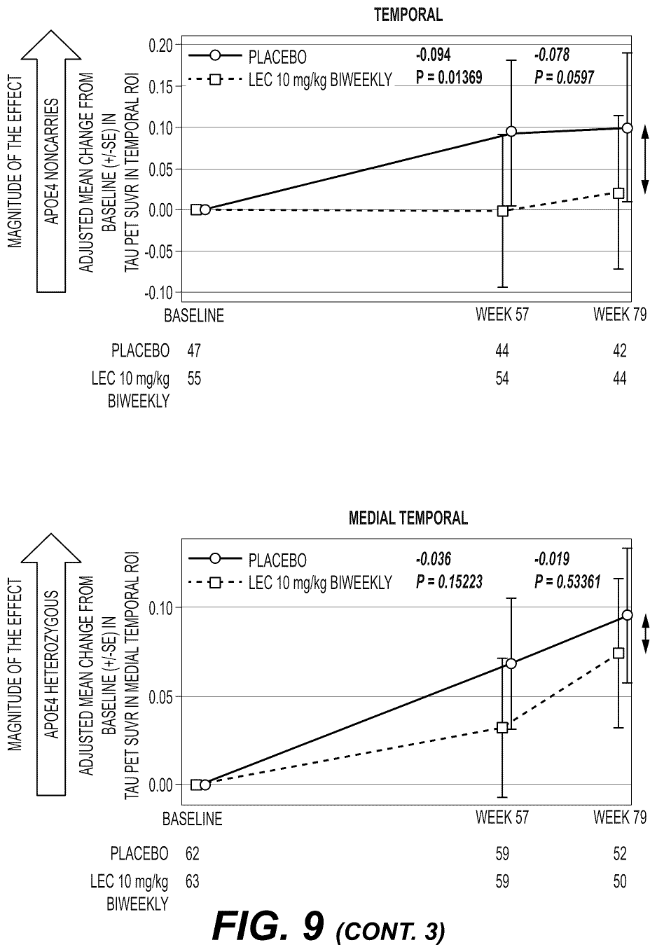

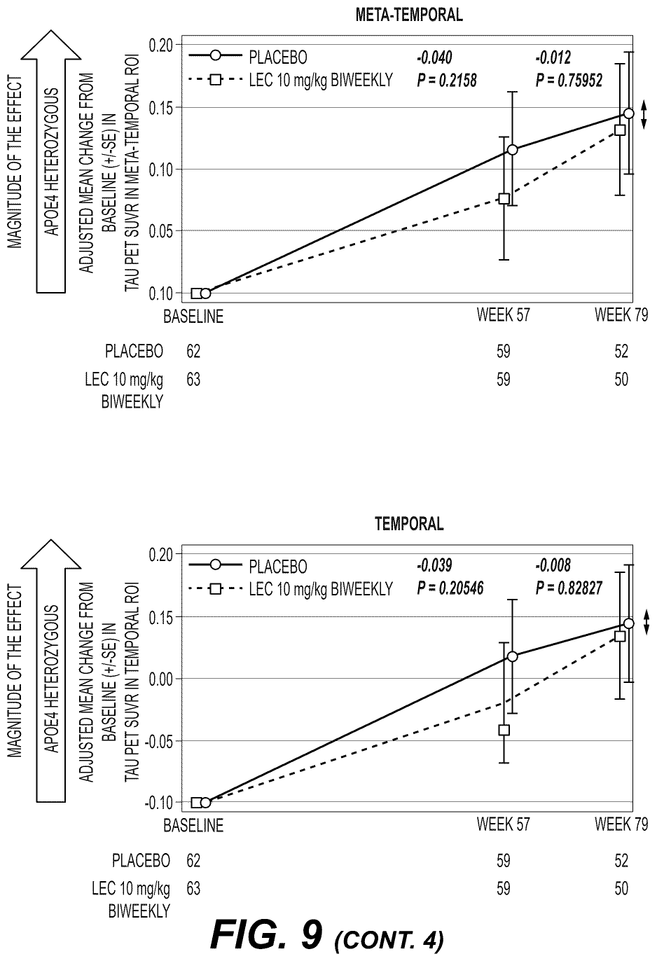

FIG. 9 shows that the effects of lecanemab in subjects homozygous for the APOE4 allele is greater than the effect on subjects who are noncarriers, which, in turn, is greater than the effect on subjects who are heterozygous for the APOE4 allele.

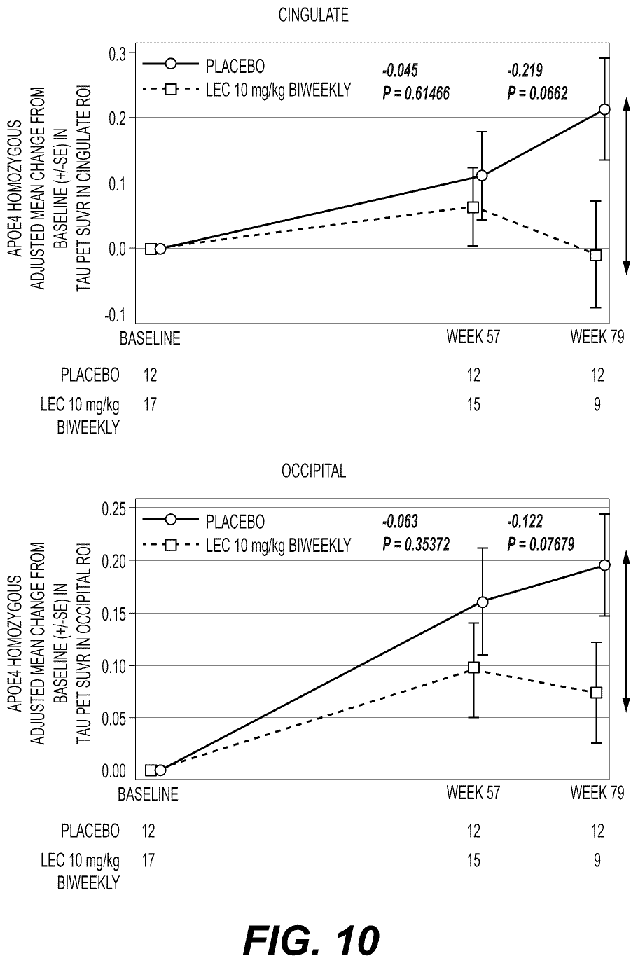

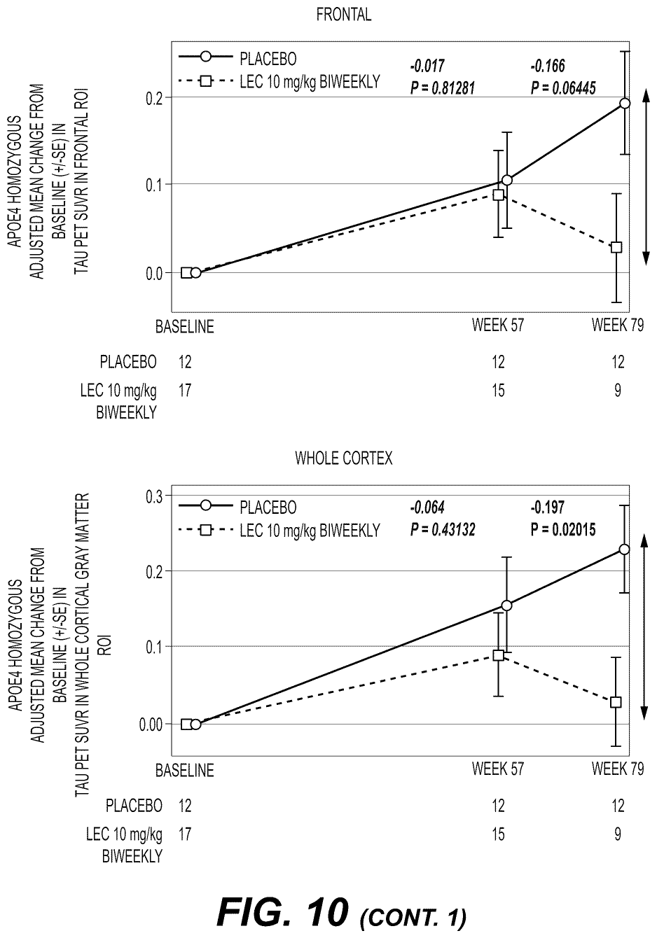

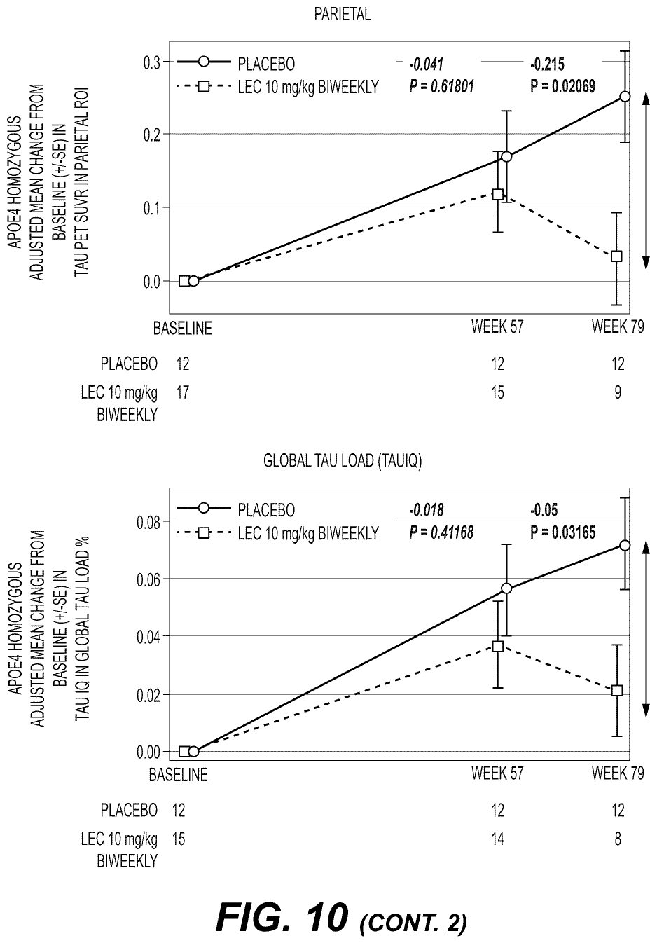

FIG. 10 shows the effects of lecanemab at 18 months across all brain regions and Global tau load (TauIQ) in subjects homozygous for the APOE4 allele carrier. A significant effect of lecanemab was observed in the cingulate cortex, occipital cortex, frontal cortex, parietal cortex, whole cortex gray matter, and in Global tau load (TauIQ).

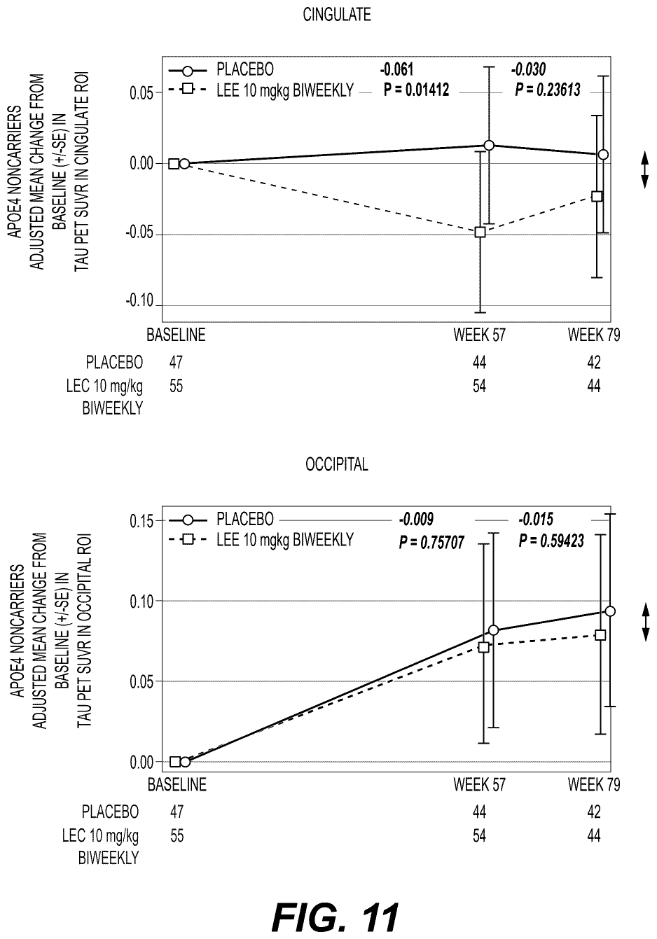

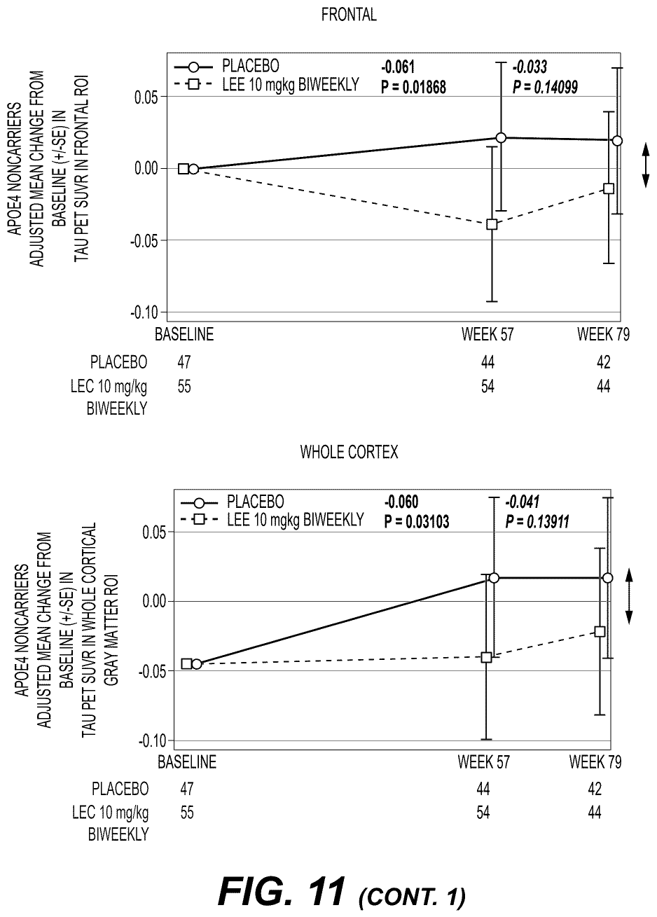

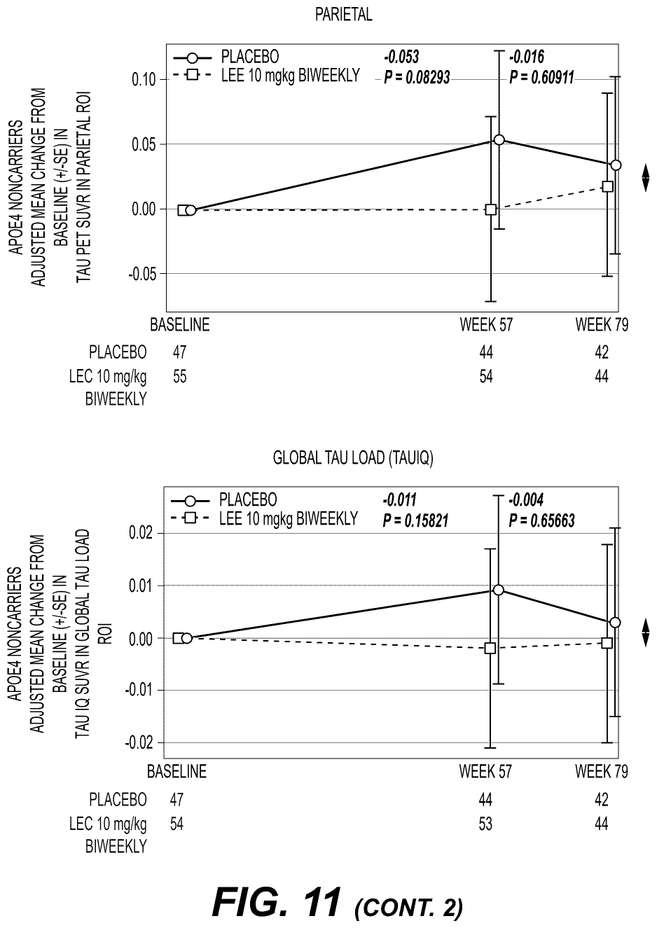

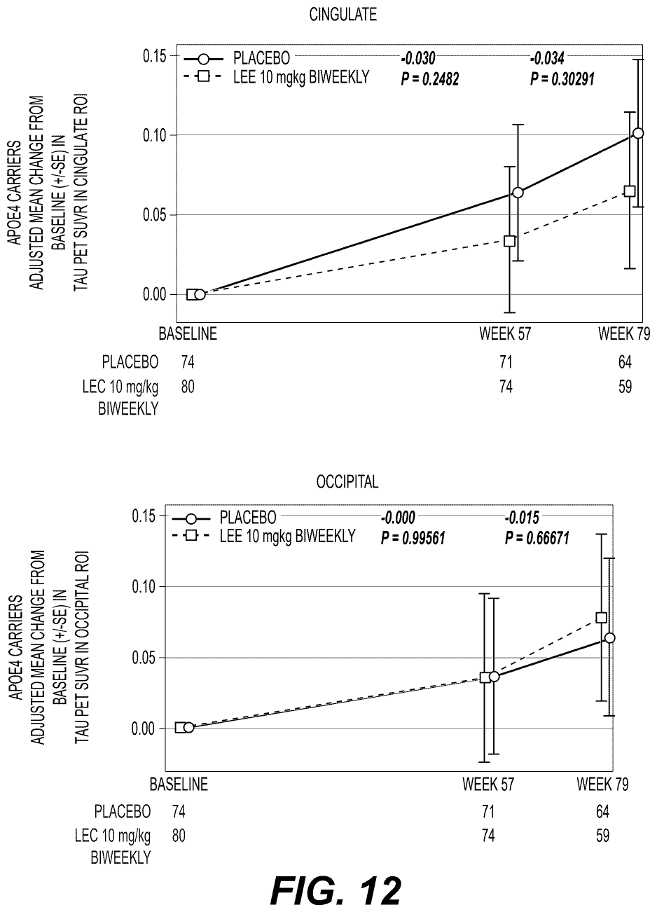

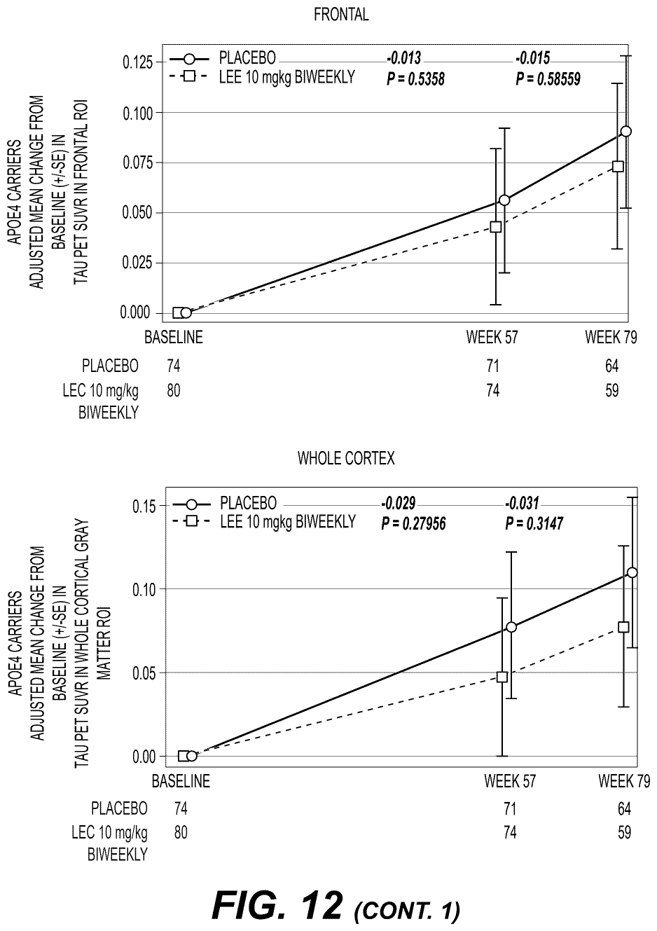

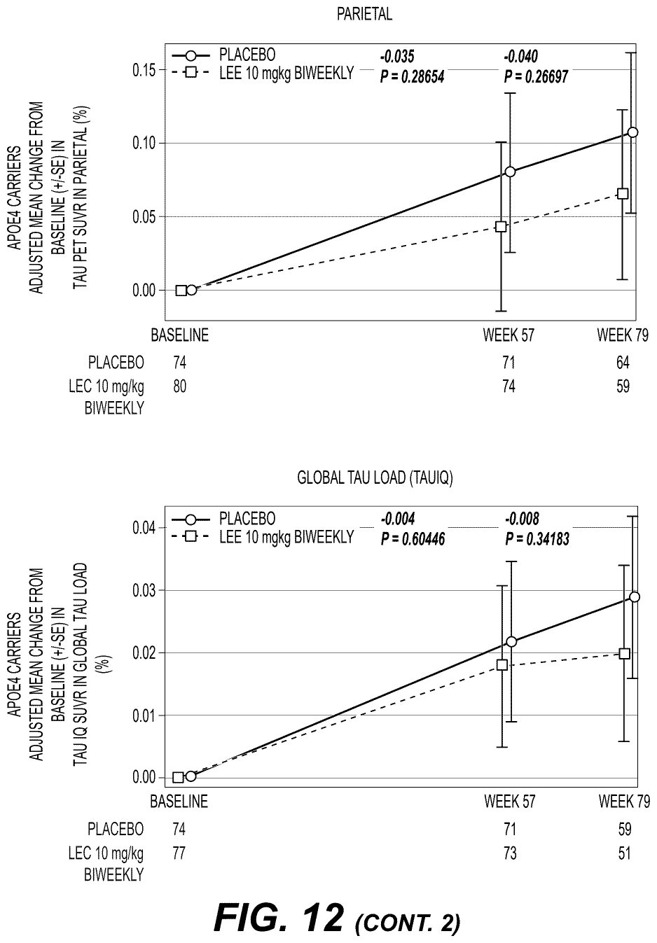

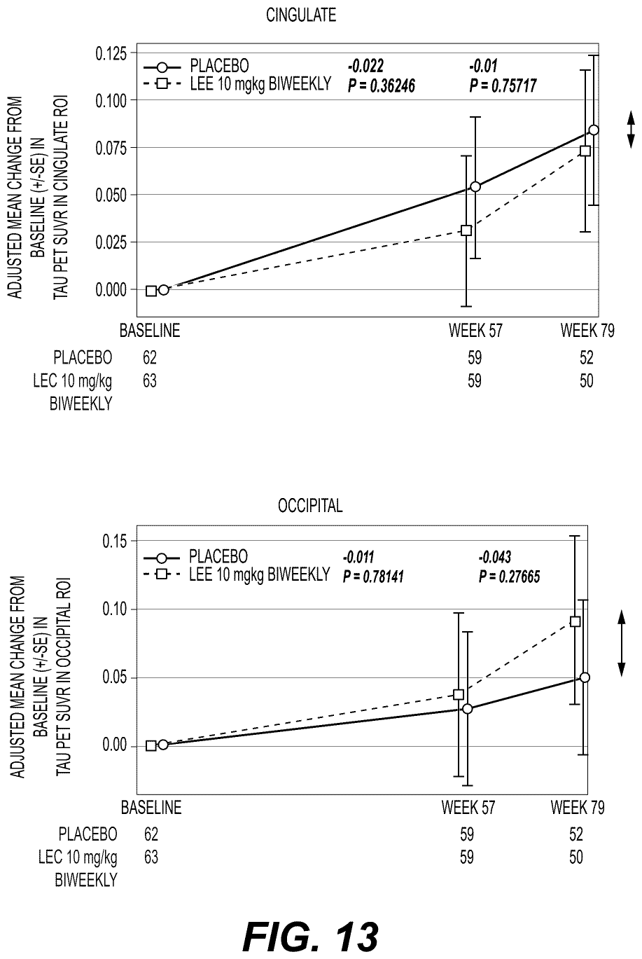

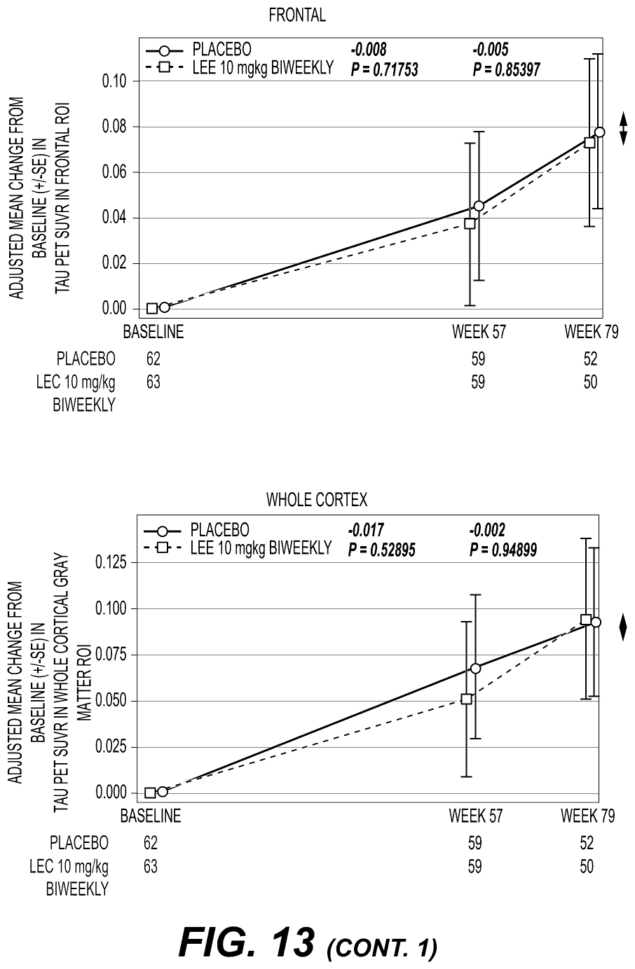

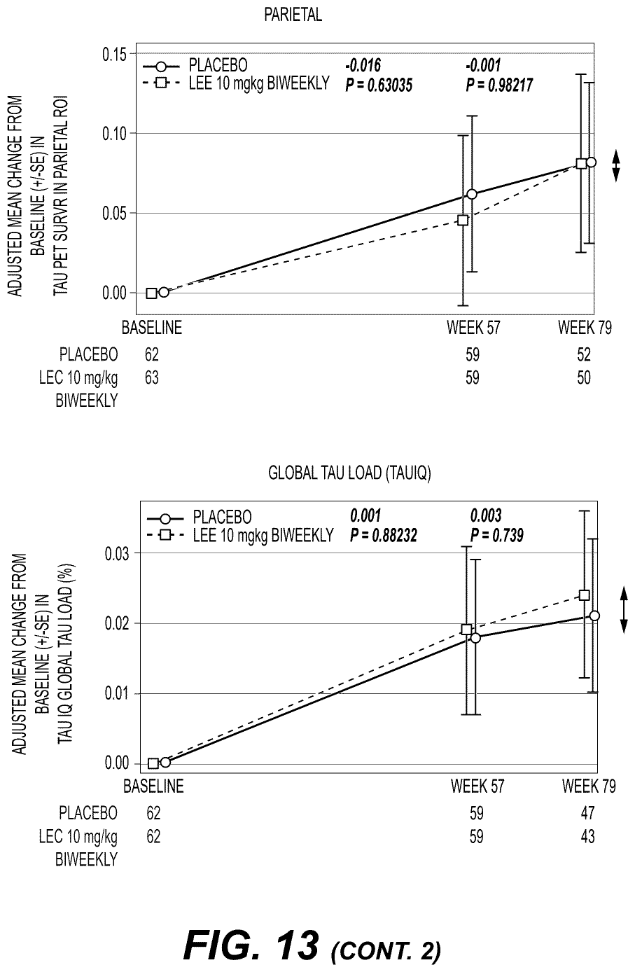

In contrast, in both noncarriers (FIG. 11), carriers (FIG. 12), and subjects heterozygous for the APOE4 allele (FIG. 13), no significant effects of lecanemab were observed at 18 months in the same brain regions and Global tau load.

### Example 8. Tau PET Results

Tau PET results were analyzed in subjects divided into quartile groups based on levels of tau PET, where Quartile 1 included subjects with the lowest tau PET levels, Quartile 4 included subjects with tau PET in the highest levels and Quartiles 2 and 3 included subjects with tau PET levels at levels in between Q1 and Q4.

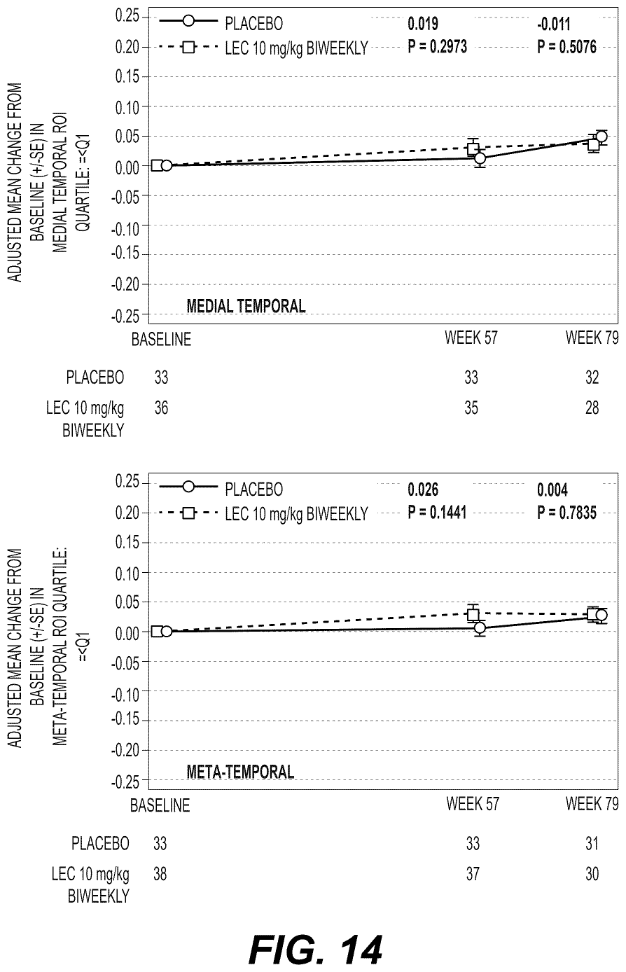

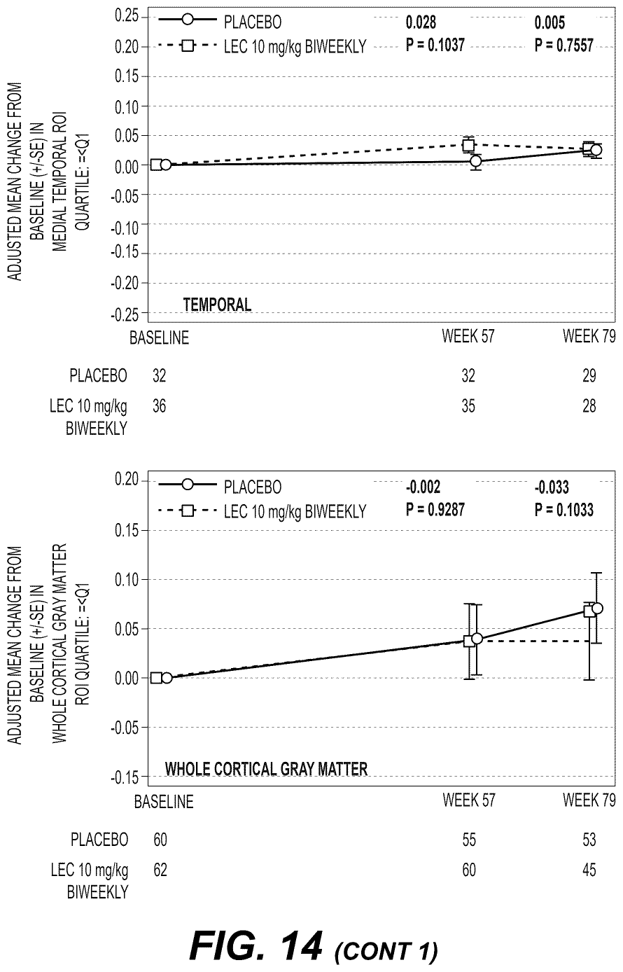

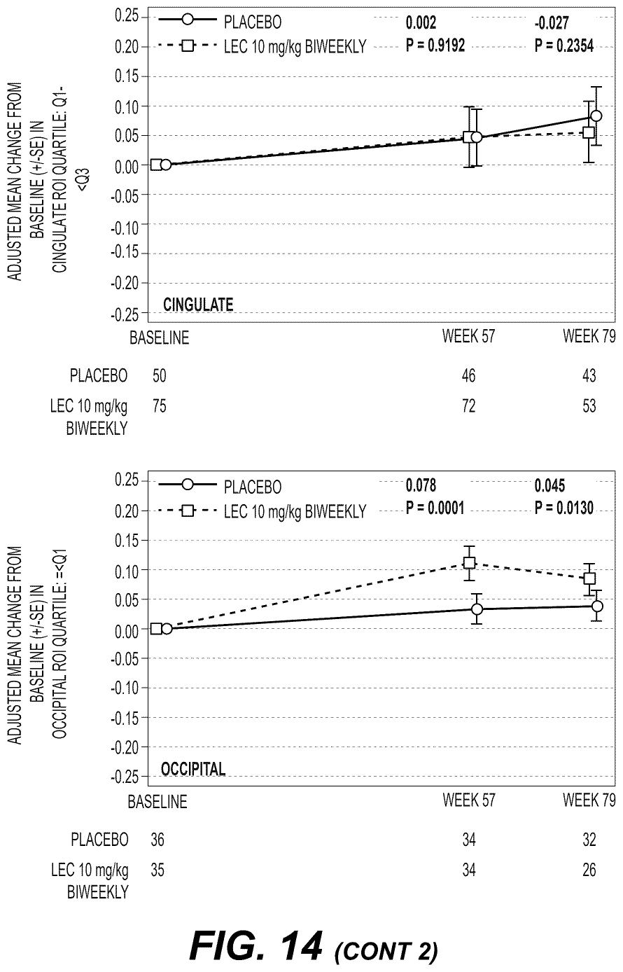

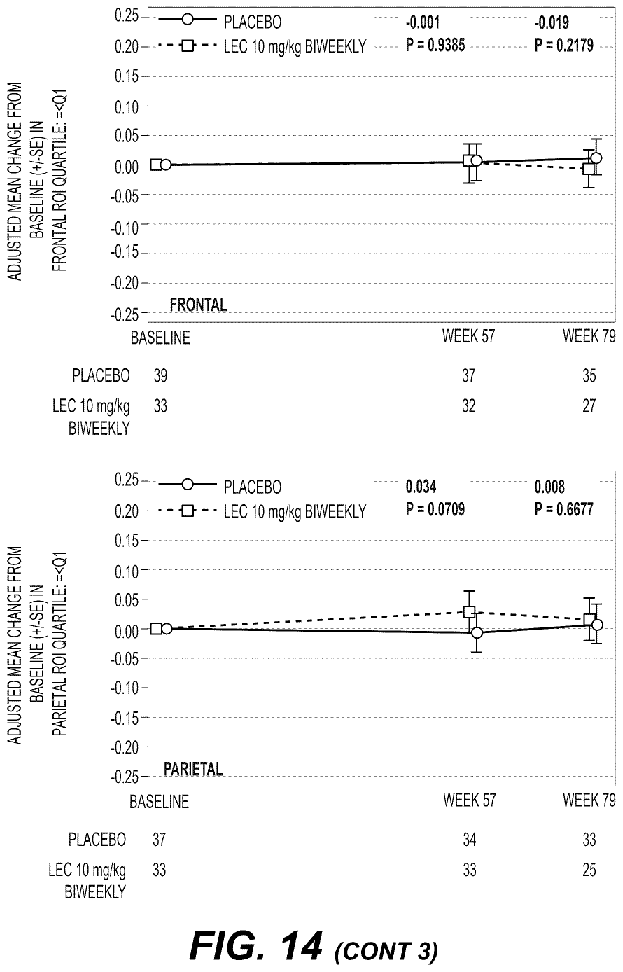

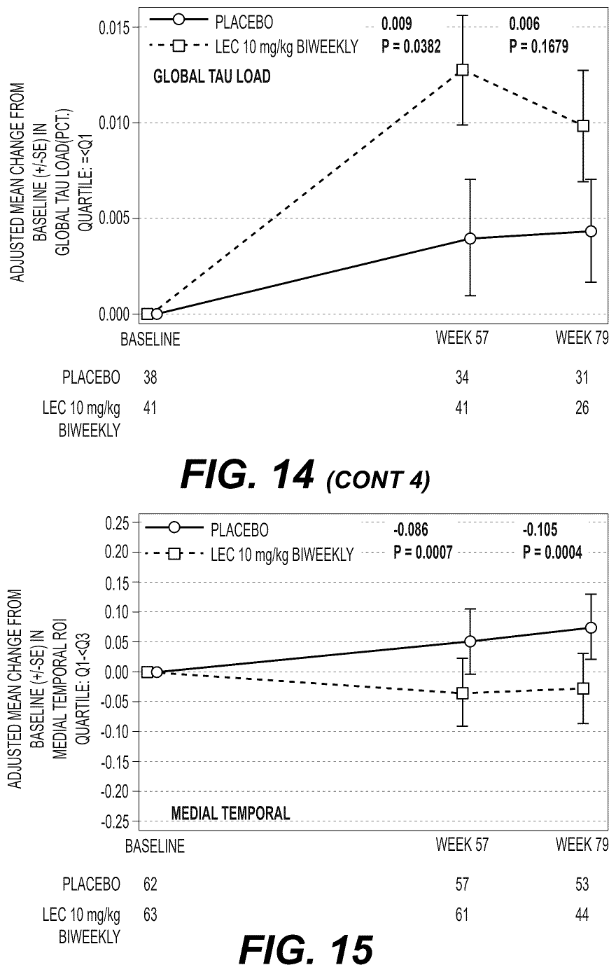

FIG. 14 shows the effects of lecanemab in subjects from below Quartile 1 (<Q1), at 13 months and 18 months in the medial temporal region, the meta-temporal region, and the temporal region, as well as the whole cortical gray matter, cingulate cortex, occipital cortex, frontal cortex, parietal cortex, and the Global tau load (TauIQ).

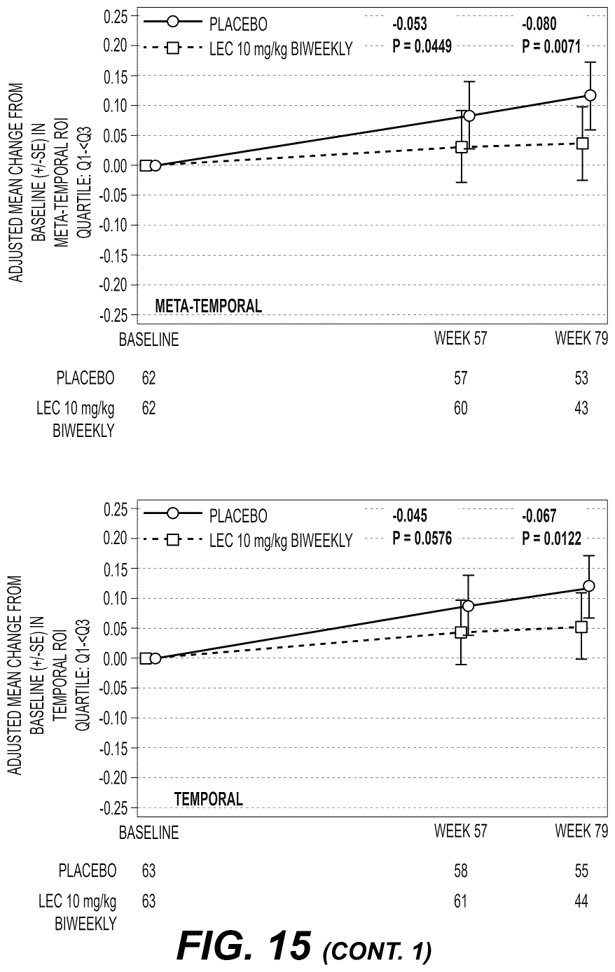

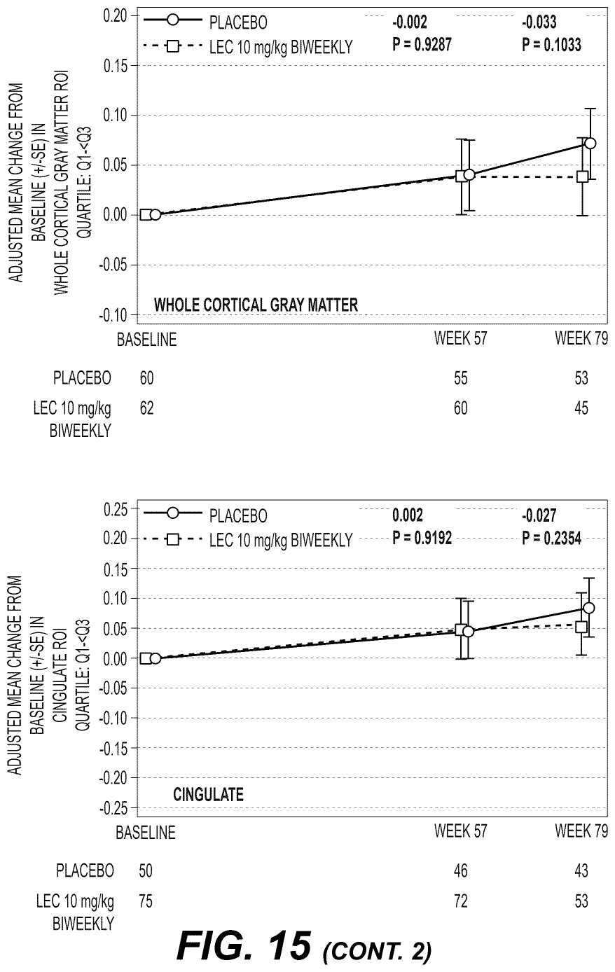

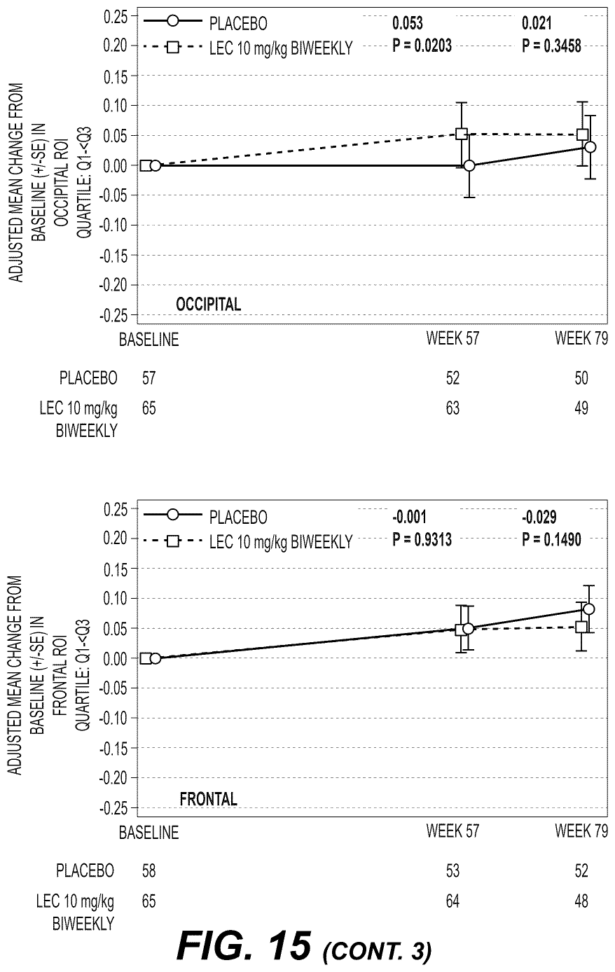

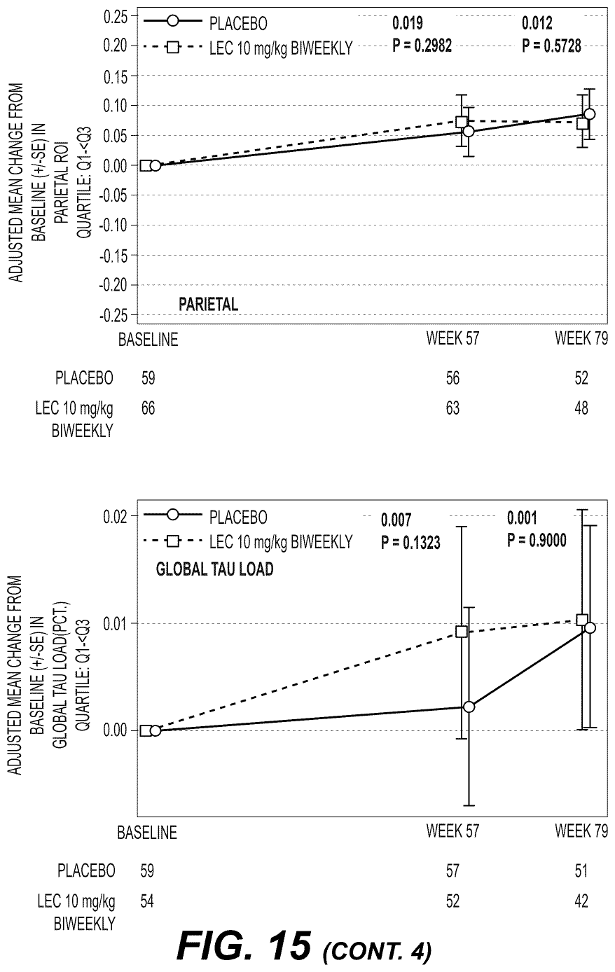

FIG. 15 shows the effects of lecanemab in subjects in Quartile 1 (Q1) up to Quartile 3 (<Q3) over the same time points and regions as FIG. 14.

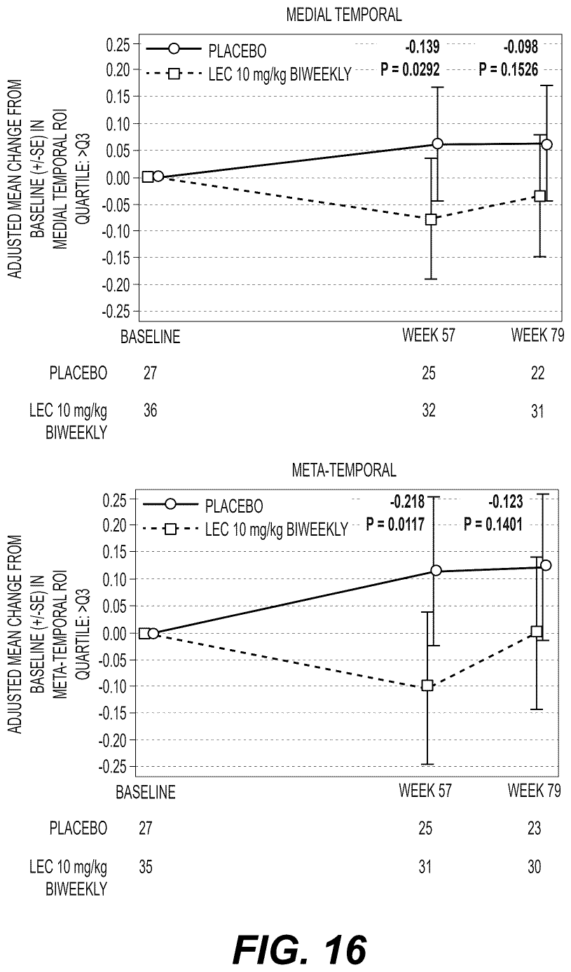

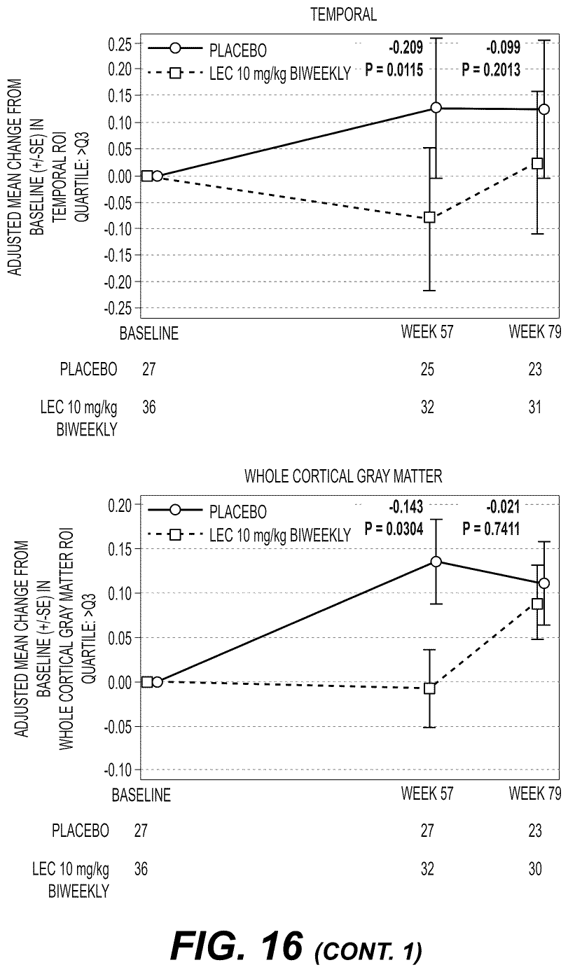

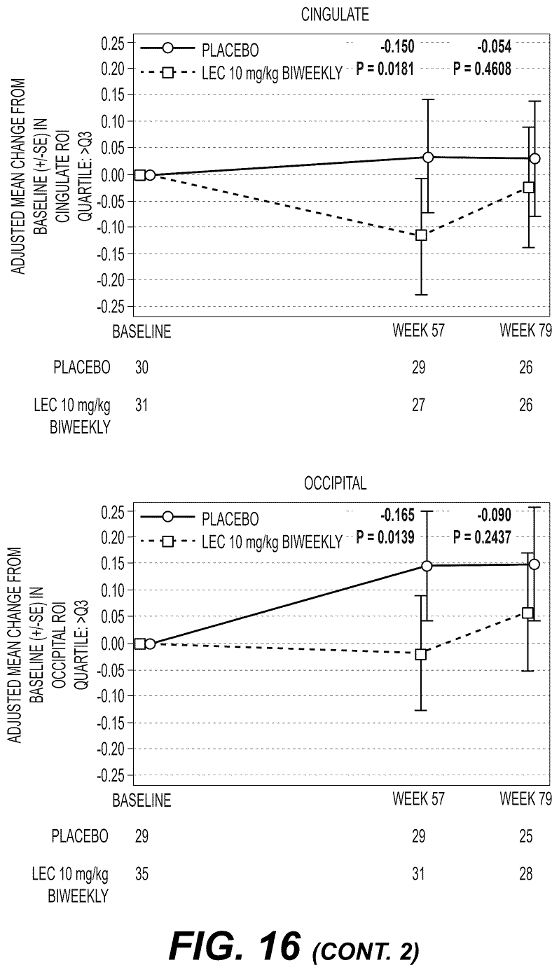

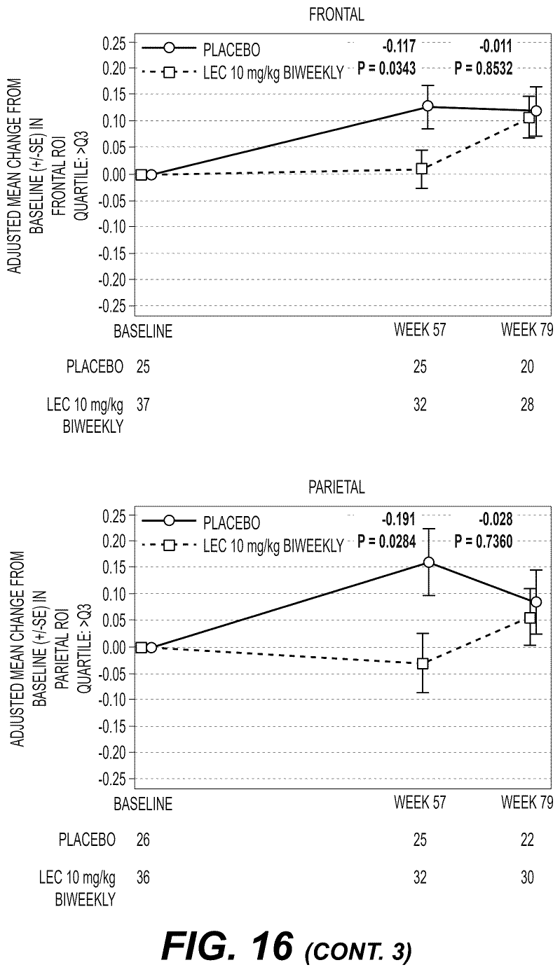

FIG. 16 shows the effects of lecanemab in subjects in above Quartile 3 (>Q3) over the same time points and regions as FIGS. 14 and 15.

### Example 9. Study 201—A Dose Finding Study, with Open Label Extension

The efficacy of lecanemab was evaluated in a double-blind, placebo-controlled, parallel-group, dose finding study (Study 201, NCT 01767311) in subjects with Early Alzheimer's disease (subjects with confirmed presence of amyloid pathology and mild cognitive impairment [64% of patients] or mild dementia stage of disease [36% of patients]). Study 201 had a 79-week double-blind, placebo-controlled period, followed by an OLE for up to 260 weeks, which was initiated after a gap period (range 9 to 59 months; mean 24 months) off treatment. This delayed-stop trial design evaluated the effects of pausing treatment.

In Study 201, 856 patients were randomized to receive one of 5 doses (2.5 mg/kg biweekly n=52, 5 mg/kg monthly n=51, 5 mg/kg biweekly n=92, 10 mg/kg monthly n=253, and 10 mg/kg biweekly n=161) of lecanemab or placebo (n=247). Of the total number of patients randomized, 71.4% were ApoE ε4 carriers and 28.6% were ApoE ε4 non-carriers. During the study the protocol was amended to no longer randomize ApoE ε4 carriers to the 10 mg/kg every two weeks dose arm. ApoE ε4 carriers who had been receiving lecanemab 10 mg/kg every two weeks for 6 months or less were discontinued from study drug. As a result, in the lecanemab 10 mg/kg every two weeks arm, 30.3% of patients were ApoE ε4 carriers and 69.7% were ApoE ε4 non-carriers. At baseline, the mean age of randomized patients was 71 years, with a range of 50 to 90 years. Fifty percent of patients were male and 90% were White.

Patients with MCI due to Alzheimer's disease were enrolled with a CDR global score of 0.5, Memory Box score of 0.5 or greater, and met the NIA-AA core clinical criteria for mild cognitive impairment. Patients with mild AD dementia were enrolled with a global CDR score of 0.5 or 1.0, Memory Box score of 0.5 or greater and met the NIA-AA core clinical criteria for probable Alzheimer's disease dementia. All patients had an MMSE score of ≥22, had objective impairment in episodic memory as indicated by at least 1 standard deviation below age-adjusted mean in the WMS-IV LMII. Patients were enrolled with or without concomitant approved therapies (cholinesterase inhibitors and the N-methyl-D-aspartate antagonist memantine) for Alzheimer's disease.

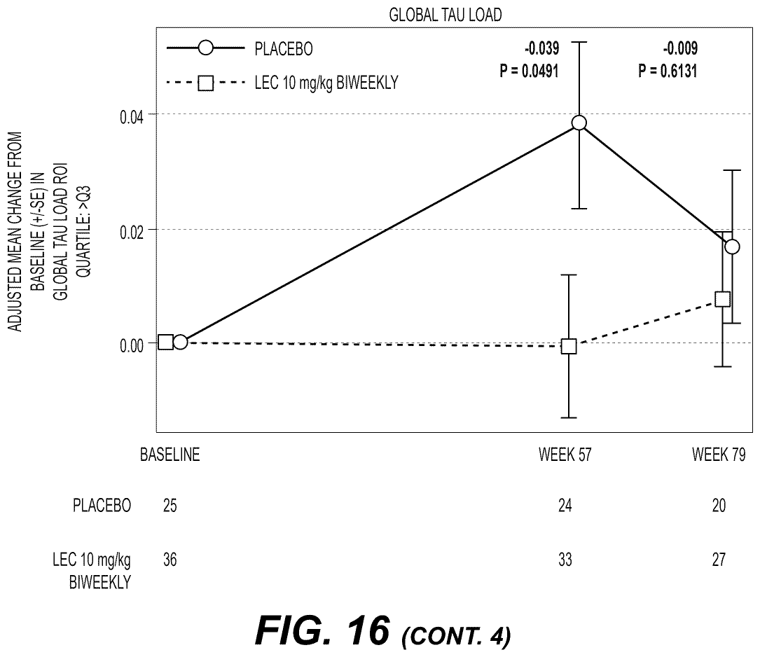

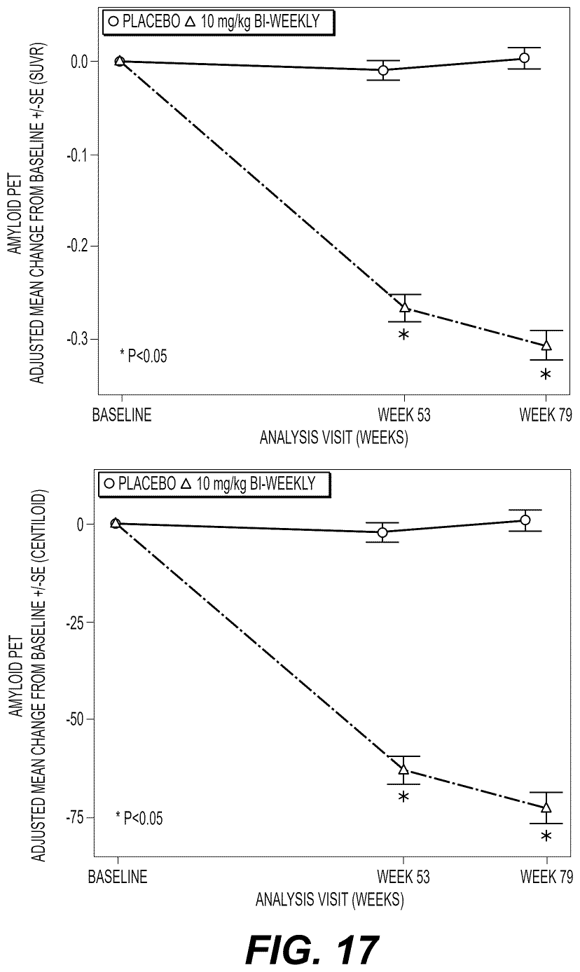

In Study 201, a subgroup of 315 patients were enrolled in the amyloid PET substudy; of these, 277 were evaluated at week 79. Results are described in FIG. 17 and Table 13. The results indicate that lecanemab led to a reduction in brain amyloid beta plaque, as measured by an adjusted mean change from baseline in amyloid beta PET composite (SUVR and Centiloids).

TABLE 13
Amyloid beta PET composite SUVR and amyloid beta PET centiloids
in subjects administered lecanemab at 13 months and 18 months.

| | LEQEMBI | |
|---|---|---|
| Biomarker Endpoints | 10 mg/kg biweekly | Placebo |
| Amyloid Beta PET Composite SUVR | N = 44 | N = 98 |
| Mean baseline | 1.373 | 1.402 |
| Adjusted mean change from | −0.266 | |
| baseline at Week 53 | | |
| Difference from placebo | −0.257 (p < 0.001) | −0.009 |
| Adjusted mean change from | −0.306 | |
| baseline at Week 79 | | |
| Difference from placebo | −0.310 (p < 0.001) | 0.004 |
| Amyloid Beta PET Centiloid | N = 44 | N = 98 |
| Mean baseline | 78.02 | 84.75 |
| Adjusted mean change from | −62.827 | |
| baseline at Week 53 | | |
| Difference from placebo | −60.673 (p < 0.001) | −2.154 |
| Adjusted mean change from | −72.495 | |
| baseline at Week 79 | | |
| Difference from placebo | −73.499 (p < 0.001) | 1.004 |
| Conversion to Amyloid | N = 44 | N = 99 |
| Negative (visual read) | | |
| % Amyloid negative at Week 53 | 65.10% | 11.50% |
| % Amyloid negative at Week 79 | 81.10% | 21.60% |
| Plasma AÃŸ42/40 | N = 43 | N = 88 |
| Mean baseline | 0.0842 | 0.0855 |
| Adjusted mean change from | 0.0049 | |
| baseline at Week 53 | | |
| Difference from placebo | 0.0048 (p = 0.0029) | 0 |
| Adjusted mean change from | 0.0075 | |
| baseline at Week 79 | | |
| Difference from placebo | 0.0054 (p = 0.0036) | 0.0021 |
| Plasma p - tau181 (pg/mL) | N = 84 | N = 179 |
| Mean baseline | 4.6474 | 4.435 |
| Adjusted mean change from | −1.2054 | |
| baseline at Week 53 | | |
| Difference from placebo | −1.2718 (p < 0.0001) | 0.0664 |
| Adjusted mean change from | −1.1127 | |
| baseline at Week 79 | | |
| Difference from placebo | −1.1960 (p < 0.0001) | 0.0832 |
| CSF p - tau181 (pg/mL) | N = 12 | N = 24 |
| Adjusted mean change from | −9.732 | |
| baseline at Week 53 | | |
| Difference from placebo | −12.990 (p = 0.078) | 3.258 |
| Adjusted mean change from | −12.315 (p = 0.044) | |
| baseline at Week 79 | | |
| Difference from placebo | −10.880 | 1.436 |

In Study 201, a subgroup of patients was enrolled in the amyloid PET or CSF substudies. In the Open Label Extension study, lecanemab was administered to subjects who were on placebo in the double-blind, placebo-controlled period of Study 201. Lecanemab treatment produced a decrease in amyloid beta plaque levels compared to OLE baseline as early as Week 13, with amyloid reduction of 0.09 SUVR at Week 13 (p<0.001) and amyloid reduction of 0.16 (p<0.001) SUVR at Week 27. Corresponding changes were seen in plasma A0342/40 and plasma p-tau1181.

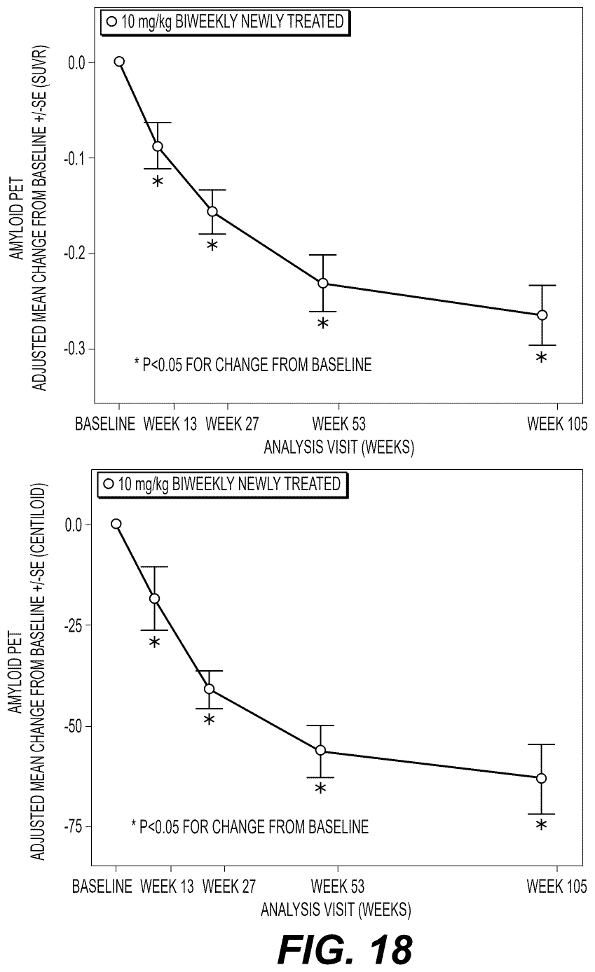

Results from the OLE amyloid PET and plasma biomarker substudies are described in FIG. 18 and Table 14.

TABLE 14
Biomarker endpoints in subjects administered lecanemab.

| | LEQEMBI |
|---|---|
| Biomarker Endpoints | 10 mg/kgbiweekly* |
| Amyloid Beta PET Composite SUVR | N = 22 |
| Mean OLE baseline | 1.37 |
| Adjusted mean change from | −0.088 (p < 0.001) |
| baseline at Week 13 | |
| Adjusted mean change from | −0.157 (p < 0.001) |
| baseline at Week 27 | |
| Adjusted mean change from | −0.232 (p < 0.001) |
| baseline at Week 53 | |
| Amyloid Beta PET Centiloid | N = 22 |
| Mean OLE baseline | 77.2 |
| Adjusted mean change from | −18.347 (p = 0.022) |
| baseline at Week 13 | |
| Adjusted mean change from | −40.921 (p < 0.001) |
| baseline at Week 27 | |
| Adjusted mean change from | −56.328 (p < 0.001) |
| baseline at Week 53 | |
| Conversion to Amyloid | N = 17 |
| Negative (visual read) | |
| % Amyloid negative at Week 13 | 42.90% |
| % Amyloid negative at Week 27 | 75.00% |
| % Amyloid negative at Week 53 | 83.30% |
| Plasma AB42/40 | N = 32 |
| Mean OLE baseline | 0.0857 |
| Mean change from baseline | 0.0028 |
| at Week 13 | |
| Mean change from baseline | 0.0044 |
| at Week 27 | |
| Mean change from baseline | 0.0097 |
| at Week 53 | |
| Plasma p - tau181 (pg/mL) | N = 27 |
| Mean OLE baseline | 5.272 |
| Mean change from baseline | 0.089 |
| at Week 13 | |
| Mean change from baseline | −0.921 |
| at Week 27 | |
| Mean change from baseline | −1.596 |
| at Week 53 | |

*Patients who were randomized to placebo in the double-blind, placebo-controlled period of Study 1, and received LEQEMBI 10 mg/kg biweekly in the Open-Label Extension

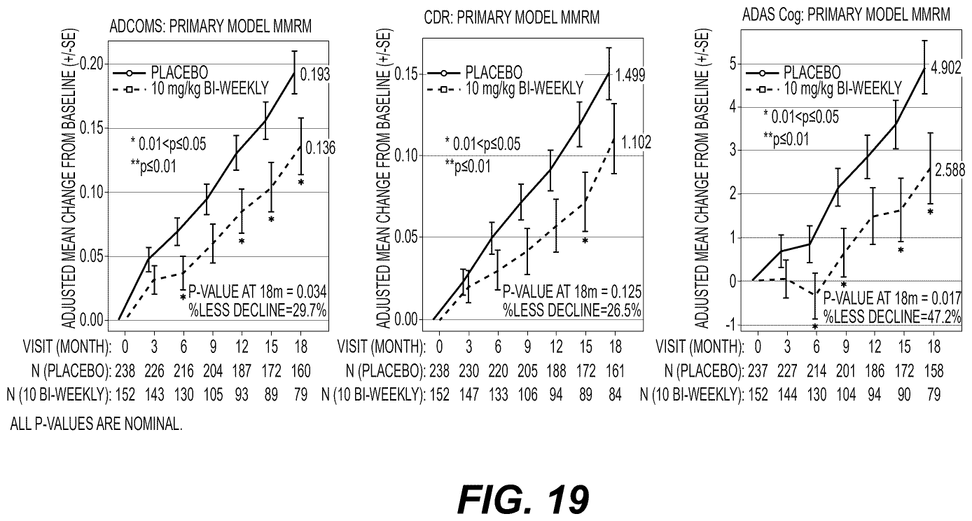

FIG. 19 and Table 15 show Study 201 outcomes. Study 201 achieved its primary objective by identifying lecanemab 10 mg/kg biweekly as the most efficacious dose regimen (ED90 dose) on the ADCOMS after 53 weeks of treatment. The primary endpoint was change from baseline on the ADCOMS at Week 53. The prespecified success criteria at the Week 53 assessment, 8000 probability that the ED90 dose had likelihood of 2500 or greater slowing of progression on the ADCOMS relative to placebo, was not achieved. Lecanemab 10 mg/kg biweekly had a 6400 likelihood of 2500 or greater slowing of progression on the ADCOMS relative to placebo at Week 53.

Key secondary efficacy endpoints included the change from baseline in amyloid PET SUVR composite at Week 79 and change from baseline in the ADCOMS, CDR-SB, and ADAS-Cog4 at Week 79.

At Week 79 lecanemab demonstrated a dose-dependent slowing of clinical decline on clinical outcomes, as shown in FIG. 19. Results are summarized in Table 15. Lecanemab 10 mg/kg biweekly showed 29.700 less decline on the ADCOMS, 26.5% less decline on CDR-SB, and 47.2 less decline on ADAS-Cog14 compared to placebo. In Study 201, a subgroup of patients was enrolled in the amyloid PET or CSF substudies.

Clinical treatment effect relative to placebo at the end Study 201 was maintained off treatment during the gap period up to the OLE baseline, while the overall rate of progression in the gap period was similar in both groups.

TABLE 15
Clinical endpoints of Study 201

| | LEQEMBI | |
|---|---|---|
| Clinical Endpoints | 10 mg/kgbiweekly | Placebo |
| ADCOMS | N = 152 | N = 238 |
| Mean baseline | 0.373 | 0.37 |
| Adjusted mean change from | 0.085 | |
| baseline at Week 53 | | |
| Difference from placebo | −0.046 (p = 0.027) | 0.131 |
| Adjusted mean change from | 0.136 | |
| baseline at Week 79 | | |
| Difference from placebo | −0.057 (p = 0.034) | 0.193 |
| CDR - SB | N = 152 | N = 238 |
| Mean baseline | 2.97 | 2.89 |
| Adjusted mean change from | 0.568 | |
| baseline at Week 53 | | |
| Difference from placebo | −0.344 (p = 0.077) | 0.911 |
| Adjusted mean change from | 1.102 | |
| baseline at Week 79 | | |
| Difference from placebo | −0.396 (p = 0.125) | 1.499 |
| ADAS - Cog 14 | N = 152 | N = 237 |
| Mean baseline | 22.06 | 22.56 |
| Adjusted mean change from | 1.481 | |
| baseline at Week 53 | | |
| Difference from placebo | −1.361 (p = 0.073) | 2.842 |
| Adjusted mean change from | 2.588 | |
| baseline at Week 79 | | |
| Difference from placebo | −2.313 (p = 0.017) | 4.902 |

N is the number of patients with baseline value.
All p-values are nominal.

### Example 10. Study 301—CLARITY AD

### A. CLARITY—A Double-Blind, Placebo-Controlled, Parallel-Group, Randomized Study

The safety and efficacy of lecanemab was evaluated in a double-blind, placebo-controlled, parallel-group, randomized study (Study 301 CLARITY AD, NCT 03887455) in patients with Alzheimer's disease (patients with confirmed presence of amyloid pathology and mild cognitive impairment due to Alzheimer's disease [62%] and mild dementia due to Alzheimer's disease [38%], consistent with Stage 3 and Stage 4 Alzheimer's disease). Patients could enroll in an optional, long-term extension of the study.

The primary efficacy outcome was change from baseline at 18 months in the Clinical Dementia Rating scale Sum of Boxes (CDR-SB). Key secondary endpoints included change from baseline at 18 months for the following measures: amyloid Positron Emission Tomography (PET) using centiloids, Alzheimer Disease Assessment Scale—Cognitive Subscale 14 (ADAS-cog14), and Alzheimer's Disease Cooperative Study-Activities of Daily Living Scale for Mild Cognitive Impairment (ADCS MCI-ADL).

A total of 1795 patients were enrolled and randomized 1:1 to receive lecanemab 10 mg/kg or placebo once every 2 weeks. Of the total number of patients randomized, 69% were ApoE ε4 carriers and 31% were ApoE ε4 non-carriers. Overall median age of patients was 72 years, with a range to 50 to 90 years. Fifty-two percent were women. Of the global population, 1381 (77%) were White, 303 (17%) Asian, and 47 (2.6%) Black; of the 947 patients in the United States, 895 (94.5%) were White, 213 (22.5%) were Hispanic ethnicity, 43 (4.5%) Black, and 7 (0.7%) Asian.

Patients with MCI due to Alzheimer's disease were enrolled with a CDR global score of 0.5, Memory Box score of 0.5 or greater. Patients with mild AD dementia were enrolled with a global CDR score of 0.5 or 1.0, Memory Box score of 0.5 or greater. All patients had a Mini-Mental State Examination (MMSE) score of ≥22, had objective impairment in episodic memory as indicated by at least 1 standard deviation below age-adjusted mean in the Wechsler-Memory Scale-IV Logical Memory II (subscale) (WMS-IV LMII). The randomization was stratified according to clinical subgroup; the presence or absence of concomitant symptomatic medication for Alzheimer's disease at baseline (cholinesterase inhibitors and the N-methyl-D-aspartate antagonist memantine); ApoE ε4 carrier status; and region.

The mean length of treatment was 16 months (range: 0.5 to 19 months).

### B. Clinical Endpoints

Lecanemab treatment met the primary endpoint and reduced clinical decline on the global cognitive and functional scale, CDR-SB, compared with placebo at 18 months by 27%, which represents a treatment difference in the score change of −0.45 (p=0.00005).

Highly statistically significant differences (p<0.01) between treatment groups were also seen in the results for ADAS-cog14 and ADCS MCI-ADL at 18 months. Results are summarized in Table 16.

TABLE 16
Results for CDR-SB, ADAS-cog14,
and ADCS MCI-ADL in Study 301

| | LEQEMBI | |
|---|---|---|
| Clinical Endpoints | 10 mg/kg biweekly | Placebo |
| CDR-SB | N = 859 | N = 875 |
| Mean baseline | 3.17 | 3.22 |
| Adjusted mean change from | 1.213 | |
| baseline at 18 months | | |
| Difference from placebo | −0.451 (p = 0.00005) | 1.663 |
| ADAS - Cog14 | N = 854 | N = 872 |
| Mean baseline | 24.45 | 24.37 |
| Adjusted mean change from | 4.140 | |
| baseline at 18 months | | |
| Difference from placebo | −1.442 (p = 0.00065) | 5.581 |
| ADCS MCI - ADL | N = 783 | N = 796 |
| Mean baseline | 41.2 | 40.9 |
| Adjusted mean change from | −3.484 | |
| baseline at 18 months | | |
| Difference from placebo | 2.016 (p < 0.00001) | −5.5 |

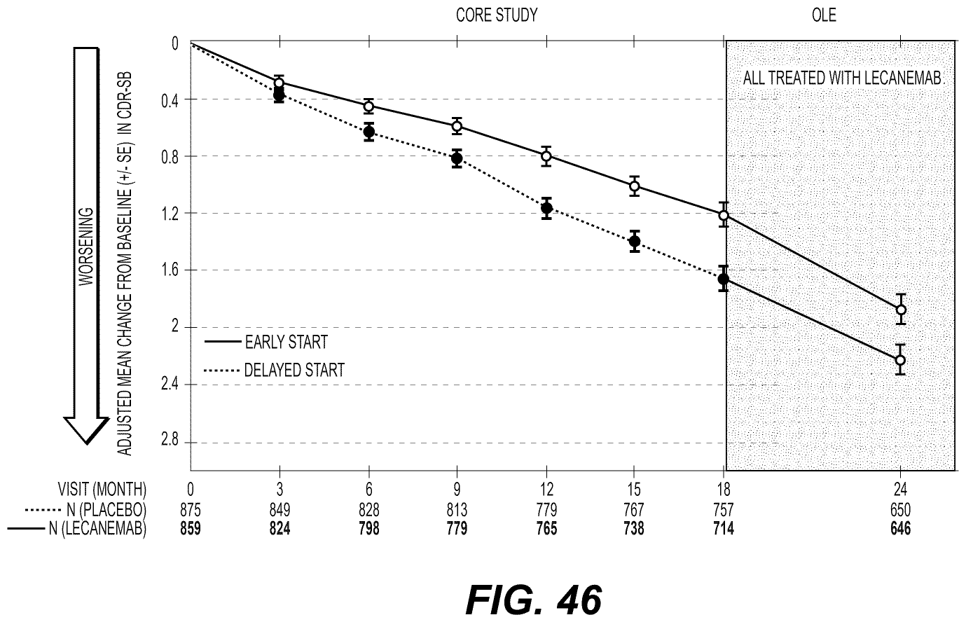

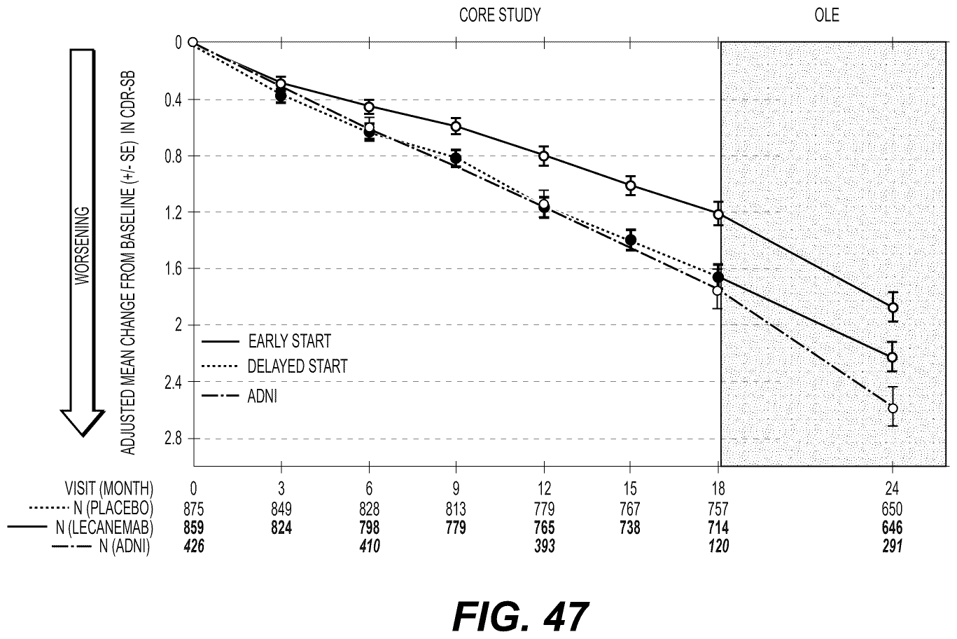

FIG. 46 and FIG. 47 also describe baseline and highly statistically significant results for primary and key secondary endpoints of Study 301.

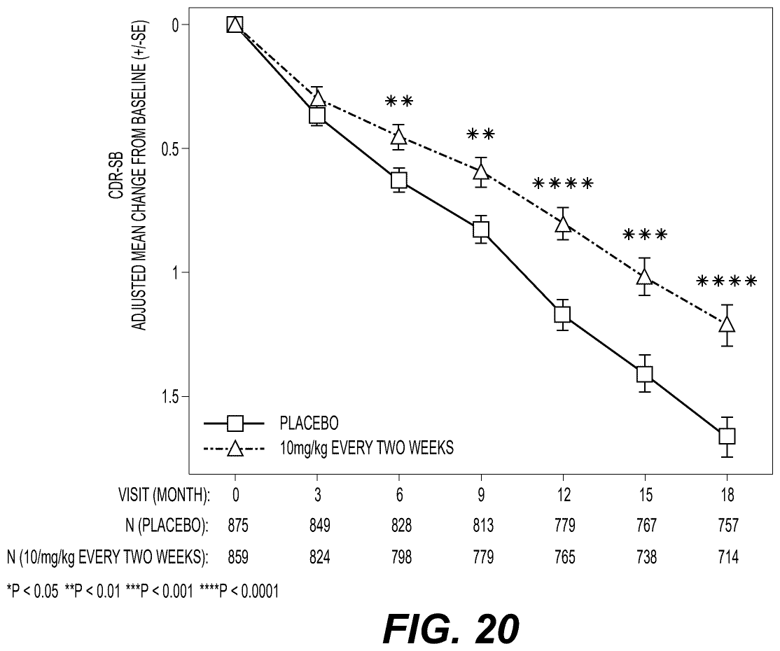

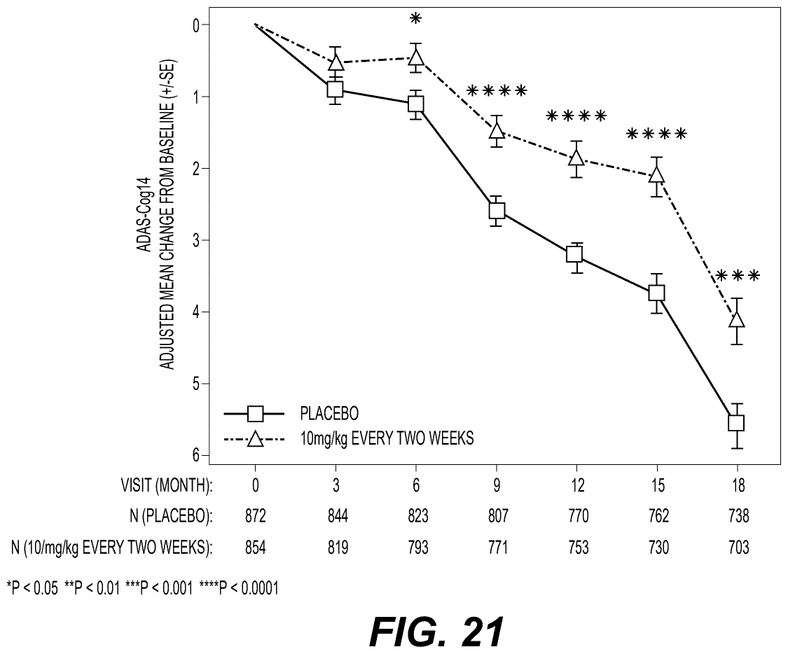

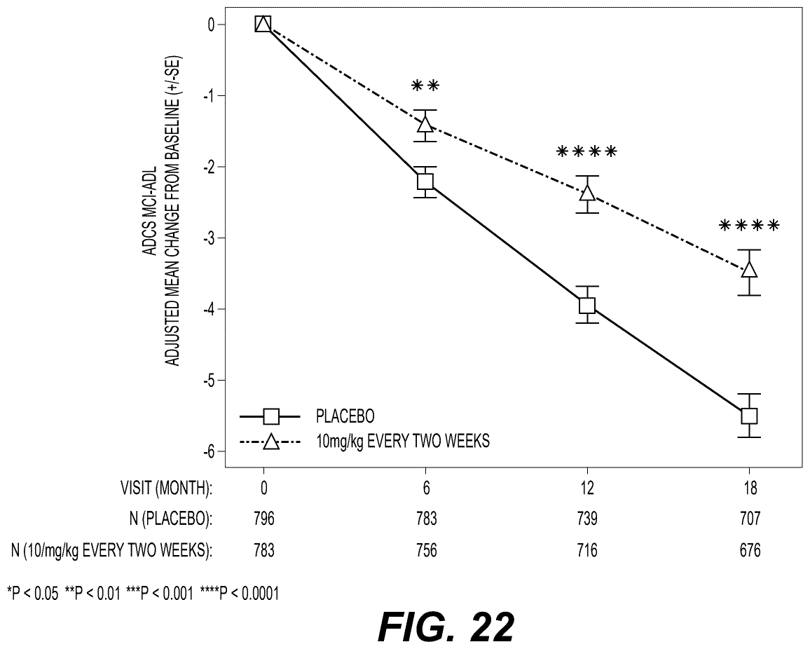

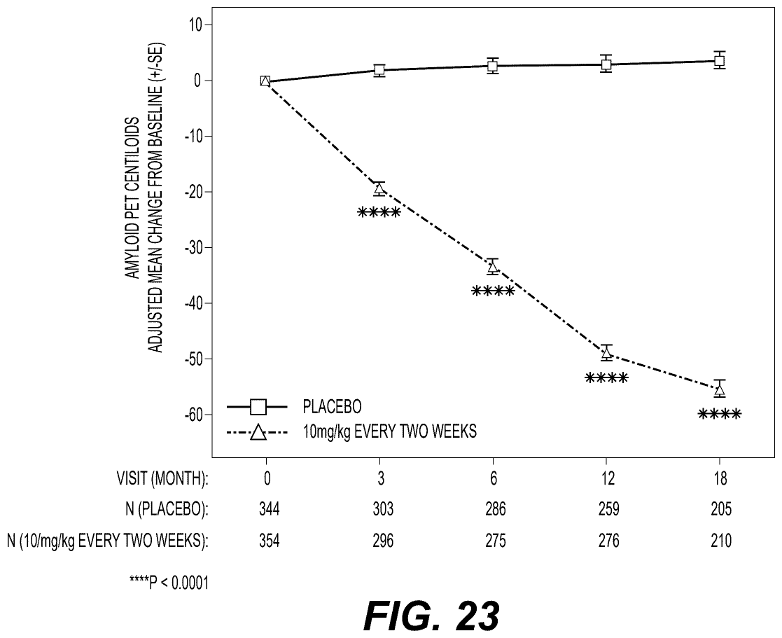

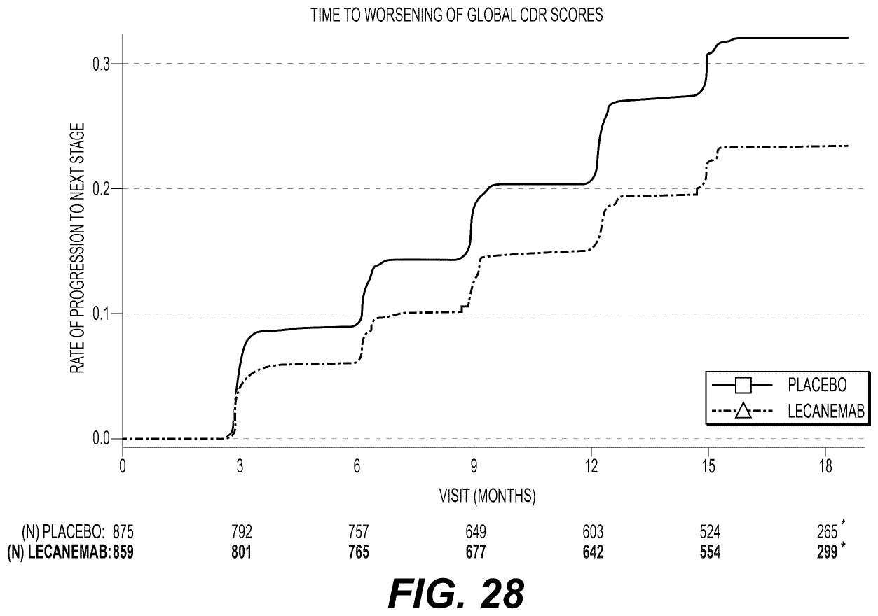

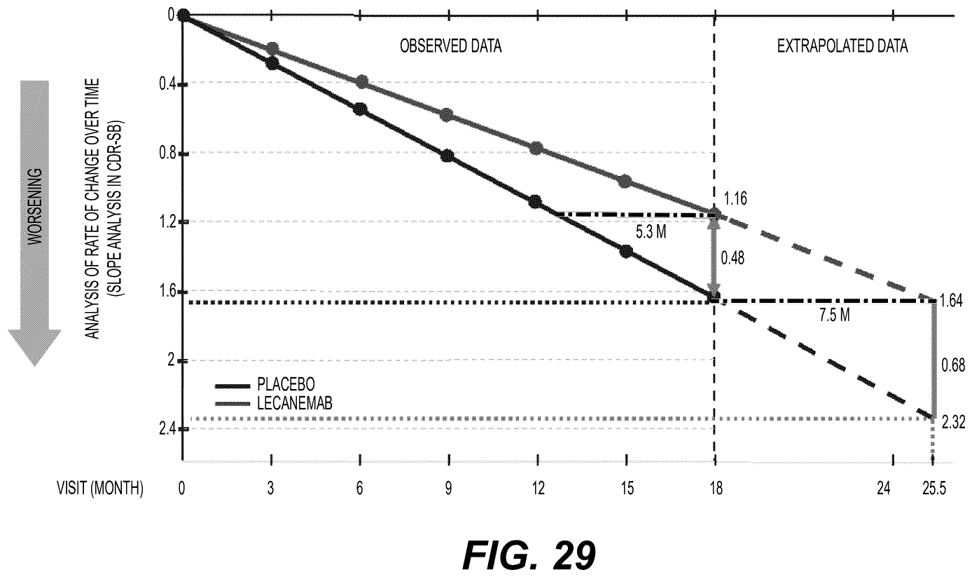

Starting as early as six months, across all time points, lecanemab treatment showed statistically significant changes in the primary and all key secondary endpoints from baseline compared to placebo (all p-values<0.01); see FIG. 20, FIG. 21, FIG. 22, FIG. 28, and FIG. 29. FIG. 23 shows the reduction in brain Aβ plaque (adjusted mean change from baseline in Aβ PET centiloids) over 3, 6, 12, and 18 months in subjects treated with lecanemab and subjects treated with a placebo.

### C. Fluid Biomarkers

Change from baseline at 18 months in fluid biomarkers of Alzheimer's disease pathophysiology showed statistical significance between the lecanemab-treated patients compared to patients on placebo. All biomarker assessments were pre-specified. All fluid biomarkers showed statistically significant improvement except for NfL, which showed a trend at 12 and 18 months (see Table 17).

TABLE 17
Biomarker Results of lecanemab in Study 301

| | lecanemab | |
|---|---|---|
| Biomarker Endpoints | 10 mg/kg biweekly | Placebo |
| Plasma Aβ42/40 ratio | N = 797 | N = 805 |
| Mean Baseline | 0.088 | 0.088 |
| Adjusted mean change from | 0.006 | −0.000 |
| Baseline at Week 53 | | |
| Difference from placebo | 0.007 (p < 0.00001) | |
| Adjusted mean change from | 0.008 | 0.001 |
| Baseline at Week 77 | | |
| Difference from placebo | 0.007 (p < 0.00001) | |
| Plasma p-tau181 (pg/mL) | N = 746 | N = 752 |
| Mean Baseline | 3.696 | 3.740 |
| Adjusted mean change from | −0.466 | 0.278 |
| Baseline at Week 53 | | |
| Difference from placebo | −0.744 (p < 0.00001) | |
| Adjusted mean change from | −0.575 | 0.201 |
| Baseline at Week 77 | | |
| Difference from placebo | −0.776 (p < 0.00001) | |
| Plasma GFAP (pg/mL) | N = 736 | N = 730 |
| Mean Baseline | 355.624 | 361.082 |
| Adjusted mean change from | −29.126 | 42.183 |
| Baseline at Week 53 | | |
| Difference from placebo | −71.309 (p < 0.00001) | |
| Adjusted mean change from | −47.080 | 36.903 |
| Baseline at Week 77 | | |
| Difference from placebo | −83.983 (p < 0.00001) | |
| Plasma NfL (pg/mL) | N = 702 | N = 731 |
| Mean Baseline | 21.899 | 22.205 |
| Adjusted mean change from | 2.014 | 2.594 |
| Baseline at Week 53 | | |
| Difference from placebo | −0.580 (P = 0.31066) | |
| Adjusted mean change from | 1.838 | 2.944 |
| Baseline at Week 77 | | |
| Difference from placebo | −1.106 (P = 0.07339) | |
| CSF Aβ[1-42] (pg/mL) | N = 134 | N = 135 |
| Mean Baseline | 546.979 | 514.416 |
| Adjusted mean change from | 247.819 | −1.026 |
| Baseline at Week 53 | | |
| Difference from placebo | 248.845 (p < 0.00001) | |
| Adjusted mean change from | 287.283 | −2.542 |
| Baseline at Week 77 | | |
| Difference from placebo | 289.824 (p < 0.00001) | |
| CSF Aβ[1-40] (pg/mL) | N = 104 | N = 108 |
| Mean Baseline | 11,986.991 | 12,334.173 |
| Adjusted mean change from | −519.879 | −452.247 |
| Baseline at Week 53 | | |
| Difference from placebo | −67.632 (p = 0.84300) | |
| Adjusted mean change from | −439.756 | −87.457 |
| Baseline at Week 77 | | |
| Difference from placebo | −352.299 (p = 0.32531) | |
| CSF t-tau (pg/mL) | N = 134 | N = 137 |
| Mean Baseline | 584.993 | 615.216 |
| Adjusted mean change from | −32.628 | 77.909 |
| Baseline at Week 53 | | |
| Difference from placebo | −110.536 (p < 0.00001) | |
| Adjusted mean change from | −30.433 | 94.496 |
| Baseline at Week 77 | | |
| Difference from placebo | −124.929 (p < 0.00001) | |
| CSF p-tau181 (pg/mL) | N = 134 | N = 137 |
| Mean Baseline | 84.922 | 92.081 |
| Adjusted mean change from | −13.196 | 8.939 |
| Baseline at Week 53 | | |
| Difference from placebo | −22.135 (p < 0.00001) | |
| Adjusted mean change from | −16.108 | 12.356 |
| Baseline at Week 77 | | |
| Difference from placebo | −28.464 (p < 0.00001) | |
| CSF Neurogranin (pg/mL) | N = 139 | N = 134 |
| Mean Baseline | 500.094 | 519.060 |
| Adjusted mean change from | −61.522 | 1.744 |
| Baseline at Week 53 | | |
| Difference from placebo | −63.266 (p < 0.00001) | |
| Adjusted mean change from | −71.367 | 18.293 |
| Baseline at Week 77 | | |
| Difference from placebo | −89.660 (p < 0.00001) | |
| CSF NfL (pg/mL) | N = 139 | N = 134 |
| Mean Baseline | 1201.036 | 1109.918 |
| Adjusted mean change from | 73.332 | 63.166 |
| Baseline at Week 53 | | |
| Difference from placebo | 10.165 (p = 0.92035) | |
| Adjusted mean change from | 51.645 | 78.389 |
| Baseline at Week 77 | | |
| Difference from placebo | −26.744 (p = 0.71111) | |

There was a statistically significant difference in the change from baseline in brain tau pathology in 3 composite regions known to accumulate tau early in the disease (temporal, medial temporal, and meta-temporal) as measured by tau PET SUVR at 18 months in lecanemab 10 mg/kg every two weeks compared to placebo (medial temporal ROI: adjusted mean treatment difference 0.068, p=0.00237; meta-temporal ROI: adjusted mean treatment difference 0.071, p=0.01195; temporal ROI: adjusted mean treatment difference 0.065, p=0.01619). The difference was seen as early as 13 months, the first timepoint measured.

These changes in Alzheimer's disease pathophysiologic biomarkers are consistent with biological disease modification with lecanemab treatment.

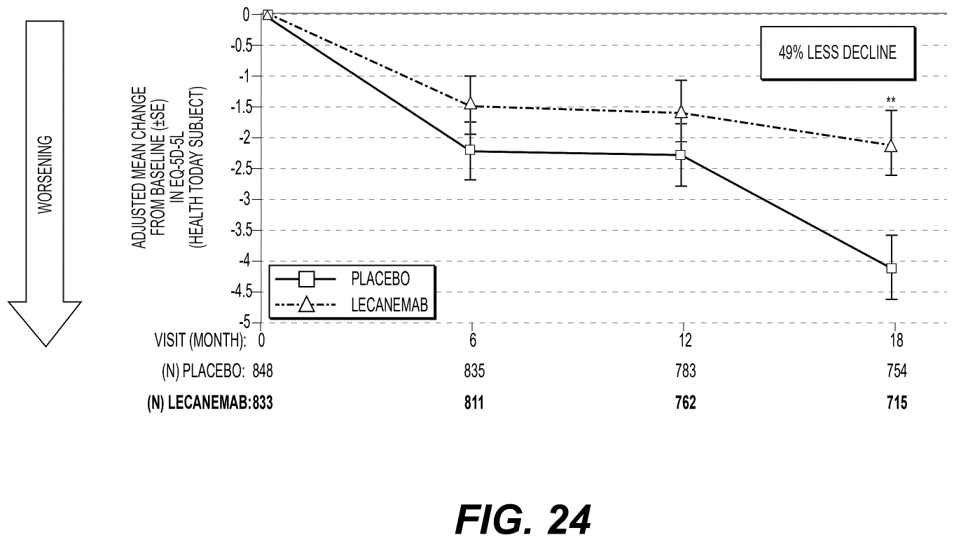

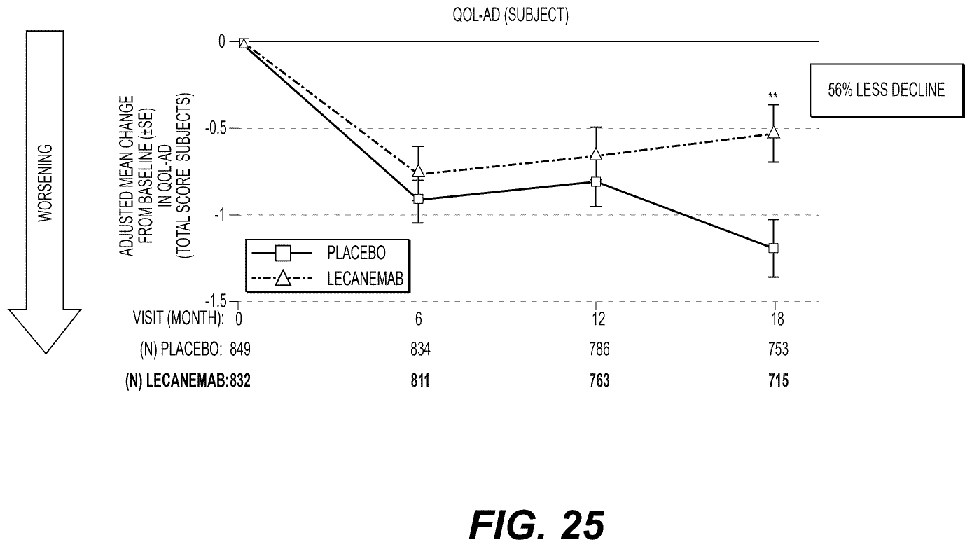

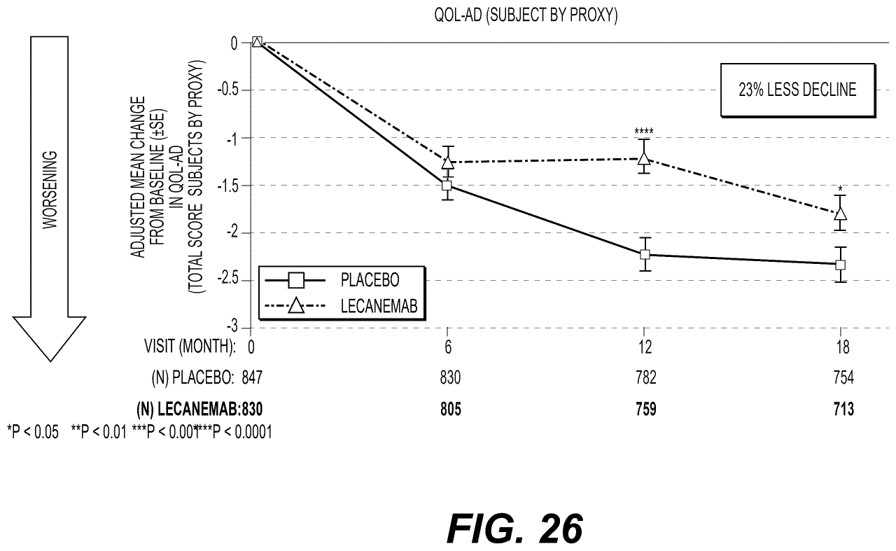

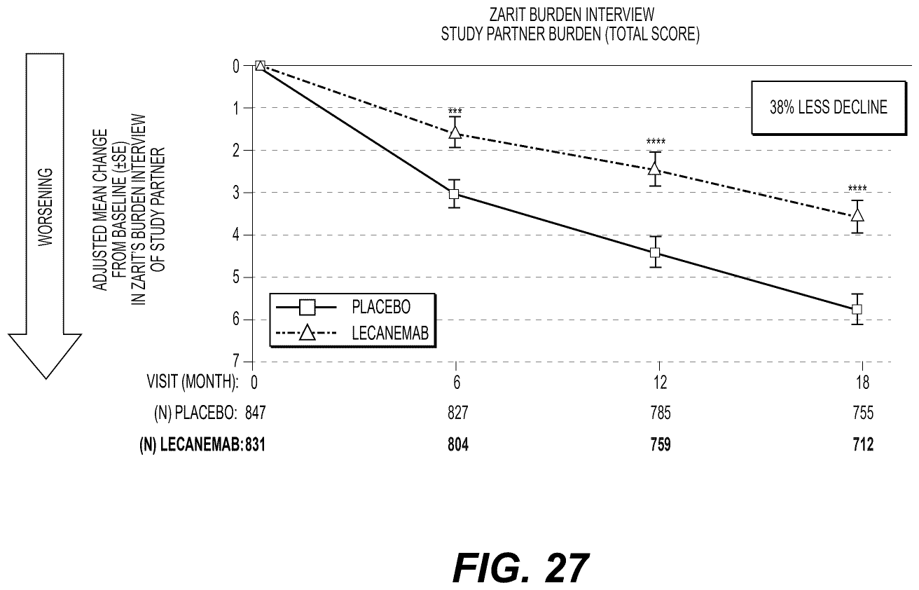

Other prespecified endpoints included the quality of life, caregiver burden, and time to worsening of global CDR scores: European Quality of Life-5 Dimensions 5 Level version [EQ 5D 5L](patient assessment), Quality of Life in Alzheimer's Disease (QOL AD)(patient and patient by proxy assessment), and Zarit Burden Interview (study partner burden) (See, FIG. 24, FIG. 25, FIG. 26, and FIG. 27). For all health-related quality of life outcomes there was a statistically significant difference between the lecanemab-treated group compared with the placebo group. Lecanemab reduced the risk of progression to the next stage of AD on the global CDR score by 31% (hazard ratio: 0.69, 95% CI: 0.572, 0.833, p=0.00011) (Table 16).

As shown in FIG. 25, at month 18, the adjusted mean change from baseline in QOL-AD by subjects treated with Lecanemab (10 mg/kg biweekly) showed 56% less decline compared to placebo. This effect is consistent across QOL-AD (subject) domains. This is consistent across APOE genotypes and range of randomization strata.

As shown in FIG. 26, at month 18, the study partner burden as measured by QOL-AD resulted in 23% less decline in subjects treated with Lecanemab (10 mg/kg biweekly). Overall, results reported by subject are generally consistent with results reported by partner. The effect is consistent across domains. This is consistent across APOE genotypes and range of randomization strata.

As shown in FIG. 27, at month 18, study partner burden as measured by Zarit Burden Interview resulted in 38% less decline in subjects treated with Lecanemab (10 mg/kg biweekly) compared to placebo. The effect was consistent across domains. This was consistent across APOE genotypes and range of randomization strata.

The results of the Clarity AD QoL measures offer additional evidence for meaningful benefits to patients and care partners of lecanemab treatment beyond benefits demonstrated through cognitive, functional, and biomarker measures.

FIG. 28 uses actual duration for time to event. The rate of progression to next stage is plotted over time for subjects receiving lecanemab or placebo. Including the number at risk within a 2-week window, N at 18 months is 497 in placebo and 527 in LEC10-BW

As shown in FIG. 28, lecanemab slows disease progression, maintaining individuals in earlier stages of disease for longer relative to placebo. Progression was defined as global CDR score progressing from 0.5 [MCI] to 1[mild AD dementia] or 1 [mild dementia] to 2 [moderate dementia]). Hazard ratio 0.69 (95% CI 0.57-0.83, p=0.00011). Patients on lecanemab had a 31% lower risk of converting to next stage of disease by global CDR. As a result, individuals remain in earlier stages of Alzheimer's disease for a longer period of time, even within the 18-month course of the study.

FIG. 29 shows a slope analysis using CDR-SB observed data and extrapolation to 2 years. Rate of change over time (mean slope) based on change from baseline in the CDR-SB was analyzed using linear mixed effects (LME) model. LME model included time, treatment by assessment time as covariate with random intercept and slope. As shown in FIG. 29, patients on lecanemab had a 32% slowing of slope annually [(95% CI. 18% to 46%), p=0.00001] vs. placebo. Projected treatment difference at 25.5 months (M) based on slope showed −0.68 treatment difference. Result is increasing separation over time between lecanemab & placebo. Lecanemab takes 25.5 months to reach same level as placebo at 18 months.

D. Change in Plasma GFAP Level with Treatment by Lecanemab

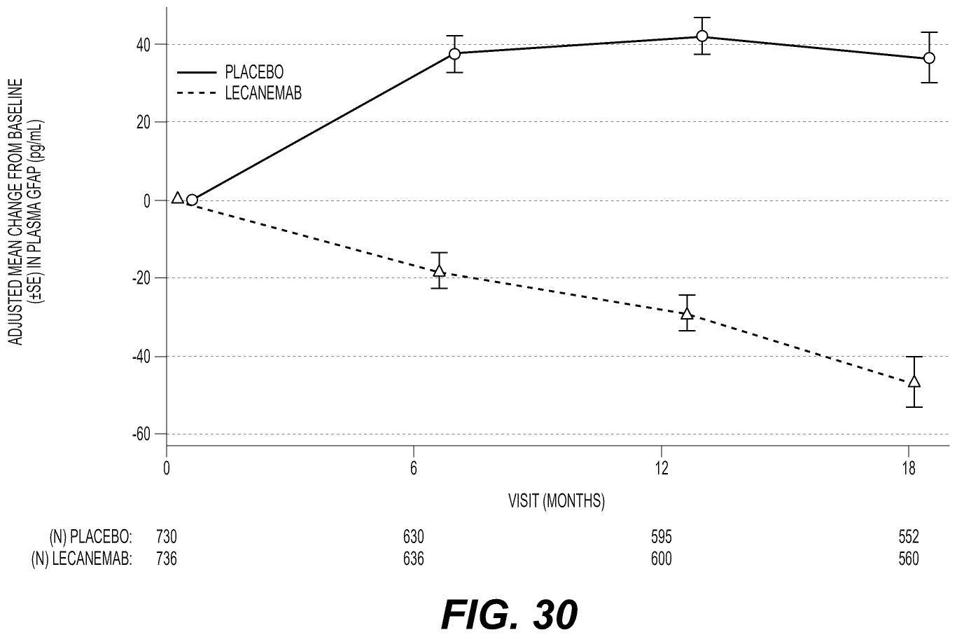

Adjusted mean change from baseline of plasma GFAP level, SE and p-value are derived using mixed model for repeated measures with treatment group, visit, treatment group by visit interaction, clinical subgroup, use of Alzheimer's disease symptomatic medication at baseline, ApoE4 carrier status, region, baseline value by visit interaction as fixed effects, and baseline value as covariate (FIG. 30).

The adjusted mean change from baseline, SE, and p-value are derived using a mixed model for repeated measures with treatment group, visit, treatment group by visit interaction, clinical subgroup, use of Alzheimer's disease symptomatic medication at baseline, ApoE4 carrier status, baseline value by visit interaction as fixed effects, and baseline value as covariate.

### E. Summary of Study 301 Clarity AD Results

Lecanemab is an anti-amyloid monoclonal antibody that binds with highest affinity to soluble Aβ protofibrils, which are more toxic than monomers or insoluble fibrils/plaque. In the phase 3 Clarity AD study, lecanemab demonstrated a consistent slowing of decline in clinical (global, cognitive, functional, and quality of life) outcomes, and reduction in brain amyloid in early Alzheimer's disease (AD).

The Clarity AD study provided an overview of the mechanistic and clinical rationale for development of lecanemab, including how lecanemab treatment impacts tau protein aggregates, a predictive biomarker for AD that is closely linked to emergence of neurodegeneration and manifestation of clinical symptoms.

As described above, Clarity AD was an 18-month treatment (core study), multicenter, double-blind, placebo-controlled, parallel-group study with open-label extension (OLE) in patients with early AD. Eligibility criteria included being 50-90 years of age with a diagnosis of mild cognitive impairment or mild dementia due to AD with biomarker confirmed amyloid pathology. Eligible participants were randomized 1:1 to receive either placebo or lecanemab 10-mg/kg IV biweekly. The primary efficacy endpoint was change from baseline at 18 months in the Clinical Dementia Rating-Sum-of-Boxes (CDR-SB). Key secondary endpoints included change from baseline at 18 months in amyloid PET standardized uptake value ratio (in patients participating in the sub-study), Alzheimer's Disease Assessment Scale-Cognitive Subscale 14 (ADAS-Cog14), and Alzheimer's Disease Cooperative Study-Activities of Daily Living Scale for Mild Cognitive Impairment (ADCS-MCI-ADL). Subgroup analysis of low baseline tau was conducted (MK-6240 tau PET SUVr <1.06). The OLE evaluates the long-term safety and tolerability of lecanemab 10 mg/kg biweekly in patients with early AD.

Overall, 1795 participants were randomized (lecanemab:898; placebo:897). Lecanemab treatment met the primary endpoint, reducing decline on the CDR-SB versus placebo at 18 months by 27.1% (difference: −0.45; P<0.001), with highly significant differences starting at six months. At 18 months, all key secondary outcomes favored lecanemab (P<0.001), reducing brain amyloid (difference: −59.1 [95% CI: −62.6, −55.6]), slowing cognition loss by 25.8% (ADAS-Cog14), and functional decline by 36.6% (ADCS-MCI-ADL). Lecanemab was generally well-tolerated, with an incidence of amyloid-related imaging abnormalities-edema (ARIA-E) of 12.6% for lecanemab and 1.7% for placebo (symptomatic: lecanemab:2.8%; placebo:0.0%). In the low tau subgroup, 60% of lecanemab participants had improvement on CDR-SB compared to 28% in placebo. Efficacy trends continued in the OLE, with no new safety signals. Indirect comparison between trials would suggest lecanemab has lower ARIA-E than some other published Aβ immunotherapies even without titration. Incidence and timing of ARIA vary among treatments, possibly related to differences in binding profiles to soluble aggregated amyloid species, amyloid plaques, and vascular amyloid.

Lecanemab demonstrated a consistent slowing of decline in clinical (global, cognitive, and functional) outcomes, and reduction in brain amyloid in early AD, including in low tau population. Targeting the protofibrils may provide an additional benefit by continuing to target Aβ species and improve biomarkers even after clearance of amyloid plaques.

As shown in Table 18, patients with low tau PET levels responded surprisingly well in primary and key secondary outcomes relative to patients treated with higher tau PET levels. Other anti-Aβ protofibril antibodies, such as the anti-Aβ protofibril antibody produced by Eli Lilly, have not tested the efficacy in patients with low tau PET levels, suggesting that an effect was not expected.

TABLE 18
Table summarizing results of Study 301 Clarity AD

| Characteristic | CLARITY AD |
|---|---|
| Study Design | Global, Placebo-Controlled, Double-blind, |
| | Randomized Trial with OLE |
| Study Population | MCI due to AD or mild AD dementia |
| | (NIA-AA criteria, CDR 0.5-1) |
| | Confirmed amyloid pathology (amyloid |
| | PET visual read or CSF) |
| | MMSE 22-30 |
| | Memory impairment (WMS-IV LMSII ≥1 |
| | SD below age-adjusted mean) |
| | Age 50-90 |
| Randomization | APOE4 status (carrier vs non-carrier) |
| Stratification | Disease stage (MCI vs mild AD) |
| | Use of symptomatic AD meds (Yes/No) |
| | Geographic region |
| Dose | LEC 10 mg/kg IV q2wk |
| Outcomes | Primary: CDR-SB |
| | Key Secondary |
| | Amyloid PET |
| | ADAS-cog14 |
| | ADCOMS |
| | ADCS-MCI-ADL |
| N | 1795 |
| Baseline | |
| Demographic | |
| Characteristics | |
| Geography | US/Can 60%; EU 24%; Asia 16% |
| Age | 72 y |
| Gender | 52% F |
| Diagnosis | MCI 62%; Mild AD 38% |
| APOE4 | None 31%; Het 54%; Hom 15% |
| AchEi | 54% |
| Black/Hispanic | 25% (US) |
| Baseline | |
| Clinical | |
| Characteristics | |
| iADRS | |
| ADAS-Cog13 | ADAS-cog14: 24.4 |
| ADCS-iADL | ADCS-ADL-MCI: 41 |
| CDR-SB | 3.2 |
| MMSE | 25.6 |
| Baseline | |
| Imaging | |
| Amyloid PET | 76 CL |
| Tau PET | Tau classification (Eisai method, |
| | MK tracer), N = 355 substudy |
| | Low (<1.06): 149 |
| | Intermediate (1.06-2.91): 196 |
| | High (>2.91): 10 |
| | Tau classification in mild AD |
| | Low (<1.06): 60 |
| | Intermediate (1.06-2.91): 76 |
| | High (>2.91): 4 |
| Results | |
| iADRS | NA |
| CDR-SB | 27% |
| | Tau PET substudy (N = 355) |
| | Overall: 38% |
| | Intermediate + high tau: 27% |
| | Intermediate + low tau: 49% |
| | Intermediate tau: 27% |
| | Low tau: 549% (improved) |
| ADCS-iADL | ADCS-ADL-MCI |
| | 37% |
| | Tau PET substudy (N = 355) |
| | Overall: 39% |
| | Intermediate + high tau: 13% |
| | Intermediate + low tau: 41% |
| | Intermediate tau: 12% |
| | Low tau: 88% |
| ADAS-cog | ADAS-cog14 |
| | 26% |
| | Mild AD: 30% |
| | Tau PET substudy (N = 355) |
| | Overall: 16% |
| | Intermediate + high tau: 9% |
| | Intermediate + low tau: 21% |
| | Intermediate tau: 14% |
| | Low tau: 52% |
| Progression to | HR 0.69 |
| next stage of | More work being done |
| disease | |
| No decline at | Not done |
| 12M on CDR-SB | |
| Amyloid | Criteria by clinical read ~<30 CL |
| clearance | 6M: 36% |
| | 12M: 54% |
| Safety | |
| ARIA-E | 12.6% |
| Symptomatic | 2.8% |
| ARIA-E | |
| Characteristic | CLARITY AD |
| ARIA-H | 17.3% |
| SAE ARIA | ARIA-E or H 1.1% |
| Infusion | LEC 26.4%; PBO 7.4% |
| reactions | |

### Example 11. Methods of Measuring Tau PET in Patients Treated with Lecanemab

### A. Background

There are multiple Tau PET radiotracers in use and the signal is highly correlated for many SUVR outcome measures, however the dynamic ranges and sensitivities of the tracers is different. Recently, a large trial (Trailblazer) was completed by Eli Lilly using the first generation tracer [¹⁸F]Flortaucipir in which the eligibility criteria included Tau PET SUVR. It was hypothesised that the greatest treatment effect would be observed in patients with an intermediate level of Tau (so subjects were excluded if their Tau PET SUVR was lower than the low cut-point or higher than the high cut-point). [¹⁸F]MK6240 is a second generation Tau PET tracer which could be used in multiple future trials but further direct head-to-head data is needed to determine equivalent SUVR cut-points.

One aim of the present study was to use cross-sectional [¹⁸F]MK6240 data along with estimated equivalent cut-points for low and high signal to those used in the Trailblazer clinical trial.

### B. Methods

Cut-points (also called thresholds, cut-off points, or cut-offs) were determined in a two-stage process;
1. Identify a subset of subjects from a given dataset that are similar in cognition to a reference data set and use the distribution in the given dataset to define cut-offs for Global Tau Load as derived using TauIQ, or as derived from a databased of MK6420 PET scans (e.g., the Lantheus/Cerveau database; see (cerveautechnologies.com)).
2. Apply cut-offs derived in stage one to the dataset to determine intermediate tau subjects and use this distribution to define cut-offs for SUVR.

Stage 1. The Trailblazer trial included only Aβ+AD subjects and therefore we obtained [¹⁸F]MK6240 scans from a database (e.g., the Lantheus/Cerveau database, see Lantheus Cerveau (cerveautechnologies.com)) for 64 subjects (out of a possible 866) which matched this criteria and had a recorded MMSE score. The distribution of MMSE scores of the 64 subjects was similar to those used for the Trailblazer trial (current data set MMSE=23.4 (3.52), Trailblazer MMSE=23.5(3.1)). Global Tau Load (an outcome measure ranging from 0-100% quantifying the global tau accumulation pattern) was calculated for each scan using the TauIQ algorithm.

Cut-off values for Global Tau Load were defined as follows from the distribution of Global Tau Load values obtained from the 64 subjects.

$$Low Tau < Mean - SD = 12.2 \%$$

$$High Tau > Mean + SD = 63.9 \%$$

Stage 2. The cut-offs were applied to the 64 subjects from the current data set and 37 subjects were found to have intermediate levels of signal. A cortical SUVR (grey matter masked), which is the whole cortical gray matter SUVR, was calculated for each scan using cortical regions from the CIC atlas (Tziortzi et al., 2011).

In order to counteract the effects of outliers, cut-off values for Cortical SUVR were defined as follows from the distribution of Cortical SUVR values obtained from the 37 subjects.

$$Low Tau < {5}^{th} percentile = 1.06$$

$$High Tau > {95}^{th} percentile = 2.91$$

The results are shown above in Table 18.

### Example 12. Tau PET Substudy

In a further analysis, Tau PET data from patients in Clarity AD with low tau PET levels at baseline were assessed for tau pathology in different brain regions. Tau pathology was assessed by tau PET over a period of 18 months after administration of lecanemab or placebo.

As described above, Clarity AD was an 18-month, global, multicenter, double-blind study in individuals with early Alzheimer's disease. Eligible participants were randomized 1:1 to receive either placebo or lecanemab 10-mg/kg IV biweekly. The primary efficacy end point was change from baseline at 18 months in the Clinical Dementia Rating-Sum-of-Boxes (CDR-SB).

A subgroup analysis of low baseline tau was conducted from patients who participated in an optional tau PET sub-study.

Lecanemab treatment impacted tau biomarkers across various biomarker platforms (CSF, plasma, imaging). Of the 342 patients in the tau PET substudy, 141 (41.2%) were low tau (MK-6240 tau PET whole cortical gray matter SUVr <1.06), 191 (55.8%) were intermediate tau (SUVr ≥1.06, ≤2.91), and 10 (2.9%) were high tau (SUVr >2.91). Lecanemab has downstream effects on tau pathology by PET and soluble measures of tau. In the low tau subgroup, lecanemab reduced decline on CDR-SB relative to placebo by −0.59 (549% improvement; p=0.022) at 18 months. In the low tau subgroup, 60% of lecanemab treated patients had improvement on CDR-SB (vs 28% on placebo) and 76% had no decline on CDR-SB (vs 55% on placebo) at 18 months. Lecanemab slowed tau pathology in temporal lobe (early Braak regions). In addition, lecanemab impacts different brain regions in low tau PET group vs intermediate tau PET group consistent with stage of disease. In those with low tau, lecanemab impacts medial temporal (which is the earliest tau region) tau progression, while in intermediate tau (which on average has tau already in temporal and parietal regions), lecanemab impacts tau progression on PET more broadly.

Biomarker assessment results from Clarity AD show that lecanemab treatment has an overall effect on tau PET for all patients, arresting tau progression/spread in low tau patients and changing the tau accumulation trajectory in patients with higher tau levels. Results from Clarity AD in the low tau subgroup supports earlier treatment with lecanemab.

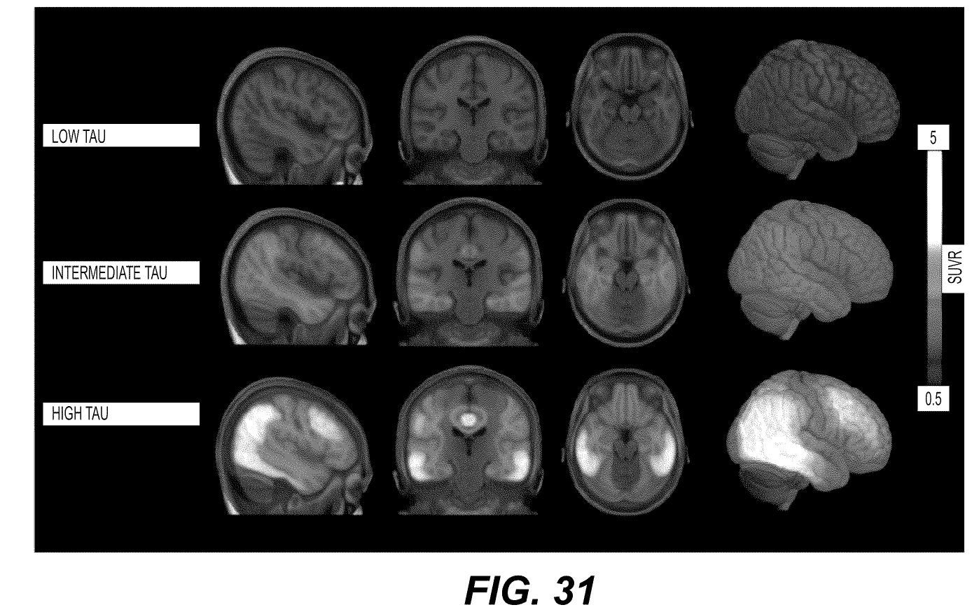

FIG. 31 shows averaged scans from patients in the Tau PET substudy. Baseline characteristics of patients in the Tau PET substudy were similar to the baseline characteristics overall study population.

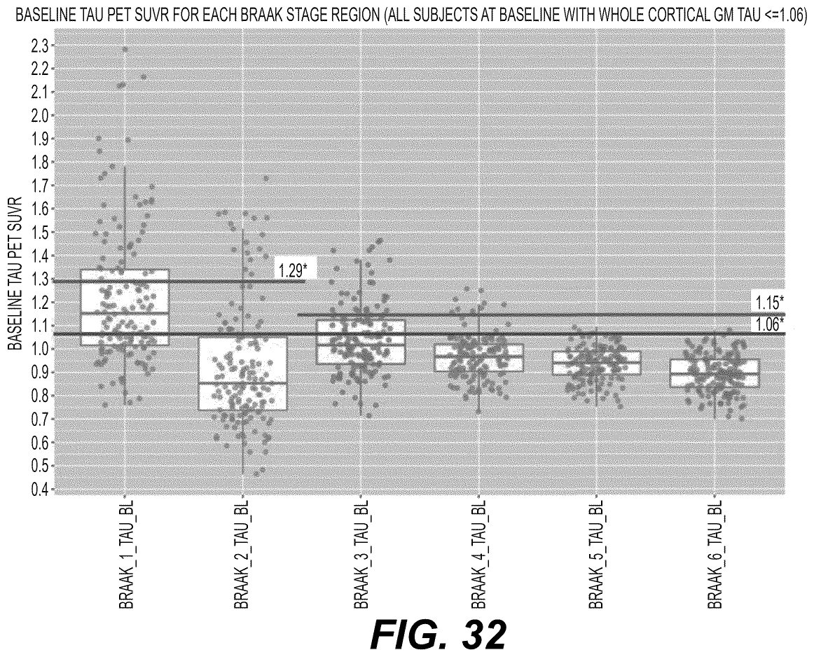

FIG. 32 shows the tau PET SUVr in Braak stage regions in subjects with low whole cortical tau aggregation (e.g., subjects in the low tau PET subgroup as determined by a SUVr <1.06 in whole cortical gray matter in MK6240 PET scans). Overall, when low tau subjects have tau accumulation in Braak stage regions, it is primarily in Regions 1-3.

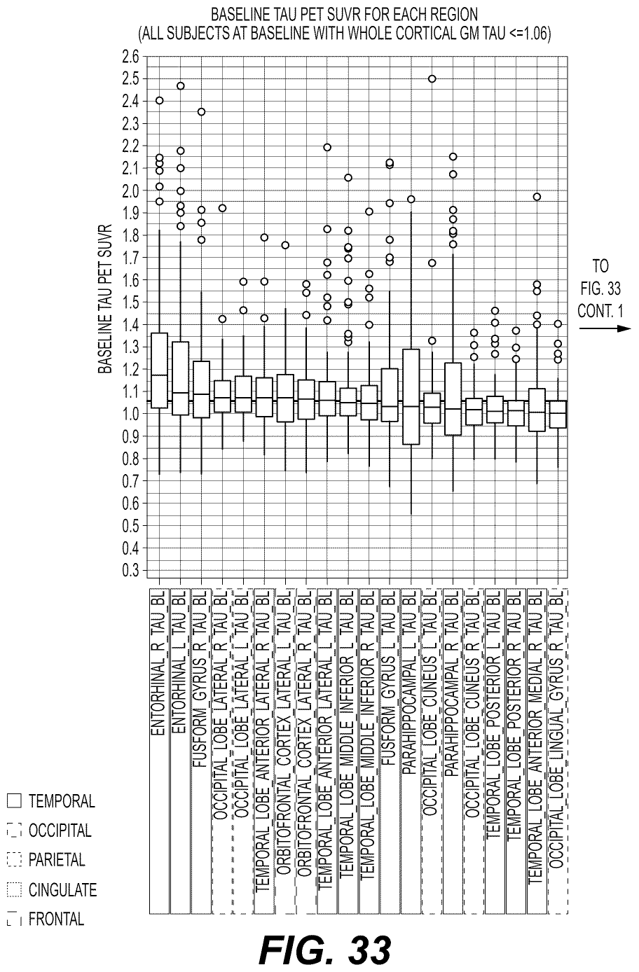

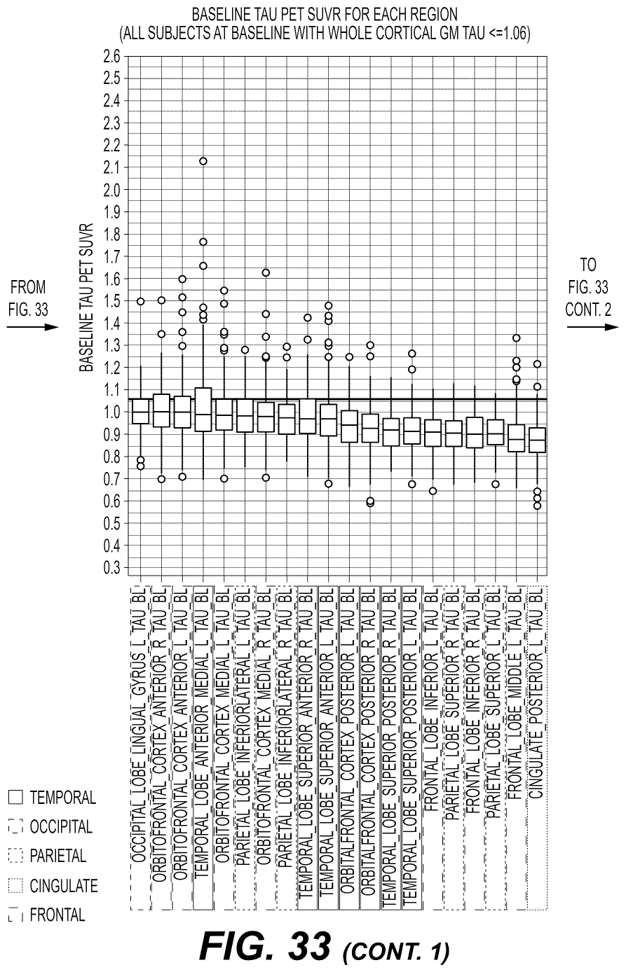

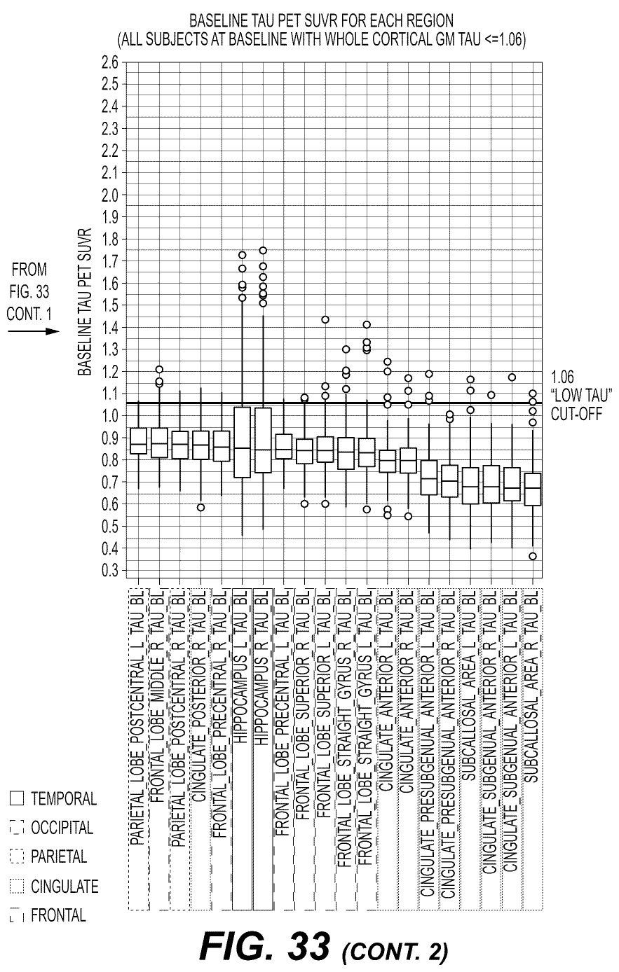

FIG. 33 shows an ordering by decreasing median baseline tau PET SUVr. This ordering, based on tau PET, recapitulates the spatio-temporal tau accumulation pattern observed in the AD patients, where tau aggregation is first observed in the early Braak stage regions (e.g., entorhinal and temporal regions), and is later observed in later Braak stage regions (e.g., occipital cortex, parietal cortex, and fronto-cingulate cortex).

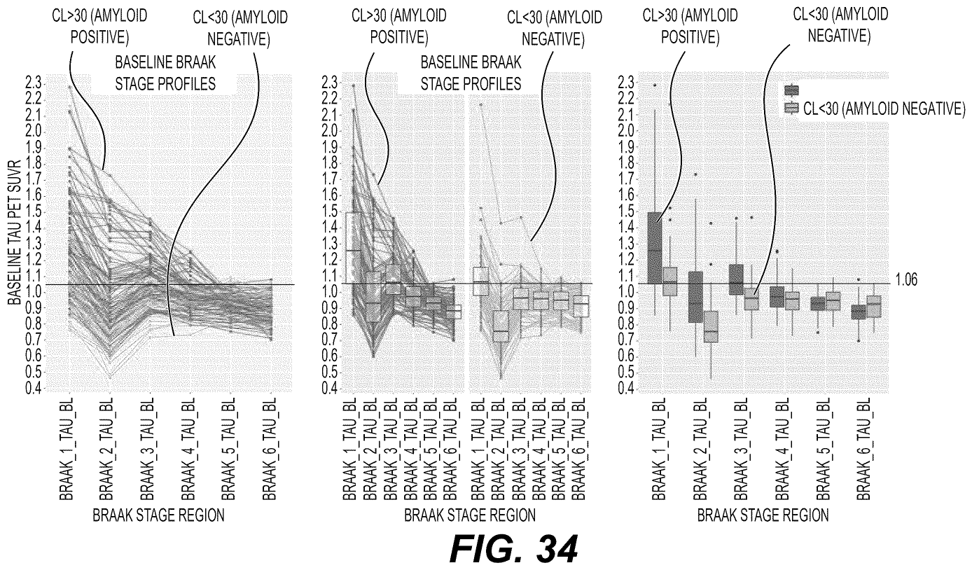

FIG. 34 shows regional tau PET SUVr results from subjects in the low tau PET subgroup who are amyloid positive (>30 CL). These subjects have elevated tau PET in early Braak stage regions. Pairwise comparisons were made using t tests with pooled SD. P value adjustment method: FDR.

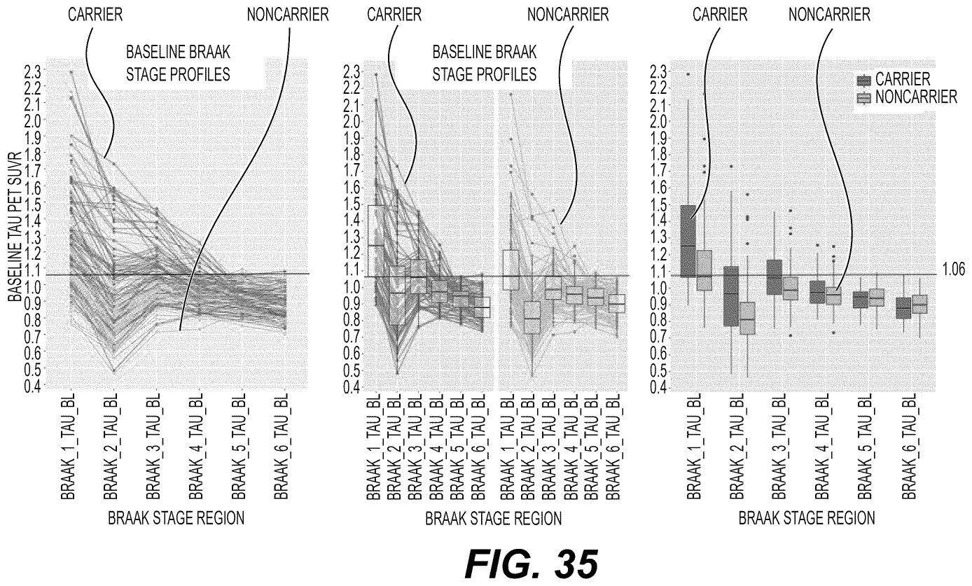

FIG. 35 shows regional tau PET SUVr results from subjects in the low tau PET subgroup who are ApoE4 carriers. These subjects have elevated tau PET SUVr in early Braak stage regions.

Table 19 summarizes the patients included in the study. Of all the patients in the Clarity study, a subgroup of patients underwent a Tau PET substudy. Some patients in the Tau PET substudy had low tau PET levels, defined based on cutoffs from MK6240 scans in the Lantheus/Cerveau database (see Lantheus Cerveau (cerveautechnologies.com)), (amyloid positive patients with a mean MMSE (SD): 23.4 (3.5)) as follows:
Low Tau: tauPET SUVr below 1.06 (n=141 [41.2%]
Intermediate Tau: tau PET SUVr between 1.06-2.91 (n=191 [55.8%])
High Tau: tau PET SUVr above 2.91 (n=10 [2.9%])

Table 19 summarizes results from subjects included in the tau PET Substudy (column entitled Tau PET Substudy¹ are subjects with a baseline tau PET; tau-PET levels were determined according to the cut-offs described above).

TABLE 19
Comparison of results from all subjects in the Clarity
AD study and subjects in the tau PET substudy.

| All Subjects | | | Tau PET Substudy ¹ | |
|---|---|---|---|---|
| | | Lecanemab | | Lecanemab |
| | Placebo | 10 mg/kgbiweekly | Placebo | 10 mg/kgbiweekly |
| | (N = 875) | (N = 859) | (N = 167) | (N = 175) |
| Age, mean (standard | 71.0 (7.8) | 71.4 (7.9) | 72.4 (7.8) | 71.8 (7.8) |
| deviation), years | | | | |
| Female, n (%) | 464 (53.0) | 443 (51.6) | 92 (55.1) | 84 (48.0) |
| CDR Global = 0.5 | 706 (80.7) | 694 (80.8) | 132 (79.0) | 127 (72.6) |
| MMSE, mean (SD) | 25.6 (2.23) | 25.5 (2.19) | 25.6 (2.09) | 25.6 (2.18) |

AD Stage

| | | | | |
|---|---|---|---|---|
| MCI | 544 (62.2) | 528 (61.5) | 108 (64.7) | 101 (57.7) |
| Mild dementia | 331 (37.8) | 331 (38.5) | 59 (35.3) | 74 (42.3) |

ApoE4 Status

| | | | | |
|---|---|---|---|---|
| Noncarrier | 275 (31.4) | 267 (31.1) | 70 (41.9) | 75 (42.9) |
| Heterozygous | 468 (53.5) | 456 (53.1) | 84 (50.3) | 77 (44.0) |
| Homozygous | 132 (15.1) | 136 (15.8) | 13 (7.8) | 23 (13.1) |
| CDR-SB, mean (SD) | 3.22 (1.343) | 3.17 (1.340) | 3.31 (1.332) | 3.40 (1.307) |
| PET Centiloids, | 75.28 (41.85) | 77.94 (44.78) | 73.84 (41.032) | 70.65 (46.844) |
| mean (SD) | | | | |
| ADAS-Cog14, mean (SD) | 24.37 (7.561) | 24.45 (7.082) | 22.88 (6.959) | 22.65 (6.723) |
| ADCS-ADL-MCI | 40.9 (6.89) | 41.2 (6.61) | 40.68 (6.669) | 40.66 (6.919) |

Table 20 shows results from subjects in the tau PET substudy, categorized into either a low tau-PET subgroup or an intermediate+high tau-PET subgroup.

TABLE 20
Comparison of results from subjects in the tau PET substudy.

| | | | | | Intermediate + | |
|---|---|---|---|---|---|---|
| | Tau PET Substudy | | Low Tau | | High Tau | |
| | Placebo | Lecanemab | Placebo | Lecanemab | Placebo | Lecanemab |
| | (N = 167) | (N = 175) | (N = 71) | (N = 70) | (N = 96) | (N = 105) |
| Age, mean (SD), | 72.4 | 71.8 | 71.8 | 72.6 | 72.8 | 71.2 |
| years | (7.8) | (7.8) | (8.6) | (7.6) | (7.1) | (7.9) |
| Female, n (%) | 92 | 84 | 36 | 30 | 56 | 54 |
| | (55.1) | (48.0) | (50.7) | (42.9) | (58.3) | (51.4) |
| Caucasian | 155 | 163 | 64 | 64 | 91 | 99 |
| | (92.8) | (93.1) | (90.1) | (91.4) | (94.8) | (94.3) |
| Years since | 1.47 | 1.47 | 1.48 | 1.68 | 1.45 | 1.33 |
| diagnosis | (1.949) | (1.444) | (1.907) | (1.482) | (1.990) | (1.409) |
| Years since onset | 4.21 | 4.32 | 3.81 | 4.77 | 4.51 | 4.01 |
| of symptoms | (3.042) | (2.443) | (2.027) | (2.488) | (3.596) | (2.377) |
| CDR Global = 0.5 | 132 | 127 | 56 | 47 | 76 | 80 |
| | (79.0) | (72.6) | (78.9) | (67.1) | (79.2) | (76.2) |
| Mild dementia | 59 | 74 | 21 | 34 | 38 | 40 |
| due to AD | (35.3) | (42.3) | (29.6) | (48.6) | (39.6) | (38.1) |
| MCl | 108 | 101 | 50 | 36 | 58 | 65 |
| | (64.7) | (57.7) | (70.4) | (51.5) | (60.4) | (61.9) |
| ApoE4 Status | | | | | | |
| Noncarrier | 70 | 75 | 36 | 42 | 34 | 33 |
| | (41.9) | (42.9) | (50.7) | (60.0) | (35.4) | (31.4) |
| Carrier | 97 | 100 | 35 | 28 | 62 | 72 |
| | (58.1) | (57.1) | (49.3) | (40.0) | (64.6) | (68.6) |
| Heterozygous | 84 | 77 | 34 | 24 | 50 | 53 |
| | (50.3) | (44.0) | (47.9) | (34.3) | (52.1) | (50.5) |
| Homozygous | 13 | 23 | 1 | 4 | 12 | 19 |
| | (7.8) | (13.1) | (1.4) | (5.7) | (12.5) | (18.1) |
| On AChEls and/ | 66 | 71 | 31 | 24 | 35 | 47 |
| or memantine | (39.5) | (40.6) | (43.7) | (34.3) | (36.5) | (44.8) |
| CDR - SB, | 3.31 | 3.40 | 3.20 | 3.44 | 3.40 | 3.38 |
| mean (SD) | (1.332) | (1.307) | (1.369) | (1.424) | (1.304) | (1.230) |
| PET Centiloids, | 73.84 | 70.65 | 50.36 | 36.35 | 90.96 | 93.51 |
| mean (SD) | (41.032) | (46.844) | (37.637) | (35.790) | (34.536) | (38.753) |
| ADAS - Cog14, | 22.88 | 22.65 | 20.56 | 21.39 | 24.59 | 23.49 |
| mean (SD) | (6.959) | (6.723) | (6.285) | (6.562) | (6.968) | (6.728) |
| ADCOMS, | 0.40 | 0.41 | 0.37 | 0.39 | 0.42 | 0.41 |
| mean (SD) | (0.144) | (0.145) | (0.145) | (0.148) | (0.141) | (0.142) |
| ADCS MCI-ADL, | 40.68 | 40.66 | 40.35 | 38.81 | 40.90 | 41.92 |
| mean (SD) | (6.669) | (6.919) | (6.832) | (6.927) | (6.581) | (6.657) |
| MMSE, | 25.65 | 25.62 | 25.92 | 25.46 | 25.45 | 25.72 |
| mean (SD) | (2.094) | (2.178) | (2.136) | (2.012) | (2.051) | (2.285) |

In each patient, the change from baseline tau PET (measured prior to administration of lecanemab or placebo) was assessed in at least one post-baseline tau PET measurement, e.g., at 12 months or 18 months after administration. Across all patients, the mean change was adjusted to account for variability.

Tau pathology, as measured by tau PET, was determined in the medial temporal lobe, meta temporal lobe, temporal lobe, frontal cortex, cingulate cortex, parietal cortex, occipital cortex, and whole cortical grey matter.

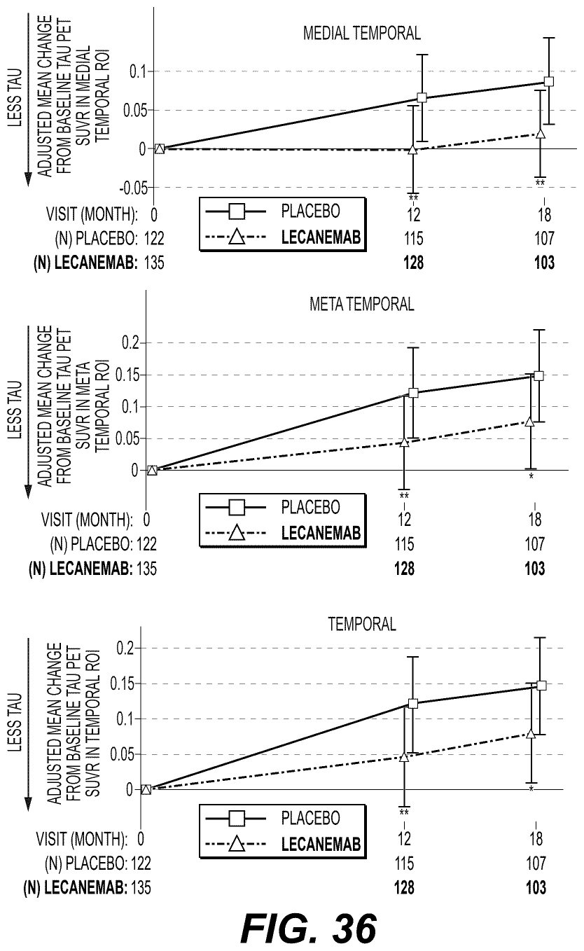

As shown in FIG. 36, lecanemab slowed tau pathology in the 3 composite regions known to accumulate tau early in the disease (temporal, medial temporal, and meta-temporal), as compared to placebo.

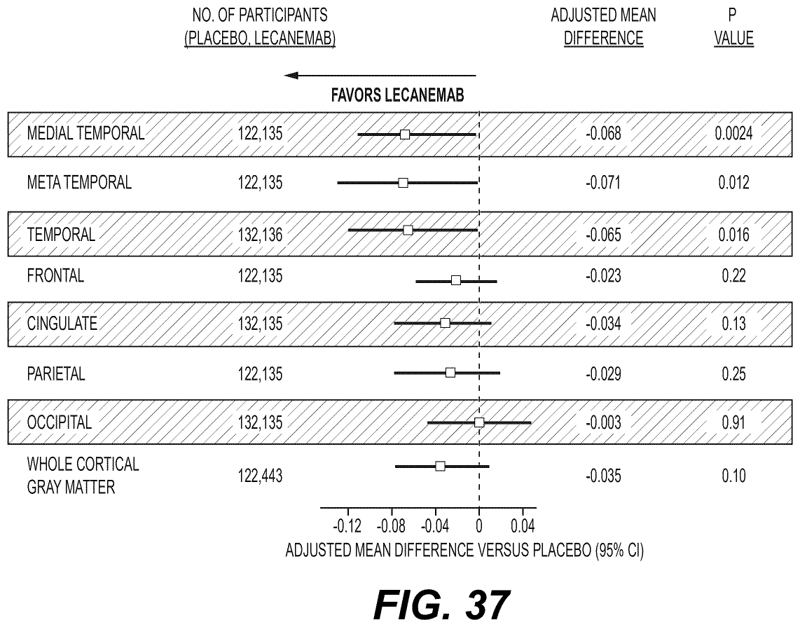

FIG. 37 summarizes the adjusted mean difference in tau pathology, as measured by tau-PET SUVr across brain regions for patients who received lecanemab as compared to patients who received a placebo. As mentioned above, lecanemab slowed tau pathology in the medial temporal lobe, meta temporal lobe, and temporal lobe, as compared to placebo. In the frontal cortex, cingulate cortex, parietal cortex, occipital cortex, and whole cortical grey matter, Lecanemab showed a trend towards lower tau pathology (adjusted mean difference versus placebo, p>0.05, 95% confidence interval). The analysis is based on a modified intention-to-treat analysis population. The adjusted mean change from baseline, SE and p-value are derived using mixed model repeat measures (MMRM) with treatment group, visit, treatment group by visit interaction, clinical subgroup, use of Alzheimer's disease symptomatic medication at baseline, ApoE4 carrier status, region, baseline value by visit interaction as fixed effects, and baseline value as covariate.

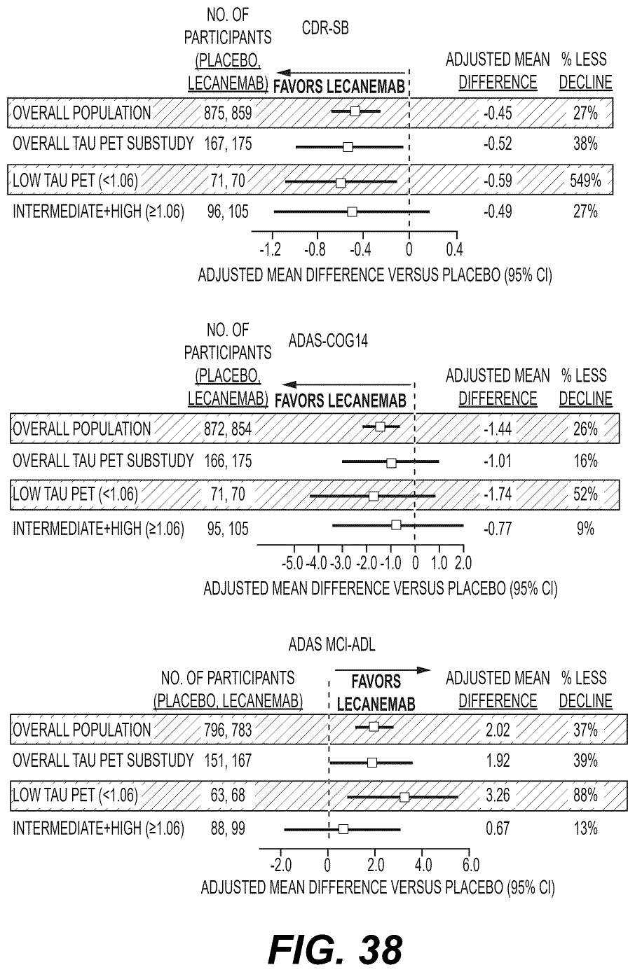

FIG. 38 shows the efficacy of lecanemab on cognitive and functional outcomes in subjects from the overall early AD patients studied in CLARITY, the representative tau PET substudy, and in subjects in low tau and intermediate+high tau subgroups. Outcomes were consistent for subgroups across clinical assessments. The longitudinal tau PET substudy was predefined. The subgroups (low and intermediate+high tau PET) were a post-hoc analysis with nominal p values and no adjustment for multiplicity.

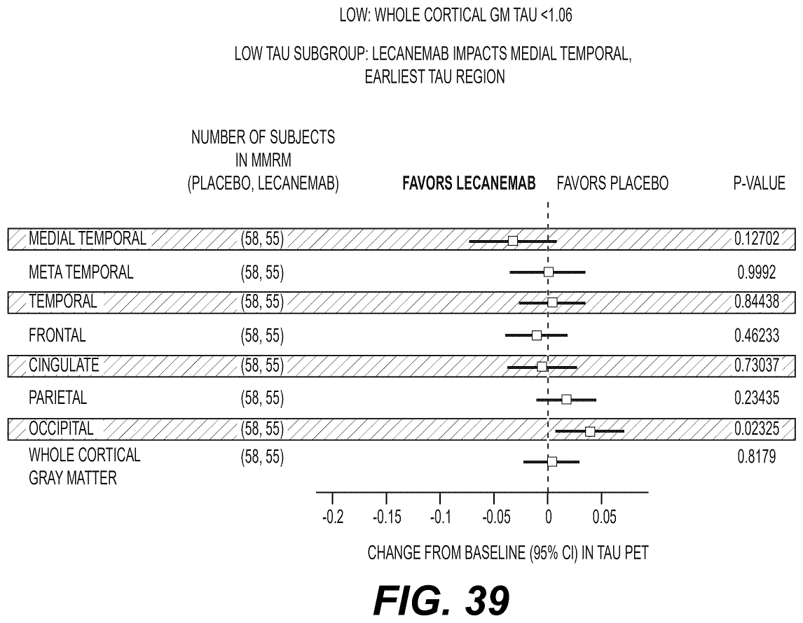

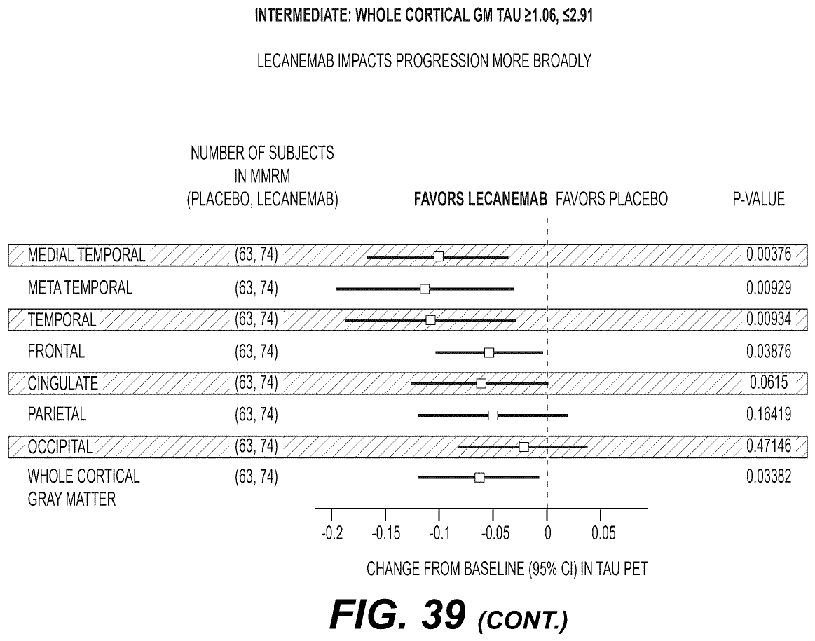

FIG. 39 shows that lecanemab impacts different brain regions in the low tau PET group, as compared to the intermediate+high tau PET group. In the low tau PET group, lecanemab impacts the medial temporal region. In the intermediate+high tau PET group, lecanemab impacts tau spread more broadly across neocortical regions. The analysis in the intermediate+high tau PET groups was a post-hoc analysis with nominal p values and no adjustment for multiplicity.

The analysis is based on modified intention-to-treat analysis population. The adjusted mean change from baseline, SE and p-value are derived using mixed model repeat measures (MMRM) with treatment group, visit, treatment group by visit interaction, clinical subgroup, use of Alzheimer's disease symptomatic medication at baseline, ApoE4 carrier status, region, baseline value by visit interaction as fixed effects, and baseline value as covariate.

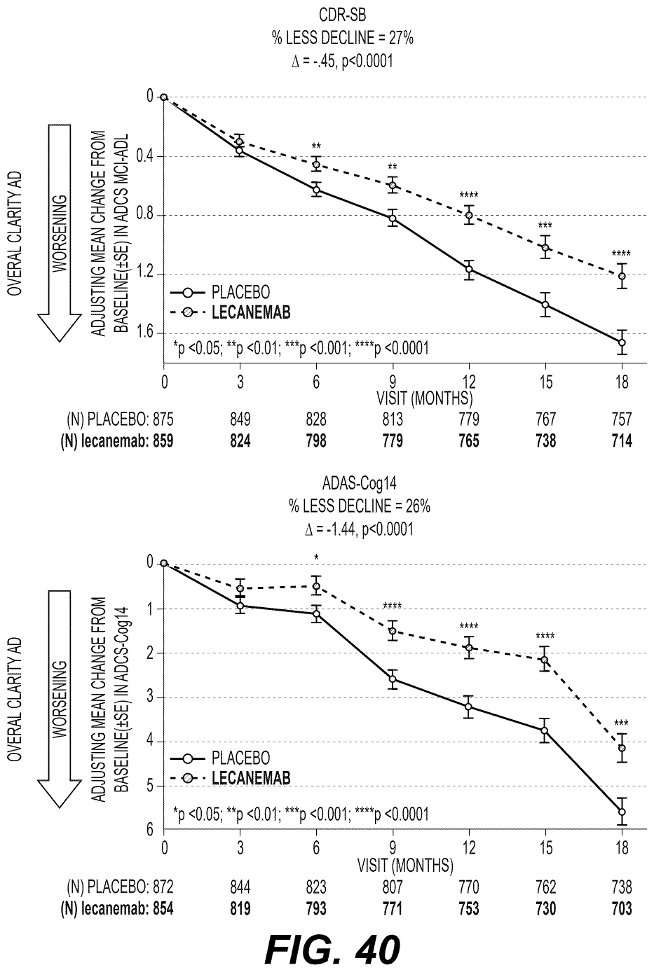

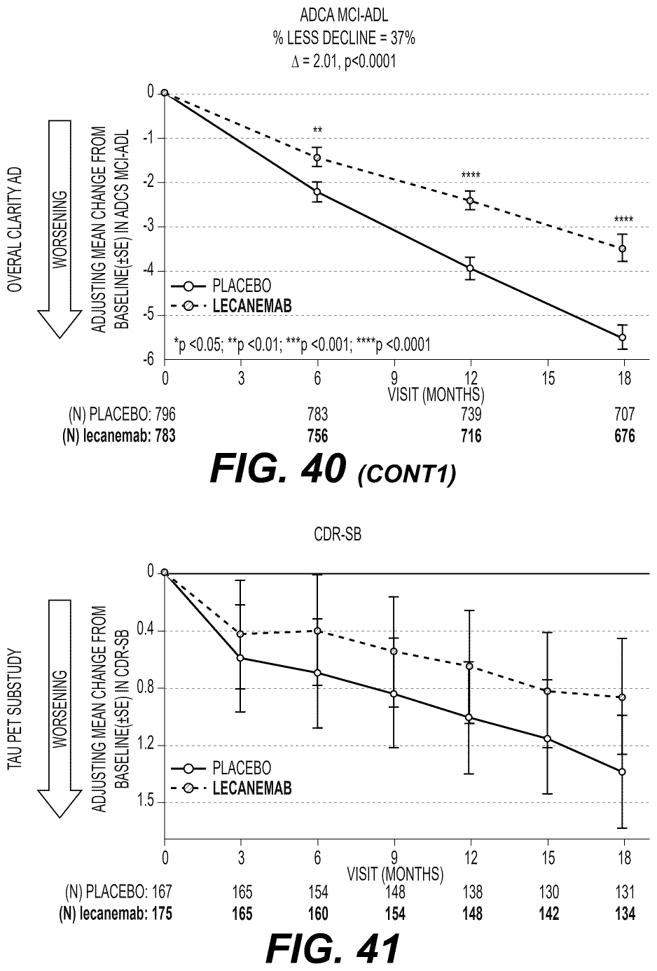

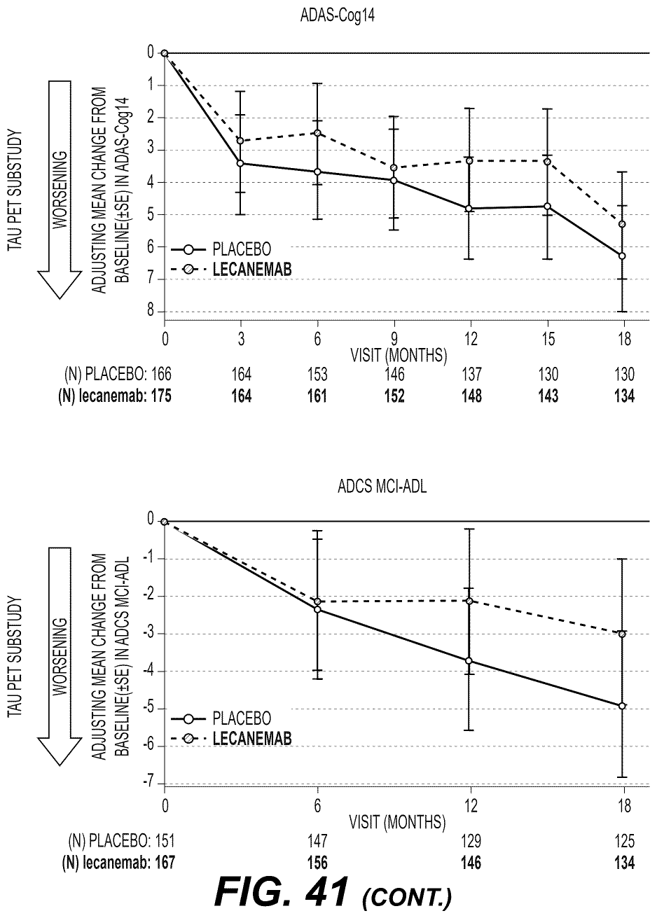

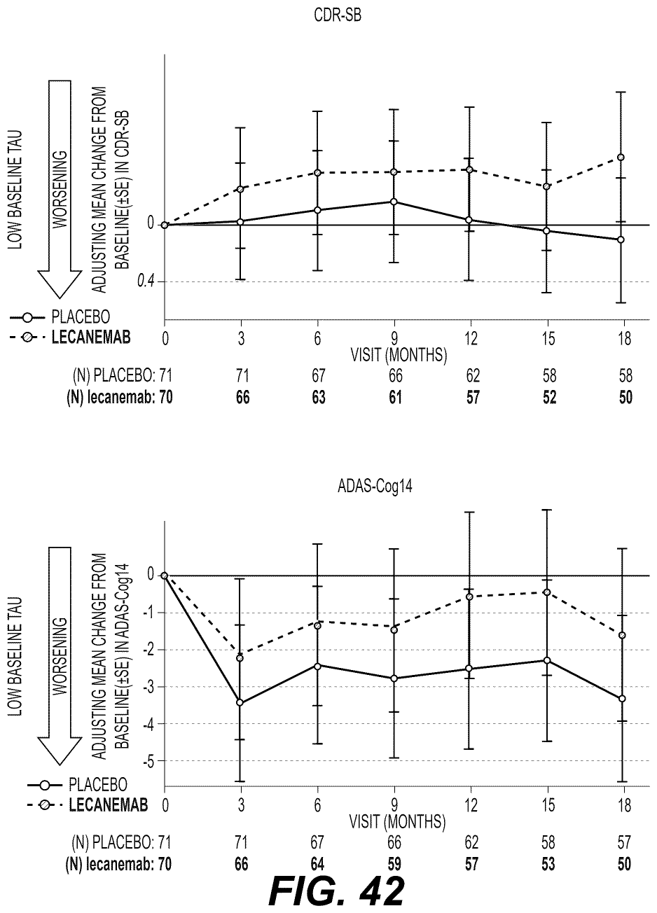

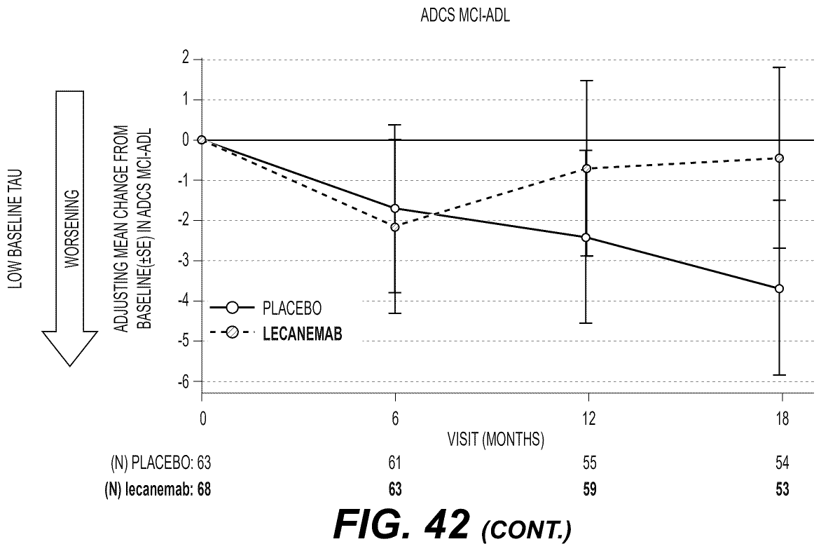

In measurements of clinical outcomes from patients in the tau PET substudy, those who received lecanemab showed a slower decline in CDR-SB, ADAS-Cog14, and ADCS MCI-ADL. Results were observed in all patients in the overall Clarity AD study (FIG. 40), and were consistent for patients in the tau PET substudy (FIG. 41) and from subjects with low baseline tau PET levels (FIG. 42).

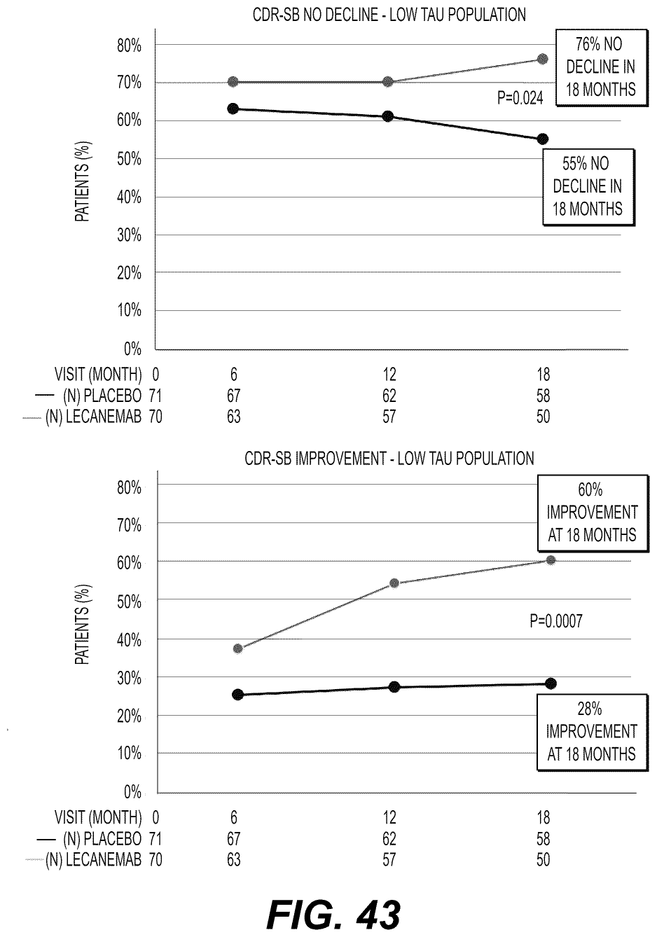

Results of cognitive tests in subjects from the low Tau PET group indicate that lecanemab stabilized or improved measures of cognitive function. Results of CDR-SB tests are shown in FIG. 43. CDR-SB was evaluated at 18 months in the low Tau PET group. 76% of subjects who received lecanemab showed no CDR-SB decline, which was significantly greater than placebo. 60% of subjects who received lecanemab showed CDR-SB improvement, which was significantly greater than placebo.

ADAS-Cog14 was evaluated at 18 months. 74% of subjects who received lecanemab showed no decline in ADAS-Cog14, as compared to 56% of subjects who received a placebo. 68% of subjects who received lecanemab showed an improvement, as compared to 32% of subjects who received who placebo.

ADCS MCI-ADL was evaluated at 18 months. 75% of subjects who received lecanemab showed no decline in ADCS MCI-ADL, as compared to 46% of subjects who received placebo. 70% of subjects who received lecanemab showed an improvement, as compared to 46% of subjects who received placebo. The analysis at 18 months was a post-hoc analysis with nominal p values and no adjustment for multiplicity.

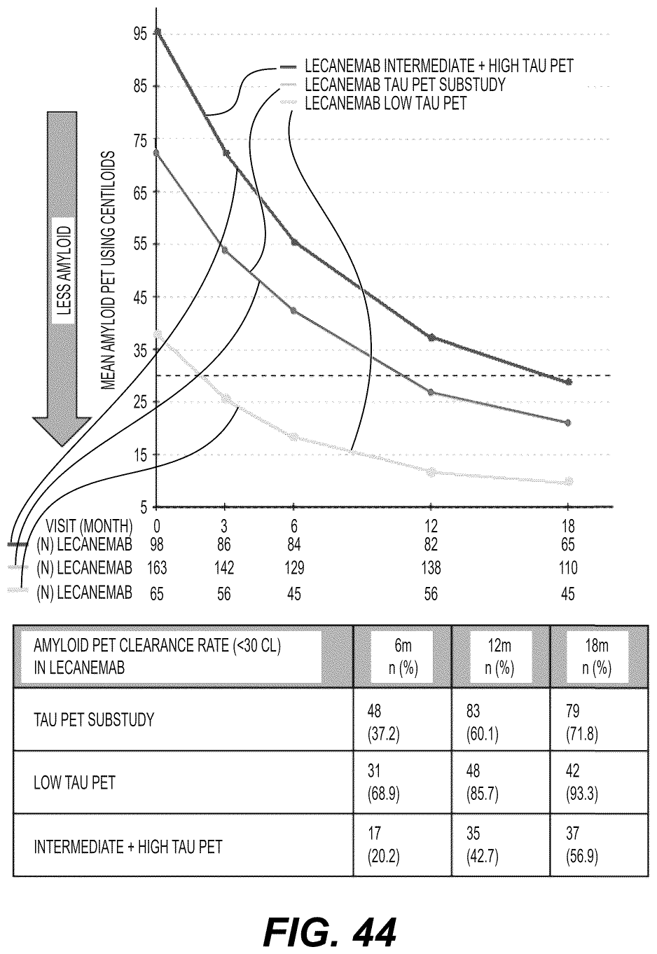

FIG. 44 shows amyloid PET levels and amyloid PET clearance rates in subjects from the Tau PET substudy. Data from low Tau PET subjects and intermediate+high tau PET subjects are shown. A consistent reduction in amyloid is observed in tau PET subgroups across clinical assessments.

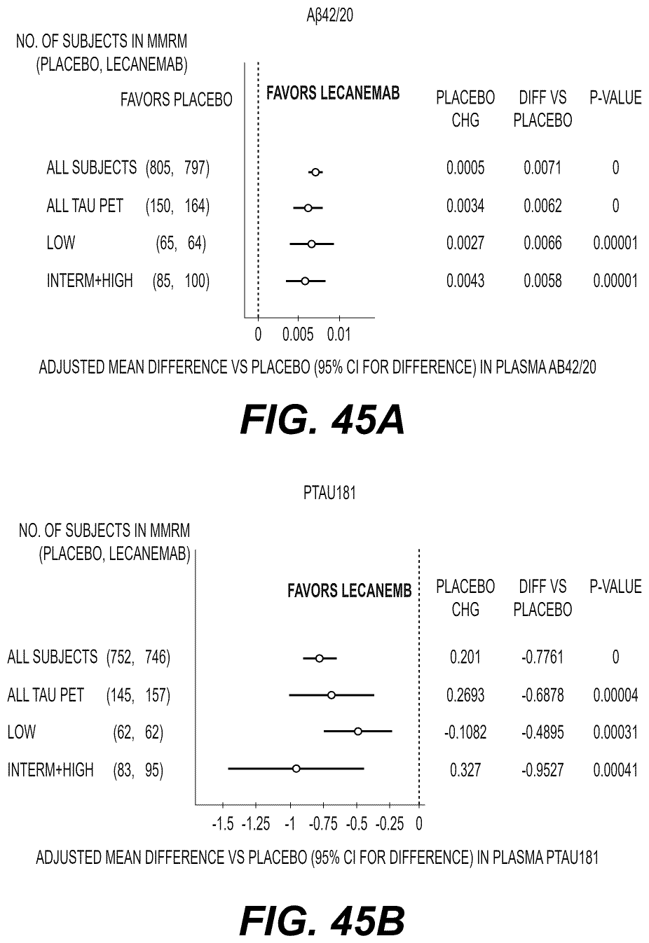

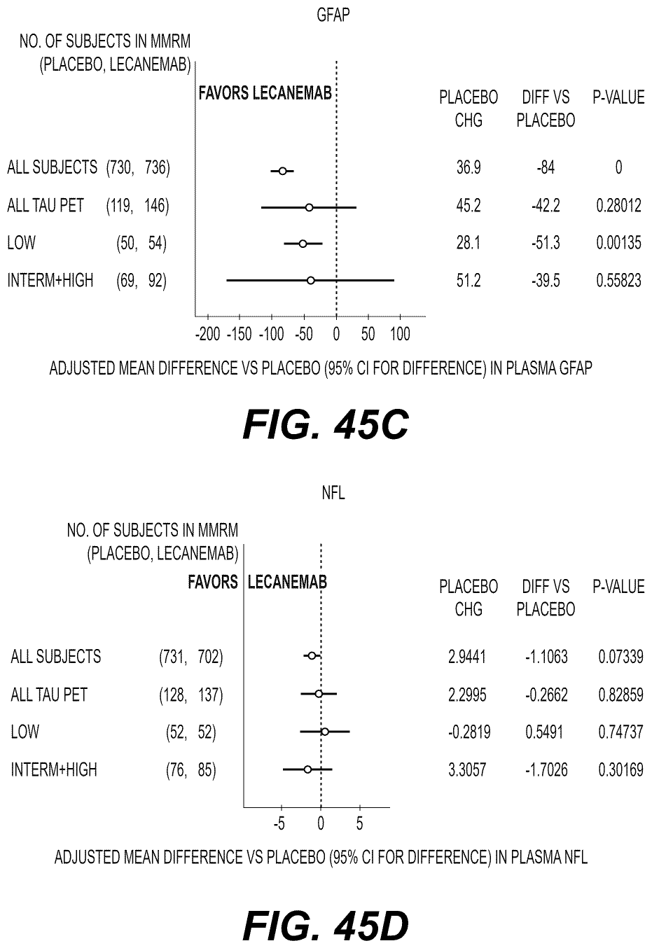

FIG. 45 shows fluid biomarkers in patients from the tau PET substudy: (A) Aβ42/40, (B) pTau181, (C) GFAP, and (D) NfL. Data is shown for patients after 18 months of treatment. The longitudinal tau PET substudy was predefined. The subgroups (low and intermediate+high tau PET) were a post-hoc analysis with nominal p values and no adjustment for multiplicity. The analysis is based on modified intention-to-treat analysis population. Adjusted mean change from baseline, SE and p-value are derived using mixed model repeat measures (MMRM) with treatment group, visit, treatment group by visit interaction, clinical subgroup, use of Alzheimer's disease symptomatic medication at baseline, ApoE4 carrier status, region, baseline value by visit interaction as fixed effects, and baseline value as covariates.

### Example 13. Extension of Efficacy Results from Clarity AD

### A. Open Label Extension—Methods

Clarity AD, the 18-month treatment (core study), multicenter, double-blind, placebo-controlled, parallel-group study in patients with early AD, includes an OLE phase in which eligible patients who completed 18 months of study drug treatment had the option of enrolling.

Assessments included the Clinical Dementia Rating-Sum-of-Boxes (CDR-SB), AD Assessment Scale-Cognitive Subscale 14 (ADAS-Cog14), Alzheimer's Disease Cooperative Study-Activities of Daily Living Scale for Mild Cognitive Impairment (ADCS-MCI-ADL) and amyloid PET. Long-term efficacy beyond 18 months of lecanemab 10 mg/kg biweekly was evaluated in the OLE utilizing the same efficacy assessments as the core study (6-month data reported). Clinical and biomarker (PET, Ab42/40 ratio, and ptau181) outcomes were evaluated by examining the OLE results for patients receiving placebo in the core study followed by lecanemab in the OLE. Analyses of participants from the optional tau PET sub-study, initiated during the core study, were conducted to evaluate the OLE treatment response segmented by core baseline tau PET levels.

### B. Results

In the core study, a total of 1795 participants were enrolled, 898 assigned to lecanemab and 897 to placebo. Of the 1486 subjects that completed 18 months of the double-blind core study, 1385 participants enrolled in the OLE. Differences between treatment and placebo across clinical endpoints increased over time during the first 18 months, but then became parallel after initiation of lecanemab in the core placebo group from 18 to 24 months, consistent with a disease modifying effect. Biomarker changes were seen in as early as 3 months in newly treated lecanemab participants in the OLE. Across efficacy assessments participants with low tau at core baseline, initially assigned to lecanemab, continued to show benefit of lecanemab treatment beyond 18 months, and participants initially assigned to the placebo arm began to demonstrate evidence of benefit subsequent to lecanemab being started in the OLE.

In initial data available from the Clarity AD OLE, treatment differences vs placebo observed in the core study were extended across clinical efficacy assessments at 24 months. These results are consistent with disease modification and support the initiation of treatment early in the disease.

FIGS. 46-53 show the results from early and late start subjects. The early start refers to subjects who received lecanemab (10 mg/kg biweekly) for 18 months during the trial (Core study), while the late (or “delayed”) start refers to subjects who received a placebo for 18 months during the trial and then started on lecanemab (10 mg/kg biweekly) during the Open Label Extension for 18-24 months. Analyses are based on modified intention-to-treat analysis population. Adjusted mean change from baseline, SE and p-value are derived using mixed model repeat measures (MMRM) with treatment group, visit, treatment group by visit interaction, clinical subgroup, use of Alzheimer's disease symptomatic medication at baseline, ApoE4 carrier status, region, baseline value by visit interaction as fixed effects, and baseline value as covariate.

FIG. 46 shows the results of CDR-SB measurements in subjects in the early start and late start groups. The separation between early and late start groups was maintained during the period of 18-24 months when all subjects were receiving lecanemab (p<0.05). The early and late start groups show a similar disease trajectory between 18-24 months.

FIG. 47 shows the test results from the OLE in the context of an observational cohort. The data indicate that delayed start subjects benefit from lecanemab treatment. The observational cohort comprises data from similar participants in the Alzheimer's Disease Neuroimaging Initiative (ADNI), who showed natural disease progression through 24 months. ADNI participants were selected to match with the Clarity AD population.

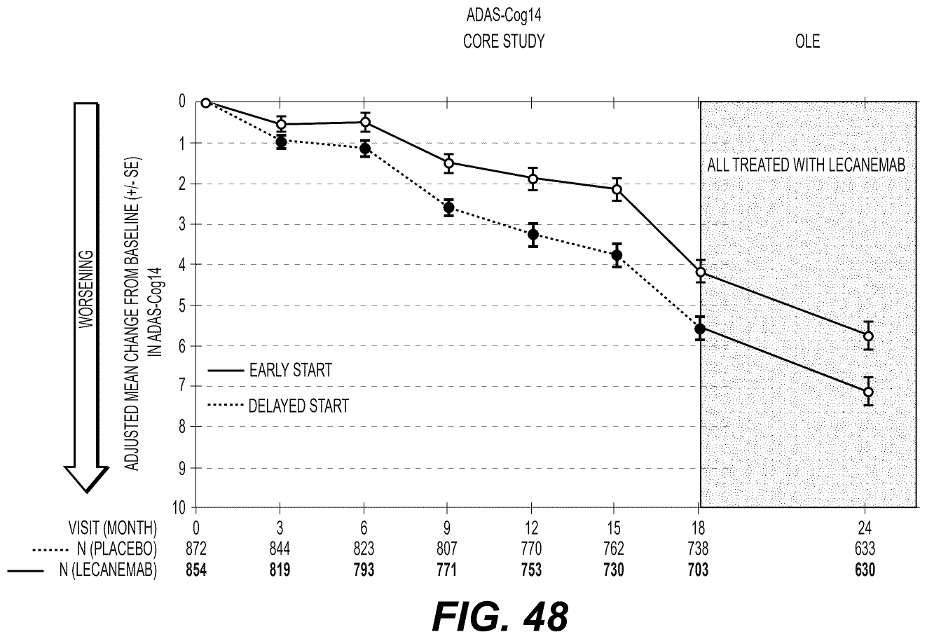

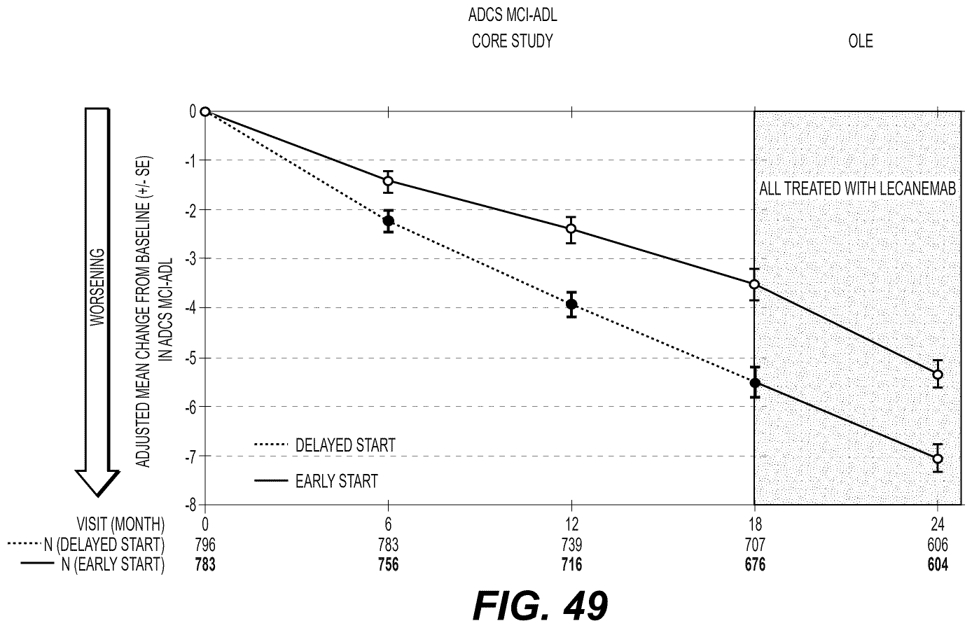

FIG. 48 and FIG. 49 shows the results of ADAS-Cog14 and ADCS MCI-ADL measurements in subjects in the early start and late start groups. In both tests, the separation between the early start and delay start subjects was maintained between 18 & 24 months when all subjects were on lecanemab (p<0.05). A similar disease trajectory was observed for the early start and delay start subjects between 18-24 months.

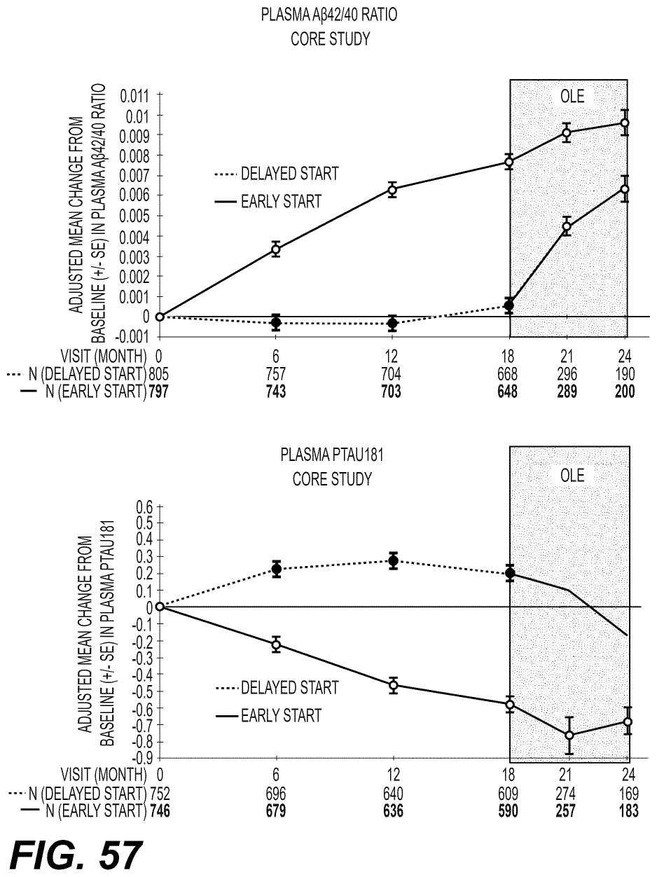

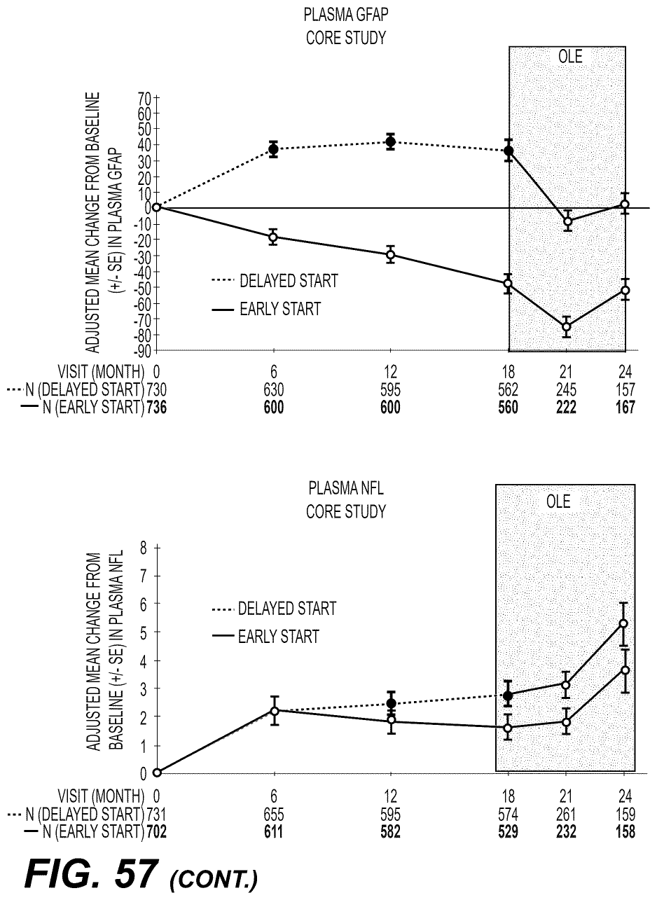

FIG. 57 shows results of biomarker analyses in patients receiving lecanemab for 24 months: (A) Aβ42/40, (B) pTau181, (C) GFAP, and (D) NfL. Subjects in the early start group who are treated continuously up to 24 months continue to benefit, while subjects in the late start group begin to benefit from treatment.

### C. Results—Low Tau PET Subgroup

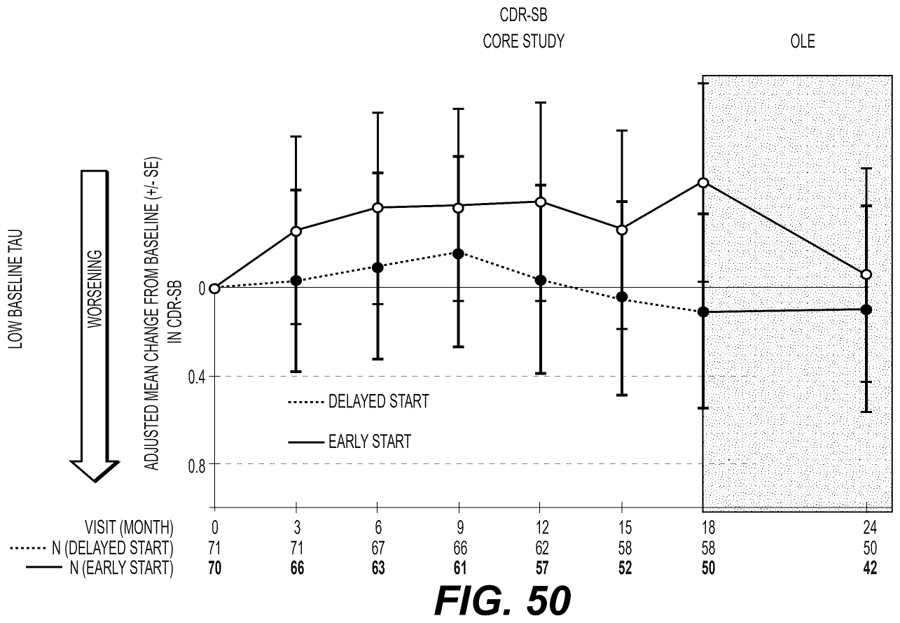

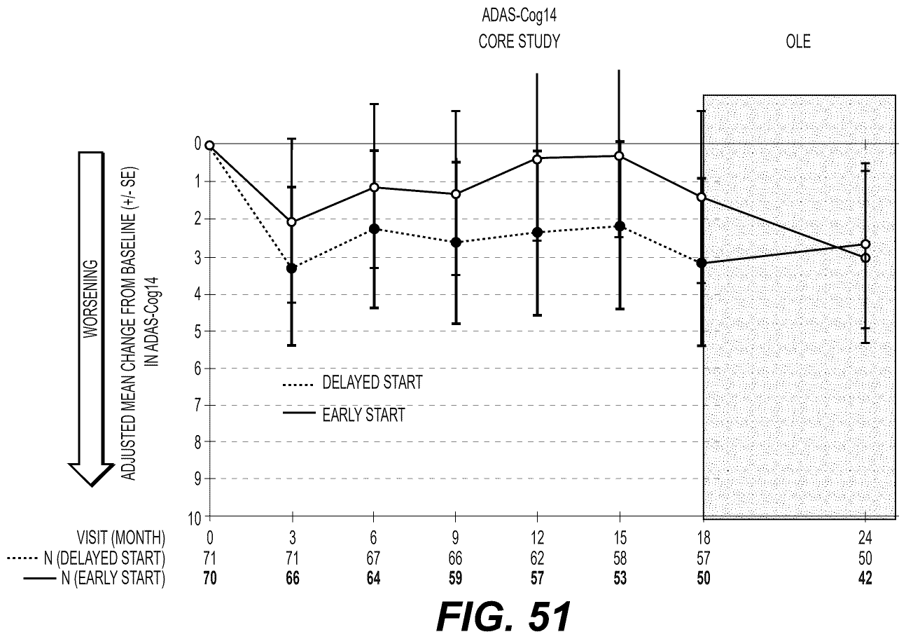

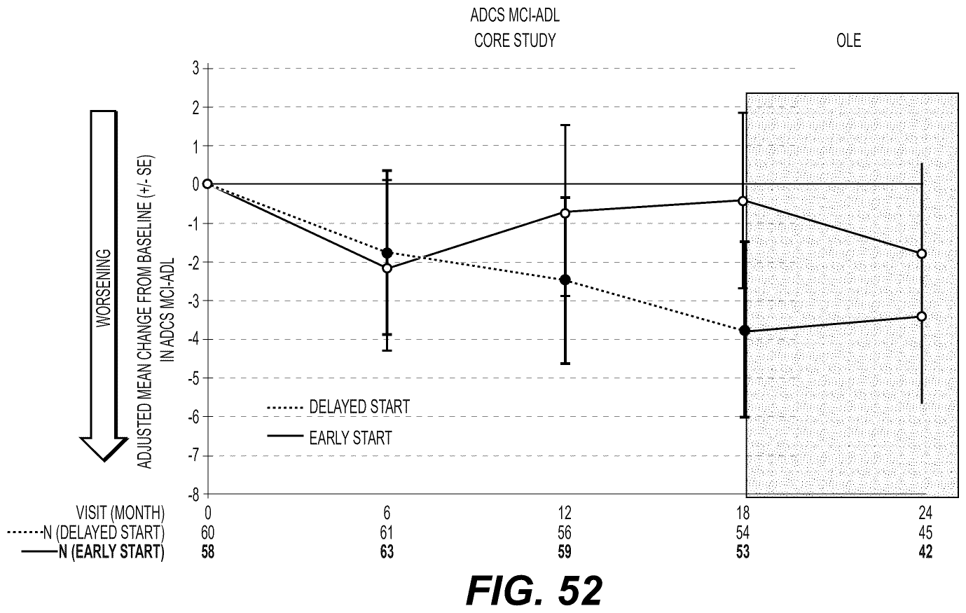

FIG. 50, FIG. 51, and FIG. 52 show the clinical outcomes in the low tau-PET subgroup through 24 months. The low tau PET subjects who received lecanemab showed sustained improvements through 24 months.

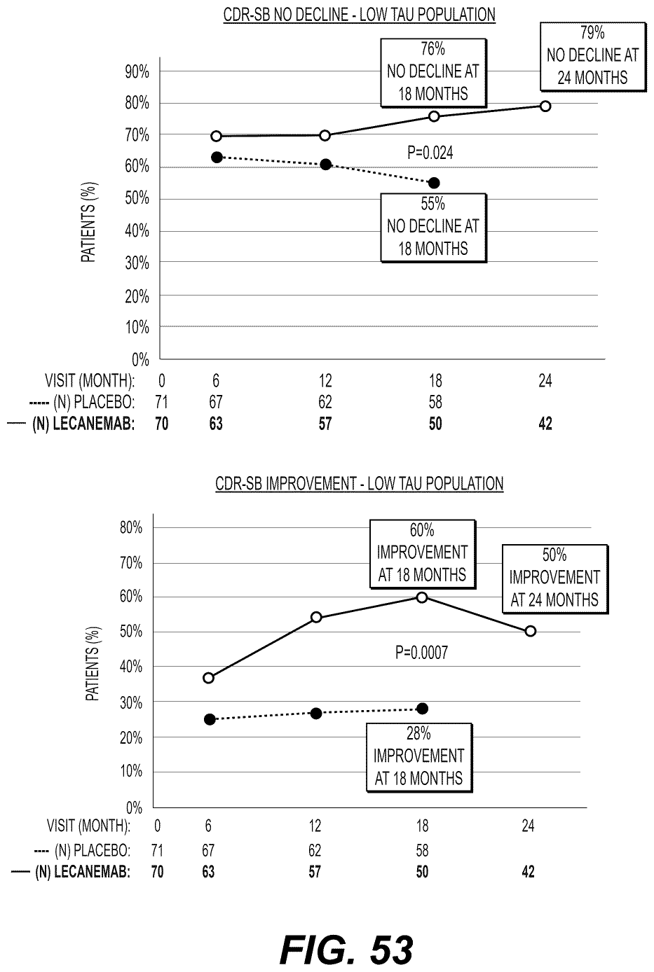

FIG. 53 is an extension of results from FIG. 41, showing the results of cognitive tests in subjects from the low tau PET group during the period of 18-24 months. Subjects in the low tau-PET group who received lecanemab through 24 months showed continued benefits in CDR-SB.

Results of CDR-SB tests are shown in FIG. 53. CDR-SB was evaluated at 24 months in the low tau PET group. 79% of subjects who received lecanemab showed no CDR-SB decline. 50% of subjects who received lecanemab showed CDR-SB improvement.

ADAS-Cog14 was evaluated at 24 months. 67% of subjects who received lecanemab showed no decline in ADAS-Cog14. 62% of subjects who received lecanemab showed an improvement.

ADCS MCI-ADL was evaluated at 24 months. 67% of subjects who received lecanemab showed no decline in ADCS MCI-ADL. 62% of subjects who received lecanemab showed an improvement.

The analysis at 18 months was a post-hoc analysis with nominal p values and no adjustment for multiplicity.

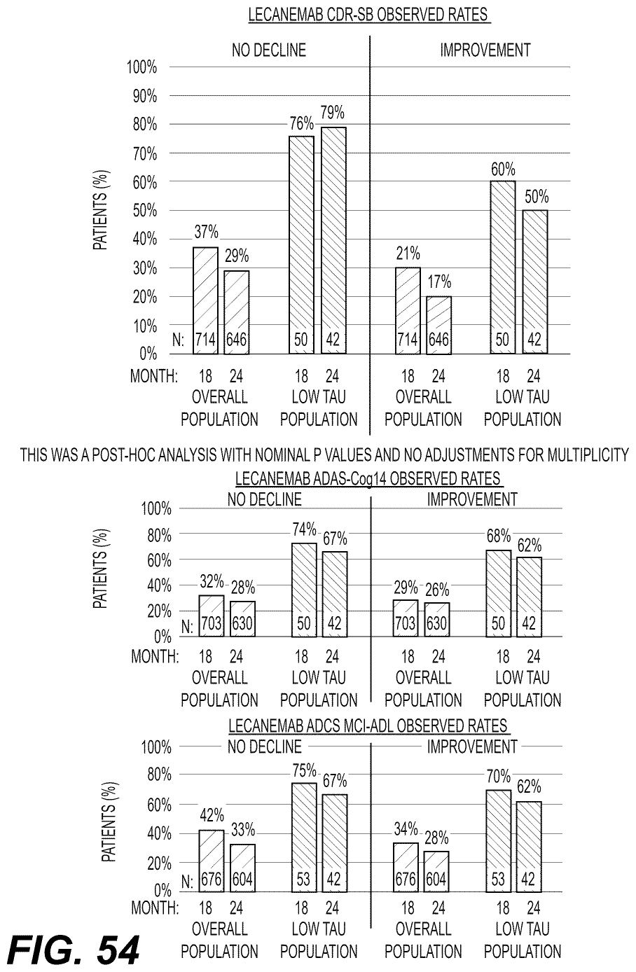

FIG. 54 shows the percentages of patients showing either “No Decline” or “Improvement” in CDR-SB, ADAS-Cog 14 and ADCS MCI-ADL analyses at up to 24 months on lecanemab. The low Tau population is compared to the overall population.

### D. Amyloid PET and Low Tau PET Levels

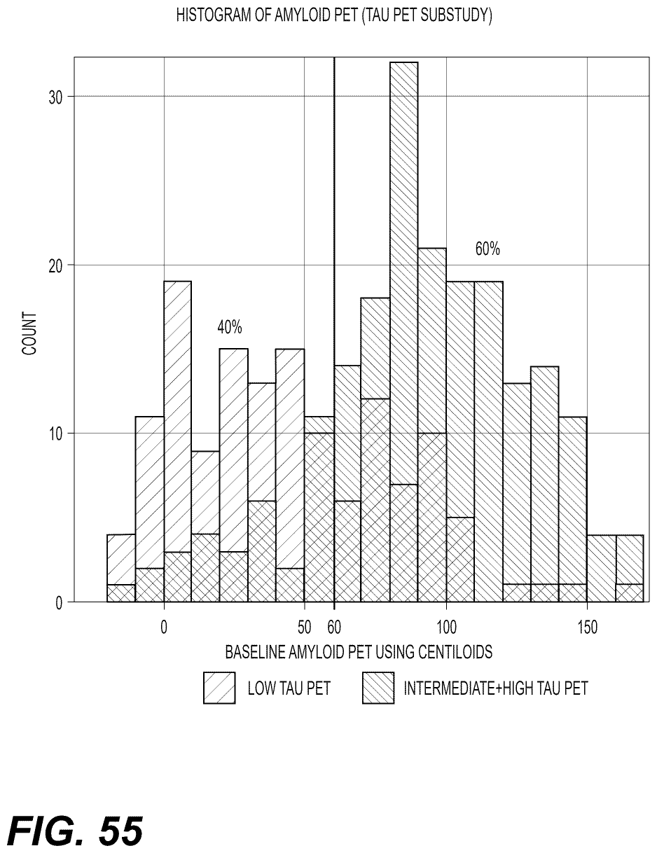

FIG. 55 shows that low tau PET levels are associated with lower levels of amyloid PET. Subjects in early stages of AD may be identified by low tau-PET levels or by an amyloid-PET level of <60 centiloids). The cutoff for low tau-PET (e.g., a SUVr <1.06 in whole cortical gray matter in MK6240 PET scans) corresponds to a baseline amyloid-PET of approximately 60CL. Accordingly, about 40% of the subjects in the low tau-PET subgroup have a baseline amyloid of less than 60 CL.

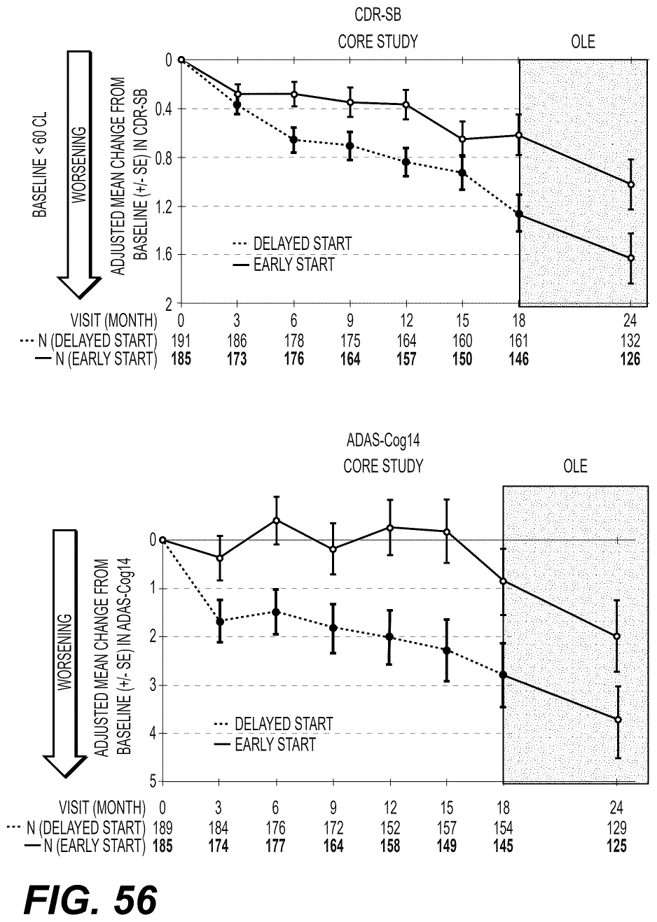

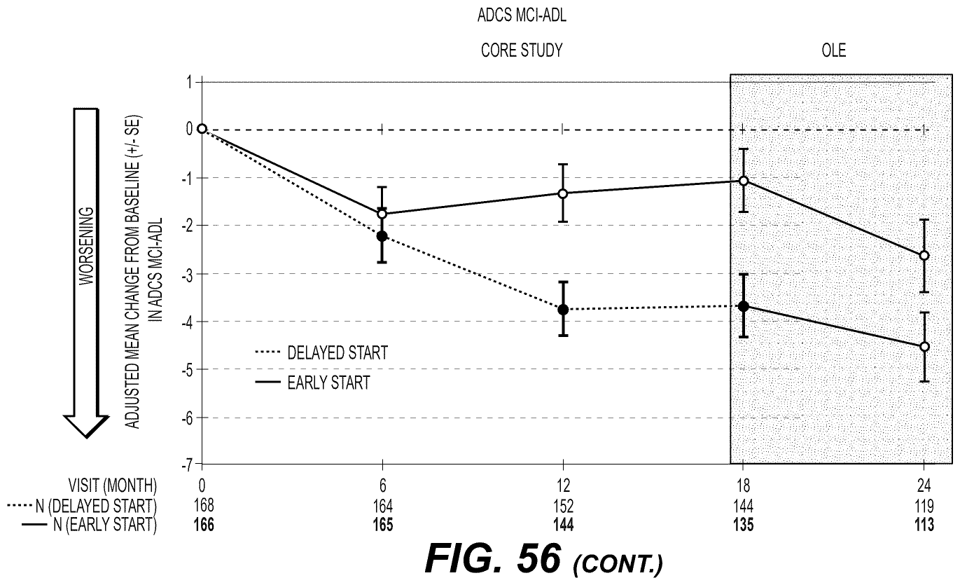

FIG. 56 shows clinical outcomes in subjects with a baseline amyloid PET of <60 CL. 60 CL is a cut-off that corresponds to low tau-PET. The subjects receiving lecanemab for 18-24 months showed continued benefits. Analysis is based on modified intention-to-treat analysis population. Adjusted mean change from baseline, SE and p-value are derived using mixed model repeat measures (MMRM) with treatment group, visit, treatment group by visit interaction, clinical subgroup, use of Alzheimer's disease symptomatic medication at baseline, ApoE4 carrier status, region, baseline value by visit interaction as fixed effects, and baseline value as covariate.

### Abbreviation Used in the Examples

Aβ amyloid beta
Aβ(1-42) amyloid beta monomer from amino acid 1 to 42
AD Alzheimer's disease
ADAS-Cog Alzheimer's Disease Assessment Scale-Cognitive Subscale
ADCOMS Alzheimer's Disease Composite Score
APOE Apolipoprotein E
APOE4 apolipoprotein e4 variant
ARIA amyloid-related imaging abnormalities
ARIA-E amyloid related imaging abnormality edema/effusion
ARIA-H amyloid related imaging abnormality hemorrhage
BAN2401 a humanized IgG1 monoclonal antibody
CDR Clinical Dementia Rating
CDR-SB Clinical Dementia Rating-Sum of Boxes
COVID-19 Coronavirus Disease 2019
CSF cerebrospinal fluid
EAD early Alzheimer's Disease
IV intravenous
LC-MS/MS liquid chromatography-tandem mass spectrometry
MCI mild cognitive impairment
MMSE Mini Mental State Examination
MRI magnetic resonance imaging
NIA-AA National Institute of Aging-Alzheimer's Association
OLE Open-label Extension
PET positron emission tomography
p-tau phospho-tau
SUVR standard uptake value ratio
t½ terminal elimination half-life
t-tau total tau
vMRI volumetric magnetic resonance imaging

1. A method of treating Alzheimer's disease (AD) in a subject having or suspected of having AD, comprising
c. selecting a subject having a low level of tau in a global brain measurement, preferably as measured by tau PET (a low tau PET level), and
d. administering to the subject a therapeutically effective dose of an anti-amyloid β (Aβ) protofibril antibody.

2. The method of claim 1, wherein treating AD comprises reducing, slowing, and/or reversing decline in a measure of cognitive function.

3. The method of claim 2, wherein the measure of cognitive function is CDR-SB, ADAS-Cog14, and/or ADCS MCI-ADL.

4. The method of claim 1, wherein treating AD comprises effecting a change (e.g., slowing, delaying, or reducing) in at least one marker of AD pathology.

5. The method of claim 4, wherein the marker is a plasma Aβ42/40 ratio, a plasma p-tau 181 level, a plasma GFAP level, and/or a plasma NfL level.

6. The method of claim 5, wherein the Aβ protofibril antibody increases the plasma Aβ42/40 ratio, as measured by an adjusted mean change from a baseline plasma Aβ42/40 ratio of at least about 0.003, 0.006, 0.007, 0.008, or 0.009.

7. The method of claim 5 or claim 6, wherein the Aβ protofibril antibody increases the Aβ42/40 ratio to about 0.092 or above.

8. The method of claim 5, wherein the Aβ protofibril antibody lowers plasma p-tau 181, as measured by an adjusted mean change from a baseline p-tau 181 level by at least about 0.2 pg/ml, 0.5 pg/ml, 0.6 pg/ml, 0.7 pg/ml, or 0.8 pg/ml.

9. The method of claim 5, wherein the Aβ protofibril antibody lowers plasma GFAP, as measured by an adjusted mean change from a baseline GFAP level by at least about 20 pg/ml, 30 pg/ml, 50 pg/ml, 60 pg/ml, or 80 pg/ml.

10. The method of claim 5, wherein the Aβ protofibril antibody increases plasma NfL by less than about 2 pg/ml, or less than about 3 pg/ml, as measured by an adjusted mean change from a baseline NfL level.

11. The method of claim 4, wherein the marker is a tau PET level or an amyloid PET level.

12. The method of claim 11, wherein the Aβ protofibril antibody reduces the tau PET level, as measured by an adjusted mean change from baseline tau PET SUVr level of less than about 0.1, e.g., 0.05.

13. The method of claim 11, wherein the Aβ protofibril antibody reduces the amyloid PET level to about 55, 40, 25, or 20 centiloids.

14. The method of claim 11, wherein the Aβ protofibril antibody reduces the tau PET and/or the amyloid PET level in a local brain region.

15. The method of claim 14, wherein the local brain region is an early Braak region (e.g., Braak regions I, II, or III).

16. The method of claim 15, wherein the early Braak region comprises entorhinal cortex, hippocampus, amygdala, parahippocampal gyrus, fusiform gyrus, and/or lingual gyrus.

17. The method of claim 14, wherein the local brain region is a composite of regions that accumulate tau in early AD.

18. The method of claim 17, wherein the composite region comprises a temporal region, a medial temporal region, and/or a meta-temporal region.

19. The method of claim 14, wherein the local brain region is the medial temporal region (e.g., the entorhinal cortex, hippocampus, parahippocampal gyrus and temporal pole (medial and/or inferio-lateral tip of the temporal lobe).

20. The method of claim 1, wherein the subject has mild cognitive impairment or mild dementia.

21. The method of claim 1, wherein the subject is at risk for developing AD.

22. The method of claim 1, wherein the subject has or is suspected of having, pre-AD.

23. The method of claim 1, wherein the subject has or is suspected of having, early AD.

24. The method of claim 1, wherein the subject has an amyloid PET level <20 CL, <40 CL, or <60 CL.

25. The method of claim 1, wherein the subject has an amyloid PET level >20 CL, >40 CL, or >60 CL).

26. The method of claim 1, wherein the subject has elevated amyloid measured by amyloid PET (e.g., is amyloid positive).

27. The method of claim 1, wherein the subject is an ApoE4 carrier.

28. The method of claim 1, wherein the low level of tau is a tau PET level (e.g., a tau-PET standardized uptake value ratio (SUVR)) below a threshold in the global brain measurement.

29. The method of claim 28, wherein the global brain measurement is a measurement of tau PET in the whole cortical gray matter.

30. The method of claim 28 or 29, wherein the tau PET level is measured using an MK6240 radiotracer.

31. The method of any one of claims 28-30, wherein the threshold tau PET level is a tau PET SUVR of about 1.1, preferably about 1.06, as measured with a MK6240 PET scan, e.g., of whole cortical gray matter.

32. The method of any one of claims 1-31, wherein the subject has a low level of tau in the global brain measurement (e.g., whole cortical gray matter) and a higher level of tau in a local brain region (e.g., medial temporal region, meta-temporal region, and/or temporal region).

33. The method of any one of claims 1-32, wherein the subject further exhibits tau in a local brain region, as measured by tau PET, e.g., an early Braak region (e.g., Braak regions I, II, or III).

34. A method of treating Alzheimer's disease (AD) in a subject having or suspected of having AD, comprising
c. selecting a subject having tau in a local brain region, preferably as measured by PET, and
d. administering to the subject a therapeutically effective dose of an anti-amyloid β (Aβ) protofibril antibody.

35. The method of claim 34, wherein the local brain region is an early Braak region (e.g., Braak regions I, II, or III).

36. The method of claim 35, wherein the early Braak region comprises transentorhinal cortex, entorhinal cortex, hippocampus, amygdala, parahippocampal gyrus, fusiform gyrus, and/or lingual gyrus.

37. The method of claim 34, wherein the local brain region is a composite of regions that accumulate tau in early AD.

38. The method of claim 37, wherein the composite region comprises a temporal region, a medial temporal region, and/or a meta-temporal region.

39. The method of claim 34, wherein the local brain region is the medial temporal region (e.g., the entorhinal cortex).

40. The method of any one of claims 1-39, wherein the anti-Aβ protofibril antibody comprises three heavy chain complementarity determining regions (HCDR1, HCDR2, and HCDR3) comprising amino acid sequences of SEQ ID NO: 1 (HCDR1), SEQ ID NO: 2 (HCDR2), and SEQ ID NO: 3 (HCDR3); and three light chain complementarity determining regions (LCDR1, LCDR2, and LCDR3) comprising amino acid sequences of SEQ ID NO: 4 (LCDR1), SEQ ID NO: 5 (LCDR2), and SEQ ID NO: 6 (LCDR3).

41. The method of any one of claims 1-40, wherein the anti-Aβ protofibril antibody comprises a heavy chain variable region comprising an amino acid sequence of SEQ ID NO: 7 and a light chain variable region comprising an amino acid sequence of SEQ ID NO: 8.

42. The method of any one of claims 1-41, wherein the anti-Aβ protofibril antibody comprises lecanemab.

43. The method of any one of claims 1-42, wherein the therapeutically effective dose of the anti-Aβ protofibril antibody comprises an intravenous infusion of 10 mg/kg relative to the weight of the subject.

44. The method of any one of claims 1-42, wherein the therapeutically effective dose of the anti-Aβ protofibril antibody comprises a subcutaneous administration of about 250 to 720 mg.

45. The method of any one of claims 1-43, wherein the therapeutically effective dose is administered weekly.

46. The method of any one of claims 1-43, wherein the therapeutically effective dose is administered every 2 weeks.

47. The method of any one of claims 1-46, wherein the therapeutically effective dose is administered for at least 13 months, at least 18 months, or at least 24 months.

48. The method of claim 47, wherein the frequency of administration is reduced after 13 months of treatment, e.g., to a frequency of every 4, 6, 8, 10, or 12 weeks.

49. The method of claim 47, wherein the frequency of administration is reduced after 18 months of treatment, e.g., to a frequency of every 4, 6, 8, 10, or 12 weeks.

50. The method of claim 47, wherein the frequency of administration is reduced after 24 months of treatment, e.g., to a frequency of every 4, 6, 8, 10, or 12 weeks.

51. The method of any one of claims 1-50, wherein the treatment further comprises administering at least one additional therapy for AD (e.g., an anti-tau antibody such as E2814).

52. The method of any one of claims 1-51, wherein the treatment further comprises administering an anti-tau antibody, preferably E2814.

53. A method of selecting a subject for treatment with an anti-amyloid 3 (Aβ) protofibril antibody, comprising:
a. obtaining a tau PET level from a global brain measurement of the subject;
b. selecting the subject for treatment if the tau PET level is less than a threshold level.

54. The method of claim 53, wherein the global brain measurement is a tau PET level in whole cortical gray matter.

55. The method of claim 53 or 54, wherein the tau PET level is measured using an MK6240 radiotracer.

56. The method of claim 54, wherein the threshold tau PET level is a tau PET SUVR of about 1.1, preferably about 1.06, as measured in a MK6240 PET scan, e.g., of whole cortical gray matter.
